SEQUENCE LISTING (113 sequences)

SEQ ID NO: 1 - protein, 9 aa, synthetic construct
  REGION 1..9: note = TIMP3 peptide functional variant(s)
SXGDWGAEX

SEQ ID NO: 2 - protein, 9 aa, Homo sapiens
SLGDWGAEA

SEQ ID NO: 3 - protein, 9 aa, synthetic construct
  REGION 1..9: note = TIMP3 peptide variant
SLGDWGAEV

SEQ ID NO: 4 - protein, 9 aa, synthetic construct
  REGION 1..9: note = TIMP3 peptide variant
SLGDWGAEI

SEQ ID NO: 5 - protein, 9 aa, synthetic construct
  REGION 1..9: note = TIMP3 peptide variant
SLGDWGAEL

SEQ ID NO: 6 - protein, 9 aa, synthetic construct
  REGION 1..9: note = TIMP3 peptide variant
SLGDWGAER

SEQ ID NO: 7 - protein, 9 aa, synthetic construct
  REGION 1..9: note = TIMP3 peptide variant
SLGDWGAEN

SEQ ID NO: 8 - protein, 9 aa, synthetic construct
  REGION 1..9: note = TIMP3 peptide variant
SLGDWGAED

SEQ ID NO: 9 - protein, 9 aa, synthetic construct
  REGION 1..9: note = TIMP3 peptide variant
SLGDWGAEC

SEQ ID NO: 10 - protein, 9 aa, synthetic construct
  REGION 1..9: note = TIMP3 peptide variant
SLGDWGAEE

SEQ ID NO: 11 - protein, 9 aa, synthetic construct
  REGION 1..9: note = TIMP3 peptide variant
SLGDWGAEQ

SEQ ID NO: 12 - protein, 9 aa, synthetic construct
  REGION 1..9: note = TIMP3 peptide variant
SLGDWGAEG

SEQ ID NO: 13 - protein, 9 aa, synthetic construct
  REGION 1..9: note = TIMP3 peptide variant
SLGDWGAEH

SEQ ID NO: 14 - protein, 9 aa, synthetic construct
  REGION 1..9: note = TIMP3 peptide variant
SLGDWGAEK

SEQ ID NO: 15 - protein, 9 aa, synthetic construct
  REGION 1..9: note = TIMP3 peptide variant
SLGDWGAEM

SEQ ID NO: 16 - protein, 9 aa, synthetic construct
  REGION 1..9: note = TIMP3 peptide variant
SLGDWGAEF

SEQ ID NO: 17 - protein, 9 aa, synthetic construct
  REGION 1..9: note = TIMP3 peptide variant
SLGDWGAEP

SEQ ID NO: 18 - protein, 9 aa, synthetic construct
  REGION 1..9: note = TIMP3 peptide variant
SLGDWGAES

SEQ ID NO: 19 - protein, 9 aa, synthetic construct
  REGION 1..9: note = TIMP3 peptide variant
SLGDWGAET

SEQ ID NO: 20 - protein, 9 aa, synthetic construct
  REGION 1..9: note = TIMP3 peptide variant
SLGDWGAEW

SEQ ID NO: 21 - protein, 9 aa, synthetic construct
  REGION 1..9: note = TIMP3 peptide variant
SLGDWGAEY

SEQ ID NO: 22 - protein, 23 aa, synthetic construct
  REGION 1..23: note = P124 long 1
MTPWLGLIVLLGSWSLGDWGAEA

SEQ ID NO: 23 - protein, 23 aa, synthetic construct
  REGION 1..23: note = P124 long 2
IVLLGSWSLGDWGAEACTCSPSH

SEQ ID NO: 24 - protein, 23 aa, synthetic construct
  REGION 1..23: note = P124 long 3
SLGDWGAEACTCSPSHPQDAFCN

SEQ ID NO: 25 - protein, 20 aa, synthetic construct
  REGION 1..20: note = P124 long V1-3
WLGLIVLLGSWSLGDWGAEV

SEQ ID NO: 26 - protein, 23 aa, synthetic construct
  REGION 1..23: note = P124 long V2
IVLLGSWSLGDWGAEVCTCSPSH

SEQ ID NO: 27 - protein, 23 aa, synthetic construct
  REGION 1..23: note = P124 long V3
SLGDWGAEVCTCSPSHPQDAFCN

SEQ ID NO: 28 - protein, 211 aa, Homo sapiens
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 29 - DNA, 20 bp, synthetic construct
  source 1..20: mol_type = other DNA
  misc_feature 1..20: note = sgRNA targeting exon 1 of the human TAP1 gene (sgTAP1)
gctgctacttctcgccgact

SEQ ID NO: 30 - DNA, 20 bp, synthetic construct
  source 1..20: mol_type = other DNA
  misc_feature 1..20: note = sgRNA targeting exon 1 of the TIMP3 gene (TIMP3 15-23)
gcacgatgagcccgagccaa

SEQ ID NO: 31 - DNA, 20 bp, synthetic construct
  source 1..20: mol_type = other DNA
  misc_feature 1..20: note = TIMP3 forward primer
caagatgccccatgtgcagt

SEQ ID NO: 32 - DNA, 20 bp, synthetic construct
  source 1..20: mol_type = other DNA
  misc_feature 1..20: note = TIMP3 reverse primer
ctctccacgaagttgcacag

SEQ ID NO: 33 - DNA, 20 bp, synthetic construct
  source 1..20: mol_type = other DNA
  misc_feature 1..20: note = TAP1 forward primer
cttgcagggagaggtgtttg

SEQ ID NO: 34 - DNA, 20 bp, synthetic construct
  source 1..20: mol_type = other DNA
  misc_feature 1..20: note = TAP1 reverse primer
gagcatgatccccaagagac

SEQ ID NO: 35 - DNA, 23 bp, synthetic construct
  source 1..23: mol_type = other DNA
  misc_feature 1..23: note = ARF5 forward primer
tgctgatgaactccagaagatgc

SEQ ID NO: 36 - DNA, 20 bp, synthetic construct
  source 1..20: mol_type = other DNA
  misc_feature 1..20: note = ARF5 reverse primer
cggctgcgtaagtgctgtag

SEQ ID NO: 37 - DNA, 23 bp, synthetic construct
  source 1..23: mol_type = other DNA
  misc_feature 1..23: note = CPSF6 forward primer
aagattgccttcatggaattgag

SEQ ID NO: 38 - DNA, 25 bp, synthetic construct
  source 1..25: mol_type = other DNA
  misc_feature 1..25: note = CPSF6 forward primer
tcgtgatctactatggtccctctct

SEQ ID NO: 39 - protein, 9 aa, Homo sapiens
FLGPWPAAS

SEQ ID NO: 40 - protein, 5 aa, Homo sapiens
VGISA

SEQ ID NO: 41 - protein, 5 aa, Homo sapiens
SGHVS

SEQ ID NO: 42 - protein, 10 aa, Homo sapiens
AVGGGADGLT

SEQ ID NO: 43 - protein, 5 aa, Homo sapiens
LSSGK

SEQ ID NO: 44 - protein, 6 aa, Homo sapiens
FQNEAQ

SEQ ID NO: 45 - protein, 12 aa, Homo sapiens
ASSLVGRGYEQF

SEQ ID NO: 46 - protein, 108 aa, Homo sapiens
KNEVEQSPQNLTAQEGEFITINCSYSVGISALHWLQQHPGGGIVSLFMLSSGKKKHGRLIATINIQEKHSSLHITASHPRDSAVYICAVGGGADGLTFGKGTHLIIQP

SEQ ID NO: 47 - DNA, 317 bp, Homo sapiens
  source 1..317: mol_type = genomic DNA
atttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgtcggaggaggtgctgacggactcacctttggcaaagggactcatctaatcatccagccctatatccagaaccctgaccctgccgtgtaccagctgagagact

SEQ ID NO: 48 - protein, 133 aa, Homo sapiens
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLVGRGYEQFFGPGTRLTVL

SEQ ID NO: 49 - DNA, 340 bp, Homo sapiens
  source 1..340: mol_type = genomic DNA
tagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctctgtgccagcagcttagtgggcaggggatacgagcagttcttcgggccagggacacggctcaccgtgctagaggacctgaaaaacgtgttcccacccgaggtcgctgtgtttgagccatcaga

SEQ ID NO: 50 - protein, 6 aa, Homo sapiens
YGATPY

SEQ ID NO: 51 - protein, 8 aa, Homo sapiens
YFSGDTLV

SEQ ID NO: 52 - protein, 10 aa, Homo sapiens
AVGPNNNDMR

SEQ ID NO: 53 - protein, 5 aa, Homo sapiens
SEHNR

SEQ ID NO: 54 - protein, 6 aa, Homo sapiens
FQNEAQ

SEQ ID NO: 55 - protein, 10 aa, Homo sapiens
ASSRGTDTQY

SEQ ID NO: 56 - protein, 112 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGPNNNDMRFGAGTRLTVKP

SEQ ID NO: 57 - DNA, 327 bp, Homo sapiens
  source 1..327: mol_type = genomic DNA
cactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtccgaataacaatgacatgcgctttggagcagggaccagactgacagtaaaaccaaatatccagaaccctgaccctgccgtgtaccagctgagagact

SEQ ID NO: 58 - protein, 131 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSRGTDTQYFGPGTRLTVL

SEQ ID NO: 59 - DNA, 334 bp, Homo sapiens
  source 1..334: mol_type = genomic DNA
taactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccgggggacagatacgcagtattttggcccaggcacccggctgacagtgctcgaggacctgaaaaacgtgttcccacccgaggtcgctgtgtttgagccatcaga

SEQ ID NO: 60 - protein, 5 aa, Homo sapiens
DSVNN

SEQ ID NO: 61 - protein, 5 aa, Homo sapiens
IPSGT

SEQ ID NO: 62 - protein, 8 aa, Homo sapiens
AVRDDKII

SEQ ID NO: 63 - protein, 5 aa, Homo sapiens
SEHNR

SEQ ID NO: 64 - protein, 6 aa, Homo sapiens
FQNEAQ

SEQ ID NO: 65 - protein, 9 aa, Homo sapiens
ASSFATEAF

SEQ ID NO: 66 - protein, 106 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVRDDKIIFGKGTRLHILP

SEQ ID NO: 67 - DNA, 307 bp, Homo sapiens
  source 1..307: mol_type = genomic DNA
tccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtgagagatgacaagatcatctttggaaaagggacacgacttcatattctccccaatatccagaaccctgaccctgccgtgtaccagctgagagact

SEQ ID NO: 68 - protein, 130 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSFATEAFFGQGTRLTVV

SEQ ID NO: 69 - DNA, 320 bp, Homo sapiens
  source 1..320: mol_type = genomic DNA
taactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagctttgcaactgaagctttctttggacaaggcaccagactcacagttgtagaggacctgaacaaggtgttcccacccgaggtcgctgtgttt

SEQ ID NO: 70 - protein, 7 aa, Homo sapiens
TSENNYY

SEQ ID NO: 71 - protein, 8 aa, Homo sapiens
QEAYKQQN

SEQ ID NO: 72 - protein, 13 aa, Homo sapiens
AFTINTGTASKLT

SEQ ID NO: 73 - protein, 5 aa, Homo sapiens
SGHTA

SEQ ID NO: 74 - protein, 6 aa, Homo sapiens
FQGNSA

SEQ ID NO: 75 - protein, 11 aa, Homo sapiens
ASRDYSYNEQF

SEQ ID NO: 76 - protein, 116 aa, Homo sapiens
AQTVTQSQPEMSVQEAETVTLSCTYDTSENNYYLFWYKQPPSRQMILVIRQEAYKQQNATENRFSVNFQKAAKSFSLKISDSQLGDTAMYFCAFTINTGTASKLTFGTGTRLQVTL

SEQ ID NO: 77 - DNA, 338 bp, Homo sapiens
  source 1..338: mol_type = genomic DNA
tgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcacaataaataccggcactgccagtaaactcacctttgggactggaacaagacttcaggtcacgctcgatatccagaaccctgaccctgccgtgtaccagctgagagact

SEQ ID NO: 78 - protein, 132 aa, Homo sapiens
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASRDYSYNEQFFGPGTRLTVL

SEQ ID NO: 79 - DNA, 325 bp, Homo sapiens
  source 1..325: mol_type = genomic DNA
agctctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccagcagagactactcctacaatgagcagttcttcgggccagggacacggctcaccgtgctagaggacctgaaaaacgtgttcccacccgaggtcgctgtgttt

SEQ ID NO: 80 - protein, 6 aa, Homo sapiens
YGATPY

SEQ ID NO: 81 - protein, 8 aa, Homo sapiens
YFSGDTLV

SEQ ID NO: 82 - protein, 10 aa, Homo sapiens
AVGPNNNDMR

SEQ ID NO: 83 - protein, 5 aa, Homo sapiens
SEHNR

SEQ ID NO: 84 - protein, 6 aa, Homo sapiens
FQNEAQ

SEQ ID NO: 85 - protein, 10 aa, Homo sapiens
ASSLGGNEQF

SEQ ID NO: 86 - protein, 112 aa, Homo sapiens; identical to SEQ ID NO: 56

SEQ ID NO: 87 - DNA, 326 bp, Homo sapiens
  source 1..326: mol_type = genomic DNA
actggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtggggccgaataacaatgacatgcgctttggagcagggaccagactgacagtaaaaccaaatatccagaaccctgaccctgccgtgtaccagctgagagact

SEQ ID NO: 88 - protein, 131 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLGGNEQFFGPGTRLTVL

SEQ ID NO: 89 - DNA, 323 bp, Homo sapiens
  source 1..323: mol_type = genomic DNA
taactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttaggggggaatgagcagttcttcgggccagggacacggctcaccgtgctagaggacctgaaaaacgtgttcccacccgaggtcgctgtgttt

SEQ ID NO: 90 - protein, 6 aa, Homo sapiens
ATGYPS

SEQ ID NO: 91 - protein, 7 aa, Homo sapiens
ATKADDK

SEQ ID NO: 92 - protein, 11 aa, Homo sapiens
ALLPSNTGKLI

SEQ ID NO: 93 - protein, 5 aa, Homo sapiens
SEHNR

SEQ ID NO: 94 - protein, 6 aa, Homo sapiens
FQNEAQ

SEQ ID NO: 95 - protein, 11 aa, Homo sapiens
ASSSPTVYEQY

SEQ ID NO: 96 - protein, 112 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALLPSNTGKLIFGQGTTLQVKP

SEQ ID NO: 97 - DNA, 326 bp, Homo sapiens
  source 1..326: mol_type = genomic DNA
cctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgctgcctagcaacacaggcaaactaatctttgggcaagggacaactttacaagtaaaaccagatatccagaaccctgaccctgccgtgtaccagctgagagact

SEQ ID NO: 98 - protein, 132 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSPTVYEQYFGPGTRLTVT

SEQ ID NO: 99 - DNA, 327 bp, Homo sapiens
  source 1..327: mol_type = genomic DNA
gtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagctcacccaccgtctacgagcagtacttcgggccgggcaccaggctcacggtcacagaggacctgaaaaacgtgttcccacccgaggtcgctgtgttt

SEQ ID NO: 100 - protein, 6 aa, Homo sapiens
YGATPY

SEQ ID NO: 101 - protein, 8 aa, Homo sapiens
YFSGDTLV

SEQ ID NO: 102 - protein, 10 aa, Homo sapiens
AVGPNNNDMR

SEQ ID NO: 103 - protein, 5 aa, Homo sapiens
SEHNR

SEQ ID NO: 104 - protein, 6 aa, Homo sapiens
FQNEAQ

SEQ ID NO: 105 - protein, 10 aa, Homo sapiens
ASSLATDTQY

SEQ ID NO: 106 - protein, 112 aa, Homo sapiens; identical to SEQ ID NO: 56

SEQ ID NO: 107 - DNA, 326 bp, Homo sapiens
  source 1..326: mol_type = genomic DNA
actggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggccccaataacaatgacatgcgctttggagcagggaccagactgacagtaaaaccaaatatccagaaccctgaccctgccgtgtaccagctgagagact

SEQ ID NO: 108 - protein, 131 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLATDTQYFGPGTRLTVL

SEQ ID NO: 109 - DNA, 323 bp, Homo sapiens
  source 1..323: mol_type = genomic DNA
taactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagcgacagatacgcagtattttggcccaggcacccggctgacagtgctcgaggacctgaaaaacgtgttcccacccgaggtcgctgtgttt

SEQ ID NO: 110 - protein, 19 aa, synthetic construct
  REGION 1..19: note = P124 long V1-4
LGLIVLLGSWSLGDWGAEV

SEQ ID NO: 111 - protein, 17 aa, synthetic construct
  REGION 1..17: note = P124 long V1-6
LIVLLGSWSLGDWGAEV

SEQ ID NO: 112 - protein, 8 aa, Homo sapiens
SIINFEKL

SEQ ID NO: 113 - protein, 9 aa, Human papillomavirus
RAHYNIVTF

(19) United States
(12) Patent Application Publication
(10) Pub. No.: US 2026/0306599 A1
(43) Pub. Date: Oct. 8, 2026

(54) TIMP3-DERIVED TEIPP NEOANTIGENS AND USES THEREOF

(71) Applicant: ACADEMISCH ZIEKENHUIS LEIDEN (H.O.D.N. LUMC), Leiden (NL)

(72) Inventors: Sjoerd Henricus VAN DER BURG, Leiden (NL); Thorbald VAN HALL, Leiden (NL); Koen Abraham MARIJT, Leiden (NL)

(21) Appl. No.: 19/477,043

(22) PCT Filed: Apr. 22, 2024
(86) PCT No.: PCT/NL2024/050207

§ 371 Date: Oct. 20, 2025

(30) Foreign Application Priority Data
Apr. 21, 2023 (NL) 2034658

(51) Int. Cl. A61K 39/00; A61P 37/04; C07K 14/705

(52) U.S. Cl. CPC A61K 39/0011; A61P 37/04; C07K 14/7051

(57) ABSTRACT

Novel nucleic acid sequences, vectors, modified cells, binding agents, peptides and pharmaceutical compositions are provided herein that are useful as a medicament, for example in the prevention or treatment of cancer or viral infections associated with impaired HLA class I antigen presentation. Corresponding methods and uses are also provided herein.

Novel nucleic acid sequences, vectors, modified cells, binding agents, peptides and pharmaceutical compositions are provided that are useful as a medicament, for example in the prevention or treatment of cancer or viral infections associated with impaired HLA class I antigen presentation. Corresponding methods and uses are also provided.

### BACKGROUND

Many T-cell based immunotherapies used for treatment of cancer in humans are based on recognition of tumor antigens presented in HLA class I (HLA-1) molecules by tumor cells (Robbins et al., 2013; Schumacher et al., 2015). Point mutated peptides constitute formidable tumor antigens due to their non-self nature for which a non-curtailed T cell repertoire is available. An absolute requirement for such T cells to exert their action against cancer is the display of HLA-I at the surface of tumor cells. However, HLA-I down modulation on cancer cells is observed in many immune-escaped cancers, often caused by epigenetic silencing of antigen processing components, like the peptide transporter TAP (Ritter et al., 2017; Setiadi et al., 2007; Garrido et al., 2016).

A novel category of tumor antigens, referred to as TEIPP (T cell epitopes associated with impaired peptide processing), are presented at the surface of tumor cells carrying defects in antigen processing (Van Hall et al., 2006; Seidel et al., 2012). TEIPPs are derived from ubiquitously expressed non-mutated ‘self’ proteins, however, their processed peptides fail to be loaded up to T-cell detectable levels into HLA-I in healthy cells. Their surface presentation is highly promoted by defects in the antigen processing machinery, especially in the absence of the peptide transporter TAP. TAP-impairment is frequently found in cancer cells and thus TEIPP peptides constitute tumor-specific antigens. In mouse tumor models in which MHC-I presentation is down modulated by defects in the peptide transporter TAP, selective presentation of TEIPP peptides and successful targeting of immune-escaped tumor variants by TEIPP specific T cells has been shown either by vaccination or by transfer of specific T cells (Doorduijn et al., 2016; Doorduijn et al., 2018). Thus, targeting TEIPP neoantigens is a potent strategy to induce anti-tumor responses for tumors with lowered TAP expression and/or impaired HLA I antigen presentation.

Impairment of HLA-1-mediated antigen presentation has been observed upon infection with certain viruses. Such an impairment and/or lowered TAP expression, may then result in surface presentation of TEIPP antigens on virally infected cells. Accordingly, TEIPP peptides may also be considered as target antigens for treating or preventing a viral infection associated with lowered TAP expression and/or impaired antigen presentation in HLA class I molecules.

US2009/0220534 and Weinzieri (Weinzierl et al., 2008) describe screening methods for identifying T cell epitopes that are presented on the surface of cells via a TAP independent mechanism.

Human TEIPP neoantigens as potential targets for T-cell based immunotherapies have been described previously in WO2019231326A1 and WO2021107775A1. However, further, potentially improved, TEIPP neoantigens for preventing and/or treating cancer are needed.

### BRIEF SUMMARY OF THE DISCLOSURE

The inventors have previously developed a hybrid forward-reverse immunologic screen to identify novel TAP-independent non-mutated neoantigens that are selectively presented by immune-escaped cancers (Marijt et al., 2018). Their approach encompassed an in silico prediction of TEIPP neoantigen-candidates from the whole human proteome, matching of candidates to the cancer-specific peptidome, and an ex vivo screen to confirm the presence of a TEIPP T cell repertoire in healthy donors.

The inventors have now identified new TEIPP neoantigen candidates. 36 peptides were selected for their capacity to activate CD8+ T cells present in peripheral blood mononuclear cells (PBMC) of healthy human donors. Following further ex vivo screening using healthy donors, an HLA-A*02:01-specific peptide was identified that showed a particularly strong immunogenicity and a general availability of T cells to this peptide in the human T-cell repertoire. This peptide was identified to be derived from TIMP Metallopeptidase Inhibitor 3 (TIMP3) and as having the sequence SLGDWGAEA (SEQ ID NO: 2).

Advantageously, the CD8+ T cells identified in the repertoire of healthy donors were predominantly in the naïve state. As disclosed herein, the CD8+ T cells did not recognize the peptides presented herein in cells with normal TAP expression, but did so when confronted with cells having impaired TAP expression. The peptides presented herein are therefore ideal candidates for inducing a T-cell based immune response in vivo against cells associated with impaired antigen presentation in HLA class I presentation molecules and/or impaired TAP function. The data presented herein therefore provides evidence that these peptides could be used as a novel therapy to induce a tumor-specific T cell based immune response in vivo (by activating the naïve cognate T cells present within the natural T cell repertoire of the patient). In addition, binding agents such as antibodies, TCRs or CARs (or modified cells expressing the same) that specifically bind to these peptides may advantageously be used as a novel immunotherapy for the prevention or treatment of cancer or viral infections associated with impaired antigen presentation in HLA class I molecules and/or impaired TAP function.

Advantageously, the peptides described herein are not derived from the mutanome of cancers, but are of ‘self’ origin and therefore constitute universal neoantigens that may be presented on the cell surface of any cell with impaired antigen presentation in HLA class I molecules.

Furthermore, the inventors have previously developed a synthetic long peptide (SLP) vaccination platform and have shown that peptides of between 10-35 amino acids (preferably between 17 and 35 amino acids) possess the capacity to trigger CD4 and CD8 T cell responses and result in eradication of premalignant lesions (Kenter et al., 2009; Toes et al., 1996; Bijker et al., 2007) as well as improve overall survival of patients with cancer when vaccinated during chemotherapy. Cross-presentation of such peptides by host dendritic cells involves multiple sequential steps, including uptake via endocytosis, cytosolic cleavage of the SLP into short peptides by the proteasome, the dominant proteolytic enzyme, transport over the ER membrane by TAP and loading onto MHC-I molecules (Rosalia et al., 2013).

The data presented herein shows that dendritic cells are able to cross-present a long version of the HLA-A*02:01 presented TIMP3-derived identified peptide SLGDVGAEA when elongated with its natural flanking sequences. A single specific amino acid change in the sequence of the SLGDVWGAEA peptide (to SLGDWGAEV; SEQ ID NO: 3) results in a higher predicted binding affinity of the peptide to HLA-A*02:01. Cross-presentation of the TIMP3-derived V-variant SLGDWGAEV peptide was less optimal when the epitope was placed at the C-terminal part of a longer peptide, indicated by a less consistent presentation to SLGDWGAEA-specific T cells by monocyte-derived dendritic cells. However, a further shortening of the N-terminal part of the longer peptide surprisingly resulted in enhanced T cell stimulation and more efficient cross-presentation by monocyte-derived dendritic cells. Surprisingly, this specific amino acid change combined with alterations in the flanking sequence allows the peptide to be used as a more effective vaccine.

The data presented herein shows that small alterations to signal peptide-epitopes retain immunogenicity of TEIPP antigens and render them suitable candidates for the SLP vaccine format. Such vaccines may represent a salvage therapy for immune-escaped cancer by activating TIMP3-specific T cells.

The invention therefore provides functional variants of SLGDWGAEA (SEQ ID NO: 2) for use as a medicament. In this context, a functional variant refers to a variant of SEQ ID NO:2 that still elicits the desired immune response (i.e. a T cell response that is able to recognize and bind to the natural TEIPP of SEQ ID NO:2). The second amino acid, L, in this peptide functions as the main anchor of the peptide in the HLA groove and therefore replacement of this amino acid with alternative amino acids is possible without adversely affecting TCR binding to the peptide:HLA complex. Accordingly, in the context of the invention, this amino acid may be varied when providing a peptide for a peptide vaccine, as the variant peptide should still elicit the desired immune response (that will be able to recognise and bind to the natural TEIPP, SLGDWGAEA). This also applies to the C-terminal amino acid (A), which, as shown herein, may also be varied without adversely affecting TCR specificity. In view of this, the invention relates to SLGDWGAEA (SEQ ID NO: 2) and functional variants thereof, which are represented by SEQ ID NO: 1 (SX₁GDVGAEX₂, wherein X₁ and X₂ are any amino acid (i.e. A or R or N or D or C or Q or E or G or H or I or L or K or M or F or P or O or S or U or T or W or Y or V). Reference to “SEQ ID NO: 1” herein therefore encompasses the amino acid sequences of SEQ ID NO: 2-21.

A peptide is therefore provided comprising the amino acid sequence of SEQ ID NO: 1. Suitably, the peptide may consist of the amino acid sequence of SEQ ID NO: 1.

Also provided herein are nucleic acid sequences encoding the peptides of the present invention.

A nucleic acid sequence is therefore provided encoding the amino acid sequence of SEQ ID NO: 1.

A vaccine is also provided comprising:
(a) a peptide comprising or consisting of the amino acid sequence of SEQ ID NO: 1, or a nucleic acid encoding the peptide; and
(b) a pharmaceutically acceptable excipient, adjuvant, diluent and/or carrier.

The vaccine encodes or comprises an immunogenic peptide. An immunogenic peptide is a peptide that is capable of eliciting an immune response, wherein the immune response is directed against the peptide or an epitope presented by the peptide, optionally with the help of a suitable vaccine adjuvant.

A vaccine as described herein is an example of a pharmaceutical composition described herein. The term “pharmaceutical composition” herein therefore encompasses the vaccines described herein.

In one embodiment, the peptide comprises the amino acid sequence SLGDVNGAEA (SEQ ID NO: 2). Suitably, the peptide may consist of the amino acid sequence SLGDVNGAEA (SEQ ID NO: 2). Suitably, the peptide may comprise or consist of the amino acid sequence of any one of SEQ ID NO: 22, 23 or 24.

In one embodiment, the peptide comprises the amino acid sequence SLGDVNGAEV (SEQ ID NO: 3). Suitably, the peptide may consist of the amino acid sequence SLGDVNGAEV (SEQ ID NO: 3). Suitably, the peptide may comprise or consist of the amino acid sequence of any one of SEQ ID NO: 25, 26, 27, 110, or 111.

In one embodiment, the peptide comprises the amino acid sequence SLGDVNGAEI (SEQ ID NO: 4). Suitably, the peptide may consist of the amino acid sequence SLGDVNGAEI (SEQ ID NO: 4).

In one embodiment, the peptide comprises the amino acid sequence SLGDVNGAEL (SEQ ID NO: 5). Suitably, the peptide may consist of the amino acid sequence SLGDVNGAEL (SEQ ID NO: 5).

In one embodiment, the peptide comprises the amino acid sequence of any one of SEQ ID NOs: 6-21. Suitably, the peptide may consist of the amino acid sequence of any one of SEQ ID NOs: 6-21.

In one embodiment, the peptide comprises the amino acid sequence of SEQ ID NO: 22. Suitably, the peptide may consist of the amino acid sequence of SEQ ID NO: 22.

In one embodiment, the peptide comprises the amino acid sequence of SEQ ID NO: 23. Suitably, the peptide may consist of the amino acid sequence of SEQ ID NO: 23.

In one embodiment, the peptide comprises the amino acid sequence of SEQ ID NO: 24. Suitably, the peptide may consist of the amino acid sequence of SEQ ID NO: 24.

In one embodiment, the peptide comprises the amino acid sequence of SEQ ID NO: 25. Suitably, the peptide may consist of the amino acid sequence of SEQ ID NO: 25.

In one embodiment, the peptide comprises the amino acid sequence of SEQ ID NO: 26. Suitably, the peptide may consist of the amino acid sequence of SEQ ID NO: 26.

In one embodiment, the peptide comprises the amino acid sequence of SEQ ID NO: 27. Suitably, the peptide may consist of the amino acid sequence of SEQ ID NO: 27.

In one embodiment, the peptide comprises the amino acid sequence of SEQ ID NO: 110. Suitably, the peptide may consist of the amino acid sequence of SEQ ID NO: 110.

In one embodiment, the peptide comprises the amino acid sequence of SEQ ID NO: 111. Suitably, the peptide may consist of the amino acid sequence of SEQ ID NO: 111.

Suitably, the peptide may have no more than 35 amino acids. Suitably, the peptide may consist of from 10 to 35 amino acids. Suitably, the peptide may consist of from 17 to 35 amino acids.

Suitably, the peptide may be conjugated to an immune stimulatory compound. Suitably, the immune stimulatory compound may comprise a TLR, NLR, RLR, CLR, or ALR ligand.

A nucleic acid encoding the peptide of the invention is also provided. Suitably, the nucleic acid may be mRNA or DNA. Suitably, the nucleic acid may be an isolated nucleic acid.

In one aspect, the invention provides a complex comprising:
a) a peptide comprising the amino acid sequence of SEQ ID NO: 1, and
b) a binding agent that specifically binds to a peptide comprising the amino acid sequence of SEQ ID NO: 1. Suitably, the complex may be an isolated complex.

Suitably, the binding agent may be an HLA-A*02 molecule. Suitably, the complex may be a complex of HLA-A*02 with any one of SEQ ID NOs: 2-21. Suitably, the complex may be an HLA-A*02:SLGDWGAEA complex, an HLA-A*02:SLGDVGAEV complex, an HLA-A*02:SLGDVGAEI complex, or an HLA-A*02:SLGDVGAEL complex.

Suitably, the binding agent may be an HLA-A*02:01 molecule. Suitably, the complex may be a complex of HLA-A*02:01 with any one of SEQ ID NOs: 2-21. Suitably, the complex may be an HLA-A*02:01:SLGDVGAEA complex, an HLA-A*02:01:SLGDWGAEV complex, an HLA-A*02:01:SLGDVGAEI complex, an HLA-A*02:01:SLGDVGAEL complex.

Suitably, the complex may be:
a. a SLGDVGAEA:HLA-A*02 complex, a SLGDWGAEV:HLA-A*02 complex, a SLGDVGAEI:HLA-A*02 complex, a SLGDVGAEL:HLA-A*02 complex, a SLGDVGAER:HLA-A*02 complex, a SLGDWGAEN:HLA-A*02 complex, a SLGDVGAED:HLA-A*02 complex, a SLGDVGAEC:HLA-A*02 complex, a SLGDVGAEQ:HLA-A*02 complex, a SLGDVGAEE:HLA-A*02 complex, a SLGDWGAEG:HLA-A*02 complex, a SLGDWGAEH:HLA-A*02 complex, a SLGDVGAEK:HLA-A*02 complex, a SLGDVGAEM:HLA-A*02 complex, a SLGDWGAEF:HLA-A*02 complex, a SLGDVGAEP:HLA-A*02 complex, a SLGDWGAES:HLA-A*02 complex, a SLGDVGAET:HLA-A*02 complex, a SLGDVGAEW:HLA-A*02 complex or a SLGDVWGAEY:HLA-A*02 complex, optionally wherein the complex is a SLGDVNGAEA:HLA-A*02 complex, a SLGDWGAEV:HLA-A*02 complex, a SLGDWGAEI:HLA-A*02 complex or a SLGDVGAEL:HLA-A*02 complex, further optionally wherein the complex is a SLGDVGAEA:HLA-A*02 complex, a SLGDVGAEV:HLA-A*02 complex or a SLGDVWGAEI:HLA-A*02 complex; or
b. a SLGDWGAEA:HLA-A*02:01 complex, a SLGDWGAEV:HLA-A*02:01 complex, a SLGDVGAEI:HLA-A*02:01 complex, a SLGDWGAEL:HLA-A*02:01 complex, a SLGDVGAER:HLA-A*02:01 complex, a SLGDWGAEN:HLA-A*02:01 complex, a SLGDVGAED:HLA-A*02:01 complex, a SLGDWGAEC:HLA-A*02:01 complex, a SLGDVGAEQ:HLA-A*02:01 complex, a SLGDWGAEE:HLA-A*02:01 complex, a SLGDVGAEG:HLA-A*02:01 complex, a SLGDWGAEH:HLA-A*02:01 complex, a SLGDVGAEK:HLA-A*02:01 complex, a SLGDWGAEM:HLA-A*02:01 complex, a SLGDVGAEF:HLA-A*02:01 complex, a SLGDWGAEP:HLA-A*02:01 complex, a SLGDVGAES:HLA-A*02:01 complex, a SLGDWGAET:HLA-A*02:01 complex, a SLGDWNGAEW:HLA-A*02:01 complex or a SLGDWNGAEY:HLA-A*02:01 complex, optionally wherein the complex is a SLGDWGAEA:HLA-A*02:01 complex, a SLGDWGAEV:HLA-A*02:01 complex, a SLGDWGAEI:HLA-A*02:01 complex or a SLGDWGAEL:HLA-A*02:01 complex, further optionally wherein the complex is a SLGDWGAEA:HLA-A*02:01 complex, a SLGDVGAEV:HLA-A*02:01 complex or a SLGDWGAEI:HLA-A*02:01 complex.

In yet another aspect, the invention provides a cell loaded with a peptide according to the invention or a cell loaded with or expressing the complex according to the invention. Suitably, the cell may be an antigen presenting cell. Suitably, the antigen presenting cell may be selected from a macrophage, dendritic cell, a monocyte, a B-cell or a synthetic form of antigen presenting cell. Suitably, the cell may be loaded with a peptide according to the invention comprising an amino acid sequence selected from the group consisting of: SLGDWGAEA (SEQ ID NO: 2), SLGDVGAEV (SEQ ID NO: 3), SLGDVGAEI (SEQ ID NO: 4) and SLGDWGAEL (SEQ ID NO: 5). For example, the cell may be loaded with a peptide comprising an amino acid sequence selected from any one of SEQ ID NO: 22 to 27, 110 or 111. Suitably, the cell may be loaded with or expressing a complex according to the invention selected from the group consisting of: a SLGDVGAEA:HLA-A*02:01 complex, a SLGDVGAEV:HLA-A*02:01 complex, a SLGDWNGAEI:HLA-A*02:01 complex and a SLGDWGAEL:HLA-A*02:01.

In another aspect, the invention provides an isolated nucleic acid composition that encodes a TIMP3 antigen-specific binding protein having a TCR α chain variable (Vα) domain and a TCR β chain variable (Vβ) domain, the composition comprising: a nucleic acid sequence that encodes a TCR Vα domain comprising a CDR3 amino acid sequence; and a nucleic acid sequence that encodes a TCR Vβ domain comprising a CDR3 amino acid sequence; wherein the CDR3 sequences together specifically bind to a peptide comprising the amino acid sequence of SEQ ID NO: 1 (e.g. when the peptide is complexed with HLA). Suitably, the CDR3 sequences together may specifically bind to a peptide comprising the amino acid sequence of any one of SEQ ID NOs: 2 21 (e.g. when the peptide is complexed with HLA). Suitably, the CDR3 sequences together may specifically bind to a peptide comprising the amino acid sequence SLGDWGAEA (SEQ ID NO: 2), SLGDVGAEV (SEQ ID NO: 3), SLGDVGAEI (SEQ ID NO: 4) or SLGDVGAEL (SEQ ID NO: 5) (e.g. when the peptide is complexed with HLA). Suitably, the CDR3 sequences together may specifically bind to the peptide when it is complexed with HLA-A*02:01. Suitably, the nucleic acid sequence may encode a T cell receptor.

In an aspect, the invention provides an isolated nucleic acid composition that encodes a TIMP3 antigen-specific binding protein having a TCR α chain variable (Vα) domain and a TCR β chain variable (Vβ) domain, the composition comprising:
(i) a nucleic acid sequence that encodes a TCR Vα domain comprising a CDR3 amino acid sequence having at least 80% sequence identity to SEQ ID NO:42, or a functional fragment thereof; and a nucleic acid sequence that encodes a TCR Vβ domain comprising a CDR3 amino acid sequence having at least 80% sequence identity to SEQ ID NO:45, or a functional fragment thereof, wherein the CDR3 sequences together specifically bind to a peptide comprising the amino acid sequence of SEQ ID NO:1 (e.g. wherein the CDR3 sequences together specifically bind to a peptide comprising the amino acid sequence of SEQ ID NO: 2 (e.g. when the peptide is complexed with HLA)); or
(ii) a nucleic acid sequence that encodes a TCR Vα domain comprising a CDR3 amino acid sequence having at least 80% sequence identity to SEQ ID NO:52, or a functional fragment thereof; and a nucleic acid sequence that encodes a TCR Vβ domain comprising a CDR3 amino acid sequence having at least 80% sequence identity to SEQ ID NO: 55, or a functional fragment thereof, wherein the CDR3 sequences together specifically bind to a peptide comprising the amino acid sequence of SEQ ID NO:1 (e.g. wherein the CDR3 sequences together specifically bind to a peptide comprising the amino acid sequence of SEQ ID NO: 2 (e.g. when the peptide is complexed with HLA)); or
(iii) a nucleic acid sequence that encodes a TCR Vα domain comprising a CDR3 amino acid sequence having at least 80% sequence identity to SEQ ID NO:62, or a functional fragment thereof; and a nucleic acid sequence that encodes a TCR Vβ domain comprising a CDR3 amino acid sequence having at least 80% sequence identity to SEQ ID NO:65, or a functional fragment thereof, wherein the CDR3 sequences together specifically bind to a peptide comprising the amino acid sequence of SEQ ID NO:1 (e.g. wherein the CDR3 sequences together specifically bind to a peptide comprising the amino acid sequence of SEQ ID NO: 2 (e.g. when the peptide is complexed with HLA)); or
(iv) a nucleic acid sequence that encodes a TCR Vα domain comprising a CDR3 amino acid sequence having at least 80% sequence identity to SEQ ID NO:72, or a functional fragment thereof; and a nucleic acid sequence that encodes a TCR Vβ domain comprising a CDR3 amino acid sequence having at least 80% sequence identity to SEQ ID NO: 75, or a functional fragment thereof, wherein the CDR3 sequences together specifically bind to a peptide comprising the amino acid sequence of SEQ ID NO:1 (e.g. wherein the CDR3 sequences together specifically bind to a peptide comprising the amino acid sequence of SEQ ID NO: 2 (e.g. when the peptide is complexed with HLA)); or
(v) a nucleic acid sequence that encodes a TCR Vα domain comprising a CDR3 amino acid sequence having at least 80% sequence identity to SEQ ID NO:82, or a functional fragment thereof; and a nucleic acid sequence that encodes a TCR Vβ domain comprising a CDR3 amino acid sequence having at least 80% sequence identity to SEQ ID NO: 85, or a functional fragment thereof, wherein the CDR3 sequences together specifically bind to a peptide comprising the amino acid sequence of SEQ ID NO:1 (e.g. wherein the CDR3 sequences together specifically bind to a peptide comprising the amino acid sequence of SEQ ID NO: 2 (e.g. when the peptide is complexed with HLA)); or
(vi) a nucleic acid sequence that encodes a TCR Vα domain comprising a CDR3 amino acid sequence having at least 80% sequence identity to SEQ ID NO:92, or a functional fragment thereof; and a nucleic acid sequence that encodes a TCR Vβ domain comprising a CDR3 amino acid sequence having at least 80% sequence identity to SEQ ID NO: 95, or a functional fragment thereof, wherein the CDR3 sequences together specifically bind to a peptide comprising the amino acid sequence of SEQ ID NO:1 (e.g. wherein the CDR3 sequences together specifically bind to a peptide comprising the amino acid sequence of SEQ ID NO: 2 (e.g. when the peptide is complexed with HLA)); or
(vii) a nucleic acid sequence that encodes a TCR Vα domain comprising a CDR3 amino acid sequence having at least 80% sequence identity to SEQ ID NO:102, or a functional fragment thereof; and a nucleic acid sequence that encodes a TCR Vβ domain comprising a CDR3 amino acid sequence having at least 80% sequence identity to SEQ ID NO: 105, or a functional fragment thereof, wherein the CDR3 sequences together specifically bind to a peptide comprising the amino acid sequence of SEQ ID NO:1 (e.g. wherein the CDR3 sequences together specifically bind to a peptide comprising the amino acid sequence of SEQ ID NO: 2 (e.g. when the peptide is complexed with HLA)).

Suitably, the peptide may comprise an amino acid sequence selected from the group consisting of: SEQ ID NOs: 2-21. Suitably, the peptide may comprise an amino acid sequence selected from the group consisting of: SEQ ID NOs: 2-5. Suitably, the peptide may comprise an amino acid sequence selected from the group consisting of: SEQ ID NOs: 2-4. Suitably, the peptide may comprise an amino acid sequence selected from the group consisting of: SEQ ID NOs: 3-5.

Suitably, the isolated nucleic acid composition that encodes a TIMP3 antigen-specific binding protein having a TCR α chain variable (Vα) domain and a TCR β chain variable (Vβ) domain according to the invention may comprise:
(i) a Vα domain CDR3 that comprises or consists of the amino acid sequence of SEQ ID NO: 42, and the CDR3 of the Vβ domain comprises or consists of the amino acid sequence of SEQ ID NO:45; or
(ii) a Vα domain CDR3 that comprises or consists of the amino acid sequence of SEQ ID NO: 52, and the CDR3 of the Vβ domain comprises or consists of the amino acid sequence of SEQ ID NO:55; or
(iii) a Vα domain CDR3 that comprises or consists of the amino acid sequence of SEQ ID NO: 62, and the CDR3 of the Vβ domain comprises or consists of the amino acid sequence of SEQ ID NO:65; or
(iv) a Vα domain CDR3 that comprises or consists of the amino acid sequence of SEQ ID NO: 72, and the CDR3 of the Vβ domain comprises or consists of the amino acid sequence of SEQ ID NO:75; or
(v) a Vα domain CDR3 that comprises or consists of the amino acid sequence of SEQ ID NO: 82, and the CDR3 of the Vβ domain comprises or consists of the amino acid sequence of SEQ ID NO:85; or
(vi) a Vα domain CDR3 that comprises or consists of the amino acid sequence of SEQ ID NO: 92, and the CDR3 of the Vβ domain comprises or consists of the amino acid sequence of SEQ ID NO:95; or
(vii) a Vα domain CDR3 that comprises or consists of the amino acid sequence of SEQ ID NO: 102, and the CDR3 of the Vβ domain comprises or consists of the amino acid sequence of SEQ ID NO:105.

Suitably, the isolated nucleic acid composition that encodes a TIMP3 antigen-specific binding protein having a TCR α chain variable (Vα) domain and a TCR β chain variable (Vβ) domain according to the invention may comprise:
(i) a Vα domain that comprises an amino acid sequence having at least 80% sequence identity to, comprising, or consisting of, SEQ ID NO: 46; and the Vβ domain comprises an amino acid sequence having at least 80% sequence identity to, comprising, or consisting of, SEQ ID NO: 48; or
(ii) a Vα domain that comprises an amino acid sequence having at least 80% sequence identity to, comprising, or consisting of, SEQ ID NO: 56; and the Vβ domain comprises an amino acid sequence having at least 80% sequence identity to, comprising, or consisting of, SEQ ID NO: 58; or
(iii) a Vα domain that comprises an amino acid sequence having at least 80% sequence identity to, comprising, or consisting of, SEQ ID NO: 66; and the Vβ domain comprises an amino acid sequence having at least 80% sequence identity to, comprising, or consisting of, SEQ ID NO: 68; or
(iv) a Vα domain that comprises an amino acid sequence having at least 80% sequence identity to, comprising, or consisting of, SEQ ID NO: 76; and the Vβ domain comprises an amino acid sequence having at least 80% sequence identity to, comprising, or consisting of, SEQ ID NO: 78; or
(v) a Vα domain that comprises an amino acid sequence having at least 80% sequence identity to, comprising, or consisting of, SEQ ID NO: 86; and the Vβ domain comprises an amino acid sequence having at least 80% sequence identity to, comprising, or consisting of, SEQ ID NO: 88; or
(vi) a Vα domain that comprises an amino acid sequence having at least 80% sequence identity to, comprising, or consisting of, SEQ ID NO: 96; and the Vβ domain comprises an amino acid sequence having at least 80% sequence identity to, comprising, or consisting of, SEQ ID NO: 98; or
(vii) a Vα domain that comprises an amino acid sequence having at least 80% sequence identity to, comprising, or consisting of, SEQ ID NO: 106; and the Vβ domain comprises an amino acid sequence having at least 80% sequence identity to, comprising, or consisting of, SEQ ID NO: 108. Suitably, the encoded TIMP3 antigen-specific binding protein having a TCR α chain variable (Vα) domain and a TCR β chain variable (Vβ) domain is capable of specifically binding to a peptide:HLA complex selected from the group consisting of: a SX₁GDWGAEX₂:HLA-A*02 complex (wherein X₁ and X₂ are any amino acid), a SLGDWGAEA:HLA-A*02 complex, a SLGDVGAEV:HLA-A*02 complex, a SLGDVGAEI:HLA-A*02 complex, a SLGDVGAEL:HLA-A*02 complex, a SLGDWGAER:HLA-A*02 complex, a SLGDVGAEN:HLA-A*02 complex, a SLGDVGAED:HLA-A*02 complex, a SLGDVGAEC:HLA-A*02 complex, a SLGDWGAEQ:HLA-A*02 complex, a SLGDVGAEE:HLA-A*02 complex, a SLGDVGAEG:HLA-A*02 complex, a SLGDVGAEH:HLA-A*02 complex, a SLGDVGAEK:HLA-A*02 complex, a SLGDVGAEM:HLA-A*02 complex, a SLGDVGAEF:HLA-A*02 complex, a SLGDVGAEP:HLA-A*02 complex, a SLGDVGAES:HLA-A*02 complex, a SLGDWGAET:HLA-A*02 complex, a SLGDWGAEW-HLA-A*02 complex, and a SLGDVGAEY:HLA-A*02 complex.

Suitably, the encoded TIMP3 antigen-specific binding protein having a TCR α chain variable (Vα) domain and a TCR β chain variable (Vβ) domain is capable of specifically binding to a peptide:HLA complex selected from the group consisting of: a SX₁GDWGAEX₂:HLA-A*02:01 complex (wherein X₁ and X₂ are any amino acid), a SLGDWGAEA:HLA-A*02:01 complex, a SLGDVGAEV:HLA-A*02:01 complex, a SLGDVGAEI:HLA-A*02:01 complex, a SLGDVGAEL:HLA-A*02:01 complex, a SLGDVGAER:HLA-A*02:01 complex, a SLGDVGAEN:HLA-A*02:01 complex, a SLGDVGAED:HLA-A*02:01 complex, a SLGDVGAEC:HLA-A*02:01 complex, a SLGDVGAEQ:HLA-A*02:01 complex, a SLGDVGAEE:HLA-A*02:01 complex, a SLGDWGAEG:HLA-A*02:01 complex, a SLGDVGAEH:HLA-A*02:01 complex, a SLGDVGAEK:HLA-A*02:01 complex, a SLGDWGAEM:HLA-A*02:01 complex, a SLGDWNGAEF:HLA-A*02:01 complex, a SLGDVGAEP:HLA-A*02:01 complex, a SLGDWGAES:HLA-A*02:01 complex, a SLGDWGAET:HLA-A*02:01 complex, a SLGDWNGAEW-HLA-A*02:01 complex, and a SLGDVGAEY:HLA-A*02:01 complex.

Suitably, the encoded TIMP3 antigen-specific binding protein having a TCR α chain variable (Vα) domain and a TCR β chain variable (Vβ) domain is capable of specifically binding to a peptide:HLA complex selected from the group consisting of: a SLGDWGAEA:HLA-A*02:01 complex, a SLGDVGAEV:HLA-A*02:01 complex, a SLGDWGAEI:HLA-A*02:01 complex, and a SLGDVGAEL:HLA-A*02:01 complex. Suitably, the encoded TIMP3 antigen-specific binding protein having a TCR α chain variable (Vα) domain and a TCR β chain variable (Vβ) domain is capable of specifically binding to a peptide:HLA complex selected from the group consisting of: a SLGDVGAEA:HLA-A*02:01 complex and a SLGDWGAEV:HLA-A*02:01 complex.

Suitably, the CDR3 sequences together may specifically bind to a peptide comprising the amino acid sequence of SEQ ID NO: 1 (e.g. when the peptide is complexed with HLA). Suitably, the CDR3 sequences together may specifically bind to a peptide comprising the amino acid sequence of any one of SEQ ID NOs: 2-21 ((e.g. when the peptide is complexed with HLA). Suitably, the peptide may comprise the amino acid sequence SLGDVGAEA (SEQ ID NO: 2), SLGDVGAEV (SEQ ID NO: 3), SLGDVGAEI (SEQ ID NO: 4) or SLGDVGAEL (SEQ ID NO: 5).

Suitably, the CDR3 sequences together may specifically bind to the peptide when it is complexed with HLA-A*02 or HLA-A*02:01.

Suitably, the encoded binding protein comprises a TCR, an antigen binding fragment of a TCR, a chimeric antigen receptor (CAR), or an ImmTAC.

In yet another aspect, the invention provides an isolated nucleic acid encoding a chimeric antigen receptor protein comprising a target binding moiety that specifically binds to a peptide comprising the amino acid sequence of SEQ ID NO: 1. Suitably, the peptide may comprise the amino acid sequence of any one of SEQ ID NOs: 2-21. Suitably, the peptide may comprise the amino acid sequence SLGDWGAEA (SEQ ID NO: 2), SLGDWGAEV (SEQ ID NO: 3), SLGDWGAEI (SEQ ID NO: 4) or SLGDWGAEL (SEQ ID NO: 5).

A modified cell transformed, transfected or transduced with a nucleic acid comprising a nucleic acid sequence encoding a peptide described herein, a nucleic acid composition described herein, a nucleic acid described herein, or a nucleic acid sequence described herein or a vector described herein is also provided. Suitably, the modified cell may be a human cell. Suitably, the modified cell may be selected from the group consisting of a CD8 T cell, a CD4 T cell, an NK cell, an NKT cell, a gamma-delta T cell, an innate lymphoid cell (ILC), a hematopoietic stem cell, a progenitor cell, a T cell line or a NK-92 cell line.

In another aspect, the invention provides a pharmaceutical composition comprising a) a peptide, b) a nucleic acid, a nucleic acid sequence, or a nucleic acid composition, c) a complex, or d) a cell according to the invention, and a pharmaceutically acceptable excipient, adjuvant, diluent and/or carrier.

For the avoidance of doubt, any one of a), b) or c) may be present in the pharmaceutical composition by virtue of them being encoded or expressed (as appropriate) by a cell that is present within the pharmaceutical composition. As an example, any one of b) or c) may be encoded by a cell that is combined with a pharmaceutically acceptable excipient, adjuvant, diluent and/or carrier to generate the pharmaceutical composition; or any of a) or b) may be expressed by a cell that is combined with a pharmaceutically acceptable excipient, adjuvant, diluent and/or carrier to generate the pharmaceutical composition. More details on this are provided below under “detailed description”.

Suitably, the composition may be formulated as a vaccine. Suitably, the composition may be a peptide vaccine. Suitably, the vaccine, peptide vaccine, or composition may comprise an immune stimulatory compound.

A pharmaceutical composition described herein is provided for use as a medicament. A vaccine or peptide vaccine described herein is provided for use as a medicament. A pharmaceutical composition described herein (comprising a nucleic acid, a nucleic acid composition, a complex, or a cell according to the invention) and a vaccine or peptide vaccine described herein are provided for use as a medicament. Suitably, the pharmaceutical composition described herein and the vaccine or peptide vaccine described herein may be combined for use as a medicament. Suitably, the combination may comprise administering the vaccine or peptide vaccine prior to, during, and/or after administration of the pharmaceutical composition.

Suitably, the pharmaceutical composition, vaccine, and/or peptide vaccine may be for use in the prevention or treatment of a pre-cancer, a cancer or a viral infection associated with impaired HLA class I antigen presentation in a human subject. Suitably, the pre-cancer or cancer may be a pre-cancer or cancer with impaired peptide processing machinery.

Suitably, the pharmaceutical composition, vaccine, and/or peptide vaccine, may be for use in treating or preventing a pre-cancer, a cancer or viral infection associated with impaired HLA class I antigen presentation in a human subject, wherein the subject has been identified as having a pre-cancer, a cancer or viral infection associated with impaired HLA class I antigen presentation by the presence of a SX₁GDVGAEX₂:HLA-A*02 complex (wherein X₁ and X₂ are any amino acid), a SLGDWGAEA:HLA-A*02 complex, a SLGDWGAEV:HLA-A*02 complex, a SLGDVWGAEI:HLA-A*02 complex, a SLGDWGAEL:HLA-A*02 complex, a SLGDWGAER:HLA-A*02 complex, a SLGDWGAEN:HLA-A*02 complex, a SLGDWGAED:HLA-A*02 complex, a SLGDWGAEC:HLA-A*02 complex, a SLGDWGAEQ:HLA-A*02 complex, a SLGDWGAEE:HLA-A*02 complex, a SLGDWGAEG:HLA-A*02 complex, a SLGDWGAEH:HLA-A*02 complex, a SLGDWGAEK:HLA-A*02 complex, a SLGDWGAEM:HLA-A*02 complex, a SLGDWGAEF:HLA-A*02 complex, a SLGDVGAEP:HLA-A*02 complex, a SLGDVGAES:HLA-A*02 complex, a SLGDVWGAET:HLA-A*02 complex, a SLGDWGAEW-HLA-A*02 complex or a SLGDWGAEY:HLA-A*02 complex in a sample isolated from the subject.

Suitably, the pharmaceutical composition, vaccine, and/or peptide vaccine, may be for use in treating or preventing a pre-cancer, a cancer or viral infection associated with impaired HLA class I antigen presentation in a human subject, wherein the subject has been identified as having a pre-cancer, a cancer or viral infection associated with impaired HLA class I antigen presentation by the presence of a SX₁GDWNGAEX₂:HLA-A*02:01 complex (wherein X₁ and X₂ are any amino acid), a SLGDWNGAEA:HLA-A*02:01 complex, a SLGDWGAEV:HLA-A*02:01 complex, a SLGDWGAEI:HLA-A*02:01 complex, a SLGDWGAEL:HLA-A*02:01 complex, a SLGDWGAER:HLA-A*02:01 complex, a SLGDWGAEN:HLA-A*02:01 complex, a SLGDWGAED:HLA-A*02:01 complex, a SLGDWGAEC:HLA-A*02:01 complex, a SLGDWGAEQ:HLA-A*02:01 complex, a SLGDWGAEE:HLA-A*02:01 complex, a SLGDWGAEG:HLA-A*02:01 complex, a SLGDWGAEH:HLA-A*02:01 complex, a SLGDWGAEK:HLA-A*02:01 complex, a SLGDWGAEM:HLA-A*02:01 complex, a SLGDWGAEF:HLA-A*02:01 complex, a SLGDWGAEP:HLA-A*02:01 complex, a SLGDWGAES:HLA-A*02:01 complex, a SLGDWGAET:HLA-A*02:01 complex, a SLGDWGAEW:HLA-A*02:01 complex or a SLGDWGAEY:HLA-A*02:01 complex in a sample isolated from the subject.

In another aspect, the invention provides a method of treating a condition in a human subject in need thereof, the method comprising administering to the subject a therapeutically effective amount of a pharmaceutical composition, vaccine, and/or peptide vaccine according to the invention. Suitably, the method of treating may be for the prevention or treatment of a pre-cancer, a cancer or a viral infection associated with impaired HLA class I antigen presentation.

A method of generating a T cell receptor is provided, comprising contacting a nucleic acid sequence or composition according to the invention with a cell under conditions in which the nucleic acid sequence is incorporated and expressed by the cell to generate the T cell receptor that specifically binds to a peptide comprising the amino acid sequence of SEQ ID NO: 1 (or to the peptide when complexed with HLA). Suitably, the peptide may comprise the amino acid sequence of any one of SEQ ID NOs: 2-21. Suitably, the method is ex vivo.

In another aspect, the invention provides a use of a peptide comprising an amino acid sequence of SEQ ID NO: 1, or a complex according to the invention, for identifying a therapeutic binding protein. Suitably the peptide may comprise or consist of an amino acid sequence of any one of SEQ ID NOs: 2-27, 110 or 111. Suitably, the therapeutic binding protein may be capable of preventing or treating a pre-cancer, a cancer or a viral infection associated with impaired HLA class I antigen presentation. Suitably, the therapeutic binding protein may comprise: a TCR, an antigen binding fragment of a TCR, or a chimeric antigen receptor (CAR), or an ImmTAC; or the therapeutic binding protein may be an antibody.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings.

Herein, peptide SLGDWGAEA (SEQ ID NO:2) is also referred to as ‘peptide 124’ or ‘p124’.

FIG. 1 shows specificity of three T cell cultures for the peptide SLGDWGAEA. T cell cultures of donors 2028, 4409 and 2752 were stimulated with the peptide SLGDWGAEA (SEQ ID NO:2) and analyzed via flow cytometry for the percentage of CD8+ T cells and the percentage therein of CD8+ T cells that stained positive with the HLA-A*02:01 tetramer presenting peptide SLGDWGAEA. All three SLGDWGAEA-specific T cell cultures comprise more than 97% CD8+ T cells that stained for more than 99% positive with SLGDVGAEA-loaded HLA-A*02:01 tetramers (indicated by HLA-A*02:01: SLGDWGAEA™ in FIG. 1). These SLGDWGAEA-specific T cells were used in subsequent experiments described below. In the present application and figures, peptide SLGDWGAEA is also referred to as ‘peptide 124’ or ‘p124’.

FIG. 2 shows preferential recognition of TAP impaired tumor but not proximal tubular (HK2) cells by p124-specific T cells. Peptide 124-specific T cells preferentially recognized 518A2 melanoma and 08.11 melanoma cells when TAP is knocked out, but not non-tumor cells such as fibroblasts, HEK-293T cells, or HK2 cells. FIG. 2A shows the following: the T cell cultures of donor 2028, 4409 and 2752, stimulated with peptide 124 (SLGDWGAEA), respectively indicated by 2028 p124, 4409 p124 and 2752 p124, were stimulated with HLA-A*0201 matched wild type (WT) melanoma cells (518A2), its variant in which TAP was knocked out (TAP KO) and with cognate peptide (10 μg/ml). FIG. 2B shows the following: in addition, cultures 2752 and 4409 were stimulated with HLA-A*0201 matched HK2 cells, fibroblasts and HEK293T cells. FIG. 2C shows the following: T cell culture 2752 was stimulated with both wild type (WT) melanoma cells (518A2, 08.11) and their variants in which TAP was knocked out (518A2 TAP KO, 8.11 TAP KO) as well as with wild type HK2 cells and its variant with TAP knocked out (HK2 TAP KO). For FIGS. 2A-C: the cultures were assessed for interferon-gamma (IFNγ) or GM-CSF production, which is shown on the y-axis in pg/ml. The dashed line indicates the maximal value of the dose titration curve.

FIG. 3 shows that no recognition of healthy cells by SLGDWGAEA-specific T cells was observed. In FIG. 3A, the p124 SLGDWGAEA-specific T cell culture of donor 4409, was stimulated with autologous EBV-transformed B cells (EBVs) (indicated by “T cells+EBV”) with as control EBVs loaded with 10 μg/ml p124 (indicated by “T cells+EBVs+p124”). Negative controls are indicated by “T cells” and “EBVs”. In FIG. 3B, the p124 SLGDWGAEA-specific T cell culture of donor 4409, was stimulated with HLA-A*0201-matched CD56+cells, CD19+B cells or monocytes (Mono's) isolated from PBMC as well as monocyte-derived dendritic cells (moDCs). As control, the Mono's were loaded with 5 μg/ml p124 (indicated by “Mono's+p124+ T cells”). FIG. 3C shows the following: the p124 SLGDWGAEA-specific T cell culture of donor 4409, was stimulated with autologous Mono's (i.e. “Mono's 4409”) or HLA-A*0201-matched Mono's from another donor (i.e. “Mono's 7110”), which when indicated were loaded with p124 peptide (5 μg/ml). T cells with 25 μg/ml peptide served as control for reactivity (indicated by “T cells+p124”). T cells only served as a negative control (indicated by “T cells only”). For FIGS. 3A-C: The amount of interferon-gamma (IFNy) produced is shown on the y-axis in pg/ml.

FIG. 4 shows expression levels of TIMP3, the gene coding for peptide 124. The expression levels of TIMP3 were determined in monocytes of two different donors 2752 and 4409 (indicated by “2752 mono's and 4409 mono's”), monocyte-derived dendritic cells of two different donors 2752 and 4409 (indicated by “2752 moDCs” and “4409 moDCs”), 518A2 melanoma cells (indicated by “518A2 WT”) and its TAP knock-out derivative (indicated by “518A2 TAP KO”) as well as HK2 cells (indicated by “HK2 WT”) and its TAP knock-out derivative (indicated by “HK2 TAP KO”). The expression of TIMP3 was tested by qPCR and measurements were performed in triplicates. Ct-values were normalized to the expression of the housekeeping genes and CPSF6.

FIG. 5 shows expression levels of TIMP3 and TAP1 in 518A2 wild type cells, 518A2 TAP KO cells and 518A2 TAP KO TIMP3 KO cells. The expression levels of TIMP3 and TAP1 were determined in 518A2 wild type (WT) melanoma cells (518A2 WT), 518A2 cells in which TAP was knocked out (518A2 TAP KO) and 3 different clones of 518A2 cells in which both TAP and TIMP3 were knocked out (518A2 TAP/TIMP3 KO (clone 1G8, 1G10, 2C9)). The expression of the indicated genes was tested by qPCR and measurements were performed in triplicates. Ct-values were normalized to the expression of the housekeeping genes and CPSF6.

FIG. 6 shows that peptide 124-specific T cell cultures have single TIMP3 protein specificity. The peptide 124-specific T-cell cultures from two different donors (indicated by “4409 p124” and “5944 p124”) were stimulated with 518A2 wild type (WT) melanoma cells (518A2 WT), 518A2 cells in which TAP was knocked out (518A2 TAP KO) and 3 different clones of 518A2 cells in which both TAP and TIMP3 were knocked out (518A2 TAP/TIMP3 KO (clone 1G8, 1G10, 2C9)). The amount of interferon-gamma (IFNy) or GM-CSF produced is shown on the y-axis in pg/ml. The dashed line indicates the maximal value of the dose titration curve. Analysis of the reactivity of p124-specific CD8+ T cells showed a complete absence of recognition when stimulated with 518A2 TAP KO TIMP3 KO cells, while 518A2 were recognized, especially when TAP was knocked out. This shows that peptide 124-specific T cells are truly specific for the TIMP3 derived peptide SLGDVGAEA.

FIG. 7 shows that peptide 124 SLGDWGAEA is cross-presented by monocyte-derived dendritic cells from long synthetic peptides comprising the natural flanking amino acids. The peptide 124-specific T-cell culture from donor 4409 was stimulated with HLA-A*0201 matched monocyte-derived dendritic cells (MoDCs) from three different donors (indicated by MoDCs 0559, MoDC 4553 and MoDC 7401), which were loaded with (10 uM for 20-24h) the indicated synthetic long peptides (SLP) (“p124 long 1” (SEQ ID NO: 22), (“p124 long 2” (SEQ ID NO: 23), (“p124 long 3” (SEQ ID NO: 24)) or with no peptide as background control. The T cell culture incubated with 25 ug/ml the p124-peptide SLGDWGAEA served as positive control for reactivity (indicated by “T cells+p124”). The amount of interferon-gamma (IFNy) or GM-CSF produced is shown on the y-axis in pg/ml.

FIG. 8 shows cross-presentation of anchor replaced TIMP3-derived variant peptide SLGDWGAEV from a longer peptide sequence comprising the natural flanking amino acids. The peptide 124-specific T-cell cultures from donors 4409 and 5944 were stimulated with HLA-A*0201 matched monocyte-derived dendritic cells (MoDCs) from three different donors (indicated by “MoDC donor 0559”, “MoDC donor 4553” and “MoDC donor 7401”), which were loaded with 10 uM for 20-24 h of the indicated synthetic long peptides (“p124 long V1-3” (SEQ ID NO: 25), “p124 long V1-4” (SEQ ID NO: 110), “p124 long V1-6” (SEQ ID NO: 111), “p124 long V2” (SEQ ID NO: 26), “p124long V3” (SEQ ID NO: 27)) or with no peptide as background control. The T cell culture incubated with 25 ug/ml of the p124-peptide SLGDWGAEA (indicated by “T cells+short p124”) or the V-variant SLGDWGAEV (indicated by “T cells+short p124V′”) served as positive control for reactivity, while T cells incubated without any added peptide served as a negative control (indicated by “T cells only”). The amount of interferon-gamma (IFNy) is shown on the y-axis in pg/ml. The dashed line for 4409 indicates the maximal value of the dose titration curve.

FIG. 9 shows that T cells transduced in order to express different unique HLA-A*02:01-restricted TIMP3₁₅₋₂₃ CD8 T cell receptors as identified by the inventors, can bind to murine TCRb antibody and MHC multimers of HLA-A*02:01-molecules presenting the peptide TIMP3₁₅₋₂₃ SLGDVWGAEA. The TCR-alpha and TCR-beta chains of each respective identified TCR were cloned into a retroviral expression vector, which was used to transduce T cells, resulting in the generation of T cells expressing the respective TCRs for peptide TIMP3₁₅₋₂₃ SLGDWNGAEA. These T cells were analyzed via flow cytometry. The introduced genes contain the murine TCR-Cβ domain which enhances correct pairing of transgenic alpha and beta chains. Expression of this domain is confirmed in TCR-transduced T cells after incubation with murine TCRb antibody. The specificity of the T cells was analyzed by analysis of positive staining with MHC multimers of HLA-A*02:01-molecules presenting the peptide TIMP3₁₅₋₂₃ SLGDWGAEA.

FIG. 10 shows the identification of HLA-presented peptides FLGPWPAAS, FLGPWPAAV, VLAPRVLRA, and SLGDWGAEA in different isolated human primary tumour samples. Various human primary tumour samples were obtained after which their HLA ligandomes were determined via mass spectrometry by standard methods in the art. The primary tumour samples comprise four melanoma samples (Mel0401, Mel1207, Mel1213, and Mel1502), three ovarian cancer samples (OVAL1, OVAL10, and OVAL23), and two cervical cancer samples (COV362-4 and COV413b). The table in FIG. 10 shows the presence (+) or absence (no) of four possible HLA-presented peptides (FLGPWPAAS, FLGPWPAAV, VLAPRVLRA, and SLGDWGAEA) in the samples after analysis of their HLA ligandomes.

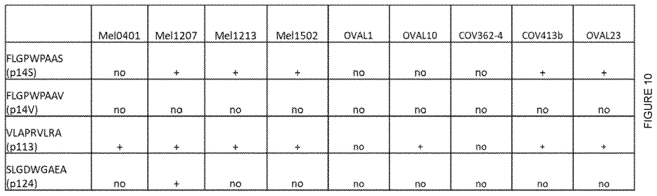

FIG. 11 shows that T cells transduced in order to express different unique HLA-A*02:01-restricted TIMP3₁₅₋₂₃ (p124)-specific CD8 T cell receptors as identified by the inventors herein can be activated by TAP-deficient tumor cells. TCR generated by TCR-gene transfer and enriched after tetramer-guided MACS sort were cultured overnight with (from left to right in the figure) HLA-A*0201-matched wild type (WT), TAP-deficient (TAP KO) and TAP and TIMP3-deficient (TAP/Ag KO) 518A2 tumor cells, cultured by themselves (T cells only), cultured with 25 ug/ml cognate peptide (T cells+peptide), or with wild type (WT) and TAP-deficient (TAP KO) 08.11 tumor cells. The amount of GM-CSF, IFNy and CCL4 produced is shown for TCR3, 4, 6, and 9. The experiments of FIG. 11 were performed using the materials and methods as set out in the section Examples below. The TCRs used in FIG. 11 are the same as in the Examples and their sequences can be found in tables 7-13.

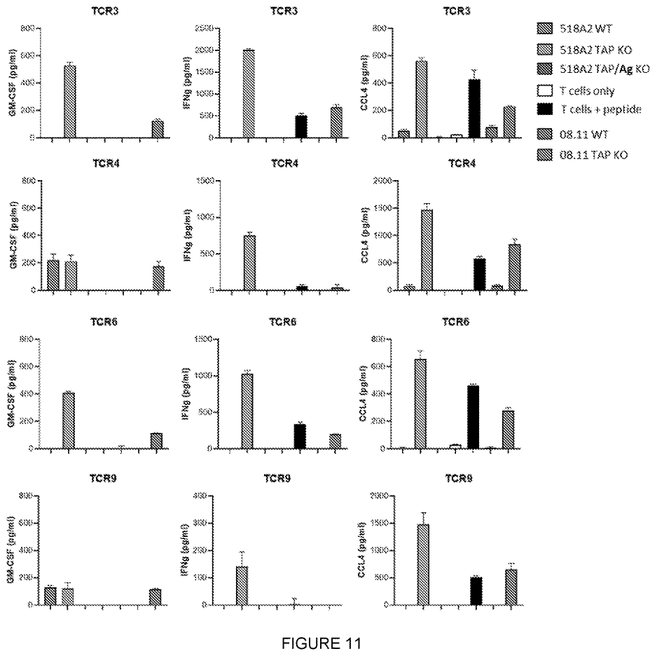

FIG. 12 shows affinity of TIMP3₁₅₋₂₃ (p124)-specific TCRs. The recognition pattern of T cells transduced with TIMP3₁₅₋₂₃ (p124)-specific TCRs was tested by stimulating them with 20000 EBV-transformed B cells loaded with the indicated doses of peptide (6.7 pg-6.7 ug/m) of the wild type TIMP3₁₅₋₂₃-peptide (p124) (SLGDWGAEA) or the V-variant peptide (p124V) (SLGDWGAEV). The amount of GM-CSF (left) and CCL4 (right) produced at each dose of peptide is shown. The dashed line indicates the dose of peptide required to half-maximally activate the T cell (IC50) and serves as an indication for the affinity of the TCR against the test peptide. The experiments of FIG. 12 were performed using the materials and methods as set out in the section Examples below. The TCRs used in FIG. 12 are the same as in the Examples and their sequences can be found in tables 7-13.

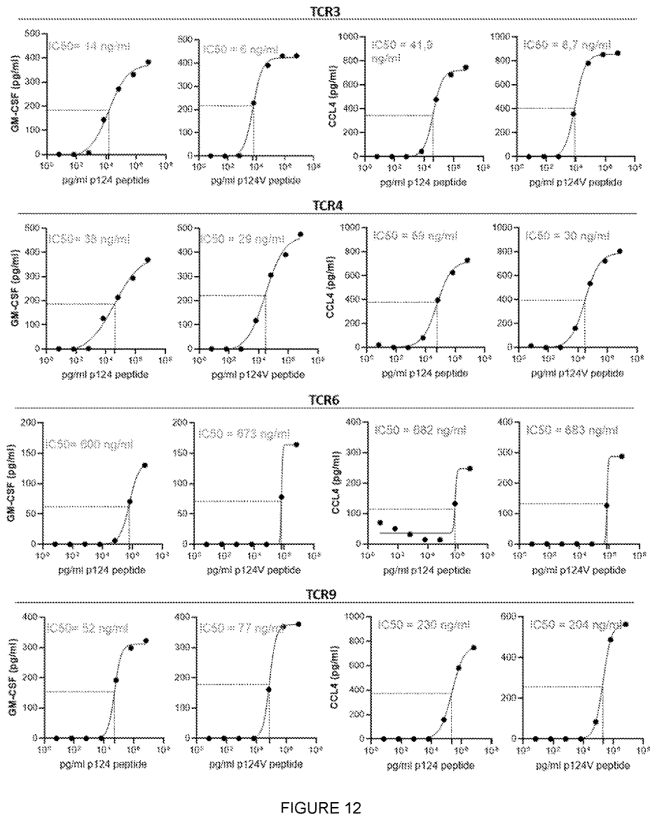

FIG. 13 shows heatmap representations of the 9-mer combinatorial peptide library (CLP) scan of TIMP3₁₅₋₂₃ (p124)-specific TCRs. Heatmaps depict the GM-CSF production by TCR2, TCR4 and TCR9 (with the highest affinity) in response to EBV (50000 cells/well) loaded with the 180-peptide mixtures in a 9-mer CPL scan, indicating which amino acids are preferentially recognized at which position in the peptide sequence. No cross-reactivity is observed. Negative and positive controls are shown in the graphs on the right hand. The experiments of FIG. 13 were performed using the materials and methods as set out in the section Examples below. The TCRs used in FIG. 13 are the same as in the Examples and their sequences can be found in tables 7-13.

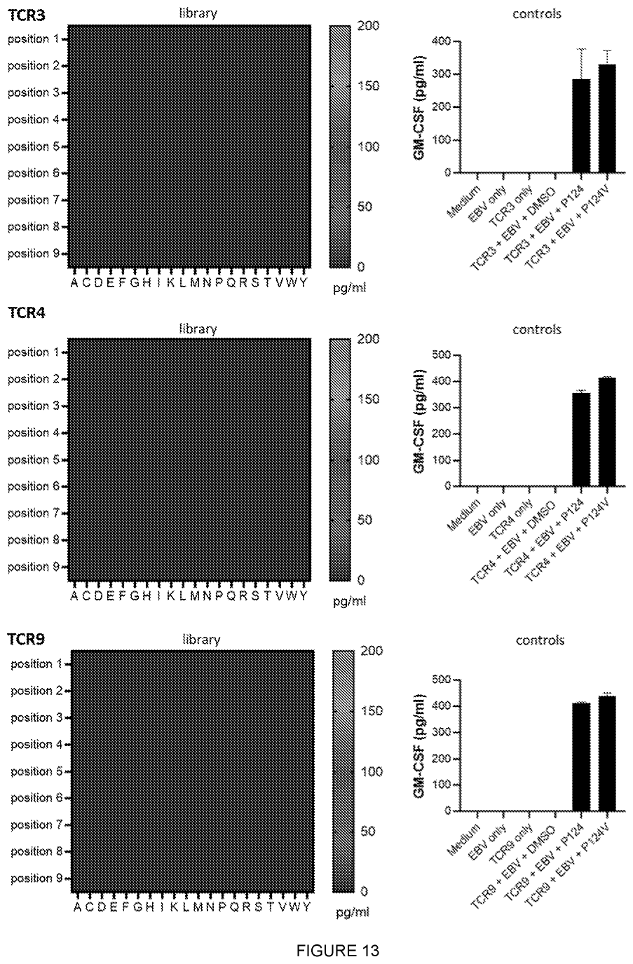

### DETAILED DESCRIPTION

### Immunogenic Peptides

A peptide comprising the amino acid sequence of SEQ ID NO: 1 is provided herein. A peptide comprising the amino acid sequence SLGDWGAEA (SEQ ID NO: 2) is provided herein. A peptide comprising the amino acid sequence SLGDWGAEV (SEQ ID NO: 3) is provided herein. A peptide comprising the amino acid sequence SLGDWGAEI (SEQ ID NO: 4) is provided herein. A peptide comprising the amino acid sequence SLGDWGAEL (SEQ ID NO: 5) is provided herein. A peptide comprising an amino acid sequence selected from the group consisting of SEQ ID NO: 2-21 is provided herein, preferably wherein the amino acid sequence is selected from the group consisting of SEQ ID NO: 2-5, more preferably wherein the amino acid sequence is selected from the group consisting of SEQ ID NO: 2-4, or from the group consisting of SEQ ID NO: 3-5. A peptide comprising the amino acid sequence of SEQ ID NO:2 includes the peptides of SEQ ID NO: 22 to 24. Reference to a peptide comprising SEQ ID NO:2 therefore expressly encompasses the peptides of SEQ ID NO:22 to 24. Similarly, a peptide comprising the amino acid sequence of SEQ ID NO:3 includes the peptides of SEQ ID NO: 25 to 27, as well as the peptides of SEQ ID NO: 110 and 111. Reference to a peptide comprising SEQ ID NO:3 therefore expressly encompasses the peptides of SEQ ID NO:25 to 27, 110 and 111.

Preferably, the peptide is an isolated peptide.

Also provided herein are nucleic acid molecules encoding the peptides of the present invention. A nucleic acid is provided encoding the amino acid sequence of SEQ ID NO: 1. A nucleic acid is provided encoding any one of the amino acid sequences of SEQ ID NO: 1-27, 110 or 111. Any suitable nucleic acid sequence encoding any one of the amino acid sequences of SEQ ID NO: 1-27, 110 or 111 may be used accordingly. These can suitably be selected by the skilled person. As used herein, an “isolated peptide” refers to a peptide that is not in its natural environment. The peptide may therefore be of synthetic origin (or alternatively, of natural original, but isolated from its natural environment). In the context of this disclosure, the natural environment of these peptides is within the human body. Accordingly, when the peptides are present e.g. in a pharmaceutical composition (comprising adjuvants etc) they are considered to be in isolated form, as they are not in their natural environment.

The peptides described herein may be part of a pharmaceutical composition, wherein the composition comprises:
(a) the peptide; and
(b) a pharmaceutically acceptable excipient, adjuvant, diluent and/or carrier. Such compositions may be referred to as vaccines (i.e. peptide vaccines) herein.

Further, the nucleic acids described herein may be part of a pharmaceutical composition, wherein the compostions comprises:
(a) a nucleic acid encoding a peptide of the invention; and
(b) a pharmaceutically acceptable excipient, adjuvant, diluent and/or carrier. Such compositions may be referred to herein as vaccines (i.e. nucleic acid vaccines) herein.

An example of such a vaccine comprising a nucleic acid sequence encoding the peptide, optionally comprising the peptide, is an mRNA vaccine. Preferably, the vaccine according to the invention is an mRNA vaccine.

The peptide according to the invention may consist of the amino acid sequence of any one of SEQ ID NOs: 1-27, 110 or 111 only. Alternatively, the peptide may include additional amino acids, and thus comprise the amino acid sequence of any one of SEQ ID NOs: 1-27, 110 or 111.

In an example, peptides comprising the amino acid sequence SLGDWGAEV (SEQ ID NO:3) (such as any one of the peptides of SEQ ID NO: 25 to 27, 110 or 111) may have a higher binding affinity to HLA-A*02 than an equivalent peptide in which the SLGDWGAEV (SEQ ID NO:3) sequence is replaced with SLGDWGAEA (SEQ ID NO:2). In a particular example, peptides comprising SLGDWGAEV (SEQ ID NO:3) (such as any one of the peptides of SEQ ID NO: 25 to 27, 110 or 111) may have a higher binding affinity to HLA-A*02:01 than an equivalent peptide in which the SLGDWGAEV (SEQ ID NO:3) sequence is replaced with SLGDWGAEA (SEQ ID NO:2). Methods of determining the binding affinity of a peptide to an HLA molecule are well known in the art (see for example, the experiments included in the “examples” section below). Methods of determining the binding affinity of a peptide to an HLA molecule are well known in the art and specifically described by Van der Burg et al. 1995 and Van der Burg et al. 1996.

In a further example, cross-presentation of peptides comprising the amino acid sequence SLGDWGAEV (SEQ ID NO:3) (such as any one of the peptides of SEQ ID NO: 25 to 27, 110 or 111) by monocyte-derived dendritic cells may be more effective (improved/higher) than the cross-presentation of an equivalent peptide in which the SLGDWGAEV (SEQ ID NO:3) sequence is replaced with SLGDWGAEA (SEQ ID NO:2). In other words, a peptide comprising the amino acid sequence SLGDWGAEV (SEQ ID NO:3) (such as any one of the peptides of SEQ ID NO: 25 to 27, 110 or 111) may be more efficiently presented by monocyte-derived dendritic cells than an equivalent peptide in which the SLGDWGAEV (SEQ ID NO:3) sequence is replaced with SLGDWGAEA (SEQ ID NO:2).

Methods of determining the efficacy of cross-presentation of a particular peptide by monocyte-derived dendritic cells are well known in the art, see for example the experiments included in the “examples” section below. In an example, cross-presentation of a 10-35 amino acid long peptide comprising the equivalent peptide epitope can be tested by the use of monocyte-derived dendritic cells (DC) and a CD8+ T cell clone recognizing the peptide SLGDWGAEA in the context of HLA class I. Monocyte-derived DC are obtained by incubating peripheral blood mononuclear cells with anti-CD14 magnetic beads for 20 min at 4° C. followed by the isolation of CD14 positive monocytes were isolated using magnetic separation columns. The CD14+ monocytes are cultured in RPMI medium supplemented with 10% FCS, GM-CSF (800 units/ml), and IL-4 (500 units/ml) for 6 days to generate immature monocyte-derived dendritic cells. On day 6, the immature monocyte-derived DCs are incubated with the synthetic long peptide at different doses (e.g. 20 μg/ml, 10 μg/m, 5 μg/ml) for 24h, and matured with LPS (20 ng/ml) stimulation on day 7. The cross-presentation of the peptide by these monocyte-derived DC is monitored by the reactivity of the CD8+ T cell clone co-cultured with the peptide-pulsed monocyte-derived DCs at different ratio's (e.g. 10 T cells to 1 DC; 5 T cells to 1 DC; 1 T cell to 1 DC). The reactivity of a CD8+ T cell clone can be tested in different ways, including measurement of cytokine production (e.g. GM-CSF or Interferon-gamma) in the supernatant of the co-culture and can be compared to control co-cultures in which the monocyte-derived DC are pulsed with an irrelevant HLA class I binding peptide or with no peptide. As a positive control the monocyte-derived DC can be pulsed with the natural short epitope SLGDVGAEA.

In a further example, the peptides described herein may have an equivalent or a higher binding affinity to HLA-A*02 (e.g. HLA-A*02:01) and/or be presented by monocyte-derived dendritic cells at a level that is equivalent or more efficient (improved/higher) than an equivalent peptide comprising SLGDVGAEA.

As would be clear to a person of skill in the art, as used above, “equivalent peptides” are peptides with an identical amino acid sequence except for the recited difference.

In some embodiments, the peptide may be no more than 35 amino acids in length; e.g. no more than 35, 34, 33, 32, 31, 30, 29, 28, 27, 26, 25, 24, 23, 22, 21, 20, 19, 18, 17, 16, 15, 14, 13, 12, 11, 10 or 9 amino acids. In one example, the peptide may be no more than 30 amino acids in length. In another example, the peptide may be no more than 29 amino acids in length. In one example, the peptide may be no more than 28 amino acids in length. In another example, the peptide may be no more than 27 amino acids in length. In another example, the peptide may be no more than 26 amino acids in length. In yet another example, the peptide may be no more than 25 amino acids in length. In another example, the peptide may be no more than 24 amino acids in length. In another example, the peptide may be no more than 23 amino acids in length. In another example, the peptide may be no more than 21 amino acids in length. In another example, the peptide may be no more than 20 amino acids in length. In another example, the peptide may be no more than 19 amino acids in length. In another example, the peptide may be no more than 17 amino acids in length.

Different lengths of peptide have been shown to be particularly effective as peptide vaccines. For example, Ossendorp (Ossendorp et al., (1998)) describe that 9-19 amino acid long peptides are able to induce a CD4+ helper T cell response. Accordingly, the peptide of the invention may be from 9 to 19 amino acids long.

Peptides that are longer than the conventional 9mer sequence presented by HLA may be more efficient in inducing an immune response. Accordingly, in line with the teaching of Ossendorp (Ossendorp et al., (1998)), the peptides described herein may be from 10 to 19 amino acids long. Bijker (Bijker et al., (2007)) demonstrate that a 9-mer HPV CTL epitope can induce a CD8+response to RAHYNIVTF but that a 35-mer peptide comprising this epitope is more efficient. This is further supported by Beyranvand-Nejad (Beyranvand-Nejad et al., (2016)), showing that a 35 mer HPV peptide works well to induce RAHYNIVTF-specific CD8+ T cells. Furthermore, Rahimian et al., (2015) show that a 27-mer HPV peptide works well to induce a response to RAHYNIVTF. Accordingly, in line with these teachings, the peptides described herein may be from 9 to 35 amino acids long. For the avoidance of doubt, in this context, the peptides have a total of 9 to 35 amino acids, which includes the sequence of any one of SEQ ID NO: 1-21. In other words, the peptides have the sequence of any one of SEQ ID NO: 1-21 and 0 to 26 additional amino acids. The 0 to 26 additional amino acids of the peptide may be located N-terminal or C-terminal to the sequence of any one of SEQ ID NO: 1-21. Alternatively, when there are 0 to 26 additional amino acids, the additional amino acids may flank the sequence of any one of SEQ ID NO: 1-21 (i.e. such that there are additional amino acid(s) N-terminal and C-terminal of the sequence of any one of SEQ ID NO: 1-21). Additional amino acids located N-terminal, C-terminal or flanking any one of SEQ ID NO: 1-21 are referred to collectively as “additional amino acids” herein.

Alternatively, for the avoidance of doubt, in this context, the peptides may have a total of 10 to 35 amino acids, which includes the sequence of any one of SEQ ID NOs 1-21. In other words, the peptides may have the sequence of any one of SEQ ID NOs 1-21 and 1 to 26 additional amino acids. The 1 to 26 additional amino acids of the peptide may be located N-terminal or C-terminal to the sequence of any one of SEQ ID NOs 1-21. Alternatively, when there are 1 to 26 additional amino acids, the additional amino acids may flank the sequence of any one of SEQ ID NOs 1-21 (i.e. such that there are additional amino acid(s) N-terminal and C-terminal of the sequence of any one of SEQ ID NOs 1-21).

In another example, the peptides described herein may be from 15 to 30 amino acids long, where there are additional 6 to 21 additional amino acids. The additional amino acids may be located N-terminal or C-terminal to sequence of any one of SEQ ID NO: 1-21. Alternatively, the additional amino acids may flank the sequence of any one of SEQ ID NO: 1-21 (i.e. such that there are additional amino acid(s) N-terminal and C-terminal of the sequence of any one of SEQ ID NO: 1-21).

In another example, the peptides described herein may be from 17 to 27 amino acids long, where there are additional 8 to 18 additional amino acids. The additional amino acids may be located N-terminal or C-terminal to sequence of any one of SEQ ID NO: 1-21. Alternatively, the additional amino acids may flank the sequence of any one of SEQ ID NO: 1-21 (i.e. such that there are additional amino acid(s) N-terminal and C-terminal of the sequence of any one of SEQ ID NO: 1-21).

In another example, the peptides described herein may be from 21 to 24 amino acids long, where there are additional 12 to 15 additional amino acids. The additional amino acids may be located N-terminal or C-terminal to sequence of any one of SEQ ID NO: 1-21. Alternatively, the additional amino acids may flank the sequence of any one of SEQ ID NO: 1-21 (i.e. such that there are additional amino acid(s) N-terminal and C-terminal of the sequence of any one of SEQ ID NO: 1-21).

In another example, the peptides described herein may be from 17 to 23 amino acids long, where there are additional 8 to 14 additional amino acids. The additional amino acids may be located N-terminal or C-terminal to sequence of any one of SEQ ID NO: 1-21. Alternatively, the additional amino acids may flank the sequence of any one of SEQ ID NO: 1-21 (i.e. such that there are additional amino acid(s) N-terminal and C-terminal of the sequence of any one of SEQ ID NO: 1-21).

Examples of peptides that comprise the amino acid sequence of SEQ ID NO:2 with additional amino acids are shown in SEQ ID NO: 22 to 24. Examples of peptides that comprise the amino acid sequence of SEQ ID NO:3 with additional amino acids are shown in SEQ ID NO: 25 to 27, 110 or 111.

In another example, the peptides described herein may be 27, 25, 23 amino acids long. In a further example, the peptides described herein may be 23 amino acids long. In another example, the peptides described herein may be 21 amino acids long. In another example, the peptides described herein may be 20 amino acids long. In a particular example, the peptides described herein may be 17 amino acids long. Preferably, the peptides described herein are 19 amino acids long. For the avoidance of doubt, in this context, the peptides have a total of e.g. 27, 25, 23, 21, 20, 19 or 17 amino acids, which includes the sequence of any one of SEQ ID NOs 1-21. In other words, the peptides have the sequence of any one of SEQ ID NOs 1-21 and a suitable number of additional amino acids (i.e. to generate a peptide that is 27, 25, 23 21, 20 or 19 or 17 amino acids long).

The N-terminus of a peptide (also known as the amino-terminus, NH₂-terminus, N-terminal end or amine-terminus) is the start of a peptide terminated by an amino acid with a free amine group (—NH₂). By convention, peptide sequences are written N-terminus to C-terminus (from left to right). The C-terminus (also known as the carboxyl-terminus, carboxy-terminus, C-terminal tail, C-terminal end, or COOH-terminus) is the end of an amino acid chain (protein or polypeptide), terminated by a free carboxyl group (—COOH).

As used herein, the terms “N-terminal” and “C-terminal” are used to describe the relative position of e.g. a sequence within a peptide. Accordingly, a sequence that is “N-terminal” is positioned closer (in relative terms) to the N-terminus than to the C-terminus of the peptide. Conversely, a domain that is “C-terminal” is positioned (in relative terms) closer to the C-terminus than to the N-terminus of the peptide. As used herein, the term “positioned” refers to the location of the sequence within the linear amino acid sequence of the peptide.

Peptides comprising an N-terminal amino acid sequence (A) and a C-terminal amino acid sequence (B) are conventionally written as A-B i.e. N-terminal to C-terminal (left to right).

Where the peptides described herein include additional amino acids located N-terminal, C-terminal or flanking the sequence of any one of SEQ ID NOs 1-21, any appropriate additional amino acid sequences may be included. For example, the additional amino acids may be amino acid sequences that are naturally located N-terminal, C-terminal or flanking the SLGDWGAEA sequence in TIMP3. In a particular example, the additional amino acids may be all located N-terminal to the sequence of any one of SEQ ID NOs 1-21 and may be the natural sequence that is found N-terminal to the SLGDWGAEA sequence in TIMP3. In another example, the additional amino acids may be all be located C-terminal to the sequence of any one of SEQ ID NOs 1-21 and may be the natural sequence that is found C-terminal to the SLGDNGAEA sequence in TIMP3. Alternatively, the additional amino acids may flank the sequence of any one of SEQ ID NOs 1-21 (i.e. such that there are additional amino acid(s) N-terminal and C-terminal of the sequence of any one of SEQ ID NOs 1-21) and may be the natural sequences that flank the SLGDWGAEA sequence in TIMP3. Preferably, the additional amino acids may be all located N-terminal to a SLGDWGAEV sequence, optionally wherein the additional amino acid sequences are amino acid sequences that are naturally located N-terminal of the SLGDNGAEA sequence in TIMP3. In this example, the peptide sequence of interest (e.g. SLGDWGAEV) is located at the C-terminus of the peptide according to the invention. Alternatively, the additional amino acids may be flanking the SLGDWGAEV sequence, optionally wherein the additional amino acid sequences are amino acid sequences that are naturally located N-terminal of the SLGDNGAEA sequence in TIMP3. In this example, the peptide sequence of interest (e.g. SLGDWGAEV) is not located at the C-terminus or the N-terminus of the peptide according to the invention (e.g. see the examples).

Peptides that comprise the amino acid sequence of any one of SEQ ID NOs 1-21 and consist of from 10 to 35 amino acids may include any appropriate additional amino acid sequences. For example, the additional amino acids may be amino acid sequences that are naturally located N-terminal, C-terminal or flanking the SLGDNGAEA sequence in TIMP3.

In another example, the additional 1 to 26 amino acids may all be located N-terminal to any one of SEQ ID NOs 1-21, preferably the SLGDVNGAEA, SLGDVNGAEV, SLGDVNGAEI or SLGDVNGAEL sequence, and may be the natural sequence that is found N-terminal to the SLGDVNGAEA sequence in TIMP3. In another example, the additional 1 to 26 amino acids may be all be located C-terminal to any one of SEQ ID NOs 1-21, preferably the SLGDVNGAEA, SLGDVNGAEV, SLGDVNGAEI or SLGDVNGAEL sequence, and may be the natural sequence that is found C-terminal to the SLGDWGAEA sequence in TIMP3. Alternatively, when there are 1 to 26 additional amino acids, the additional amino acids may flank any one of SEQ ID NOs 1-21, preferably the SLGDVNGAEA, SLGDVNGAEV, SLGDVNGAEI or SLGDWGAEL sequence (i.e. such that there are additional amino acid(s) N-terminal and C-terminal of any one of SEQ ID NOs 1-21, preferably the SLGDVNGAEA, SLGDVNGAEV, SLGDVNGAEI or SLGDVNGAEL sequence) and may be the natural sequence that flank the SLGDVNGAEA sequence in TIMP3.

In another particular example, peptides that comprise the amino acid sequence of SEQ ID NO: 1 (e.g. the amino acid sequence of SLGDVNGAEA (SEQ ID NO:2), SLGDVNGAEV (SEQ ID NO:3), SLGDVNGAEI (SEQ ID NO:4) or SLGDVNGAEL (SEQ ID NO:5)) and consist of from 15 to 30 amino acids may include any appropriate additional amino acid sequences. For example, the additional amino acids may be amino acid sequences that are naturally located N-terminal, C-terminal or flanking the SLGDVNGAEA sequence in TIMP3. For example, the additional 6 to 21 amino acids may all be located N-terminal to the sequence of SEQ ID NO: 1 (e.g. the SLGDVNGAEA, SLGDVNGAEV, SLGDVNGAEI or SLGDVNGAEL sequence) and may be the natural sequence that is found N-terminal to the SLGDVNGAEA sequence in TIMP3. In another example, the additional 6 to 21 amino acids may be all be located C-terminal to the sequence of SEQ ID NO: 1 (e.g. the SLGDWGAEA, SLGDVNGAEV, SLGDVNGAEI or SLGDVNGAEL sequence) and may be the natural sequence that is found C-terminal to the SLGDVNGAEA sequence in TIMP3. Alternatively, when there are 6 to 21 additional amino acids, the additional amino acids may flank the sequence of SEQ ID NO: 1 (e.g. the SLGDVNGAEA, SLGDVNGAEV, SLGDVNGAEI or SLGDVNGAEL sequence) (i.e. such that there are additional amino acid(s) N-terminal and C-terminal of the sequence) and may be the natural sequence that flank the SLGDVNGAEA sequence in TIMP3.

In another example, peptides that comprise the amino acid sequence of SEQ ID NO: 1 (e.g. the amino acid sequence of SLGDVNGAEA (SEQ ID NO:2), SLGDVNGAEV (SEQ ID NO:3), SLGDVNGAEI (SEQ ID NO:4) or SLGDVNGAEL (SEQ ID NO:5)) and consist of from 17 to 27 amino acids may include any appropriate additional amino acid sequences. For example, the additional amino acids may be amino acid sequences that are naturally located N-terminal, C-terminal or flanking the SLGDVNGAEA sequence in TIMP3. For example, the additional 8 to 18 amino acids may all be located N-terminal to the sequence of SEQ ID NO: 1 (e.g. the SLGDVNGAEA, SLGDVNGAEV, SLGDVNGAEI or SLGDVNGAEL sequence) and may be the natural sequence that is found N-terminal to the SLGDVGAEA sequence in TIMP3. In another example, the additional 8 to 18 amino acids may be all be located C-terminal to the sequence of SEQ ID NO: 1 (e.g. the SLGDWGAEA, SLGDWGAEV, SLGDWGAEI or SLGDWGAEL sequence) and may be the natural sequence that is found C-terminal to the SLGDWGAEA sequence in TIMP3. Alternatively, when there are 8 to 18 additional amino acids, the additional amino acids may flank the sequence of SEQ ID NO: 1 (e.g. the SLGDWGAEA, SLGDWGAEV, SLGDWGAEI or SLGDWGAEL sequence) (i.e. such that there are additional amino acid(s) N-terminal and C-terminal of the sequence) and may be the natural sequence that flank the SLGDVGAEA sequence in TIMP3.

In a further example, peptides that comprise the amino acid sequence of SEQ ID NO: 1 (e.g. the amino acid sequence of SLGDWGAEA (SEQ ID NO:2), SLGDWGAEV (SEQ ID NO:3), SLGDWGAEI (SEQ ID NO:4) or SLGDWGAEL (SEQ ID NO:5)) and consist of from 21 to 24 amino acids may include any appropriate additional amino acid sequences. For example, the additional amino acids may be amino acid sequences that are naturally located N-terminal, C-terminal or flanking the SLGDWNGAEA sequence in TIMP3. For example, the additional 12 to 15 amino acids may all be located N-terminal to the sequence of SEQ ID NO: 1 (e.g. the SLGDVGAEA, SLGDVGAEV, SLGDVGAEI or SLGDVGAEL sequence) and may be the natural sequence that is found N-terminal to the SLGDVGAEA sequence in TIMP3. In another example, the additional 12 to 15 amino acids may be all be located C-terminal to the sequence of SEQ ID NO: 1 (e.g. the SLGDWGAEA, SLGDWGAEV, SLGDWGAEI or SLGDWGAEL sequence) and may be the natural sequence that is found C-terminal to the SLGDWGAEA sequence in TIMP3. Alternatively, when there are 12 to 15 additional amino acids, the additional amino acids may flank the sequence of SEQ ID NO: 1 (e.g. the SLGDWGAEA, SLGDWGAEV, SLGDWGAEI or SLGDWGAEL sequence) (i.e. such that there are additional amino acid(s) N-terminal and C-terminal of the sequence) and may be the natural sequence that flank the SLGDVGAEA sequence in TIMP3.

In a further example, peptides that comprise the amino acid sequence of SEQ ID NO: 1 (e.g. the amino acid sequence of SLGDWGAEA (SEQ ID NO:2), SLGDWGAEV (SEQ ID NO:3), SLGDWGAEI (SEQ ID NO:4) or SLGDWGAEL (SEQ ID NO:5)) and consist of from 17 to 23 amino acids may include any appropriate additional amino acid sequences. For example, the additional amino acids may be amino acid sequences that are naturally located N-terminal, C-terminal or flanking the SLGDWNGAEA sequence in TIMP3. For example, the additional 8 to 14 amino acids may all be located N-terminal to the sequence of SEQ ID NO: 1 (e.g. the SLGDVGAEA, SLGDVGAEV, SLGDVGAEI or SLGDVGAEL sequence) and may be the natural sequence that is found N-terminal to the SLGDWGAEA sequence in TIMP3. In another example, the additional 8 to 14 amino acids may be all be located C-terminal to the sequence of SEQ ID NO: 1 (e.g. the SLGDWGAEA, SLGDWGAEV, SLGDWGAEI or SLGDWGAEL sequence) and may be the natural sequence that is found C-terminal to the SLGDWGAEA sequence in TIMP3. Alternatively, when there are 8 to 14 additional amino acids, the additional amino acids may flank the sequence of SEQ ID NO: 1 (e.g. the SLGDWGAEA, SLGDWGAEV, SLGDWGAEI or SLGDWGAEL sequence) (i.e. such that there are additional amino acid(s) N-terminal and C-terminal of the sequence) and may be the natural sequence that flank the SLGDWGAEA sequence in TIMP3.

Suitable natural sequences from TIMP3 are provided in the Examples section below.

For example, the peptide may comprise N-terminal additional amino acids, for example it may comprise the sequence: MTPWLGLIVLLGSWSLGDWGAEA (SEQ ID NO:22). This is an example of a 23mer with N-terminal additional amino acids, although other lengths may also be acceptable e.g. 17 mer, 19 mer, 20 mer, 21 mer, 24 mer, 25 mer, 27 mer etc.

In another example, the peptide may comprise additional amino acid(s) N-terminal and C-terminal of the peptide, for example it may comprise the sequence: IVLLGSWSLGDWGAEACTCSPSH (SEQ ID NO:23). This is an example of a 23mer with N-terminal and C-terminal additional amino acids, although other lengths may also be acceptable e.g. 17 mer, 19 mer, 20 mer, 21 mer, 24 mer, 25 mer, 27 mer etc.

In another example the peptide may comprise C-terminal additional amino acids, for example it may comprise the sequence SLGDWGAEACTCSPSHPQDAFCN (SEQ ID NO:24). This is an example of a 23mer with C-terminal additional amino acids, although other lengths may also be acceptable e.g. 17 mer, 19 mer, 20 mer, 21 mer, 24 mer, 25 mer, 27 mer etc.

For example, the peptide may comprise N-terminal additional amino acids, for example it may comprise the sequence: WLGLIVLLGSWSLGDWGAEV (SEQ ID NO:25). This is an example of a 20mer with N-terminal additional amino acids, although other lengths may also be acceptable e.g. 17 mer, 19 mer, 21 mer, 23 mer, 24 mer, 25 mer, 27 mer etc.

For example, the peptide may comprise N-terminal additional amino acids, for example it may comprise the sequence: LGLIVLLGSWSLGDWGAEV (SEQ ID NO:110). This is an example of a 19mer with N-terminal additional amino acids, although other lengths may also be acceptable e.g. 17 mer, 20 mer, 21 mer, 23 mer, 24 mer, 25 mer, 27 mer etc.

For example, the peptide may comprise N-terminal additional amino acids, for example it may comprise the sequence: LIVLLGSWSLGDWGAEV (SEQ ID NO:111). This is an example of a 17mer with N-terminal additional amino acids, although other lengths may also be acceptable e.g. 19 mer, 20 mer, 21 mer, 23 mer, 24 mer, 25 mer, 27 mer etc.

In another example, the peptide may comprise additional amino acid(s) N-terminal and C-terminal of the peptide, for example it may comprise the sequence: IVLLGSWSLGDWGAEVCTCSPSH (SEQ ID NO:26). This is an example of a 23mer with N-terminal and C-terminal additional amino acids, although other lengths may also be acceptable e.g. 17 mer, 19 mer, 20 mer, 21 mer, 24 mer, 25 mer, 27 mer etc.

In another example the peptide may comprise C-terminal additional amino acids, for example it may comprise the sequence SLGDWGAEVCTCSPSHPQDAFCN (SEQ ID NO:27). This is an example of a 23mer with C-terminal additional amino acids, although other lengths may also be acceptable e.g. 17 mer, 19 mer, 20 mer, 21 mer, 24 mer, 25 mer, 27 mer etc.

In an example, the peptide may comprise the amino acid sequence of SEQ ID NO: 22. In another example, the peptide may consist of the amino acid sequence of SEQ ID NO: 22. In an example, the peptide may comprise the amino acid sequence of SEQ ID NO: 23. In another example, the peptide may consist of the amino acid sequence of SEQ ID NO: 23. In another example, the peptide may comprise the amino acid sequence of SEQ ID NO: 24. In yet another example, the peptide may consist of the amino acid sequence of SEQ ID NO: 24. In a further example, the peptide may comprise the amino acid sequence of SEQ ID NO: 25. In another example, the peptide may consist of the amino acid sequence of SEQ ID NO: 25. In a further example, the peptide may comprise the amino acid sequence of SEQ ID NO: 26. In another example, the peptide may consist of the amino acid sequence of SEQ ID NO: 26. In a further example, the peptide may comprise the amino acid sequence of SEQ ID NO: 27. In another example, the peptide may consist of the amino acid sequence of SEQ ID NO: 27. In a further example, the peptide may comprise the amino acid sequence of SEQ ID NO: 110. In another example, the peptide may consist of the amino acid sequence of SEQ ID NO: 110. In a further example, the peptide may comprise the amino acid sequence of SEQ ID NO: 111. In another example, the peptide may consist of the amino acid sequence of SEQ ID NO: 111.

Alternative appropriate natural sequences from TIMP3 may also be identified by a person of skilled in the art. For example, they may be identified using the full length TIMP3 sequence found in SEQ ID NO:28:

MTPWLGLIVLLGSWSLGDWGAEACTCSPSHPQDAFCNSDIVIRAKV
VGKKLVKEGPFGTLVYTIKQMKMYRGFTKMPHVQYIHTEASESLC
GLKLEVNKYQYLLTGRVYDGKMYTGLCNFVERWDQLTLSQRKGLN
YRYHLGCNCKIKSCYYLPCFVTSKNECLWTDMLSNFGYPGYQSKH
YACIRQKGGYCSWYRGWAPPDKSIINATDP

In an alternative example, the additional amino acids may be amino acid sequences that are not naturally located N-terminal, C-terminal or flanking the SLGDWGAEA sequence in TIMP3. Both natural and non-natural flanking sequences have been shown to be useful in peptide vaccines and therefore either may be used in the peptides described herein. For example, the SIINFEKL epitope of the OVA antigen has been successfully used as a peptide vaccine when flanked by its natural sequence (Bijker et al., (2007), a non-natural C-terminal flanking sequence (Varypataki et al., (2015) or without an N-terminal flanking sequence but with a glycine linker attached to a helper epitope at the C-terminal position, so completely outside the context of its own natural flanking sequences (Masuko et al., (2015)). Furthermore, Chen (Chen et al., (2016)) describe fifteen CTL epitope vaccines with small non-natural linkers to the next epitope resulting in priming to the epitopes. Accordingly, N-terminal and/or C-terminal non-natural additional amino acid sequences may be acceptable in a peptide vaccine format.

The peptide may be a “natural peptide” i.e. a peptide composed of natural amino acids. Such peptides are composed of conventional amino acids defined by the genetic code, linked to each other by a normal peptide bond. Natural peptides may, for example, be produced by a cell (via protein expression, e.g. using a nucleic acid or vector described herein), or they may be made synthetically (i.e. outside of a cell, using chemical synthesis).

Alternatively, the peptide may be a “synthetic peptide”. A synthetic peptide may comprise a mix of natural amino acids and amino acids other than conventional amino acids defined by the genetic code (“synthetic amino acids”). Alternatively, it may be composed of synthetic amino acids only. Examples of synthetic amino acids are well known in the literature.

Natural peptides and synthetic peptides may be modified. In other words, the peptide may comprise amino acids modified by natural processes, such as post-translational maturation processes or by chemical processes, which are well known to a person skilled in the art. Such modifications are fully detailed in the literature. These modifications can appear anywhere in the peptide: in the peptide skeleton, in the amino acid chain or at the carboxy- or amino-terminal ends. Non-limiting examples of peptide modifications include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature. Accordingly, the terms “peptide”, “polypeptide”, “protein” may include for example lipopeptides, lipoproteins, glycopeptides, glycoproteins and the like. As a further non-limiting example, the peptide can be branched following ubiquitination or be cyclic with or without branching. This type of modification can be the result of natural or synthetic post-translational processes that are well known to a person skilled in the art.

The peptides described herein may be conjugated directly, or via a linker, to a therapeutic moiety, a polymer, a polypeptide, a ligand and/or any other moiety e.g. a detectable moiety. Such peptides are referred to herein as “peptide conjugates”.

The peptides described herein may be conjugated to an immune stimulatory compound. The peptides described herein may be conjugated to a non-specific immune stimulatory compound. The peptide conjugate may comprise a peptide covalently attached to an immune stimulatory compound. The peptide conjugate may comprise a peptide covalently attached to a non-specific immune stimulatory compound. The peptides described herein may alternatively be comprsed in a composition or vaccine comprising an immune stimulatory compound. Thus, herein is provided a composition or vaccine comprising any one of the peptides according to the invention (or a nucliec acid molecule encoding such a peptide) and an immune stimulatory compound. Also provided herein is a composition or vaccine comprising any one of the peptides according to the invention or a nucleic acid sequence encoding the same, and an immune stimulatory compound. Immune stimulatory compounds are compounds that stimulate the immune system by inducing activation or increasing activity of any of its components. Immune stimulatory compounds comprise specific immune stimulatory compounds and non-specific immune stimulatory compounds. Specific immune stimulatory compounds provide antigenic specificity in immune response, for example any antigen. Non-specific immune stimulatory compounds act irrespective of antigenic specificity to augment immune response of other antigen or stimulate components of the immune system without antigenic specificity, for example adjuvants. For example, immune stimulatory compounds may activate receptors of the innate immune system. For example, immune stimulatory compounds may activate pattern recognition receptors.

The immune stimulatory compound may comprise a damage-associated molecular pattern (DAMP). The immune stimulatory compound may comprise a pathogen-associated molecular pattern (PAMP). The immune stimulatory compound may comprise a ligand for a nucleotide-binding oligomerization domain-like receptor (NLR), such as NOD1 or NOD2. The peptides described herein may be conjugated to a NLR ligand. The peptide conjugate may comprise a peptide covalently attached to a NLR ligand. The immune stimulatory compound may comprise a ligand for a RIG-I-like receptor (RLR), such as RIG-I, MDA5, or LGP2. The peptides described herein may be conjugated to a RLR ligand. The peptide conjugate may comprise a peptide covalently attached to a RLR ligand. The immune stimulatory compound may comprise a ligand for a C-type lectin receptor (CLR), such as Dectin-1 or Dectin-2. The peptides described herein may be conjugated to a CLR ligand. The peptide conjugate may comprise a peptide covalently attached to a CLR ligand. The immune stimulatory compound may comprise aligand for an absent in melanoma-2-like receptor (ALR), such as any ALR. The peptides described herein may be conjugated to an ALR ligand. The peptide conjugate may comprise a peptide covalently attached to an ALR ligand.

Specific ligands binding to respective receptors are known in the art (for example disclosed in “Pattern recognition receptors in health and diseases”, Li & Wu, Signal Transduction and Targeted Therapy, 6; 291, 2021) and such ligands or any effective variants thereof may be suitably selected by the skilled person to function as an immune stimulatory compound according to the present invention.

The immune stimulatory compound may comprise a Toll-Like Receptor (TLR) ligand. The immune stimulatory compound may be a TLR ligand. The peptides described herein may be conjugated to a TLR ligand. The peptide conjugate may comprise a peptide covalently attached to a TLR ligand.

TLR ligands may also be referred to as TLR agonists. As used herein a “TLR agonist” is an agonist of a TLR, i.e. it binds to a TLR and activates the TLR, in particular to produce a biological response. A “TLR peptide agonist” as used herein in a TLR agonist that is a peptide. It is understood that the above regarding TLR agonists can be used interchangeably for NLR, RLR, CLR, and ALR.

Peptide conjugates comprising TLR agonists covalently bound to peptides, in particular TLR agonists that are covalently bound to synthetic peptides, are well known in the art. For example, Zom (Zom et al., (2018)) described a conjugate of the TLR2-ligand Pam3CSK4 to synthetic long peptides (SLPs). Furthermore, Zom (Zom et al., (2016)) described the conjugation of human papillomavirus type 16 (HPV16)-encoded synthetic long peptides to a Pam3CSK4-based TLR2 agonist.

Toll-like receptors (TLRs) are transmembrane proteins that are characterized by extracellular, transmembrane, and cytosolic domains. The extracellular domains containing leucine-rich repeats (LRRs) with horseshoe-like shapes are involved in recognition of common molecular patterns derived from diverse microbes. Toll-like receptors include TLRs1-10. Compounds capable of activating TLR receptors and modifications and derivatives thereof are well documented in the art. TLR1 may be activated by bacterial lipoproteins and acetylated forms thereof, TLR2 may in addition be activated by Gram positive bacterial glycolipids, LPS, LPA, LTA, fimbriae, outer membrane proteins, heat shock proteins from bacteria or from the host, and Mycobacterial lipoarabinomannans. TLR3 may be activated by dsRNA, in particular of viral origin, or by the chemical compound poly(LC). TLR4 may be activated by Gram negative LPS, LTA, Heat shock proteins from the host or from bacterial origin, viral coat or envelope proteins, taxol or derivatives thereof, hyaluronan containing oligosaccharides and fibronectins. TLR5 may be activated with bacterial flagellae or flagellin. TLR6 may be activated by mycobacterial lipoproteins and group B streptococcus heat labile soluble factor (GBS-F) or staphylococcus modulins. TLR7 may be activated by imidazoquinolines. TLR9 may be activated by unmethylated CpG DNA or chromatin—IgG complexes.

TLRs are expressed either on the cell surface (TLR1, 2, 4, 5, 6, and 10) or on membranes of intracellular organelles, such as endosomes (TLR3, 4, 7, 8, and 9). The natural ligands for the endosomal receptors are nucleic acid-based molecules (except for TLR4). The cell surface-expressed TLR1, 2, 4, 5, 6, and 10 recognize molecular patterns of extracellular microbes (Monie et al., (2009)). TLRs are expressed on several cell types but virtually all TLRs are expressed on DCs allowing these specialized cells to sense all possible pathogens and danger signals.

TLR2, 4, and 5 are constitutively expressed at the surface of DCs. TLR2 can detect a wide variety of ligands derived from bacteria, viruses, parasites, and fungi. The ligand specificity is often determined by the interaction of TLR2 with other TLRs, such as TLR1, 6, or 10, or non-TLR molecules, such as dectin-1, CD14, or CD36. The formation of a heterodimer with TLR1 enables TLR2 to identify triacyl lipoproteins or lipopeptides from (myco)bacterial origin, such as Pam3CSK4 and peptidoglycan (PGA) (Gay et al., (2007); Spohn et al., (2004)). Heterodimerization of TLR2 and 6 enables the detection of diacyl lipopeptides and zymosan. Lipopolysaccharide (LPS) and its derivatives are ligands for TLR4 and flagellin for TLR5 (Bryant et al., (2010)). TLR2 interacts with a broad and structurally diverse range of ligands, including molecules expressed by microbes and fungi. Multiple TLR2 agonists have been identified, including natural and synthetic lipopeptides (e.g. Mycoplasma fermentas macrophage-activating lipopeptide (MALP-2)), peptidoglycans (PG such as those from S. aureus), lipopolysaccharides from various bacterial strains (LPS), polysaccharides (e.g. zymosan), glycosylphosphatidyl-inositol-anchored structures from gram positive bacteria (e.g. lipoteichoic acid (LTA) and lipoarabinomannan from mycobacteria and lipomannas from M. tuberculosis). Certain viral determinants may also trigger via TLR2 (Barbalat et al., (2009)). Bacterial lipopeptides are structural components of cel walls. They consist of an acylated s-glycerylcysteine moiety to which a peptide can be conjugated via the cysteine residue. Examples of TLR2 agonists, which are bacterial lipopeptides, include MALP-2 and its synthetic analogue di-palmitoyl-S-glyceryl cysteine (Pam2Cys) or tri-palmitoyl-S-glyceryl cysteine (Pam3Cys).

A diversity of ligands interact with TLR4, including Monophosphoryl Lipid A from Salmonella minnesota R595 (MPLA), lipopolysaccharides (LPS), mannans (Candida albicans), glycoinositolphospholipids (Trypanosoma), viral envelope proteins (RSV and MMTV) and endogenous antigens including fibrinogen and heat-shock proteins. Such agonists of TLR4 are for example described in Akira (Akira et al., 2006) or in Kumar (Kumar et al., 2009). LPS, which is found in the outer membrane of gram negative bacteria, is the most widely studied of the TLR4 ligands. Suitable LPS-derived TLR4 agonist peptides are described for example in WO 2013/120073 (A1).

TLR5 is triggered by a region of the flagellin molecule expressed by nearly all motile bacteria. Thus, flagellin, or peptides or proteins derived from flagellin and/or variants or fragments of flagellin are also suitable as TLR peptide agonists comprised by the peptide conjugate of the present invention.

Non-limiting examples of TLR peptide agonists thus include the TLR2 lipopeptide agonists MALP-2, Pam2Cys and Pam3Cys or modifications thereof, different forms of the TLR4 agonist LPS, e.g. N. meningitidis wild-type L3-LPS and mutant penta-acylated LpxL1-LPS, and the TLR5 agonist flagellin. A further non-limiting example of a TLR2 peptide agonist is annexin II or an immunomodulatory fragment thereof, which is described in detail in WO 2012/048190 A1 and U.S. patent application Ser. No. 13/0331546.

In a further non-limiting example, high-mobility group box 1 protein (HMGB1) and peptide fragments thereof are assumed to be TLR4 agonists. Such HMGB1-derived peptides are for example disclosed in US 2011/0236406 A1.

The peptide conjugate according to the present invention may comprise at least one TLR agonist, preferably the peptide conjugate may comprise more than one TLR agonist, in particular 2, 3, 4, 5, 6, 7, 8, 9, 10 or more TLR agonists.

The at least one TLR, NLR, RLR, CLR, or ALR agonist comprised within the peptide conjugate according to the present invention may be the same or different. Preferably, the various TLR, NLR, RLR, CLR, and/or ALR agonists comprised within the peptide conjugate of the present invention are different from each other.

It is understood that a number of different TLR agonists activating the same or different TLR receptors may be advantageously comprised within a single peptide conjugate according to the present invention.

The immune stimulatory compound may be an adjuvant. In some examples, the adjuvant may be combined with any one of the TLR, NLR, RLR, CLR, or ALR agonists as disclosed herein.

The adjuvant may be selected from the group of mineral salts, emulsions, and microparticles.

Preferably, the mineral salt may be an aluminium salt. The emulsion may be a water-in-oil emulsion or an oil-in-water emulsion. Examples of suitable emulsions are Complete Freund's adjuvant, Incomplete Freund's adjuvant, MF59, ASO3, and ISA51. Montanide ISA51 (Seppic, France) is a water-in-oil emulsion composed of a mineral oil and a surfactant from the mannide monooleate family, and is a preferred adjuvant for the vaccines herein. Examples of microparticles are virus-like particles and virosomes. Virus-like particles are non-infectious nanoparticles without genetic information having an outer region comprised of immunogenic epitopes. Typically, they are icosahedral or rod-shaped nanoparticles (diameter 20-200 nm) comprising a shell of capsid protein.

The pharmaceutical composition described herein (e.g. vaccine) may be administered to a human subject (e.g. as a peptide vaccine) in order to treat or prevent a cancer or viral infection associated with impaired HLA class I antigen presentation. For example, the pharmaceutical composition described herein (e.g. vaccine) may be administered to the subject (e.g. as a peptide vaccine) in order to induce or enhance their immune response. The pharmaceutical composition (e.g. vaccine) may therefore be administered to the subject (e.g. as a peptide vaccine) to induce T cell activation (e.g. in vivo T cell activation) in the subject, wherein the activated T cells are specific for the peptide (and thus will specifically target the cancerous or virally infected cells).

The pharmaceutical composition (e.g. vaccine) may be administered as a peptide vaccine for treating or preventing a cancer or viral infection associated with impaired HLA class I antigen presentation. The pharmaceutical composition (e.g. vaccine) may be administered to induce or enhance activation of T cells specific for cancerous or virally infected cells.

Nucleic acid sequences and vectors encoding the peptides described herein may be administered as a nucleic acid vaccine for treating or preventing a cancer or viral infection associated with impaired HLA class I antigen presentation. The isolated nucleic acid sequences and vectors may be administered to induce or enhance activation of T cells specific for cancerous or virally infected cells.

Peptide vaccines and nucleic acid vaccines are examples of vaccines.

Cross-presentation of long peptides (e.g. SLPs as described herein) by dendritic cells involves endocytosis, cytosolic cleavage of the SLP into short peptides by the proteasome, transport over the ER membrane by TAP and loading onto MHC-l molecules (Rosalia et al. 2013).

The peptides described herein (and corresponding nucleic acid sequences or vectors encoding the same) may be particularly useful as an immunotherapy for human subjects that are positive for HLA-A*02.

HLA-A*02 is a globally common human leukocyte antigen serotype within the HLA-A serotype group. Several subtypes exist within the HLA-A*02 group, including HLA-A*02:01, HLA-A*02:02, HLA-A*02:03, HLA-A*02:04, HLA-A*02:05, HLA-A*02:06, HLA-A*02:09, HLA-A*02:11, HLA-A*02:12, HLA-A*02:16, HLA-A*02:19, HLA-A*02:50. The data presented herein focuses on HLA-A*02:01, however, as would be clear to a person of skill in the art, other subtypes within the HLA-A*02 group (including but not limited to those listed herein) may also bind to any one of the amino acid sequences of SEQ ID NOs: 1-21 (see for example Ressing et al., 1999, particularly Tables 3 and 2). All HLA-A*02 subtypes are therefore encompassed herein, although HLA-A*02:01 is preferred (see Table 1 below). The HLA-A*02 subtypes HLA-A*02:01, HLA-A*02:02, HLA-A*02:03, HLA-A*02:04, and HLA-A*02:09 are also preferred for applications of the invention herein. These HLA-A2 variants were shown to display comparable binding characteristics in accordance with the A2 supertype (Ressing et al., 1999 and M. F. Del Guercio et al., J. Immunol. 1995. 154: 685-693).

TABLE 1
Predicted binding affinity of SLGDWGAEA and c-terminal anchor variants
in HLA-A*02:01. Predicted binding affinity of SLGDWGAEA, SLGDWGAEV,
SLGDWGAEL, SLGDWGAEI, and SLGDWGAEM is determined using the NetMHCcons1.1
algorithm (https://services.healthtech.dtu.dk/service.php?NetMHCcons-1.1).
The prediction value under affinity (in nanomolar, nM, IC50 values)
measures the predicted binding affinity. The rank (in %) is determined by
comparing the predicted binding affinity to a set of 200.000 random natural
peptides. Strong and weak binding peptides are indicated. Recognition
indicates the concentration of the peptide for half maximal interferon-
gamma production of SLGDWGAEA-specific T cells stimulated with the
different variant peptides. ND indicates not detectable.

| | SEQ | Binding | | | |
|---|---|---|---|---|---|
| | ID | affinity | Rank | | Recognition |
| Sequence | NO | (nM)¹ | (%) | Binding level | (IC50)² |
| SLGDWGAEA | 2 | 69.9 | 2 | Weak | 92.525 ng/ml |
| SLGDWGAEV | 3 | 11.4 | 0.5 | Strong | 1.471 ng/ml |
| SLGDWGAEL | 5 | 30.4 | 1.5 | Strong | ND |
| SLGDWGAEI | 4 | 46.3 | 1.5 | Strong | 284.929 ng/ml |
| SLGDWGAEM | 15 | 126 | 3 | Weak | ND |

### Nucleic Acid Sequences

Isolated nucleic acid sequences that encode peptides of the invention are described herein, as well as nucleic acid sequences encoding binding agents are described herein.

As used herein “nucleic acid sequence”, “polynucleotide”, “nucleic acid” and “nucleic acid molecule” are used interchangeably to refer to an oligonucleotide sequence or polynucleotide sequence. The term nucleic acid sequence may therefore be replaced by the term nucleic acid herein. The nucleotide sequence may be of genomic, synthetic or recombinant origin, and may be double-stranded or single-stranded (representing the sense or antisense strand). The term “nucleotide sequence” includes genomic DNA, cDNA, synthetic DNA, and RNA (e.g. mRNA) and analogs of the DNA or RNA generated, e.g., by the use of nucleotide analogs. In one example, the nucleotide sequence lacks introns. In other words, it is an intronless nucleic acid sequence. For example, the nucleotide sequence may be a DNA sequence that does not comprise intron sequences.

As used herein, “isolated nucleic acid sequence” or “isolated nucleic acid composition” refers to a nucleic acid that is not in its natural environment when it is linked to its naturally associated sequence(s) that is/are also in its/their natural environment. In other words, an isolated nucleic acid sequence/composition is not a native nucleotide sequence/composition, wherein “native nucleotide sequence/composition” means an entire nucleotide sequence that is in its native environment and when operatively linked to an entire promoter with which it is naturally associated, which promoter is also in its native environment. Such a nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons). The nucleic acid sequences of the invention may be a non-naturally occurring nucleic acid sequence (e.g. it may be that the entire sequence does not occur in its entirety in nature). For example, the nucleic acid sequence of the invention may be operably linked to a promoter, wherein the promoter is not naturally associated with equivalent human nucleic acid sequences in nature (e.g. human TCR sequences or fragments thereof); i.e. it is not the entire promoter that is naturally associated with the nucleic acid in its natural environment. In this context, such promoters may be considered exogenous promoters. Examples of appropriate promoters are described elsewhere.

### Vectors and Modified Cells

In one aspect, the invention provides a vector that comprises a nucleic acid sequence described herein (e.g. a nucleic acid sequence that encodes a peptide comprising an amino acid sequence of SEQ ID NO:1).

A vector system is also provided which includes a nucleic acid composition described herein. The vector system may have one or more vectors. As discussed previously, the binding protein components that are encoded by the nucleic acid composition may be encoded by one or more nucleic acid sequences in the nucleic acid composition. In examples where all of the binding protein components are encoded by a single nucleic acid sequence, the nucleic acid sequence may be present within a single vector (and thus the vector system described herein may comprise of one vector only). In examples where the binding protein components are encoded by two or more nucleic acid sequences (wherein the plurality of nucleic acid sequences, together, encode all of the components of the binding protein) these two or more nucleic acid sequences may be present within one vector (e.g. in different open reading frames of the vector), or may be distributed over two or more vectors. In this example, the vector system will comprise a plurality of distinct vectors (i.e. vectors with different nucleotide sequences).

Accordingly, in one example, a vector system is provided, comprising a nucleic acid composition described herein.

Any appropriate vector can be used. By way of example only, the vector may be a plasmid, a cosmid, or a viral vector, such as a retroviral vector or a lentiviral vector. Adenovirus, adeno-associated virus, vaccinia virus, canary poxvirus, herpes virus, minicircle vectors and naked (synthetic) DNA/RNA may also be used (for details on minicircle vectors, see for example non-viral Sleeping Beauty transposition from minicircle vectors as published by Monjezi et al., Leukemia 2016). Alternatively, single stranded or double stranded DNA or RNA can be used to transfect lymphocytes with a TCR of interest (see Roth et al 2018 Nature vol 559; page 405).

In one example, the vector is a plasmid, a viral vector, or a cosmid, optionally wherein the vector is selected from the group consisting of a retrovirus, lentivirus, adeno-associated virus, adenovirus, vaccinia virus, canary poxvirus, herpes virus, minicircle vector and synthetic DNA or RNA.

As used herein, the term “vector” refers to a nucleic acid sequence capable of transporting another nucleic acid sequence to which it has been operably linked. The vector can be capable of autonomous replication or it can integrate into a host DNA. The vector may include restriction enzyme sites for insertion of recombinant DNA and may include one or more selectable markers or suicide genes. The vector can be a nucleic acid sequence in the form of a plasmid, a bacteriophage or a cosmid. Preferably the vector is suitable for expression in a cell (i.e. the vector is an “expression vector”). Preferably, the vector is suitable for expression in a human antigen presenting cell. Preferably, the vector is suitable for expression in a human T cell such as a CD8⁺ T cell or CD4⁺ T cell, or stem cell, iPS cell, or NK cell. In certain aspects, the vector is a viral vector, such as a retroviral vector, a lentiviral vector or an adeno-associated vector. Optionally, the vector is selected from the group consisting of an adenovirus, vaccinia virus, canary poxvirus, herpes virus, minicircle vector and synthetic DNA or synthetic RNA.

Preferably the (expression) vector is capable of propagation in a host cell and is stably transmitted to future generations. Suitable vectors and expression vectors are well known in the art.

The vector may comprise regulatory sequences. Optionally, the vector comprises the nucleic acid sequence of interest operably linked to a promoter. The promoter may be one that is not naturally found in the host cell (e.g. it may be an exogenous promoter). “Operably linked” refers to a single or a combination of the below-described control elements together with a coding sequence in a functional relationship with one another, for example, in a linked relationship so as to direct expression of the coding sequence.

A person of skill in the art will be well aware of the molecular techniques available for the preparation of (expression) vectors and how the (expression) vectors may be transduced or transfected into an appropriate host cell (thereby generating a modified cell as described herein). The (expression) vector of the present invention can be introduced into cells by conventional techniques such as transformation, transfection or transduction.

“Transformation”, “transfection” and “transduction” refer generally to techniques for introducing foreign (exogenous) nucleic acid sequences into a host cell, and therefore encompass methods such as electroporation, microinjection, gene gun delivery, transduction with retroviral, lentiviral or adeno-associated vectors, lipofection, superfection etc. The specific method used typically depends on both the type of vector and the cell. Appropriate methods for introducing nucleic acid sequences and vectors into host cells such as human cells are well known in the art; see for example Sambrook et al (1989) Molecular Cloning, A Laboratory Manual, Cold Spring Harbor Laboratory, Cold Spring Harbor, N.Y; Ausubel et al (1987) Current Protocols in Molecular Biology, John Wiley and Sons, Inc., NY; Cohen et al (1972) Proc. Natl. Acad. Sci. USA 69, 2110; Luchansky et al (1988) Mol. Microbiol. 2, 637-64. Further conventional methods that are suitable for preparing expression vectors and introducing them into appropriate host cells are described in detail in WO02016/071758 for example.

It is understood that it some embodiments, the host cell is contacted with the vector (e.g. viral vector) in vitro, ex vivo, and in some embodiments, the host cell is contacted with the vector (e.g. viral vector) in vivo.

The term “host cell” includes any cell into which the nucleic acid sequences or vectors described herein may be introduced (e.g. transduced). Once a nucleic acid molecule or vector has been introduced into the cell, it may be referred to as a “modified cell” herein. Once the nucleic acid molecule or vector is introduced into the host cell, the resultant modified cell should be capable of expressing the encoded polypeptide (and e.g. correctly localising the encoded polypeptide such as an encoded binding protein for its intended function e.g. transporting the encoded binding protein to the cell surface).

The nucleic acid composition or vector system may be introduced into the cell using any conventional method known in the art. For example, the nucleic acid composition or vector system may be introduced using CRISPR technology. Insertion of the nucleic acid sequences at the endogenous TCR locus by engineering with CRISPR/Cas9 and homologous directed repair (HDR) or non-homologous end joining (NHEJ) is therefore encompassed. Other conventional methods such as transfection, transduction or transformation of the cell may also be used.

The term “modified cell” refers to a genetically altered (e.g. transformed, transduced or transfected) cell. The modified cell includes at least one exogenous nucleic acid sequence (i.e. a nucleic acid sequence that is not naturally found in the host cell). The term refers to the particular subject cell and also to the progeny or potential progeny of such a cell. Because certain modifications may occur in succeeding generations due to either mutation or environmental influences, such progeny may not, in fact, be identical to the parent cell, but are still included within the scope of the term as used herein.

In one example, a modified cell comprises a nucleic acid composition or a vector system provided herein. In another example, a modified cell comprises a nucleic acid comprising a nucleic acid sequence encoding a peptide provided herein. Any suitable nucleic acid sequence encoding the peptide may be used and can be selected accordingly by the skilled person.

Although the host cell (and thus the modified cell) may be a bacterial cell, it is typically a eukaryotic cell, and particularly a human cell which can overexpress the antigen for uptake by antigen presenting cells (APCs), more particularly an antigen presenting cell, such as dendritic cells (DCs), B cells, monocytes, macrophages. The host cell (and thus the modified cell) may be an autologous cell, which refers to a cell derived from the same individual to which it is later administered. In other words, the host cell (and thus the modified cell) may be a cel from a subject to be treated. Suitably, the host cell (and thus the modified cell) may be isolated from a blood sample e.g. by leukaphoresis. The modified cell is typically a human cell. The host cell (and thus the modified cell) may be any cell that is able to confer anti-tumour immunity after TCR gene transfer. Non limiting examples of appropriate cells include autologous or allogeneic CD8 T cells, CD4 T cells, Natural Killer (NK) cells, NKT cells, gamma-delta T cells, inducible pluripotent stem cells (iPSCs), hematopoietic stem cells or other progenitor cells and any other autologous or allogeneic cell or cell line (NK-92 for example or T cell lines) that is able to confer anti-tumor immunity after TCR gene transfer.

Accordingly, in one example the modified cell is selected from the group consisting of a CD8 T cell, a CD4 T cell, an NK cell, an NK-T cell, a gamma-delta T cell, an innate lymphoid cell (ILC), a hematopoietic stem cell, an inducible pluripotent stem cell, a progenitor cell, a T cell line and a NK-92 cell line.

In the context of the methods of treatment described herein, the host cell (and thus the modified cell) that is to be administered to the subject can either be autologous or allogeneic.

Advantageously, the modified cell is capable of expressing the polypeptide encoded by the nucleic acid sequence or vector described herein such that the modified cell provides an immunotherapy that specifically targets cancerous cells or virally infected cells associated with impaired HLA class I antigen presentation and this can be used to treat or prevent cancer or viral infections associated with impaired HLA class I antigen presentation. More details on this use are given below.

### Methods for Preparing Peptides

As described above, the peptide according to the present invention may be a natural peptide or a synthetic peptide. In another aspect, the peptide of the present invention may be modified.

Methods of preparing a peptide of the invention are also provided herein. In one aspect, the methods of preparing a peptide of the invention provided herein may be natural methods. In another aspect, the method of preparing a peptide of the invention may be synthetic methods. Alternatively, the method of preparing a peptide of the invention may comprise natural and synthetic methods.

The methods of preparing a peptide of the invention provided herein may be natural methods. Such methods comprise cultivating a modified cell that has been transformed, transfected or transduced with a nucleic acid (e.g. vector) encoding the peptide of interest in a culture medium and separating the peptide from the culture medium or from the modified cell lysate after cell lysis. In this context, the modified cell is used to express the peptide of interest. Examples of such cells include, but are not limited to, bacterial cells, e.g. E. coli, and eukaryotic cells, e.g., yeast cells, animal cells or plant cells. In one example the cells are mammalian, e.g., human, CHO, HEK293T, PER.C6, NS0, myeloma or hybridoma cells. Dendritic cells and dendritic cell lines are particularly preferred.

Typically, as described above, the nucleic acid encoding the peptide of interest is present within a vector, such as an expression vector. In some examples, an appropriate secretion signal can be integrated in the vector, so that the peptide encoded by the nucleic acid will be directed, for example towards the lumen of the endoplasmic reticulum, towards the periplasmic space, on the membrane or towards the extracellular environment. The choice of appropriate secretion signal may facilitate subsequent protein purification. Selection of appropriate secretion signals are well within the capabilities of a person with average skill in the art. Typically, the choice of a culture medium depends in particular on the choice of the cell type and/or the cell line that is used to express the peptide of interest. A person of skill in the art is well aware of suitable culture media, which are appropriate for a selected cell type and/or cell line.

The cells are cultivated in the appropriate culture medium for a period that is sufficient to induce expression of the encoded peptide. Suitable time periods and conditions for culturing cells are well known in the art and depend on the specific cell type and/or cell line that is used.

Once the peptide is expressed by the cells, it may be purified using standard methods. For example, commercially available kits and/or reagents for protein extraction may be used, for example BugBuster™ from Novagen. Alternative standard methods such as affinity chromatography, ion-exchange chromatography, hydrophobic interaction chromatography, and immunoaffinity methods may also be used.

Alternatively, the peptides of the invention may be prepared by synthetic methods. Such methods are well described in the literature. Non-limiting examples include liquid phase peptide synthesis methods or solid peptide synthesis methods, e.g. solid peptide synthesis methods according to Merrifield, t-Boc solid-phase peptide synthesis, Fmoc solid-phase peptide synthesis, BOP (Benzotriazole-1-yl-oxy-tris-(dimethylamino)-phosphonium hexafluorophosphate) based solid-phase peptide synthesis, etc.

### Peptide-Loaded Cells

A cell loaded with a peptide described herein is also provided. These cells may advantageously be used in the therapeutic methods described below.

As used herein a cell “loaded” with peptide refers to a cell wherein the peptide is in association with an MHC (major histocompatibility complex) on the surface of the cell. Typically, cells loaded with peptide do not express the peptide themselves, but present exogenous peptides in the context of MHC. Cells may be pulsed with exogenous peptide in order to “load” them with peptide. Cells loaded with peptides may therefore also be referred to as cells comprising the peptide of interest (e.g. exogenous peptide), wherein the peptide of interest is part of an MHC complex on the surface of the cell. In other words, such cells comprise extracellular (or cell surface) MHC complexed with the peptide of interest. The presence of the peptide within the MHC of an antigen presenting cell is referred to as “antigen presentation” herein. Antigen presentation is the expression of antigen molecules on the surface of a macrophage or other antigen-presenting cell in association with MHC class II molecules when the antigen is being presented to a CD4+ helper T cell or in association with MHC class I molecules when presentation is to CD8+ cytotoxic T cells.

Alternatively, or additionally, the cell may be a modified cell transformed, transfected or transduced with a nucleic acid comprising a nucleic acid sequence encoding a peptide described herein. Such a modified cell expresses the peptide and is therefore also capable of loading the same cell or other cells with the peptide.

Cells loaded with the peptide as defined herein may be cells from a subject to be treated. In particular, they may be cells that have been isolated from a subject to be treated. Alternatively, cell lines, e.g. antigen presenting cell lines, may also be used.

Preferably, the cell loaded with the peptide as defined herein is an antigen-presenting cell (APC). Preferably, the antigen presenting cell is selected from the group consisting of a dendritic cell (DC), a macrophage, a monocyte, a B-cell and a synthetic form of antigen presenting cell. Dendritic cells, in particular dendritic cells (conventional and/or plasmacytoid) isolated from a subject to be treated, are most preferred.

Methods to isolate antigen-presenting cells, in particular dendritic cells, from a subject are known to the skilled person. They include harvesting monocytes or hematopoietic stem cells from bone marrow, cord blood, or peripheral blood. They also include the use of embryonic stem (ES) cells and induced pluripotent stem cells (iPS). Antigen presenting cells, in particular dendritic cells or their precursors, can be enriched by methods including elutriation and magnetic bead based separation, which may involve enrichment for CD14+ precursor cells.

Methods to load the complex as defined herein into the cells, preferably into the above-mentioned antigen presenting cells, more preferably into dendritic cells, and further to prepare such cells before administration to a subject are known to one skilled in the art. For example, preparation of dendritic cells can include their culture or differentiation using cytokines that may include for example GM-CSF and IL-4. Dendritic cell lines may also be employed.

Loading of the peptide into the cells, preferably into APC, more preferably into the dendritic cells, can involve co-incubation of the peptide with the cells in culture. Further culture of the cells, e.g. the dendritic cells, thus loaded to induce efficient maturation can include addition of cytokines including IL-1s, IL-6, TNFα, PGE2, IFNα, and adjuvants. Appropriate methods and reagents are well known to a person of skill in the art.

### Pharmaceutical Compositions

A pharmaceutical composition is provided comprising a) a peptide, b) a nucleic acid and/or a vector, c) a complex or a binding agent or d) a cell described herein, and a pharmaceutically acceptable excipient, adjuvant, diluent and/or carrier.

For the avoidance of doubt, any one of a), b) or c) may be present in the pharmaceutical composition by virtue of them being encoded or expressed (as appropriate) by a cell that is present within the pharmaceutical composition. As an example, any one of b) or c) may be encoded by a cell that is combined with a pharmaceutically acceptable excipient, adjuvant, diluent and/or carrier to generate the pharmaceutical composition; or any of a) or b) may be expressed by a cell that is combined with a pharmaceutically acceptable excipient, adjuvant, diluent and/or carrier to generate the pharmaceutical composition. More details on this are provided below.

A particularly suitable composition may be selected based on the HLA serotype of the human subject, as described in detail elsewhere herein.

A nucleic acid, vector, complex, cell, binding agent, and/or peptide as described herein may therefore be provided as part of a pharmaceutical composition. Advantageously, such compositions may be administered to a human subject in order to treat or prevent a cancer or viral infection associated with impaired HLA class I antigen presentation (e.g. by inducing or enhancing a specific immune response to such cancerous or virally infected cells).

The terms “pharmaceutical composition” and “composition” are used interchangeably herein, unless the context specifically requires otherwise.

A pharmaceutical composition may comprise a nucleic acid sequence, vector, complex, binding agent, cell, and/or peptide described herein along with a pharmaceutically acceptable excipient, adjuvant, diluent and/or carrier.

For the avoidance of doubt, the nucleic acid sequence, vector, complex, binding agent and/or peptide may be present in the pharmaceutical composition as part of a cell. In other words, the nucleic acid sequence or vector may be incorporated into a cell; or the binding agent, complex or peptide may be expressed by a cell. The cell may be any suitable cell, for example a bacterial cell, or a eukaryotic cell such as a mammalian cell e.g. a dendritic cell (DC)—(in such cases, the mammalian cell is typically an ex vivo cell). A pharmaceutical composition comprising a nucleic acid sequence, vector, complex, binding agent and/or peptide described herein therefore encompasses a pharmaceutical composition comprising a cell (e.g. a bacterial cell, DC, etc) that encodes the nucleic acid sequence or vector, or is capable of expressing the peptide, complex or binding agent.

Suitably, the cell (e.g. bacterial cell, DC, etc) may be a cell that has been modified to introduce into the cell the appropriate nucleic acid sequence/vector (e.g. by transduction, transfection or transformation) such that the modified cell encodes the nucleic acid sequence/vector and becomes capable of expressing the nucleic acid sequence, vector, complex, peptide or binding agent of interest. Such cells may be combined with a pharmaceutically acceptable excipient, adjuvant, diluent and/or carrier to generate a pharmaceutical composition of the invention. The cell may modified ex vivo. For example, it may be an autologous cell that has been derived from the subject that is to be treated with the pharmaceutical composition described herein (e.g. for treating or preventing a cancer or viral infection associated with impaired HLA class I antigen presentation). The cells may be modified ex vivo to introduce e.g. the nucleic acid sequence, or vector into the cell such that the modified cell encodes the nucleic acid sequence/vector and becomes capable of expressing the nucleic acid sequence or vector to generate the peptide, complex or binding agent of interest. The modified cells may then be administered to the subject as a pharmaceutical composition.

Compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected nucleic acid sequence, vector, cell, binding agent or peptide without causing any undesirable biological effects or interacting in a deleterious manner with any of the other components of the pharmaceutical composition in which it is contained.

Excipients are natural or synthetic substances formulated alongside an active ingredient (e.g. a nucleic acid sequence, a nucleic acid composition, vector or vector system, modified cell, or isolated nucleic acid as provided herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include saline, water, aqueous dextrose, glycerol, ethanol, and the like. Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art. In a separate embodiment of the invention, an excipient does not include water.

A pharmaceutical composition may comprise an immune stimulatory compound as disclosed elsewhere herein.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

The pharmaceutical compositions described herein may be administered to a subject as a monotherapy or as part of a combination therapy. For example, combinations of the vaccines described herein with an immune checkpoint inhibitor or other immunomodulatory compounds may also be particularly useful for targeting immune-escaped TAP-deficient cancers, as has been shown for the combination of cancer-virus vaccination with PD-1 blockade.

Accordingly, the pharmaceutical compositions provided herein may be used in combination with an immune checkpoint inhibitor that blocks PD-1, CTLA-4, PD-L1, TIM3, TIGIT, VISTA, NKG2A or LAG-3.

In a particular example, the pharmaceutical compositions provided herein may be used in combination with an immune check point inhibitor that is selected from an antibody that blocks CTLA-1 OR PD-1/PD-L1 OR NKG2A. Such antibodies are showing real promise in the clinic in the treatment of patients with a variety of malignancies.

As a specific example, the immune checkpoint inhibitor may be an inhibitor of PD-1 and/or PD-L1 activity. In other words, the immune checkpoint inhibitor may result in PD-1 or PD-L1 blockade. The inhibitor of PD-1 and/or PD-L1 activity may be e.g. an antibody that blocks PD-L1 binding to PD1 (or vice versa).

Administration of the pharmaceutical composition and the immune checkpoint inhibitor may be in any order. Preferably, the pharmaceutical composition is administered at the same time or after the immune checkpoint inhibitor. Alternatively, the pharmaceutical composition is administered at the same time or before the immune checkpoint inhibitor.

A pharmaceutical composition described herein (comprising a nucleic acid, a nucleic acid composition, a complex, or a cell according to the invention) and a vaccine or peptide vaccine described herein are provided for use as a medicament. Suitably, the pharmaceutical composition described herein and the vaccine or peptide vaccine described herein may be combined for use as a medicament. Suitably, the combination may comprise administering the vaccine or peptide vaccine prior to, during, and/or after administration of the pharmaceutical composition.

### Treatment of a Subject

Pharmaceutical compositions or vaccines described herein may advantageously be used as a medicament. The compositions or vaccines may be used to treat or prevent a pre-cancer, a cancer or a viral infection associated with impaired HLA class I antigen presentation in a human subject. Preferably, the human subject is positive for HLA-A*02, such as HLA-A*02:01.

The term “pre-cancer” or “pre-cancerous” refers to a condition or a growth that typically precedes or develops into a cancer. A “pre-cancerous” growth or “pre-cancer” will have cells that are characterized by abnormal cell cycle regulation, proliferation, or differentiation, which can be determined by markers of cell cycle regulation, cellular proliferation, or differentiation.

The method of treatment or prevention of a pre-cancer, a cancer or a viral infection associated with impaired HLA class I antigen presentation described herein results in an induced or enhanced immune response (e.g. a cell mediated response) in the subject (e.g. a targeted immune response to cancerous or virally infected cells that present the HLA-A restricted peptide). The phrase “induced or enhanced immune response” refers to an increase in the immune response (e.g. a cell mediated immune response such as a T cell mediated immune response) of the subject during or after treatment compared to their immune response prior to treatment. An “induced or enhanced” immune response therefore encompasses any measurable increase in the immune response that is directly or indirectly targeted to the pre-cancer, cancer or viral infection being treated (or prevented).

Compositions of the invention may be used to treat or prevent a cancer associated with impaired HLA class I antigen presentation. A person of skill in the art will be fully aware of cancers that are associated with impaired HLA class I antigen presentation and thus may be treated in accordance with the invention.

In another example, the pharmaceutical composition or vaccine may be for use in stimulating a cell mediated immune response to a target cell population or tissue in a human subject. The pharmaceutical composition or vaccine may also be for use in providing anti-tumor immunity to a human subject.

Suitably, the cancer is cancer with impaired peptide processing machinery. In one example, the cancer is melanoma. In another example, the cancer is lung cancer.

Compositions or vaccines of the invention may be used to treat or prevent a pre-cancer associated with impaired HLA class I antigen presentation. A person of skill in the art will be fully aware of pre-cancers that are associated with impaired HLA class I antigen presentation and thus may be treated in accordance with the invention.

Suitably, the pre-cancer is a pre-cancer with impaired peptide processing machinery.

Compositions or vaccines of the invention may also be used to treat or prevent a viral infection associated with impaired HLA class I antigen presentation. A person of skill in the art will be fully aware of viral infections that are associated with impaired HLA class I antigen presentation and thus may be treated in accordance with the invention.

As used herein, a pre-cancer, cancer or viral infection “associated with impaired HLA class I antigen presentation” refers to a pre-cancer, cancer or viral infection that results in a change in the HLA class I antigen presentation pathway in the pre-cancerous, cancerous or virally infected cell, which results in a reduction in HLA class I antigen presentation in these cells. In this context, a reduction encompasses a decrease of at least 10%, at least 20%, at least 30%, at least 40%, at least 50%, at least 60% etc in the presentation of non-TEIPP HLA class I-restricted antigens at the cell surface of these cells (at a given time) compared to control cells (e.g. derived from the same subject that are not pre-cancerous, not cancerous and are not virally infected).

There are several molecular pathways that may be altered in the pre-cancerous, cancerous or virally infected cell to impair HLA class I antigen presentation. By way of example, it is known that 1-2% of melanomas have deleterious mutations in TAP1 or TAP2, and that a high frequency of metastatic melanomas display low TAP1 expression due to epigenetic silencing (Ritter et al., 2017; Setiadi et al., 2007; Garrido et al., 2016). TAP downregulation has also been observed in lung cancer specimens (“Different Expression Levels of the TAP Peptide Transporter Lead to Recognition of Different Antigenic Peptides by Tumor-Specific CTL”, A. Durgeau, F. El Hage, I. Vergnon, P. Validire, V. de Montpréville, B. Besse, J. Soria, T. van Hall and F. Mami-Chouaib, J Immunol Dec. 1, 2011, 187 (11) 5532-5539, “Loss of antigen-presenting molecules (MHC class I and TAP-1) in lung cancer”, Korkolopoulou P., L. Kaklamanis, F. Pezzella, A. L. Harris, K. C. Gatter. 1996. Br. J. Cancer 73: 148-153., and “Restoration of the expression of transporters associated with antigen processing in lung carcinoma increases tumor-specific immune responses and survival”, Lou Y., T. Z. Vitalis, G. Basha, B. Cai, S. S. Chen, K. B. Choi, A. P. Jeffries, W. M. Elliott, D. Atkins, B. Seliger, W. A. Jefferies. 2005. Cancer Res. 65: 7926-7933).

A pre-cancer, cancer or viral infection associated with impaired HLA class I antigen presentation may therefore be a pre-cancer, cancer or viral infection wherein the tumor cells or infected cells have a mutated TAP1 or TAP2 gene. In one example, the mutation reduces TAP1 or TAP2 expression (such that the pre-tumor cell, tumor cell or virally infected cell has low TAP1 or TAP2 expression). In another example, the mutation reduces TAP1 or TAP2 activity in the cell (such that the pre-tumor cell, tumor cell or virally infected cell has reduced/low TAP1 or TAP2 activity). In other example, the mutation reduces TAP1 or TAP2 protein levels in the cell (e.g. the pre-tumor cell, the tumor cell or virally infected cell has reduced/low TAP1 or TAP2 protein expression and/or reduced/low TAP1 or TAP2 protein stability).

TAP1 or TAP2 expression may also be reduced/low in a pre-cancerous cell, cancerous cell or virally infected cell due to epigenetic silencing. Methods for detecting TAP1 or TAP2 epigenetic silencing are well known in the art. TAP1 or TAP2 expression, activity, protein level and/or protein stability may also be reduced/low in a pre-cancerous cell, cancerous cell or virally infected cell for other reasons than mutation of the TAP1 or TAP2 genes (e.g. due to the pre-cancer/cancer/virus altering the molecular machinery and pathways of the cell).

The pre-cancer, cancer or viral infection may therefore be a pre-cancer, cancer or viral infection associated with reduced (or low) TAP1 or TAP2 protein expression, activity, level, or stability.

Methods for determining the presence of mutations in TAP1 or TAP2 are well known in the art. Furthermore, methods for determining TAP1 or TAP2 expression levels, TAP1 or TAP2 activity levels, TAP1 or TAP2 protein levels, and TAP1 or TAP2 protein stability are well known in the art. For example, the expression level may be detected by measuring mRNA (e.g. using Northern blot analysis, RNA probes (e.g. using spatial transcriptomics (e.g. as offered by NanoString) or using in situ hybridization (e.g. RNAscope®)), or rtPCR). The level of protein may be detected using TAP1 or TAP2 specific antibodies (e.g. with a detectable label) and methods such as enzyme linked immunosorbent assays (ELISAs), immunoprecipitation, immunofluorescence, enzyme immunoassay (EIA), radioimmunoassay (RIA), spatial proteomics (e.g. as offered by NanoString), and Western blot analysis may be used. Other standard methods for determining these parameters are well known in the art.

As stated above, the pre-cancer, cancer or viral infection may be a pre-cancer, cancer or viral infection associated with reduced (or low) TAP1 or TAP2 protein expression, activity, level, or stability.

As used herein, “reduced (or low) TAP1 or TAP2 protein expression, activity, level, or stability” refers to a decrease in the protein expression activity, level, or stability compared to a control or a reference level (e.g. at least a 5%, 10%, 15%, 20%, 30%, 40%, 50%, 60%, 70%, 80%, 90%, 100% decrease). As used herein “reference level” or “control”, refers to a cell sample having a normal level of TAP1 or TAP2 protein expression, activity, level, or stability, for example a sample from a healthy subject not having or suspected of having pre-cancer, cancer or a viral infection or alternatively a cell sample from the same subject being tested, where the control or reference level cell sample is not (and is not suspected of being) pre-cancerous, cancerous or virally infected. Alternatively, the reference level may be a TAP1 or TAP2 protein expression, activity, level, or stability value from a reference database, which may be used to generate a pre-determined cut off value, i.e. a diagnostic score that is statistically predictive of a symptom or disease or lack thereof or may be a pre-determined reference level based on a standard population sample, or alternatively, a pre-determined reference level based on a subject's base line level of expression, i.e. prior to developing or being suspected of having pre-cancer, cancer or a viral infection. For example, reduced or low protein expression may be determined using immunohistochemistry, using anti-TAP1 or anti-TAP2 antibodies, such as the anti-TAP1 Antibody, clone mAb 148.3 (MABF125 EMD Millipore). In one example, evaluation of TAP1 or TAP2 normal level protein expression in a sample as compared to reduced or low level of expression is determined by the ‘De Ruiter’ evaluation method. For example, in such a method the sample is a pre-cancer, pre-tumour, cancer or tumour sample. Alternatively, where the sample is a viral sample, the presence of immune modulatory viral gene products, for example CMV, HSV or BVS, reduces the expression and/or activity of TAP function and the presence of such gene products can be used as a marker of reduced or low TAP1 or TAP2 expression and/or activity.

Other molecular pathways that may be altered in the pre-cancerous, cancerous or virally infected cell to impair HLA class I antigen presentation include for example a deficiency in tapasin (a chaperone protein involved in TAP-mediated peptide loading of MHC class I molecules) and inhibition of proteasome-mediated degradation of proteins into peptides for MHC class I presentation (see for example US2009/0220534 for more details).

As used herein, the terms “treat”, “treating” and “treatment” are taken to include an intervention performed with the intention of preventing the development or altering the pathology of a condition, disorder or symptom (i.e. in this case a pre-cancer, cancer or viral infection associated with impaired HLA class I antigen presentation). Accordingly, “treatment” refers to both therapeutic treatment and prophylactic or preventative measures, wherein the object is to prevent or slow down (lessen) the targeted condition, disorder or symptom. “Treatment” therefore encompasses a reduction, slowing or inhibition of the amount or concentration of pre-malignant, malignant or virally infected cells, for example as measured in a sample obtained from the subject, of at least 5%, 10%, 20%, 30%, 40%, 50%, 60%, 70%, 80%, 90% or 100% when compared to the amount or concentration of malignant cells (or pre-malignant cells or virally infected cells) before treatment. Methods of measuring the amount or concentration of malignant cells (or pre-malignant cells or virally infected cells) include, for example, qRT-PCR, and quantification of specific biomarkers in a sample obtained from the subject.

As used here in the term “subject” refers to an individual, e.g., a human, having or at risk of having a specified condition, disorder or symptom. The subject may be a patient i.e. a subject in need of treatment in accordance with the invention. The subject may have received treatment for the condition, disorder or symptom. Alternatively, the subject has not been treated prior to treatment in accordance with the present invention. Preferably, the subject is a human subject, preferably a HLA*02 positive human subject, more preferably a HLA*02:01 positive human subject.

The compositions or vaccines described herein can be administered to the subject by any conventional route, including injection or by gradual infusion over time. The administration may, for example, be by infusion or by intramuscular, intravascular, intracavity, intracerebral, intralesional, rectal, subcutaneous, intradermal, epidural, intrathecal, percutaneous administration.

The compositions or vaccines described herein may be in any form suitable for the above modes of administration. For example, compositions comprising cells may be in any form suitable for infusion. As further examples, suitable forms for parenteral injection (including, subcutaneous, intramuscular, intravascular or infusion) include a sterile solution, suspension or emulsion; suitable forms for topical administration include an ointment or cream; and suitable forms for rectal administration include a suppository. Alternatively, the route of administration may be by direct injection into the target area, or by regional delivery or by local delivery. The identification of suitable dosages of the compositions of the invention is well within the routine capabilities of a person of skill in the art.

Advantageously, the compositions of the invention may be formulated for use as a vaccine (e.g. a composition comprising a peptide, wherein the peptide comprises the amino acid sequence of SEQ ID NO: 1 (or the corresponding nucleic acid sequence or vector) may be formulated as a pharmaceutical composition that is suitable for use as a (peptide) vaccine). Alternatively, compositions comprising cells may also be formulated as pharmaceutical compositions that are suitable for use as a vaccine. Suitable cell, binding agent (e.g. antibody), peptide and nucleic acid vaccine formulations are well known in the art.

Advantageously, the compositions described herein may be formulated for use in T cell receptor (TCR) gene transfer, an approach that is rapid, reliable and capable of generating large quantities of T cells with specificity for TIMP3 antigenic peptides (e.g. the peptides shown in any one of SEQ ID NOs: 1-27, 110 or 111, particularly the peptide of SEQ ID NO: 2), regardless of the patient's pre-existing immune repertoire. Using TCR gene transfer, modified cells suitable for infusion may be generated within a few days.

The pharmaceutical composition or vaccine is preferably for, and therefore formulated to be suitable for, administration to a subject, preferably a human or animal subject. Preferably, the administration is parenteral, e.g. intravenous, subcutaneous, intramuscular, intradermal intracutaneous and/or intratumoral administration, i.e. by injection.

Preferably, the pharmaceutical composition or vaccine comprises or consists of an amount of active ingredient (e.g. nucleic acid sequence, peptide, vector, binding agent, or cell) that constitutes a pharmaceutical dosage unit. A pharmaceutical dosage unit is defined herein as the amount of active ingredients (i.e. the total amount of peptide in a peptide-based vaccine for example) that is applied to a subject at a given time point. A pharmaceutical dosage unit may be applied to a subject in a single volume, i.e. a single shot, or may be applied in 2, 3, 4, 5 or more separate volumes or shots that are applied preferably at different locations of the body, for instance in the right and the left limb. It is to be understood herein that the separate volumes of a pharmaceutical dosage may differ in composition, i.e. may comprise different kinds or composition of active ingredients and/or adjuvants.

A single injection volume or shot (i.e. volume applied on one location at a certain time point), comprising a total pharmaceutical dosage, or part thereof in case multiple shots applied at substantially the same time point, may between 100 and 2 mL, or between 100 and 1 mL. The single injection volume may be 100 μl, 200 μl, 300 μl, 400 μl, 500 μl, 600 μl, 700 μl, 800 μl, 900 μl, 1 mL, 1.1 mL, 1.2 mL, 1.3 mL, 1.4 mL, 1.5 mL, 1.6 mL, 1.7 mL, 1.8 mL, 1.9 mL, 2 mL, 3 mL or any value in between.

The pharmaceutical dosage unit, or total amount of active ingredient applied to a subject at a given time point will depend on the type of vaccine (e.g. peptide, cell, nucleic acid etc). As an example, the pharmaceutical dosage unit, or total amount of peptide applied to a subject at a given time point, either in a single or in multiple injections at a certain time point, comprises an amount of peptide in the range from 0.1 μg to 20 mg, such as about 0.1 μg, 0.5 μg, 1 μg, 5 μg, 10 μg, 15 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 650 μg, 700 μg, 750 μg, 800 μg, 850 μg, 900 μg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg or about 20 mg or any value in between. Preferred ranges of pharmaceutical dosage units are from 0.1 μg to 20 mg, 1 μg to 10 mg, 10 μg to 5 mg, 0.5 mg to 2 mg, 0.5 mg to 10 mg or 1 mg to 5 mg or 2 to 4 mg.

The compositions or vaccines described herein are for administration in an effective amount. An “effective amount” is an amount that alone, or together with further doses, produces the desired (therapeutic or non-therapeutic) response. The effective amount to be used will depend, for example, upon the therapeutic (or non-therapeutic) objectives, the route of administration, and the condition of the patient/subject. For example, the suitable dosage of the composition of the invention for a given patient/subject will be determined by the attending physician (or person administering the composition), taking into consideration various factors known to modify the action of the composition of the invention for example severity and type of haematological malignancy, body weight, sex, diet, time and route of administration, other medications and other relevant clinical factors. The dosages and schedules may be varied according to the particular condition, disorder or symptom the overall condition of the patient/subject. Effective dosages may be determined by either in vitro or in vivo methods. The pharmaceutical compositions of the present invention are advantageously presented in unit dosage form.

### Binding Agents

Binding agents are described herein that specifically bind to a peptide comprising (or consisting of) the amino acid sequence of SEQ ID NO: 1. The binding agent is useful in the prevention or treatment of a pre-cancer, cancer or viral infection associated with impaired HLA class I antigen presentation in a human subject. Also, binding agents are described herein that specifically bind to a peptide comprising (or consisting of) the amino acid sequence of any one of SEQ ID NOs: 2-21, preferably any one of SEQ ID NOs: 2-5, more preferably any one of SEQ ID NOs: 2-4 or any one of SEQ ID NO: 3-5.

The binding agent may specifically bind to an epitope within the amino acid sequence provided by SEQ ID NO: 1. The binding agent may specifically bind to an epitope within the amino acid sequence provided by any one of SEQ ID NOs: 2-21, preferably any one of SEQ ID NOs: 2-5, more preferably any one of SEQ ID NOs: 2-4 or any one of SEQ ID NO: 3-5. As used herein the term “epitope” refers to a site on a target molecule (in this case the recited peptide) to which a binding agent binds. Epitopes are groupings of molecules such as amino acids or sugar side chains and usually have specific structural characteristics, as well as specific charge characteristics. A single peptide (antigen) may have more than one epitope. Epitopes can be formed both from contiguous or adjacent noncontiguous residues (e.g., amino acid residues) of the target molecule. Epitopes formed from contiguous residues (e.g., amino acid residues) typically are also called linear epitopes. An epitope typically includes at least 5 and up to about 12 residues, mostly between 6 and 10 residues (e.g. amino acid residues). Epitopes may also be conformational (i.e. non-linear). In one example, the binding agent specifically binds to an epitope generated by the peptide itself. In another example, the binding agent (e.g. antibody) binds to an epitope generated by the combination of the peptide and the HLA molecule that presents it (i.e. an epitope that is generated when the peptide is presented on the cell surface by HLA class I, e.g. HLA*02:01).

The binding agent of the invention may be any appropriate binding agent that specifically binds to a peptide comprising (or consisting of) the amino acid sequence of SEQ ID NO: 1. The binding agent of the invention may be any appropriate binding agent that specifically binds to a peptide comprising (or consisting of) the amino acid sequence of any one of SEQ ID NOs: 2-21, preferably any one of SEQ ID NOs: 2-5, more preferably any one of SEQ ID NOs: 2-4 or any one of SEQ ID NO: 3-5.

An example of a suitable binding agent of the invention includes an HLA-A*02 molecule that specifically binds to the peptide comprising (or consisting of) the amino acid sequence of SEQ ID NO:1. Another example of a suitable binding agent of the invention includes an HLA-A*02:01, HLA-A*02:02, HLA-A*02:03, HLA-A*02:04, or HLA-A*02:09, preferably an HLA-A*02:01, molecule that specifically binds to the peptide comprising (or consisting of) the amino acid sequence of SEQ ID NO:1. Another example of a suitable binding agent of the invention includes an HLA-A*02 molecule that specifically binds to the peptide comprising (or consisting of) the amino acid sequence of any one of SEQ ID NOs: 2-21, preferably any one of SEQ ID NOs: 2-5, more preferably any one of SEQ ID NOs: 2-4 or any one of SEQ ID NO: 3-5. A further example of a suitable binding agent of the invention includes an HLA-A*02:01, HLA-A*02:02, HLA-A*02:03, HLA-A*02:04, or HLA-A*02:09, preferably an HLA-A*02:01, molecule that specifically binds to the peptide comprising (or consisting of) the amino acid sequence of any one of SEQ ID NOs: 2-21, preferably any one of SEQ ID NOs: 2-5, more preferably any one of SEQ ID NOs: 2-4 or any one of SEQ ID NO: 3-5. Such HLA-A*02, HLA-A*02:02, HLA-A*02:03, HLA-A*02:04, or HLA-A*02:09, or HLA-A*02:01 molecules may be useful, for example, as part of a multimeric structure for use in administration to a subject for stimulating T cells in the subject (for example in the form of a synthetic DC).

Accordingly, in one example the binding agent that specifically binds to a peptide comprising (or consisting of) the amino acid sequence of SEQ ID NO: 1 comprises an HLA-A*02 molecule. Typically, in this context, the HLA-A*02 molecule specifically binds to a peptide comprising (or consisting of) the amino acid sequence of SEQ ID NO: 1. In another example the binding agent that specifically binds to a peptide comprising (or consisting of) the amino acid sequence of SEQ ID NO: 1 comprises an HLA-A*02:01, HLA-A*02:02, HLA-A*02:03, HLA-A*02:04, or HLA-A*02:09, preferably an HLA-A*02:01, molecule. Typically, in this context, the HLA-A*02:01, HLA-A*02:02, HLA-A*02:03, HLA-A*02:04, or HLA-A*02:09, preferably an HLA-A*02:01, molecule specifically binds to a peptide comprising (or consisting of) the amino acid sequence of SEQ ID NO: 1.

In another example the binding agent that specifically binds to a peptide comprising (or consisting of) the amino acid sequence of any one of SEQ ID NOs: 2-21, preferably any one of SEQ ID NOs: 2-5, more preferably any one of SEQ ID NOs: 2-4 or any one of SEQ ID NO: 3-5, comprises an HLA-A*02 molecule. Typically, in this context, the HLA-A*02 molecule specifically binds to a peptide comprising (or consisting of) the amino acid sequence of any one of SEQ ID NOs: 2-21, preferably any one of SEQ ID NOs: 2-5, more preferably any one of SEQ ID NOs: 2-4 or any one of SEQ ID NO: 3-5. In another example the binding agent that specifically binds to a peptide comprising (or consisting of) the amino acid sequence of any one of SEQ ID NOs: 2-21, preferably any one of SEQ ID NOs: 2-5, more preferably any one of SEQ ID NOs: 2-4 or any one of SEQ ID NO: 3-5, comprises an HLA-A*02:01, HLA-A*02:02, HLA-A*02:03, HLA-A*02:04, or HLA-A*02:09, preferably an HLA-A*02:01, molecule. Typically, in this context, the HLA-A*02:01, HLA-A*02:02, HLA-A*02:03, HLA-A*02:04, or HLA-A*02:09, preferably an HLA-A*02:01, molecule specifically binds to a peptide comprising (or consisting of) the amino acid sequence of any one of SEQ ID NOs: 2-21, preferably any one of SEQ ID NOs: 2-5, more preferably any one of SEQ ID NOs: 2-4 or any one of SEQ ID NO: 3-5.

Such binding agents may be useful as pharmaceutical compositions, as described elsewhere herein.

Provided herein is a complex comprising: a) a peptide comprising the amino acid sequence of SEQ ID NO: 1, and b) a binding agent that specifically binds to a peptide comprising the amino acid sequence of SEQ ID NO: 1; optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWNGAEA (SEQ ID NO: 2), and b) a binding agent that specifically binds to a peptide comprising the amino acid sequence SLGDWNGAEA (SEQ ID NO: 2); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWNGAEV (SEQ ID NO: 3), and b) a binding agent that specifically binds to a peptide comprising the amino acid sequence SLGDWGAEV (SEQ ID NO: 3); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWNGAEI (SEQ ID NO: 4), and b) a binding agent that specifically binds to a peptide comprising the amino acid sequence SLGDVWGAEI (SEQ ID NO: 4); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWNGAEL (SEQ ID NO: 5), and b) a binding agent that specifically binds to a peptide comprising the amino acid sequence SLGDVWGAEL (SEQ ID NO: 5); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWNGAER (SEQ ID NO: 6), and b) a binding agent that specifically binds to a peptide comprising the amino acid sequence SLGDWGAER (SEQ ID NO: 6); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWNGAEN (SEQ ID NO: 7), and b) a binding agent that specifically binds to a peptide comprising the amino acid sequence SLGDWGAEN (SEQ ID NO: 7); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDVWGAED (SEQ ID NO: 8), and b) a binding agent that specifically binds to a peptide comprising the amino acid sequence SLGDWGAED (SEQ ID NO: 8); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWGAEC (SEQ ID NO: 9), and b) a binding agent that specifically binds to a peptide comprising the amino acid sequence SLGDWGAEC (SEQ ID NO: 9); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWGAEE (SEQ ID NO: 10), and b) a binding agent that specifically binds to a peptide comprising the amino acid sequence SLGDVWGAEE (SEQ ID NO: 10); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWNGAEQ (SEQ ID NO: 11), and b) a binding agent that specifically binds to a peptide comprising the amino acid sequence SLGDWGAEQ (SEQ ID NO: 11); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWNGAEG (SEQ ID NO: 12), and b) a binding agent that specifically binds to a peptide comprising the amino acid sequence SLGDWGAEG (SEQ ID NO: 12); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWGAEH (SEQ ID NO: 13), and b) a binding agent that specifically binds to a peptide comprising the amino acid sequence SLGDVWGAEH (SEQ ID NO: 13); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWGAEK (SEQ ID NO: 14), and b) a binding agent that specifically binds to a peptide comprising the amino acid sequence SLGDWGAEK (SEQ ID NO: 14); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDVWGAEM (SEQ ID NO: 15), and b) a binding agent that specifically binds to a peptide comprising the amino acid sequence SLGDWGAEM (SEQ ID NO: 15); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWGAEF (SEQ ID NO: 16), and b) a binding agent that specifically binds to a peptide comprising the amino acid sequence SLGDWGAEF (SEQ ID NO: 16); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWGAEP (SEQ ID NO: 17), and b) a binding agent that specifically binds to a peptide comprising the amino acid sequence SLGDVGAEP (SEQ ID NO: 17); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWGAES (SEQ ID NO: 18), and b) a binding agent that specifically binds to a peptide comprising the amino acid sequence SLGDWGAES (SEQ ID NO: 18); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWNGAET (SEQ ID NO: 19), and b) a binding agent that specifically binds to a peptide comprising the amino acid sequence SLGDWGAET (SEQ ID NO: 19); optionally wherein the binding agent is an HLA-A*02:01, HLA-A*02:02, HLA-A*02:03, HLA-A*02:04, or HLA-A*02:09, preferably an HLA-A*02:01, molecule.

Also provided herein is a complex comprising: a) a peptide comprising the amino acid sequence SLGDWNGAEW (SEQ ID NO: 20), and b) a binding agent that specifically binds to a peptide comprising the amino acid sequence SLGDWGAEW (SEQ ID NO: 20); optionally wherein the binding agent is an HLA-A*02:01, HLA-A*02:02, HLA-A*02:03, HLA-A*02:04, or HLA-A*02:09, preferably an HLA-A*02:01,molecule.

Also provided herein is a complex comprising: a) a peptide comprising the amino acid sequence SLGDWGAEY (SEQ ID NO: 21), and b) a binding agent that specifically binds to a peptide comprising the amino acid sequence SLGDWGAEY (SEQ ID NO: 21); optionally wherein the binding agent is an HLA-A*02:01, HLA-A*02:02, HLA-A*02:03, HLA-A*02:04, or HLA-A*02:09, preferably an HLA-A*02:01, molecule.

Preferably, the binding agent is complexed with or bound to the peptide.

The HLA-A*02:01, HLA-A*02:02, HLA-A*02:03, HLA-A*02:04, or HLA-A*02:09, preferably an HLA-A*02:01, molecules described herein may be useful, for example, as part of a multimeric structure for use in administration to a subject for stimulating T cells in the subject, for example in the form of an artificial antigen presenting cell (aAPC). aAPCs are synthetic structures loaded with tumor antigens. aAPCs are designed to mimic dendritic cells (DCs), hence also called synthetic DCs, with the goal of triggering an efficient and specific T cell response directed against a tumor in the context of cancer treatment.

In one example, the complex may be present in a cell, where a binding agent is loaded with the peptide according to the invention. In another example, a cell expresses the complex according to the invention. The cell may be an antigen presenting cell. The antigen presenting cell may be selected from a macrophage, dendritic cell, a monocyte, a B-cell or a synthetic form of antigen presenting cell.

Binding agents as described above may be useful as pharmaceutical compositions, as described elsewhere herein.

In one example, the binding agent is an isolated binding agent. As used herein, an “isolated binding agent” refers to a binding agent that is not in its natural environment. The binding agent may therefore be a recombinant binding agent, or the binding agent may be of synthetic origin (or alternatively, of natural original, but isolated from its natural environment). In the context of this disclosure, the natural environment of binding agents such as HLA-A2*02 or HLA-A2*02:01 molecules is within the human body. Accordingly, when the binding agent (e.g. HLA-A2*02 or HLA-A2*02:01 molecules) are present e.g. in a pharmaceutical composition (comprising adjuvants etc) they are considered to be in isolated form, as they are not in their natural environment.

As used herein the terms “specific binding” and “binding specifically” (or other equivalent terms) are used interchangeably to indicate that other biomolecules do not significantly bind to the region (that is specifically binding to the peptide of interest (e.g. the recited peptide comprising the amino acid sequence of SEQ ID NO:1)). In some embodiments, the level of binding to a biomolecule other than the peptide of interest results in a negligible (e.g., not determinable) binding affinity by means of ELISA or an affinity determination.

By “negligible binding” a binding is meant, which is at least about 85%, particularly at least about 90%, more particularly at least about 95%, even more particularly at least about 98%, but especially at least about 99% and up to 100% less than the binding to the peptide of interest (e.g. the recited peptide comprising the amino acid sequence of SEQ ID NO:1).

The binding affinity of the binding agent to the peptide of interest (e.g. the recited peptide comprising the amino acid sequence of SEQ ID NO:1) may be determined using a standard binding assay, such as surface plasmon resonance technique (BIAcore®, GE-Healthcare Uppsala, Sweden). The term “surface plasmon resonance,” as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51: 19-26; Jonsson, U., et al. (1991) Biotechniques 11:620-627; Johnsson, B., et al. (1995) J. Mol. Recognit. 8: 125-131; and Johnnson, B., et al. (1991) Anal. Biochem. 198:268-277.

Nucleic Acid Compositions that Encode Binding Protein Components

An isolated nucleic acid composition that encodes a TIMP metallopeptidase inhibitor 3 (TIMP3) antigen-specific binding protein having a TCR α chain variable (Vα) domain and a TCR β chain variable (Vβ) domain is provided herein, the composition comprising:
(a) a nucleic acid sequence that encodes a TCR Vα domain comprising a CDR3 amino acid sequence; and
(b) a nucleic acid sequence that encodes a TCR Vβ domain comprising a CDR3 amino acid sequence,
wherein the CDR3 sequences together specifically bind to a peptide comprising the amino acid sequence of SEQ ID NO:1 (e.g. when the peptide is complexed with HLA).

A TIMP3 antigen according to the invention comprises a peptide comprising an amino acid sequence selected from the group consisting of: SEQ ID NOs 1-21, preferably selected from the group consisting of: SEQ ID NOs 1-5 or SEQ ID NOs 2-5, more preferably selected from the group consisting of: SEQ ID NOs 2-4 or any one of SEQ ID NO: 3-5. Hence, the CDR3 sequences according to the invention preferably together specifically bind to a peptide comprising the amino acid sequence of any one of SEQ ID NOs 1-21, more preferably the amino acid sequence of any one of SEQ ID NOs 1-5 or any one of SEQ ID NOs 2-5, even more preferably the amino acid sequence of any one of SEQ ID NOs 2-4 or any one of SEQ ID NO: 3-5 (e.g. when the peptide is complexed with HLA).

As would be clear to a person of skill in the art, the CDR3 amino acid sequences described herein specifically bind to their target (in this case a peptide comprising the amino acid sequence of SEQ ID NO:1, for example a SLGDVNGAEA peptide, a SLGDVNGAEV peptide, a SLGDVNGAEI peptide or a SLGDVNGAEL peptide), when the target (i.e. the appropriate peptide) is presented in the context of HLA. The binding proteins (and CDR3 sequences specifically described herein) are therefore capable of specifically binding to an appropriate peptide:HLA complex. These complexes are described in more detail elsewhere herein.

The invention provides an isolated nucleic acid composition that encodes a binding protein comprising T cell receptor (TCR) components that specifically bind a TIMP3 antigen (e.g. to a peptide comprising the sequence of SEQ ID NO: 1, such as a SLGDVNGAEA peptide, a SLGDVNGAEV peptide, a SLGDVNGAEI peptide or a SLGDVNGAEL peptide). The encoded binding protein is therefore capable of specifically binding to a peptide comprising the amino acid sequence of SEQ ID NO: 1 (e.g. a peptide comprising an amino acid sequence selected from the group consisting of: SEQ ID NOs 2-21, preferably selected from the group consisting of: SEQ ID NOs 2-5, more preferably selected from the group consisting of: SEQ ID NOs 2-4 or from the group consisting of SEQ ID NO: 3-5) and does not bind to a peptide that does not contain the amino acid sequence of SEQ ID NO: 1 (e.g. it does not bind to a peptide that does not contain a peptide comprising an amino acid sequence selected from the group consisting of SEQ ID NOs 2-21, preferably selected from the group consisting of: SEQ ID NOs 2-5, more preferably selected from the group consisting of SEQ ID NOs 2-4 or from the group consisting of SEQ ID NO: 3-5).

The nucleic acid composition comprises (a) a nucleic acid sequence that encodes a TCR Vα domain with the specified features described herein and (b) a nucleic acid sequence that encodes a TCR Vβ domain with the specified features described herein. The encoded TCR components form a TIMP3 antigen-specific binding protein.

The nucleic acid sequences of (a) and (b) above may be distinct nucleic acid sequences within the nucleic acid composition. The TCR components of the binding protein may therefore be encoded by two (or more) nucleic acid sequences (with distinct nucleotide sequences) which, together, encode all of the TCR components of the binding protein. In other words, some of the TCR components may be encoded by one nucleic acid sequence in the nucleic acid composition, and others may be encoded by another (distinct) nucleic acid sequence within the nucleic acid composition.

Alternatively, the nucleic acid sequences of (a) and (b) may be part of a single nucleic acid sequence. The TCR components of the binding protein may therefore all be encoded by a single nucleic acid sequence (for example with a single open reading frame, or with multiple (e.g. 2 or more, three or more etc.) open reading frames).

Nucleic acid sequences described herein may form part of a larger nucleic acid sequence that encodes a larger component part of a functioning binding protein. For example, a nucleic acid sequence that encodes a TCR Vα domain with the specified features described herein may be part of a larger nucleic acid sequence that encodes a functional TCR α chain (including the constant domain). As another example, a nucleic acid sequence that encodes a TCR Vβ domain with the specified features described herein may be part of a larger nucleic acid sequence that encodes a functional TCR β chain (including the constant domain). As a further example, both nucleic acid sequences (a) and (b) above may be part of a larger nucleic acid sequence that encodes a combination of a functional TCR α chain (including the constant domain) and a functional TCR β chain (including the constant domain), optionally wherein the sequence encoding the functional TCR α chain is separated from the sequence encoding the functional TCR β chain by a linker sequence that enables coordinate expression of two proteins or polypeptides in the same nucleic acid sequence. More details on this are provided below.

The nucleic acid sequences described herein may alternatively encode a small component of a T cell receptor e.g. a TCR Vα domain, or a TCR Vβ domain, only. The nucleic acid sequences may be considered as “building blocks” that provide essential components for peptide binding specificity. The nucleic acid sequences described herein may be incorporated into a distinct nucleic acid sequence (e.g. a vector) that encodes the other elements of a functional binding protein such as a TCR, such that when the nucleic acid sequence described herein is incorporated, a new nucleic acid sequence is generated that encodes e.g. a TCR α chain and/or a TCR β chain that specifically binds to a TIMP3 antigen (e.g. wherein the TIMP3 antigen comprises an amino acid sequence selected from the group consisting of: SEQ ID NOs 1-21, preferably selected from the group consisting of SEQ ID NOs 1-5, or SEQ ID NOs 2-5, more preferably selected from the group consisting of SEQ ID NOs 2-4 or from the group consisting of SEQ ID NO: 3-5). The nucleic acid sequences described herein therefore have utility as essential components that confer binding specificity for a TIMP3 antigen, and thus can be used to generate a larger nucleic acid sequence encoding a binding protein with the required antigen binding activity and specificity.

The nucleic acid sequences described herein may be codon optimised for expression in a host cell, for example they may be codon optimised for expression in a human cell, such as a cell of the immune system, an inducible pluripotent stem cell (iPSC), a hematopoietic stem cell, a T cell, a primary T cell, a T cell line, a NK cell, an innate lymphoid cell (ILC), or a natural killer T cell (Scholten et al, Clin. Immunol. 119: 135, 2006). The T cell can be a CD4+ or a CD8+ T cell. Codon optimisation is a well-known method in the art for maximizing expression of a nucleic acid sequence in a particular host cell. For instance, one or more cysteine residues may also be introduced into the encoded TCR alpha and beta chain components (e.g. to reduce the risk of mispairing with endogenous TCR chains).

In one example, the nucleic acid sequences described herein are codon optimised for expression in a suitable host cell, and/or are modified to introduce codons encoding one or more cysteine amino acids (e.g. into the constant domain of the encoded TCR alpha chain and/or the encoded TCR beta chain) to reduce the risk of mispairing with endogenous TCR chains. In one example, the nucleic acid sequences described herein are codon optimised for expression in a suitable host cell, optionally wherein the host cell is a human cell.

In certain examples, a TCR constant domain is modified to enhance pairing of desired TCR chains. For example, enhanced pairing between a heterologous TCR α chain and a heterologous TCR β chain due to a modification may result in the preferential assembly of a TCR comprising two heterologous chains over an undesired mispairing of a heterologous TCR chain with an endogenous TCR chain (see, e.g., Govers et al, Trends Mol. Med. 16(2):11 (2010)). Exemplary modifications to enhance pairing of heterologous TCR chains include the introduction of complementary cysteine residues in each of the heterologous TCR α chain and P chain. A binding protein that is encoded by the nucleic acid compositions described herein is specific for a TIMP3 antigen and comprises TIMP3 antigen specific-TCR components. However, the encoded binding protein is not limited to being a TCR. Other appropriate binding proteins that comprise the specified TIMP3 antigen specific-TCR components are also encompassed. For example, the encoded binding protein may comprise a TCR, an antigen binding fragment of a TCR, a chimeric antigen receptor (CAR), or an ImmTAC. TCRs, antigen binding fragments thereof, CARs and ImmTACs are well defined in the art. A non-limiting example of an antigen binding fragment of a TCR is a single chain TCR (scTCR) or a chimeric dimer composed of the antigen binding fragments of the TCR α and TCR β chain linked to transmembrane and intracellular domains of a dimeric complex so that the complex is a chimeric dimer TCR (cdTCR). An ImmTAC comprises a TCR connected to an anti-CD3 antibody. ImmTACs are therefore bispecific, combining TIMP3-recognizing TCR components with immune activating complexes.

In certain examples, an antigen-binding fragment of a TCR comprises a single chain TCR (scTCR), which comprises both the TCR Vα and TCR Vβ domains, but only a single TCR constant domain. In other examples, an antigen-binding fragment of a TCR comprises a chimeric TCR dimer in which the antigen binding fragment of the TCR is linked to an alternative transmembrane and intracellular signalling domain (where the alternative transmembrane and intracellular signalling domain are not naturally found in TCRs). In further examples, an antigen-binding fragment of a TCR or a chimeric antigen receptor is chimeric (e.g., comprises amino acid residues or motifs from more than one donor or species), humanized (e.g., comprises residues from a non-human organism that are altered or substituted so as to reduce the risk of immunogenicity in a human), or human.

“Chimeric antigen receptor” (CAR) refers to a fusion protein that is engineered to contain two or more naturally-occurring amino acid sequences linked together in a way that does not occur naturally or does not occur naturally in a host cell, which fusion protein can function as a receptor when present on a surface of a cell. CARs described herein include an extracellular portion comprising an antigen binding domain (i.e., obtained or derived from an immunoglobulin or immunoglobulin-like molecule, such as an scFv derived from an antibody or TCR specific for an antigen (e.g. a cancer antigen etc), or an antigen binding domain derived or obtained from a killer immunoreceptor from an NK cell) linked to a transmembrane domain and one or more intracellular signalling domains (optionally containing co-stimulatory domain(s)) (see, e.g., Sadelain et al, Cancer Discov., 3(4):388 (2013); see also Harris and Kranz, Trends Pharmacol. Sci., 37(3):220 (2016), and Stone et al, Cancer Immunol. Immunother., 63(11): 1163 (2014)).

Methods for producing engineered TCRs are described in, for example, Bowerman et al, Mol. Immunol, 5(15):3000 (2009). Methods for making CARs are well known in the art and are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; and Brentjens et al, 2007, Clin. Cancer Res. 73:5426.

The binding proteins described herein may also be expressed as part of a transgene construct that encodes additional accessory proteins, such as a safety switch protein, a tag, a selection marker, a CD8 co-receptor β-chain, α-chain or both, or any combination thereof.

A T cell receptor (TCR) is a molecule found on the surface of T cells (T lymphocytes) that is responsible for recognising a peptide that is bound to (presented by) a major histocompatibility complex (MHC) molecule on a target cell. The invention is directed to nucleic acid compositions that encode binding proteins comprising TCR components that interact with a particular peptide in the context of the appropriate serotype of MHC, i.e. a TIMP3 antigen in the context of HLA-A*02 or HLA-A*02:01, HLA-A*02:02, HLA-A*02:03, HLA-A*02:04, or HLA-A*02:09, preferably HLA-A*02:01 (in other words, the encoded binding protein is capable of specifically binding to a TIMP3antigen: specific HLA complex).

In an example, the invention is directed to nucleic acid compositions that encode binding proteins comprising TCR components that interact with a particular peptide in the context of the appropriate serotype of MHC, i.e. the peptide of SEQ ID NO: 1 in the context of HLA-A*02:01; SLGDWGAEA (SEQ ID NO: 2) in the context of HLA-A*02:01; SLGDWGAEV (SEQ ID NO: 3) in the context of HLA-A*02:01; SLGDWGAEI (SEQ ID NO: 4) in the context of HLA-A*02:01; SLGDWGAEL (SEQ ID NO: 5) in the context of HLA-A*02:01; SLGDWGAER (SEQ ID NO: 6) in the context of HLA-A*02:01; SLGDWGAEN (SEQ ID NO: 7) in the context of HLA-A*02:01; SLGDWGAED (SEQ ID NO: 8) in the context of HLA-A*02:01; SLGDWGAEC (SEQ ID NO: 9) in the context of HLA-A*02:01; SLGDWGAEE (SEQ ID NO: 10) in the context of HLA-A*02:01; SLGDWGAEQ (SEQ ID NO: 11) in the context of HLA-A*02:01; SLGDWGAEG (SEQ ID NO: 12) in the context of HLA-A*02:01; SLGDWNGAEH (SEQ ID NO: 13) in the context of HLA-A*02:01; SLGDWGAEK (SEQ ID NO: 14) in the context of HLA-A*02:01; SLGDWGAEM (SEQ ID NO: 15) in the context of HLA-A*02:01; SLGDWGAEF (SEQ ID NO: 16) in the context of HLA-A*02:01; SLGDWGAEP (SEQ ID NO: 17) in the context of HLA-A*02:01; SLGDWGAES (SEQ ID NO: 18) in the context of HLA-A*02:01; SLGDWNGAET (SEQ ID NO: 19) in the context of HLA-A*02:01, SLGDWGAEW (SEQ ID NO: 20) in the context of HLA-A*02:01; and/or SLGDWGAEY (SEQ ID NO: 21) in the context of HLA-A*02:01. In another example the TCR components interact with a peptide comprising an amino acid sequence selected from the group consisting of: SEQ ID NOs: 1-21 in the context of HLA-A*02:01.

HLA-A*02:01 is a globally common human leukocyte antigen serotype within the HLA-A serotype group. Peptides that are presented by HLA-A*02:01 to TCRs are described as being “HLA-A*02:01 restricted”. Other respective serotypes are described accordingly.

As described herein, the inventors have identified several TIMP3 derived peptides presented on cells in HLA-A*02:01. Specifically, the inventors identified the TIMP3 derived peptide SEQ ID NO: 2 (SLGDWGAEA).

Accordingly, the TIMP3 antigen specifically bound by a binding protein described herein may comprise an amino acid sequence selected from the group consisting of: SEQ ID NOs: 1-21, preferably selected from the group consisting of: SEQ ID NOs 1-5 or SEQ ID NOs 2-5. The antigen may be an antigenic fragment (i.e. a portion) of an amino acid sequence selected from the group consisting of: SEQ ID NOs: 1-21, preferably selected from the group consisting of SEQ ID NOs 1-5 or SEQ ID NOs 2-5, more preferably selected from the group consisting of: SEQ ID NOs 2-4 or from the group consisting of SEQ ID NO: 3-5, it may consist of an amino acid sequence selected from the group consisting of: SEQ ID NOs: 1-21, preferably selected from the group consisting of: SEQ ID NOs 1-5 or SEQ ID NOs 2-5, more preferably selected from the group consisting of: SEQ ID NOs 2-4 or from the group consisting of SEQ ID NO: 3-5, or it may comprise (i.e. include within a longer sequence) an amino acid sequence selected from the group consisting of: SEQ ID NOs: 1-21, preferably selected from the group consisting of: SEQ ID NOs 1-5 or SEQ ID NOs 2-5, more preferably selected from the group consisting of: SEQ ID NOs 2-4 or from the group consisting of SEQ ID NO: 3-5.

The inventors identified that the TIMP3 derived peptide SLGDWGAEA (SEQ ID NO: 2) is capable of being presented by HLA-A*02:01. As mentioned elsewhere herein, the second amino acid, L, in this peptide may be varied without adversely affecting TCR binding to the peptide:HLA complex. This also applies to the C-terminal amino acid (A), which, as shown herein, may also be varied without adversely affecting TCR specificity. In view of this, the invention relates to SLGDWGAEA (SEQ ID NO: 2) and functional variants thereof, which are represented by SEQ ID NO: 1 (SX₁GDVGAEX₂, wherein X₁ and X₂ are any amino acid).

Accordingly, in one example, the binding protein described herein is capable of specifically binding to a peptide:HLA complex selected from the group consisting of a SX₁GDVGAEX₂:HLA-A*02:01 complex (wherein X₁ and X₂ are any amino acid), a SLGDWGAEA:HLA-A*02:01 complex, a SLGDVGAEV:HLA-A*02:01 complex, a SLGDVGAE:HLA-A*02:01 complex, a SLGDVGAEL:HLA-A*02:01 complex, a SLGDVGAER:HLA-A*02:01 complex, a SLGDWGAEN:HLA-A*02:01 complex, a SLGDWGAED:HLA-A*02:01 complex, a SLGDVGAEC:HLA-A*02:01 complex, a SLGDVGAEQ:HLA-A*02:01 complex, a SLGDVGAEE:HLA-A*02:01 complex, a SLGDVGAEG:HLA-A*02:01 complex, a SLGDVGAEH:HLA-A*02:01 complex, a SLGDVGAEK:HLA-A*02:01 complex, a SLGDVWGAEM:HLA-A*02:01 complex, a SLGDVWGAEF:HLA-A*02:01 complex, a SLGDVGAEP:HLA-A*02:01 complex, a SLGDVGAES:HLA-A*02:01 complex, a SLGDVWGAET:HLA-A*02:01 complex, a SLGDWGAEW-HLA-A*02:01 complex, and a SLGDWGAEY:HLA-A*02:01 complex, preferably selected from the group consisting of: a SX₁GDVWGAEX₂:HLA-A*02:01 complex (wherein X₁ and X₂ are any amino acid), a SLGDVGAEA:HLA-A*02:01 complex, a SLGDWGAEV:HLA-A*02:01 complex, a SLGDVGAEI:HLA-A*02:01 complex and a SLGDWGAEL:HLA-A*02:01 complex, more preferably selected from the group consisting of: a SX₁GDVGAEX₂:HLA-A*02:01 complex (wherein X₁ and X₂ are any amino acid), a SLGDWGAEA:HLA-A*02:01 complex, a SLGDVGAEV:HLA-A*02:01 complex and a SLGDVGAE:HLA-A*02:01 complex. In other examples, for the above complexes HLA-A*02:01 is replaced by HLA-A*02:02, HLA-A*02:03, HLA-A*02:04, or HLA-A*02:09.

In yet another example, the encoded binding protein is capable of specifically binding to a peptide:HLA complex selected from the group consisting of: a SX₁GDWGAEX₂:HLA-A*02 complex (wherein X₁ and X₂ are any amino acid), a SLGDWGAEA:HLA-A*02 complex, a SLGDVGAEV:HLA-A*02 complex, a SLGDVGAE:HLA-A*02 complex, a SLGDVGAEL:HLA-A*02 complex, a SLGDWGAER:HLA-A*02 complex, a SLGDVGAEN:HLA-A*02 complex, a SLGDVGAED:HLA-A*02 complex, a SLGDVGAEC:HLA-A*02 complex, a SLGDWGAEQ:HLA-A*02 complex, a SLGDVGAEE:HLA-A*02 complex, a SLGDVGAEG:HLA-A*02 complex, a SLGDVGAEH:HLA-A*02 complex, a SLGDVGAEK:HLA-A*02 complex, a SLGDVGAEM:HLA-A*02 complex, a SLGDVGAEF:HLA-A*02 complex, a SLGDVGAEP:HLA-A*02 complex, a SLGDVGAES:HLA-A*02 complex, a SLGDVGAET:HLA-A*02 complex, a SLGDWGAEW-HLA-A*02 complex, and a SLGDVGAEY:HLA-A*02 complex, preferably selected from the group consisting of: a SX₁GDWGAEX₂:HLA-A*02 complex (wherein X₁ and X₂ are any amino acid), a SLGDVGAEA:HLA-A*02 complex, a SLGDWGAEV:HLA-A*02 complex, a SLGDVGAEI:HLA-A*02 complex and a SLGDVGAEL:HLA-A*02 complex, more preferably selected from the group consisting of: a SX₁GDVWGAEX₂:HLA-A*02 complex (wherein X₁ and X₂ are any amino acid), a SLGDVGAEA:HLA-A*02 complex, a SLGDVGAEV:HLA-A*02 complex and a SLGDVGAE:HLA-A*02 complex.

In one example, the TIMP3 derived peptide of the peptide:HLA complex comprises an antigenic fragment of an amino acid sequence selected from the group consisting of: SEQ ID NOs: 1-21, preferably selected from the group consisting of: SEQ ID NOs 1-5 or SEQ ID NOs 2-5, more preferably selected from the group consisting of SEQ ID NOs 2-4 or from the group consisting of SEQ ID NO: 3-5. In a further example, the TIMP3 derived peptide of the peptide:HLA complex comprises or consists of an amino acid sequence selected from the group consisting of: SEQ ID NOs: 1-21, preferably selected from the group consisting of: SEQ ID NOs 1-5 or SEQ ID NOs 2-5, preferably selected from the group consisting of SEQ ID NOs 2-4 or from the group consisting of SEQ ID NO: 3-5.

Advantageously, the encoded binding protein is capable of interacting with (e.g. specifically binding to) the natural TIMP3 amino acid sequence of SEQ ID NO:2 (e.g. wherein it does not bind to a peptide that does not include the amino acid sequence of SEQ ID NO:1).

The TCR is composed of two different polypeptide chains. In humans, 95% of TCRs consist of an alpha (a) chain and a beta (P) chain (encoded by TRA and TRB respectively). When the TCR engages with a peptide in the context of HLA (e.g. in the context of HLA-A*02:01), the T cell is activated through signal transduction.

The alpha and beta chains of the TCR α re highly variable in sequence. Each chain is composed of two extracellular domains, a variable domain (V) and a constant domain (C). The constant domain is proximal to the T cell membrane followed by a transmembrane region and a short cytoplasmic tail while the variable domain binds to the peptide/HLA complex.

An isolated nucleic acid composition that encodes a TIMP3 antigen-specific binding protein is provided herein having a TCR α chain variable (Vα) domain and a TCR β chain variable (Vβ) domain. In one example the nucleic acid composition described herein may comprise a TCR α chain constant domain and/or a TCR β chain constant domain.

The variable domain of each chain has three hypervariable regions (also called complementarity determining regions (CDRs)). Accordingly, the TCR alpha variable domain (referred to herein as a TCR Vα domain, TCR V alpha domain, Vα domain or V alpha domain, alpha variable domain etc) comprises a CDR1, a CDR2 and CDR3 region. Similarly, the TCR beta variable domain (referred to herein as a TCR Vβ domain, TCR V beta domain, Vβ domain or V beta domain, beta variable domain etc) also comprises a (different) CDR1, CDR2, and CDR3 region. In each of the alpha and beta variable domains it is CDR3 that is mainly responsible for recognizing the peptide being presented by the HLA molecules.

As will be clear to a person of skill in the art, the phrase “TCR α chain variable domain” refers to the variable (V) domain (extracellular domain) of a TCR alpha chain, and thus includes three hypervariable regions (CDR1, CDR2 and the specified CDR3), as well as the intervening sequences, but does not include the constant (C) domain of the alpha chain, which does not form part of the variable domain.

As will be clear to a person of skill in the art, the phrase “TCR β chain variable domain” refers to the variable (V) domain (extracellular domain) of a TCR beta chain, and thus includes three hypervariable regions (CDR1, CDR2 and the specified CDR3), as well as the intervening sequences, but does not include the constant (C) domain of the beta chain, which does not form part of the variable domain.

### TCR Components

The isolated nucleic acid composition described herein encodes a TIMP3 antigen-specific binding protein. As discussed herein, the inventors have identified several TCRs that interact with the natural TIMP3 amino acid sequence SLGDWGAEA (SEQ ID NO: 2) when presented by HLA-A*02 (e.g. HLA-A*02:01).

(i) TCR Clone TCR-2: TCR Components that Interact with SLGDWGAEA (SEQ ID NO: 2)

As provided elsewhere herein, the inventors identified TCR clone TCR-2 which interacts with SLGDVGAEA (SEQ ID NO: 2) in the context of HLA-A*02:01. The sequences provided herein that correspond to TCR clone TCR-2 are SEQ ID NOs 40 to 49.

In one embodiment, an isolated nucleic acid composition that encodes a TIMP3 antigen-specific binding protein having a TCR α chain variable (Vα) domain and a TCR β chain variable (Vβ) domain is provided, the composition comprising: a nucleic acid sequence that encodes a TCR Vα domain comprising a CDR3 amino acid sequence having at least 80% sequence identity to SEQ ID NO:42, or a functional fragment thereof; and a nucleic acid sequence that encodes a TCR Vβ domain comprising a CDR3 amino acid sequence having at least 80% sequence identity to SEQ ID NO:45, or a functional fragment thereof, wherein the CDR3 sequences together specifically bind to a peptide comprising the amino acid sequence of SEQ ID NO:1 (e.g. when the peptide is complexed with HLA).

An example of an appropriate TCR Vα domain CDR3 amino acid sequence that confers specific binding to a TIMP3 antigen, in particular to a peptide comprising the amino acid sequence of SEQ ID NO:1 (e.g. to SLGDWGAEA (SEQ ID NO: 2)), is shown in SEQ ID NO:42.

As would be clear to a person of skill in the art, variants of the CDR3 amino acid sequences provided herein may also be functional (i.e. retain their ability to confer specific binding to a peptide comprising the amino acid sequence of SEQ ID NO:1 (e.g. to the peptide SLGDWGAEA (SEQ ID NO: 2)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 42, i.e. they may have at least 80%, at least 81%, at least 90%, or 100% sequence identity to SEQ ID NO: 42. In other words, appropriate (functional) Vα domain CDR3 amino acid sequences may vary from the sequence shown in SEQ ID NO:42 by one or several (e.g. two etc) amino acids.

Suitably, percent identity is calculated as the percentage of identity to the entire length of the reference sequence (e.g. SEQ ID NO:42).

As stated above, functional variants of a CDR3 amino acid sequence provided herein retain their ability to confer specific binding to a peptide comprising the amino acid sequence of SEQ ID NO:1 (e.g. the peptide shown in SEQ ID NO:2) when the CDR3 is part of TCR Vα domain. Functional variants may be naturally occurring, synthetic, or synthetically improved functional variants of a recited CDR3 amino acid sequence. The term “variant” also encompasses homologues and fragments. Functional variants will typically contain only conservative substitutions of one, two or more amino acids, or substitution, deletion or insertion of non-critical amino acids in non-critical regions of the CDR3.

Non-functional variants are amino acid sequence variants of the CDR3 amino acid sequence that do not specifically bind to a peptide comprising the amino acid sequence of SEQ ID NO:1 (e.g. the peptide shown in SEQ ID NO:2). Non-functional variants will typically contain a non-conservative substitution, a deletion, or insertion or premature truncation of the amino acid sequence of the CDR3 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 42. In examples where the TCR Vα domain CDR3 has the amino acid sequence of SEQ ID NO:42, the CDR3 may be encoded by any appropriate nucleic acid sequence.

The encoded TCR Vα domain may comprise, in addition to the specified CDR3, a CDR1 comprising an amino acid sequence of SEQ ID NO: 40, or a functional variant thereof.

A functional variant of a CDR1 sequence refers to a variant that retains the ability to specifically bind to the peptide comprising the amino acid sequence of SEQ ID NO:1 (e.g. the peptide shown in SEQ ID NO:2)). Such functional variants may be naturally occurring, synthetic, or synthetically improved functional variants of the original CDR1 sequence. The term “variant” also encompasses homologues and fragments. Functional variants will typically contain only conservative substitutions of one or more amino acids, or substitution, deletion or insertion of non-critical amino acids in non-critical regions of the protein.

Non-functional variants are amino acid sequence variants of the original CDR1 sequence that do not specifically bind to a peptide comprising the amino acid sequence of SEQ ID NO:1 (e.g. the peptide shown in SEQ ID NO:2). Non-functional variants will typically contain a non-conservative substitution, a deletion, or insertion or premature truncation of the amino acid sequence of the original CDR1, or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 40, i.e. it may have at least 80%, at least 83%, or 100% sequence identity to SEQ ID NO: 40. In other words, appropriate functional Vα domain CDR1 amino acid sequences may vary from the sequence shown in SEQ ID NO: 1 by one or several amino acids.

In one example, the CDR1 of the Vα domain comprises or consists of the amino acid sequence of SEQ ID NO:40. In examples where the TCR Vα domain CDR1 has the amino acid sequence of SEQ ID NO:40, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO:41, or a functional variant thereof (i.e. wherein the variant retains the ability to specifically bind to HLA-A*02, most preferably HLA-A*02:01).

Functional variants may be naturally occurring, synthetic, or synthetically improved functional variants of the original CDR2 sequence. The term “variant” also encompasses homologues and fragments. Functional variants will typically contain only conservative substitutions of one or more amino acids, or substitution, deletion or insertion of non-critical amino acids in non-critical regions of the protein.

Non-functional variants are amino acid sequence variants of the original CDR2 sequence that do not specifically bind to HLA-A*02, preferably HLA-A*02:01. Non-functional variants will typically contain a non-conservative substitution, a deletion, or insertion or premature truncation of the amino acid sequence of the original CDR2 sequence or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 41, i.e. it may have at least 80%, at least 85%, or 100% sequence identity to SEQ ID NO: 41. In other words, appropriate (functional) Vα domain CDR2 amino acid sequences may vary from the sequence shown in SEQ ID NO:41 by one or several amino acids. Suitably, percent identity is calculated as the percentage of identity to the entire length of the reference sequence (e.g. SEQ ID NO:41). As stated previously, the variant may comprise an amino acid substitution such as a conservative amino acid substitution compared to the original CDR2 sequence. As stated above, a functional variant of a CDR2 retains the ability to specifically bind to HLA-A*02, preferably HLA-A*02:01.

In one example, the CDR2 of the Vα domain comprises or consists of the amino acid sequence of SEQ ID NO: 41. In examples where the TCR Vα domain CDR2 has the amino acid sequence of SEQ ID NO:41, the CDR2 may be encoded by any appropriate nucleic acid sequence. The encoded TCR Vα domain may therefore comprise the CDRs mentioned in detail above (by SEQ ID specifically i.e. SEQ ID NO:42, SEQ ID NO: 40 and SEQ ID NO: 41, or functional variants thereof), with appropriate intervening sequences between the CDRs.

The encoded TCR Vα domain may comprise an amino acid sequence of SEQ ID NO:46, or a functional variant thereof (i.e. wherein the variant TCR Vα domain retains the ability to specifically bind to a peptide comprising the amino acid sequence of SEQ ID NO:1 (e.g. the peptide shown in SEQ ID NO:2) when part of a binding protein described herein). Such functional variants may be naturally occurring, synthetic, or synthetically improved functional variants of SEQ ID NO:46. Functional variants will typically contain only conservative substitutions of one or more amino acids, or substitution, deletion or insertion of non-critical amino acids in non-critical regions of the protein.

Non-functional variants are amino acid sequence variants that do not specifically bind to a peptide comprising the amino acid sequence of SEQ ID NO:1 (e.g. the peptide shown in SEQ ID NO:2). Non-functional variants will typically contain a non-conservative substitution, a deletion, or insertion or premature truncation of the amino acid sequence of the original TCR Vα domain or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 46, whilst retaining the ability to specifically bind to a peptide comprising the amino acid sequence of SEQ ID NO:1 (e.g. the peptide shown in SEQ ID NO:2). In other words, a functional TCR Vα domain with one or several amino acid substitutions compared to the sequence of SEQ ID NO:46 is also encompassed. As stated previously, the amino acid substitution may be a conservative amino acid substitution. The variability in sequence compared to SEQ ID NO:46 may all be in regions of the TCR Vα domain that do not form CDRs (i.e. the variant may have the CDRs of SEQ ID NO: 42, SEQ ID NO: 40 and/or SEQ ID NO: 41, and still have 25% (or less) sequence variability compared to SEQ ID NO:46). In other words, the sequence of the CDRs of SEQ ID NO: 46 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 46).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 46, wherein the TCR Vα domain comprises a CDR3 having an amino acid sequence of SEQ ID NO: 42. In this example, the TCR Vα domain CDR1 may have an amino acid sequence of SEQ ID NO: 40 and the TCR Vα domain CDR2 may have an amino acid sequence of SEQ ID NO: 41.

As another example, the encoded TCR Vα domain may comprise an amino acid sequence having the amino acid sequence of SEQ ID NO: 46, with 0 to 10 (or 0 to 5) amino acid substitutions, insertions or deletions), wherein the TCR Vα domain comprises a CDR3 having an amino acid sequence of SEQ ID NO: 42. In this example, the TCR Vα domain CDR1 may have an amino acid sequence of SEQ ID NO: 40 and the TCR Vα domain CDR2 may have an amino acid sequence of SEQ ID NO: 41.

In examples where the TCR Vα domain has the amino acid sequence of SEQ ID NO:46, the TCR Vα domain may be encoded by the nucleic acid sequence of SEQ ID NO:47, or a genetically degenerate sequence thereof (i.e. other nucleic acid sequences that encode the same protein as a result of the degeneracy of the genetic code).

The phrase “genetically degenerate sequence thereof” is used interchangeably with “derivative thereof” herein.

For the avoidance of doubt, the nucleic acid sequence encoding the TCR Vα domain may also encode a TCR α chain constant domain. An example of a suitable constant domain (for either a TCR α chain or a TCR β chain) is encoded in the MP71-TCR-flex retroviral vector. However, the invention is not limited to this specific constant domain, and encompasses any appropriate TCR α chain constant domain. The constant domain may be murine derived, human derived or humanised. Methods for identifying or generating appropriate constant domains are well known to a person of skill in the art and are well within their routine capabilities.

By way of example only, the constant domain may be encoded by or derived from a vector, such as a lentiviral, retroviral or plasmid vector but also adenovirus, adeno-associated virus, vaccinia virus, canary poxvirus or herpes virus vectors in which murine or human constant domains are pre-cloned. Recently, minicircles have also been described for TCR gene transfer (non-viral Sleeping Beauty transposition from minicircle vectors as published by R Monjezi, et al., 2017). Moreover, naked (synthetic) DNA/RNA can also be used to introduce the TCR. As an example, a pMSGV retroviral vector with pre-cloned TCR-Ca and Cb genes as described in LV Coren et al., BioTechniques 2015 may be used to provide an appropriate constant domain. Alternatively, single stranded or double stranded DNA or RNA can be inserted by homologous directed repair into the TCR locus (see Roth et al 2018 Nature vol 559; page 405). As a further option, non-homologous end joining is possible.

In one example, the nucleic acid composition provided herein comprises a nucleic acid sequence that encodes a TCR Vα domain comprising a CDR3 amino acid sequence having at least 80% sequence identity to SEQ ID NO:42, or a functional fragment thereof.

In another example, the CDR3 of the Vα domain of a nucleic acid composition provided herein comprises or consists of the amino acid sequence of SEQ ID NO: 42.

In another example, the Vα domain of a nucleic acid composition provided herein comprises an amino acid sequence having at least 80% sequence identity to, comprising, or consisting of, SEQ ID NO: 46.

As provided above, the inventors identified TCR clone TCR-2 which interacts with SLGDWGAEA (SEQ ID NO: 2) in the context of HLA-A*02:01. The sequences provided herein that correspond to TCR clone TCR-2 are SEQ ID NOs: 40 to 49.

Accordingly, an example of an appropriate TCR Vβ domain CDR3 amino acid sequence that confers specific binding to a TIMP3 antigen, in particular to a peptide comprising the amino acid sequence of SEQ ID NO:1 (e.g. to SEQ ID NO: 2)), is shown in SEQ ID NO:45. As would be clear to a person of skill in the art, variants of the amino acid sequence shown in SEQ ID NO:45 may also be functional (i.e. retain their ability to confer specific binding to a peptide comprising the amino acid sequence of SEQ ID NO:1 (e.g. the peptide shown in SEQ ID NO:2) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 45, i.e. they may have at least 80%, at least 83%, at least 91%, or 100% sequence identity to SEQ ID NO: 45. In other words, appropriate (functional) Vβ domain CDR3 amino acid sequences may vary from the sequence shown in SEQ ID NO: 45 by one or several (e.g. two) amino acids.

In one example, the CDR3 of the Vβ domain comprises or consists of the amino acid sequence of SEQ ID NO: 45. In examples where the TCR Vβ domain CDR3 has the amino acid sequence of SEQ ID NO:45, the CDR3 may be encoded by any appropriate nucleic acid sequence.

The encoded TCR Vβ domain may comprise, in addition to the specified CDR3, a CDR1 comprising an amino acid sequence of SEQ ID NO: 43, or a functional variant thereof (i.e. wherein the variant retains the ability to specifically bind to a peptide comprising the amino acid sequence of SEQ ID NO:1 (e.g. the peptide shown in SEQ ID NO:2).

For example, appropriate functional Vβ domain CDR1 amino acid sequences may have at least 80% sequence identity to SEQ ID NO: 43, i.e. it may have at least 80%, or 100% sequence identity to SEQ ID NO: 43. In other words, appropriate (functional) Vβ domain CDR1 amino acid sequences may vary from the sequence shown in SEQ ID NO:43 by one or several amino acids. In one example, the CDR1 of the Vβ domain comprises or consists of the amino acid sequence of SEQ ID NO: 43. In examples where the TCR Vα domain CDR1 has the amino acid sequence of SEQ ID NO:43, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 44, or a functional variant thereof (i.e. wherein the variant retains the ability to specifically bind to HLA-A*02, most preferably HLA-A*02:01).

For example, appropriate functional Vβ domain CDR2 amino acid sequences may have at least 80% sequence identity to SEQ ID NO: 44, i.e. it may have at least 80%, at least 83%, or 100% sequence identity to SEQ ID NO: 44. In other words, appropriate (functional) Vβ domain CDR2 amino acid sequences may vary from the sequence shown in SEQ ID NO: 44 by one or several amino acids.

In one example, the CDR2 of the Vβ domain comprises or consists of the amino acid sequence of SEQ ID NO: 44. In examples where the TCR Vβ domain CDR2 has the amino acid sequence of SEQ ID NO:44, the CDR2 may be encoded by any appropriate nucleic acid sequence.

The encoded TCR Vβ domain may therefore comprise the CDRs mentioned in detail above (by SEQ ID specifically i.e. SEQ ID NO:45, SEQ ID NO: 43 and SEQ ID NO: 44, or functional variants thereof), with appropriate intervening sequences between the CDRs.

The encoded TCR Vβ domain may have an amino acid sequence of SEQ ID NO: 48, or a functional variant thereof (i.e. wherein the variant TCR Vβ domain retains the ability to specifically bind to a peptide comprising the amino acid sequence of SEQ ID NO:1 (e.g. the peptide shown in SEQ ID NO:2) when part of a binding protein described herein).

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 48, whilst retaining the ability to specifically bind to a peptide comprising the amino acid sequence of SEQ ID NO:1 (e.g. the peptide shown in SEQ ID NO:2). In other words, a functional TCR Vβ domain with one or several amino acid substitutions compared to the sequence of SEQ ID NO: 48 is also encompassed. The variability in sequence compared to SEQ ID NO:48 may all be in regions of the TCR Vβ domain that do not form CDRs (i.e. the variant may have the CDRs of SEQ ID NO: 45, SEQ ID NO: 43 and/or SEQ ID NO: 44, and still have 25% (or less) sequence variability compared to SEQ ID NO: 48). In other words, the sequence of the CDRs of SEQ ID NO: 48 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 48).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 48, wherein the TCR Vβ domain comprises a CDR3 having an amino acid sequence of SEQ ID NO: 45. In this example, the TCR Vβ domain CDR1 may have an amino acid sequence of SEQ ID NO:43 and the TCR Vβ domain CDR2 may have an amino acid sequence of SEQ ID NO: 44.

In examples where the TCR Vβ domain has the amino acid sequence of SEQ ID NO:48, the TCR Vβ domain may be encoded by the nucleic acid sequence of SEQ ID NO:49, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

In an example, the nucleic acid composition provided herein comprises a nucleic acid sequence that encodes a TCR Vβ domain comprising a CDR3 amino acid sequence having at least 80% sequence identity to SEQ ID NO:45, or a functional fragment thereof. In another example, the CDR3 of the Vβ domain of a nucleic acid composition provided herein comprises or consists of the amino acid sequence of SEQ ID NO:45.

In a further example, the Vβ domain of a nucleic acid composition provided herein comprises an amino acid sequence having at least 80% sequence identity to, comprising, or consisting of, SEQ ID NO: 48.

The TCR Vβ domain sequences derived from TCR clone TCR-2 discussed above are particularly compatible with the TCR Vα domain sequences derived from TCR clone TCR-2 discussed elsewhere herein.

Accordingly, in one example, a nucleic acid composition described herein encodes a binding protein specific for a peptide comprising the amino acid sequence of SEQ ID NO:1 having TCR Vα domain comprising a CDR3 amino acid sequence having at least 80% sequence identity to SEQ ID NO:42, or a functional fragment thereof; and a nucleic acid sequence that encodes a TCR Vβ domain comprising a CDR3 amino acid sequence having at least 80% sequence identity to SEQ ID NO:45, or a functional fragment thereof.

In a particular example, a nucleic acid composition described herein encodes a binding protein specific for a peptide comprising the amino acid sequence of SEQ ID NO:1 having a TCR Vα domain with a CDR3 amino acid sequence comprising or consisting of the amino acid sequence of SEQ ID NO: 42; and a TCR Vβ domain with a CDR3 comprising or consisting of the amino acid sequence of SEQ ID NO:45. In addition, the peptide comprising the amino acid sequence of SEQ ID NO:1 may comprise or consist of the sequence shown in SEQ ID NO: 2. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 46; and the Vβ domain may comprise an amino acid sequence having at least 80% sequence identity to, comprising, or consisting of, SEQ ID NO: 48. In one example, the Vα domain comprises the amino acid sequence of SEQ ID NO: 46 and the Vβ domain comprises the amino acid sequence of SEQ ID NO: 48. In such cases, the Vα domain may be encoded by a nucleic acid sequence comprising the sequence of SEQ ID NO: 47; and the Vβ domain may be encoded by a nucleic acid sequence comprising the sequence of SEQ ID NO: 49.

In this particular example, the TCR Vα domain may include a CDR1 amino acid sequence comprising or consisting of the amino acid sequence of SEQ ID NO:40 and a CDR2 amino acid sequence comprising or consisting of the amino acid sequence of SEQ ID NO:41. Furthermore, the TCR Vβ domain may include a CDR1 amino acid sequence comprising or consisting of the amino acid sequence of SEQ ID NO:43 and a CDR2 amino acid sequence comprising or consisting of the amino acid sequence of SEQ ID NO: 44.

For the avoidance of doubt, this particular example encompasses components of TCR clone TCR-2 exemplified herein. The different components of TCR clone TCR-2 and their respective SEQ ID Nos are summarised in Table 7 below.

As stated in more detail elsewhere herein, the nucleic acid composition described herein encodes both a TCR Vα domain and a TCR Vβ domain, which form the binding protein that is capable of specifically binding to a TIMP3 antigen. In examples where the TCR Vα domain and the TCR Vβ domain are encoded by the same nucleic acid sequence, the TCR Vα domain and TCR Vβ domain may be joined together via a linker, e.g. a linker that enables expression of two proteins or polypeptides from the same vector. By way of example, a linker comprising a porcine teschovirus-1 2A (P2A) sequence may be used, such as 2A sequences from foot-and-mouth disease virus (F2A), equine rhinitis A virus (E2A) or Thosea asigna virus (T2A) as published by A. L. Szymczak et al., Nature Biotechnology 22, 589-594 (2004) or 2A-like sequences. 2A and 2A-like sequences are linkers that are cleavable once the nucleic acid molecule has been transcribed and translated. Another example of a linker is an internal ribosomal entry sites (IRES) which enables translation of two proteins or polypeptides from the same transcript. Any other appropriate linker may also be used. As a further example, the nucleic acid sequence encoding the TCR Vα domain and nucleic acid sequence encoding the TCR Vβ domain may be cloned into a vector with dual internal promoters (see e.g. S Jones et al., Human Gene Ther 2009). The identification of appropriate linkers and vectors that enable expression of both the TCR Vα domain and the TCR Vβ domain is well within the routine capabilities of a person of skill in the art.

Additional appropriate polypeptide domains may also be encoded by the nucleic acid sequences that encode the TCR Vα domain and/or the TCR Vβ domain. By way of example only, the nucleic acid sequence may comprise a membrane targeting sequence that provides for transport of the encoded polypeptide to the cell surface membrane of the modified cell. Other appropriate additional domains are well known and are described, for example, in WO2016/071758.

In one example, the nucleic acid composition described herein may encode a soluble TCR. For example, the nucleic acid composition may encode the variable domain of the TCR alpha and beta chains respectively together with an immune-modulator molecule such as a CD3 agonist (e.g. an anti-CD3 scFv). The CD3 antigen is present on mature human T cells, thymocytes and a subset of natural killer cells. It is associated with the TCR α nd is involved in signal transduction of the TCR. Antibodies specific for the human CD3 antigen are well known. One such antibody is the murine monoclonal antibody OKT3, which is the first monoclonal antibody approved by the FDA. Other antibodies specific for CD3 have also been reported (see e.g. WO2004/106380; U.S. Patent Application Publication No. 2004/0202657; U.S. Pat. No. 6,750,325). Immune mobilising mTCR Against Cancer (ImmTAC; Immunocore Limited, Milton Partk, Abington, Oxon, United Kingdom) are bifunctional proteins that combine affinity monoclonal T cell receptor (mTCR) targeting with a therapeutic mechanism of action (i.e., an anti-CD3 scFv). In another example, a soluble TCR of the invention may be combined with a radioisotope or a toxic drug. Appropriate radioisotopes and/or toxic drugs are well known in the art and are readily identifiable by a person of ordinary skill in the art.

In one example, the nucleic acid composition may encode a chimeric single chain TCR wherein the TCR alpha chain variable domain is linked to the TCR beta chain variable domain and a constant domain which is e.g. fused to the CD3 zeta signalling domain. In this example, the linker is non-cleavable. In an alternative embodiment, the nucleic acid composition may encode a chimeric two chain TCR in which the TCR alpha chain variable domain and the TCR beta chain variable domain are each linked to a CD3 zeta signalling domain or other transmembrane and intracellular domains. Methods for preparing such single chain TCRs and two chain TCRs are well known in the art; see for example RA Willemsen et al, Gene Therapy 2000.

(ii) TCR Clone TCR-3: TCR Components that Interact with SLGDVWGAEA (SEQ ID NO: 2)

As provided elsewhere herein, the inventors have also identified TCR clone TCR-3 which interacts with SLGDWGAEA (SEQ ID NO: 2) in the context of HLA-A*02:01. The sequences provided herein that correspond to TCR clone TCR-3 are SEQ ID NO:s 50 to 59.

In one embodiment, an isolated nucleic acid composition that encodes a TIMP3 antigen-specific binding protein having a TCR α chain variable (Vα) domain and a TCR β chain variable (Vβ) domain is provided, the composition comprising: a nucleic acid sequence that encodes a TCR Vα domain comprising a CDR3 amino acid sequence having at least 80% sequence identity to SEQ ID NO:52, or a functional fragment thereof; and a nucleic acid sequence that encodes a TCR Vβ domain comprising a CDR3 amino acid sequence having at least 80% sequence identity to SEQ ID NO: 55, or a functional fragment thereof, wherein the CDR3 sequences together specifically bind to a peptide comprising the amino acid sequence of SEQ ID NO:1 (e.g. when the peptide is complexed with HLA).

Accordingly, another example of an appropriate TCR Vα domain CDR3 amino acid sequence that confers specific binding to a TIMP3 antigen, in particular a peptide comprising the amino acid sequence of SEQ ID NO:1 (e.g. to SLGDWGAEA (SEQ ID NO: 2)), is shown in SEQ ID NO:52. For example, appropriate (functional) Vα domain CDR3 amino acid sequences may have at least 80% sequence identity to SEQ ID NO:52, i.e. they may have at least 80%, at least 83%, at least 91%, or 100% sequence identity to SEQ ID NO:52. In other words, appropriate (functional) Vα domain CDR3 amino acid sequences may vary from the sequence shown in SEQ ID NO:52 by one or several (e.g. two etc) amino acids.

In one example, the CDR3 of the Vα domain comprises or consists of the amino acid sequence of SEQ ID NO: 52. In examples where the TCR Vα domain CDR3 has the amino acid sequence of SEQ ID NO:52, the CDR3 may be encoded by any appropriate nucleic acid sequence.

The encoded TCR Vα domain may comprise, in addition to the specified CDR3, a CDR1 comprising an amino acid sequence of SEQ ID NO: 50, or a functional variant thereof (i.e. wherein the variant retains the ability to specifically bind to a peptide comprising the amino acid sequence of SEQ ID NO:1 (e.g. the peptide shown in SEQ ID NO:2).

For example, appropriate functional Vα domain CDR1 amino acid sequences may have at least 80% sequence identity to SEQ ID NO: 50, i.e. it may have at least 80%, at least 83%, or 100% sequence identity to SEQ ID NO: 50. In other words, appropriate functional Vα domain CDR1 amino acid sequences may vary from the sequence shown in SEQ ID NO: 50 by one or several amino acids. In one example, the CDR1 of the Vα domain comprises or consists of the amino acid sequence of SEQ ID NO:50. In examples where the TCR Vα domain CDR1 has the amino acid sequence of SEQ ID NO:50, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO:51, or a functional variant thereof (i.e. wherein the variant retains the ability to specifically bind to HLA-A*02, most preferably HLA-A*02:01).

For example, appropriate functional Vα domain CDR2 amino acid sequences may have at least 80% sequence identity to SEQ ID NO:51, i.e. it may have at least 80%, at least 88%, or 100% sequence identity to SEQ ID NO:51. In other words, appropriate (functional) Vα domain CDR2 amino acid sequences may vary from the sequence shown in SEQ ID NO:51 by one or several amino acids. In one example, the CDR2 of the Vα domain comprises or consists of the amino acid sequence of SEQ ID NO: 51. In examples where the TCR Vα domain CDR2 has the amino acid sequence of SEQ ID NO:51, the CDR2 may be encoded by any appropriate nucleic acid sequence.

The encoded TCR Vα domain may therefore comprise the CDRs mentioned in detail above (by SEQ ID specifically i.e. SEQ ID NO:52, SEQ ID NO:50 and SEQ ID NO: 51, or functional variants thereof), with appropriate intervening sequences between the CDRs.

The encoded TCR Vα domain may comprise an amino acid sequence of SEQ ID NO:56, or a functional variant thereof (i.e. wherein the variant TCR Vα domain retains the ability to specifically bind a peptide comprising the amino acid sequence of SEQ ID NO:1 (e.g. the peptide shown in SEQ ID NO:2) when part of a binding protein described herein).

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO:56, whilst retaining the ability to specifically bind to a peptide comprising the amino acid sequence of SEQ ID NO:1 (e.g. the peptide shown in SEQ ID NO:2). In other words, a functional TCR Vα domain with one or several amino acid substitutions compared to the sequence of SEQ ID NO:56 is also encompassed. The variability in sequence compared to SEQ ID NO:56 may all be in regions of the TCR Vα domain that do not form CDRs (i.e. the variant may have the CDRs of SEQ ID NO: 52, SEQ ID NO:50 and/or SEQ ID NO:51, and still have 25% (or less) sequence variability compared to SEQ ID NO:56). In other words, the sequence of the CDRs of SEQ ID NO: 56 may be retained whilst the rest of the sequence is varied, as appropriate within the “at least 75% identity” parameters specified above.

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 56, wherein the TCR Vα domain comprises a CDR3 having an amino acid sequence of SEQ ID NO: 52. In this example, the TCR Vα domain CDR1 may have an amino acid sequence of SEQ ID NO: 50 and the TCR Vα domain CDR2 may have an amino acid sequence of SEQ ID NO: 51.

As another example, the encoded TCR Vα domain may comprise an amino acid sequence having the amino acid sequence of SEQ ID NO: 56, with 0 to 10 (or 0 to 5) amino acid substitutions, insertions or deletions, wherein the TCR Vα domain comprises a CDR3 having an amino acid sequence of SEQ ID NO: 52. In this example, the TCR Vα domain CDR1 may have an amino acid sequence of SEQ ID NO: 50 and the TCR Vα domain CDR2 may have an amino acid sequence of SEQ ID NO: 51.

In examples where the TCR Vα domain has the amino acid sequence of SEQ ID NO:56, the TCR Vα domain may be encoded by the nucleic acid sequence of SEQ ID NO: 57, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

In one example, the nucleic acid composition provided herein comprises a nucleic acid sequence that encodes a TCR Vα domain comprising a CDR3 amino acid sequence having at least 80% sequence identity to SEQ ID NO:52, or a functional fragment thereof.

In another example, the CDR3 of the Vα domain of a nucleic acid composition provided herein comprises or consists of the amino acid sequence of SEQ ID NO: 52.

In another example, the Vα domain of a nucleic acid composition provided herein comprises an amino acid sequence having at least 80% sequence identity to, comprising, or consisting of, SEQ ID NO: 56.

As provided elsewhere herein, the inventors have identified TCR clone TCR-3 which interacts with SLGDWGAEA (SEQ ID NO: 2) in the context of HLA-A*02:01. The sequences provided herein that correspond to TCR clone TCR-3 are SEQ ID NOs: 50 to 59.

An example of an appropriate TCR Vβ domain CDR3 amino acid sequence that confers specific binding to a TIMP3 antigen, in particular a peptide comprising the amino acid sequence of SEQ ID NO:1 (e.g. to SEQ ID NO:2), is shown in SEQ ID NO:55. As would be clear to a person of skill in the art, variants of the amino acid sequence shown in SEQ ID NO:55 may also be functional (i.e. retain their ability to confer specific binding to a peptide comprising the amino acid sequence of SEQ ID NO:1 (e.g. the peptide shown in SEQ ID NO:2) when the CDR3 is part of TCR Vs domain).

For example, appropriate (functional) Vβ domain CDR3 amino acid sequences may have at least 80% sequence identity to SEQ ID NO: 55, i.e. they may have at least 80%, at least 84%, at least 92%, or 100% sequence identity to SEQ ID NO: 55. In other words, appropriate (functional) Vβ domain CDR3 amino acid sequences may vary from the sequence shown in SEQ ID NO: 55 by one or several (e.g. two) amino acids. In one example, the CDR3 of the Vβ domain comprises or consists of the amino acid sequence of SEQ ID NO: 55. In examples where the TCR Vβ domain CDR3 has the amino acid sequence of SEQ ID NO:55, the CDR3 may be encoded by any appropriate nucleic acid sequence.

The encoded TCR Vβ domain may comprise, in addition to the specified CDR3, a CDR1 comprising an amino acid sequence of SEQ ID NO: 53, or a functional variant thereof (i.e. wherein the variant retains the ability to specifically bind to a peptide comprising the amino acid sequence of SEQ ID NO:1 (e.g. the peptide shown in SEQ ID NO:2).

For example, appropriate functional Vβ domain CDR1 amino acid sequences may have at least 80% sequence identity to SEQ ID NO: 53, i.e. it may have at least 80%, or 100% sequence identity to SEQ ID NO: 53. In other words, appropriate (functional) Vβ domain CDR1 amino acid sequences may vary from the sequence shown in SEQ ID NO:53 by one or several amino acids. In one example, the CDR1 of the Vβ domain comprises or consists of the amino acid sequence of SEQ ID NO: 53. In examples where the TCR Vα domain CDR1 has the amino acid sequence of SEQ ID NO:53, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 54, or a functional variant thereof (i.e. wherein the variant retains the ability to specifically bind to HLA-A*02, most preferably HLA-A*02:01).

For example, appropriate functional Vβ domain CDR2 amino acid sequences may have at least 80% sequence identity to SEQ ID NO: 54, i.e. it may have at least 80%, at least 83%, or 100% sequence identity to SEQ ID NO: 54. In other words, appropriate (functional) Vβ domain CDR2 amino acid sequences may vary from the sequence shown in SEQ ID NO: 54 by one or several amino acids.

In one example, the CDR2 of the Vβ domain comprises or consists of the amino acid sequence of SEQ ID NO: 54. In examples where the TCR Vβ domain CDR2 has the amino acid sequence of SEQ ID NO:54, the CDR2 may be encoded by any appropriate nucleic acid sequence.

The encoded TCR Vβ domain may therefore comprise the CDRs mentioned in detail above (by SEQ ID specifically i.e. SEQ ID NO:55, SEQ ID NO: 53 and SEQ ID NO: 54, or functional variants thereof), with appropriate intervening sequences between the CDRs.

The encoded TCR Vβ domain may have an amino acid sequence of SEQ ID NO: 58, or a functional variant thereof (i.e. wherein the variant TCR Vβ domain retains the ability to specifically bind to a peptide comprising the amino acid sequence of SEQ ID NO:1 (e.g. the peptide shown in SEQ ID NO:2) when part of a binding protein described herein).

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 58, whilst retaining the ability to specifically bind to a peptide comprising the amino acid sequence of SEQ ID NO:1 (e.g. the peptide shown in SEQ ID NO:2). In other words, a functional TCR Vβ domain with one or several amino acid substitutions compared to the sequence of SEQ ID NO: 58 is also encompassed. The variability in sequence compared to SEQ ID NO:58 may all be in regions of the TCR Vβ domain that do not form CDRs (i.e. the variant may have the CDRs of SEQ ID NO: 55, SEQ ID NO: 53 and/or SEQ ID NO: 54, and still have 25% (or less) sequence variability compared to SEQ ID NO: 58). In other words, the sequence of the CDRs of SEQ ID NO: 58 may be retained whilst the rest of the sequence is varied, as appropriate within the “at least 75% identity” parameters specified above.

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 58, wherein the TCR Vβ domain comprises a CDR3 having an amino acid sequence of SEQ ID NO: 55. In this example, the TCR Vβ domain CDR1 may have an amino acid sequence of SEQ ID NO:53 and the TCR Vβ domain CDR2 may have an amino acid sequence of SEQ ID NO: 54.

In examples where the TCR Vβ domain has the amino acid sequence of SEQ ID NO:58, the TCR Vβ domain may be encoded by the nucleic acid sequence of SEQ ID NO:59, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

In an example, the nucleic acid composition provided herein comprises a nucleic acid sequence that encodes a TCR Vβ domain comprising a CDR3 amino acid sequence having at least 80% sequence identity to SEQ ID NO:55, or a functional fragment thereof.

In another example, the CDR3 of the Vβ domain of a nucleic acid composition provided herein comprises or consists of the amino acid sequence of SEQ ID NO:55.

In a further example, the Vβ domain of a nucleic acid composition provided herein comprises an amino acid sequence having at least 80% sequence identity to, comprising, or consisting of, SEQ ID NO: 58.

The TCR Vβ domain sequences derived from TCR clone TCR-3 discussed above are particularly compatible with the TCR Vα domain sequences derived from TCR clone TCR-3 discussed elsewhere herein.

Accordingly, in one example, a nucleic acid composition described herein encodes a binding protein specific for a peptide comprising the amino acid sequence of SEQ ID NO:1 having TCR Vα domain comprising a CDR3 amino acid sequence having at least 80% sequence identity to SEQ ID NO:52, or a functional fragment thereof; and a nucleic acid sequence that encodes a TCR Vβ domain comprising a CDR3 amino acid sequence having at least 80% sequence identity to SEQ ID NO:55, or a functional fragment thereof.

In a particular example, a nucleic acid composition described herein encodes a binding protein specific for a peptide comprising the amino acid sequence of SEQ ID NO:1 having a TCR Vα domain with a CDR3 amino acid sequence comprising or consisting of the amino acid sequence of SEQ ID NO: 52; and a TCR Vβ domain with a CDR3 comprising or consisting of the amino acid sequence of SEQ ID NO:55. In addition, the peptide comprising the amino acid sequence of SEQ ID NO:1 may comprise or consist of the sequence shown in SEQ ID NO: 2. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 56; and the Vβ domain may comprise an amino acid sequence having at least 80% sequence identity to, comprising, or consisting of, SEQ ID NO: 58. In one example, the Vα domain comprises the amino acid sequence of SEQ ID NO: 56 and the Vβ domain comprises the amino acid sequence of SEQ ID NO: 58. In such cases, the Vα domain may be encoded by a nucleic acid sequence comprising the sequence of SEQ ID NO: 57; and the Vβ domain may be encoded by a nucleic acid sequence comprising the sequence of SEQ ID NO: 59.

In this particular example, the TCR Vα domain may include a CDR1 amino acid sequence comprising or consisting of the amino acid sequence of SEQ ID NO:50 and a CDR2 amino acid sequence comprising or consisting of the amino acid sequence of SEQ ID NO:51. Furthermore, the TCR Vβ domain may include a CDR1 amino acid sequence comprising or consisting of the amino acid sequence of SEQ ID NO:53 and a CDR2 amino acid sequence comprising or consisting of the amino acid sequence of SEQ ID NO: 54.

For the avoidance of doubt, this particular example encompasses components of TCR clone TCR-3 exemplified herein. The different components of TCR clone TCR-3 and their respective SEQ ID Nos are summarised in Table 8 below.

As stated in more detail elsewhere herein, the nucleic acid composition described herein encodes both a TCR Vα domain and a TCR Vβ domain, which form the binding protein that is capable of specifically binding to a TIMP3 antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(iii) TCR Clone TCR-4: TCR Components that Interact with SLGDVGAEA (SEQ ID NO: 2)

As provided elsewhere herein, the inventors have also identified TCR clone TCR-4 which interacts with SLGDWGAEA (SEQ ID NO: 2) in the context of HLA-A*02:01. The sequences provided herein that correspond to TCR clone TCR-4 are SEQ ID NO:s 60 to 69.

In one embodiment, an isolated nucleic acid composition that encodes a TIMP3 antigen-specific binding protein having a TCR α chain variable (Vα) domain and a TCR β chain variable (Vβ) domain is provided, the composition comprising: a nucleic acid sequence that encodes a TCR Vα domain comprising a CDR3 amino acid sequence having at least 80% sequence identity to SEQ ID NO:62, or a functional fragment thereof; and a nucleic acid sequence that encodes a TCR Vβ domain comprising a CDR3 amino acid sequence having at least 80% sequence identity to SEQ ID NO:65, or a functional fragment thereof, wherein the CDR3 sequences together specifically bind to a peptide comprising the amino acid sequence of SEQ ID NO:1 (e.g. when the peptide is complexed with HLA).

An example of an appropriate TCR Vα domain CDR3 amino acid sequence that confers specific binding to a TIMP3 antigen, in particular a peptide comprising the amino acid sequence of SEQ ID NO:1 (e.g. to SLGDWGAEA SEQ ID NO:2), is shown in SEQ ID NO: 62.

For example, appropriate (functional) Vα domain CDR3 amino acid sequences may have at least 80% sequence identity to SEQ ID NO: 62, i.e. they may have at least 80%, at least 84%, at least 92%, or 100% sequence identity to SEQ ID NO: 62. Suitably, percent identity is calculated as the percentage of identity to the entire length of the reference sequence (e.g. SEQ ID NO: 62). In other words, appropriate (functional) Vα domain CDR3 amino acid sequences may vary from the sequence shown in SEQ ID NO: 62 by one or several (e.g. two etc) amino acids.

In one example, the CDR3 of the Vα domain comprises or consists of the amino acid sequence of SEQ ID NO: 62. In examples where the TCR Vα domain CDR3 has the amino acid sequence of SEQ ID NO: 62, the CDR3 may be encoded by any appropriate nucleic acid sequence.

The encoded TCR Vα domain may comprise, in addition to the specified CDR3, a CDR1 comprising an amino acid sequence of SEQ ID NO: 60, or a functional variant thereof (i.e. wherein the variant retains the ability to specifically bind to a peptide comprising the amino acid sequence of SEQ ID NO:1 (e.g. the peptide shown in SEQ ID NO:2).

For example, appropriate functional Vα domain CDR1 amino acid sequences may have at least 80% sequence identity to SEQ ID NO: 60, i.e. it may have at least 80%, at least 83%, or 100% sequence identity to SEQ ID NO: 60. Suitably, percent identity is calculated as the percentage of identity to the entire length of the reference sequence (e.g. SEQ ID NO: 60). In other words, appropriate functional Vα domain CDR1 amino acid sequences may vary from the sequence shown in SEQ ID NO: 60 by one or several amino acids. In one example, the CDR1 of the Vα domain comprises or consists of the amino acid sequence of SEQ ID NO: 60. In examples where the TCR Vα domain CDR1 has the amino acid sequence of SEQ ID NO: 60, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 61, or a functional variant thereof (i.e. wherein the variant retains the ability to specifically bind to HLA-A*02, most preferably HLA-A*02:01).

For example, appropriate functional Vα domain CDR2 amino acid sequences may have at least 80% sequence identity to SEQ ID NO: 61, i.e. it may have at least 80%, at least 85%, or 100% sequence identity to SEQ ID NO: 61. In other words, appropriate (functional) Vα domain CDR2 amino acid sequences may vary from the sequence shown in SEQ ID NO: 61 by one or several amino acids.

In one example, the CDR2 of the Vα domain comprises or consists of the amino acid sequence of SEQ ID NO: 61. In examples where the TCR Vα domain CDR2 has the amino acid sequence of SEQ ID NO: 61, the CDR2 may be encoded by any appropriate nucleic acid sequence.

The encoded TCR Vα domain may therefore comprise the CDRs mentioned in detail above (by SEQ ID specifically i.e. SEQ ID NO:62, SEQ ID NO: 60 and SEQ ID NO: 61, or functional variants thereof), with appropriate intervening sequences between the CDRs.

The encoded TCR Vα domain may comprise an amino acid sequence of SEQ ID NO: 66, or a functional variant thereof (i.e. wherein the variant TCR Vα domain retains the ability to specifically bind to a peptide comprising the amino acid sequence of SEQ ID NO:1 (e.g. the peptide shown in SEQ ID NO:2) when part of a binding protein described herein).

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 66, whilst retaining the ability to specifically bind to a peptide comprising the amino acid sequence of SEQ ID NO:1 (e.g. the peptide shown in SEQ ID NO:2). In other words, a functional TCR Vα domain with one or several amino acid substitutions compared to the sequence of SEQ ID NO: 66 is also encompassed. The variability in sequence compared to SEQ ID NO: 66 may all be in regions of the TCR Vα domain that do not form CDRs (i.e. the variant may have the CDRs of SEQ ID NO: 62, SEQ ID NO: 60 and/or SEQ ID NO: 61, and still have 25% (or less) sequence variability compared to SEQ ID NO:66). In other words, the sequence of the CDRs of SEQ ID NO: 66 may be retained whilst the rest of the sequence is varied, as appropriate within the “at least 75% identity” parameters specified above.

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 66, wherein the TCR Vα domain comprises a CDR3 having an amino acid sequence of SEQ ID NO: 62. In this example, the TCR Vα domain CDR1 may have an amino acid sequence of SEQ ID NO: 60 and the TCR Vα domain CDR2 may have an amino acid sequence of SEQ ID NO: 61.

As another example, the encoded TCR Vα domain may comprise an amino acid sequence having the amino acid sequence of SEQ ID NO: 66, with 0 to 10 (or 0 to 5) amino acid substitutions, insertions or deletions, wherein the TCR Vα domain comprises a CDR3 having an amino acid sequence of SEQ ID NO: 62. In this example, the TCR Vα domain CDR1 may have an amino acid sequence of SEQ ID NO: 60 and the TCR Vα domain CDR2 may have an amino acid sequence of SEQ ID NO: 61.

In examples where the TCR Vα domain has the amino acid sequence of SEQ ID NO: 66, the TCR Vα domain may be encoded by the nucleic acid sequence of SEQ ID NO: 67, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

In one example, the nucleic acid composition provided herein comprises a nucleic acid sequence that encodes a TCR Vα domain comprising a CDR3 amino acid sequence having at least 80% sequence identity to SEQ ID NO:62, or a functional fragment thereof.

In another example, the CDR3 of the Vα domain of a nucleic acid composition provided herein comprises or consists of the amino acid sequence of SEQ ID NO: 62.

In another example, the Vα domain of a nucleic acid composition provided herein comprises an amino acid sequence having at least 80% sequence identity to, comprising, or consisting of, SEQ ID NO: 66.

As provided above, the inventors identified TCR clone TCR-4 which interacts with SLGDWGAEA (SEQ ID NO: 2) in the context of HLA-A*02:01. The sequences provided herein that correspond to TCR clone TCR-4 are SEQ ID NO:s 60 to 69.

An example of an appropriate TCR Vβ domain CDR3 amino acid sequence that confers specific binding to a TIMP3 antigen, in particular a peptide comprising the amino acid sequence of (SEQ ID NO:1 (e.g. to SEQ ID NO:2), is shown in SEQ ID NO:65.

For example, appropriate (functional) Vβ domain CDR3 amino acid sequences may have at least 80% sequence identity to SEQ ID NO: 65, i.e. they may have at least 80%, at least 86%, at least 93%, or 100% sequence identity to SEQ ID NO: 65. Suitably, percent identity is calculated as the percentage of identity to the entire length of the reference sequence (e.g. SEQ ID NO: 65).

In other words, appropriate (functional) Vβ domain CDR3 amino acid sequences may vary from the sequence shown in SEQ ID NO: 65 by one or several (e.g. two) amino acids.

In one example, the CDR3 of the Vβ domain comprises or consists of the amino acid sequence of SEQ ID NO: 65. In examples where the TCR Vβ domain CDR3 has the amino acid sequence of SEQ ID NO:65, the CDR3 may be encoded by any appropriate nucleic acid sequence.

The encoded TCR Vβ domain may comprise, in addition to the specified CDR3, a CDR1 comprising an amino acid sequence of SEQ ID NO: 63, or a functional variant thereof (i.e. wherein the variant retains the ability to specifically bind to a peptide comprising the amino acid sequence of SEQ ID NO:1 (e.g. the peptide shown in SEQ ID NO:2).

For example, appropriate functional Vβ domain CDR1 amino acid sequences may have at least 80% sequence identity to SEQ ID NO: 63, i.e. it may have at least 80%, or 100% sequence identity to SEQ ID NO: 63. Suitably, percent identity is calculated as the percentage of identity to the entire length of the reference sequence (e.g. SEQ ID NO: 63). In other words, appropriate (functional) Vβ domain CDR1 amino acid sequences may vary from the sequence shown in SEQ ID NO:63 by one or several amino acids.

In one example, the CDR1 of the Vβ domain comprises or consists of the amino acid sequence of SEQ ID NO: 63. In examples where the TCR Vα domain CDR1 has the amino acid sequence of SEQ ID NO:63, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 64, or a functional variant thereof (i.e. wherein the variant retains the ability to specifically bind to HLA-A*02, most preferably HLA-A*02:01).

For example, appropriate functional Vβ domain CDR2 amino acid sequences may have at least 80% sequence identity to SEQ ID NO: 64, i.e. it may have at least 80%, at least 83%, or 100% sequence identity to SEQ ID NO: 64. In other words, appropriate (functional) Vβ domain CDR2 amino acid sequences may vary from the sequence shown in SEQ ID NO: 64 by one or several amino acids.

In one example, the CDR2 of the Vβ domain comprises or consists of the amino acid sequence of SEQ ID NO: 64. In examples where the TCR Vβ domain CDR2 has the amino acid sequence of SEQ ID NO:64, the CDR2 may be encoded by any appropriate nucleic acid sequence.

The encoded TCR Vβ domain may therefore comprise the CDRs mentioned in detail above (by SEQ ID specifically i.e. SEQ ID NO:65, SEQ ID NO: 63 and SEQ ID NO: 64, or functional variants thereof), with appropriate intervening sequences between the CDRs.

The encoded TCR Vβ domain may have an amino acid sequence of SEQ ID NO: 68, or a functional variant thereof (i.e. wherein the variant TCR Vβ domain retains the ability to specifically bind to a peptide comprising the amino acid sequence of SEQ ID NO:1 (e.g. the peptide shown in SEQ ID NO:2) when part of a binding protein described herein).

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 68, whilst retaining the ability to specifically bind to a peptide comprising the amino acid sequence of SEQ ID NO:1 (e.g. the peptide shown in SEQ ID NO:2). In other words, a functional TCR Vβ domain with one or several amino acid substitutions compared to the sequence of SEQ ID NO: 68 is also encompassed. The variability in sequence compared to SEQ ID NO:68 may all be in regions of the TCR Vβ domain that do not form CDRs (i.e. the variant may have the CDRs of SEQ ID NO: 65, SEQ ID NO: 63 and/or SEQ ID NO: 64, and still have 25% (or less) sequence variability compared to SEQ ID NO: 68). In other words, the sequence of the CDRs of SEQ ID NO: 68 may be retained whilst the rest of the sequence is varied, as appropriate within the “at least 75% identity” parameters specified above.

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 68, wherein the TCR Vβ domain comprises a CDR3 having an amino acid sequence of SEQ ID NO: 65. In this example, the TCR Vβ domain CDR1 may have an amino acid sequence of SEQ ID NO:63 and the TCR Vβ domain CDR2 may have an amino acid sequence of SEQ ID NO: 64.

In examples where the TCR Vβ domain has the amino acid sequence of SEQ ID NO:68, the TCR Vβ domain may be encoded by the nucleic acid sequence of SEQ ID NO:69, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

In an example, the nucleic acid composition provided herein comprises a nucleic acid sequence that encodes a TCR Vβ domain comprising a CDR3 amino acid sequence having at least 80% sequence identity to SEQ ID NO:65, or a functional fragment thereof.

In another example, the CDR3 of the Vβ domain of a nucleic acid composition provided herein comprises or consists of the amino acid sequence of SEQ ID NO:65.

In a further example, the Vβ domain of a nucleic acid composition provided herein comprises an amino acid sequence having at least 80% sequence identity to, comprising, or consisting of, SEQ ID NO: 68.

The TCR Vβ domain sequences derived from TCR clone TCR-4 discussed above are particularly compatible with the TCR Vα domain sequences derived from TCR clone TCR-4 discussed elsewhere herein.

Accordingly, in one example, a nucleic acid composition described herein encodes a binding protein specific for a peptide comprising the amino acid sequence of SEQ ID NO:1 having TCR Vα domain comprising a CDR3 amino acid sequence having at least 80% sequence identity to SEQ ID NO:62, or a functional fragment thereof; and a nucleic acid sequence that encodes a TCR Vβ domain comprising a CDR3 amino acid sequence having at least 80% sequence identity to SEQ ID NO:65, or a functional fragment thereof.

In a particular example, a nucleic acid composition described herein encodes a binding protein specific for a peptide comprising the amino acid sequence of SEQ ID NO:1 having a TCR Vα domain with a CDR3 amino acid sequence comprising or consisting of the amino acid sequence of SEQ ID NO: 62; and a TCR Vβ domain with a CDR3 comprising or consisting of the amino acid sequence of SEQ ID NO:65. In addition, the a peptide comprising the amino acid sequence of SEQ ID NO:1 may comprise or consist of the sequence shown in SEQ ID NO: 2. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 66; and the Vβ domain may comprise an amino acid sequence having at least 80% sequence identity to, comprising, or consisting of, SEQ ID NO: 68. In one example, the Vα domain comprises the amino acid sequence of SEQ ID NO: 66 and the Vβ domain comprises the amino acid sequence of SEQ ID NO: 68. In such cases, the Vα domain may be encoded by a nucleic acid sequence comprising the sequence of SEQ ID NO: 67; and the Vβ domain may be encoded by a nucleic acid sequence comprising the sequence of SEQ ID NO: 69.

In this particular example, the TCR Vα domain may include a CDR1 amino acid sequence comprising or consisting of the amino acid sequence of SEQ ID NO: 60 and a CDR2 amino acid sequence comprising or consisting of the amino acid sequence of SEQ ID NO:61. Furthermore, the TCR Vβ domain may include a CDR1 amino acid sequence comprising or consisting of the amino acid sequence of SEQ ID NO:63 and a CDR2 amino acid sequence comprising or consisting of the amino acid sequence of SEQ ID NO: 64.

For the avoidance of doubt, this particular example encompasses components of TCR clone TCR-4 exemplified herein. The different components of TCR clone TCR-4 and their respective SEQ ID Nos are summarised in Table 9 below.

As stated in more detail elsewhere herein, the nucleic acid composition described herein encodes both a TCR Vα domain and a TCR Vβ domain, which form the binding protein that is capable of specifically binding to a TIMP3 antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(iv) TCR Clone TCR-5: TCR Components that Interact with SLGDVGAEA (SEQ ID NO: 2)

As provided elsewhere herein, the inventors have also identified TCR clone TCR-5 which interacts with SLGDWGAEA (SEQ ID NO: 2) in the context of HLA-A*02:01. The sequences provided herein that correspond to TCR clone TCR-5 are SEQ ID NO:s 70 to 79.

In one embodiment, an isolated nucleic acid composition that encodes a TIMP3 antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:72, or a functional fragment thereof; and a nucleic acid sequence that encodes a TCR Vβ domain comprising a CDR3 amino acid sequence having at least 80% sequence identity to SEQ ID NO: 75, or a functional fragment thereof, wherein the CDR3 sequences together specifically bind to a peptide comprising the amino acid sequence of SEQ ID NO:1 (e.g. when the peptide is complexed with HLA).

An example of an appropriate TCR Vα domain CDR3 amino acid sequence that confers specific binding to a TIMP3 antigen, in particular a peptide comprising the amino acid sequence of SEQ ID NO:1 (e.g. to SLGDVGAEA (SEQ ID NO: 2)), is shown in SEQ ID NO: 72

For example, appropriate (functional) Vα domain CDR3 amino acid sequences may have at least 80% sequence identity to SEQ ID NO: 72, i.e. they may have at least 80%, at least 84%, at least 92%, or 100% sequence identity to SEQ ID NO: 72. Suitably, percent identity is calculated as the percentage of identity to the entire length of the reference sequence (e.g. SEQ ID NO: 72).

In other words, appropriate (functional) Vα domain CDR3 amino acid sequences may vary from the sequence shown in SEQ ID NO: 72 by one or several (e.g. two etc) amino acids.

In one example, the CDR3 of the Vα domain comprises or consists of the amino acid sequence of SEQ ID NO: 72. In examples where the TCR Vα domain CDR3 has the amino acid sequence of SEQ ID NO: 72, the CDR3 may be encoded by any appropriate nucleic acid sequence.

The encoded TCR Vα domain may comprise, in addition to the specified CDR3, a CDR1 comprising an amino acid sequence of SEQ ID NO: 70, or a functional variant thereof (i.e. wherein the variant retains the ability to specifically bind to a peptide comprising the amino acid sequence of SEQ ID NO:1 (e.g. the peptide shown in SEQ ID NO:2).

For example, appropriate functional Vα domain CDR1 amino acid sequences may have at least 80% sequence identity to SEQ ID NO: 70, i.e. it may have at least 80%, at least 83%, or 100% sequence identity to SEQ ID NO: 70. Suitably, percent identity is calculated as the percentage of identity to the entire length of the reference sequence (e.g. SEQ ID NO: 70). In other words, appropriate functional Vα domain CDR1 amino acid sequences may vary from the sequence shown in SEQ ID NO: 70 by one or several amino acids.

In one example, the CDR1 of the Vα domain comprises or consists of the amino acid sequence of SEQ ID NO: 70. In examples where the TCR Vα domain CDR1 has the amino acid sequence of SEQ ID NO: 70, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 71, or a functional variant thereof (i.e. wherein the variant retains the ability to specifically bind to HLA-A*02, most preferably HLA-A*02:01).

For example, appropriate functional Vα domain CDR2 amino acid sequences may have at least 80% sequence identity to SEQ ID NO: 71, i.e. it may have at least 80%, at least 83%, or 100% sequence identity to SEQ ID NO: 71. In other words, appropriate (functional) Vα domain CDR2 amino acid sequences may vary from the sequence shown in SEQ ID NO: 71 by one or several amino acids.

In one example, the CDR2 of the Vα domain comprises or consists of the amino acid sequence of SEQ ID NO: 71. In examples where the TCR Vα domain CDR2 has the amino acid sequence of SEQ ID NO: 71, the CDR2 may be encoded by any appropriate nucleic acid sequence.

The encoded TCR Vα domain may therefore comprise the CDRs mentioned in detail above (by SEQ ID specifically i.e. SEQ ID NO:72, SEQ ID NO: 70 and SEQ ID NO: 71, or functional variants thereof), with appropriate intervening sequences between the CDRs.

The encoded TCR Vα domain may comprise an amino acid sequence of SEQ ID NO: 76, or a functional variant thereof (i.e. wherein the variant TCR Vα domain retains the ability to specifically bind to a peptide comprising the amino acid sequence of SEQ ID NO:1 (e.g. the peptide shown in SEQ ID NO:2) when part of a binding protein described herein).

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 76, whilst retaining the ability to specifically bind to a peptide comprising the amino acid sequence of SEQ ID NO:1 (e.g. the peptide shown in SEQ ID NO:2). In other words, a functional TCR Vα domain with one or several amino acid substitutions compared to the sequence of SEQ ID NO: 76 is also encompassed. The variability in sequence compared to SEQ ID NO: 76 may all be in regions of the TCR Vα domain that do not form CDRs (i.e. the variant may have the CDRs of SEQ ID NO: 72, SEQ ID NO: 70 and/or SEQ ID NO: 71, and still have 25% (or less) sequence variability compared to SEQ ID NO: 76). In other words, the sequence of the CDRs of SEQ ID NO: 76 may be retained whilst the rest of the sequence is varied, as appropriate within the “at least 75% identity” parameters specified above.

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 76, wherein the TCR Vα domain comprises a CDR3 having an amino acid sequence of SEQ ID NO: 72. In this example, the TCR Vα domain CDR1 may have an amino acid sequence of SEQ ID NO: 70 and the TCR Vα domain CDR2 may have an amino acid sequence of SEQ ID NO: 71.

As another example, the encoded TCR Vα domain may comprise an amino acid sequence having the amino acid sequence of SEQ ID NO: 76, with 0 to 10 (or 0 to 5) amino acid substitutions, insertions or deletions, wherein the TCR Vα domain comprises a CDR3 having an amino acid sequence of SEQ ID NO: 72. In this example, the TCR Vα domain CDR1 may have an amino acid sequence of SEQ ID NO: 70 and the TCR Vα domain CDR2 may have an amino acid sequence of SEQ ID NO: 71.

In examples where the TCR Vα domain has the amino acid sequence of SEQ ID NO: 76, the TCR Vα domain may be encoded by the nucleic acid sequence of SEQ ID NO: 77, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

In one example, the nucleic acid composition provided herein comprises a nucleic acid sequence that encodes a TCR Vα domain comprising a CDR3 amino acid sequence having at least 80% sequence identity to SEQ ID NO:72, or a functional fragment thereof.

In another example, the CDR3 of the Vα domain of a nucleic acid composition provided herein comprises or consists of the amino acid sequence of SEQ ID NO: 72.

In another example, the Vα domain of a nucleic acid composition provided herein comprises an amino acid sequence having at least 80% sequence identity to, comprising, or consisting of, SEQ ID NO: 76.

As provided above, the inventors identified TCR clone TCR-5 which interacts with SLGDWGAEA (SEQ ID NO: 2) in the context of HLA-A*02:01. The sequences provided herein that correspond to TCR clone TCR-5 are SEQ ID NO:s 70 to 79.

An example of an appropriate TCR Vβ domain CDR3 amino acid sequence that confers specific binding to a peptide comprising the amino acid sequence of SEQ ID NO:1 (e.g. the peptide shown in SEQ ID NO:2) is shown in SEQ ID NO:75.

For example, appropriate (functional) Vβ domain CDR3 amino acid sequences may have at least 80% sequence identity to SEQ ID NO: 75, i.e. they may have at least 80%, at least 85%, at least 92%, or 100% sequence identity to SEQ ID NO: 75. Suitably, percent identity is calculated as the percentage of identity to the entire length of the reference sequence (e.g. SEQ ID NO: 75).

In other words, appropriate (functional) Vβ domain CDR3 amino acid sequences may vary from the sequence shown in SEQ ID NO: 75 by one or several (e.g. two) amino acids.

In one example, the CDR3 of the Vβ domain comprises or consists of the amino acid sequence of SEQ ID NO: 75. In examples where the TCR Vβ domain CDR3 has the amino acid sequence of SEQ ID NO:75, the CDR3 may be encoded by any appropriate nucleic acid sequence.

The encoded TCR Vβ domain may comprise, in addition to the specified CDR3, a CDR1 comprising an amino acid sequence of SEQ ID NO: 73, or a functional variant thereof (i.e. wherein the variant retains the ability to specifically bind to a peptide comprising the amino acid sequence of SEQ ID NO:1 (e.g. the peptide shown in SEQ ID NO:2).

For example, appropriate functional Vβ domain CDR1 amino acid sequences may have at least 80% sequence identity to SEQ ID NO: 73, i.e. it may have at least 80%, at least 83%, or 100% sequence identity to SEQ ID NO: 73. In other words, appropriate (functional) Vβ domain CDR1 amino acid sequences may vary from the sequence shown in SEQ ID NO:73 by one or several amino acids.

In one example, the CDR1 of the Vβ domain comprises or consists of the amino acid sequence of SEQ ID NO: 73. In examples where the TCR Vα domain CDR1 has the amino acid sequence of SEQ ID NO:73, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 74, or a functional variant thereof (i.e. wherein the variant retains the ability to specifically bind to HLA-A*02, most preferably HLA-A*02:01).

For example, appropriate functional Vβ domain CDR2 amino acid sequences may have at least 80% sequence identity to SEQ ID NO: 74, i.e. it may have at least 80%, at least 83, or 100% sequence identity to SEQ ID NO: 74. Suitably, percent identity is calculated as the percentage of identity to the entire length of the reference sequence (e.g. SEQ ID NO: 74). In other words, appropriate (functional) Vβ domain CDR2 amino acid sequences may vary from the sequence shown in SEQ ID NO: 74 by one or several amino acids.

In one example, the CDR2 of the Vβ domain comprises or consists of the amino acid sequence of SEQ ID NO: 74. In examples where the TCR Vβ domain CDR2 has the amino acid sequence of SEQ ID NO:74, the CDR2 may be encoded by any appropriate nucleic acid sequence.

The encoded TCR Vβ domain may therefore comprise the CDRs mentioned in detail above (by SEQ ID specifically i.e. SEQ ID NO:75, SEQ ID NO: 73 and SEQ ID NO: 74, or functional variants thereof), with appropriate intervening sequences between the CDRs.

The encoded TCR Vβ domain may have an amino acid sequence of SEQ ID NO: 78, or a functional variant thereof (i.e. wherein the variant TCR Vβ domain retains the ability to specifically bind to a peptide comprising the amino acid sequence of SEQ ID NO:1 (e.g. the peptide shown in SEQ ID NO:2) when part of a binding protein described herein).

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 78, whilst retaining the ability to specifically bind to a peptide comprising the amino acid sequence of SEQ ID NO:1 (e.g. the peptide shown in SEQ ID NO:2). In other words, a functional TCR Vβ domain with one or several amino acid substitutions compared to the sequence of SEQ ID NO: 78 is also encompassed. The variability in sequence compared to SEQ ID NO:78 may all be in regions of the TCR Vβ domain that do not form CDRs (i.e. the variant may have the CDRs of SEQ ID NO: 75, SEQ ID NO: 73 and/or SEQ ID NO: 74, and still have 25% (or less) sequence variability compared to SEQ ID NO: 78). In other words, the sequence of the CDRs of SEQ ID NO: 78 may be retained whilst the rest of the sequence is varied, as appropriate within the “at least 75% identity” parameters specified above.

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 78, wherein the TCR Vβ domain comprises a CDR3 having an amino acid sequence of SEQ ID NO: 75. In this example, the TCR Vβ domain CDR1 may have an amino acid sequence of SEQ ID NO:73 and the TCR Vβ domain CDR2 may have an amino acid sequence of SEQ ID NO: 74.

In examples where the TCR Vβ domain has the amino acid sequence of SEQ ID NO:78, the TCR Vβ domain may be encoded by the nucleic acid sequence of SEQ ID NO: 79, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

In an example, the nucleic acid composition provided herein comprises a nucleic acid sequence that encodes a TCR Vβ domain comprising a CDR3 amino acid sequence having at least 80% sequence identity to SEQ ID NO:75, or a functional fragment thereof.

In another example, the CDR3 of the Vβ domain of a nucleic acid composition provided herein comprises or consists of the amino acid sequence of SEQ ID NO:75.

In a further example, the Vβ domain of a nucleic acid composition provided herein comprises an amino acid sequence having at least 80% sequence identity to, comprising, or consisting of, SEQ ID NO: 78.

The TCR Vβ domain sequences derived from TCR clone TCR-5 discussed above are particularly compatible with the TCR Vα domain sequences derived from TCR clone TCR-5 discussed elsewhere herein.

Accordingly, in one example, a nucleic acid composition described herein encodes a binding protein specific for a peptide comprising the amino acid sequence of SEQ ID NO:1 having TCR Vα domain comprising a CDR3 amino acid sequence having at least 80% sequence identity to SEQ ID NO:72, or a functional fragment thereof; and a nucleic acid sequence that encodes a TCR Vβ domain comprising a CDR3 amino acid sequence having at least 80% sequence identity to SEQ ID NO:75, or a functional fragment thereof.

In a particular example, a nucleic acid composition described herein encodes a binding protein specific for a peptide comprising the amino acid sequence of SEQ ID NO:1 having a TCR Vα domain with a CDR3 amino acid sequence comprising or consisting of the amino acid sequence of SEQ ID NO: 72; and a TCR Vβ domain with a CDR3 comprising or consisting of the amino acid sequence of SEQ ID NO:75. In addition, the peptide comprising the amino acid sequence of SEQ ID NO:1 may comprise or consist of the sequence shown in SEQ ID NO: 2. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 76; and the Vβ domain may comprise an amino acid sequence having at least 80% sequence identity to, comprising, or consisting of, SEQ ID NO: 78. In one example, the Vα domain comprises the amino acid sequence of SEQ ID NO: 76 and the Vβ domain comprises the amino acid sequence of SEQ ID NO: 78. In such cases, the Vα domain may be encoded by a nucleic acid sequence comprising the sequence of SEQ ID NO: 77; and the Vβ domain may be encoded by a nucleic acid sequence comprising the sequence of SEQ ID NO: 79.

In this particular example, the TCR Vα domain may include a CDR1 amino acid sequence comprising or consisting of the amino acid sequence of SEQ ID NO:70 and a CDR2 amino acid sequence comprising or consisting of the amino acid sequence of SEQ ID NO:71. Furthermore, the TCR Vβ domain may include a CDR1 amino acid sequence comprising or consisting of the amino acid sequence of SEQ ID NO:73 and a CDR2 amino acid sequence comprising or consisting of the amino acid sequence of SEQ ID NO: 74.

For the avoidance of doubt, this particular example encompasses components of TCR clone TCR-5 exemplified herein. The different components of TCR clone TCR-5 and their respective SEQ ID Nos are summarised in Table 10 below.

As stated in more detail elsewhere herein, the nucleic acid composition described herein encodes both a TCR Vα domain and a TCR Vβ domain, which form the binding protein that is capable of specifically binding to a TIMP3 antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(v) TCR Clone TCR-6: TCR Components that Interact with SLGDWMAEA (SEQ ID NO: 2)

As provided elsewhere herein, the inventors have also identified TCR clone TCR-6 which interacts with SLGDWGAEA (SEQ ID NO: 2) in the context of HLA-A*02:01. The sequences provided herein that correspond to TCR clone TCR-6 are SEQ ID NO:s 80 to 89.

In one embodiment, an isolated nucleic acid composition that encodes a TIMP3 antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:82, or a functional fragment thereof; and a nucleic acid sequence that encodes a TCR Vβ domain comprising a CDR3 amino acid sequence having at least 80% sequence identity to SEQ ID NO: 85, or a functional fragment thereof, wherein the CDR3 sequences together specifically bind to a peptide comprising the amino acid sequence of SEQ ID NO:1 (e.g. when the peptide is complexed with HLA).

An example of an appropriate TCR Vα domain CDR3 amino acid sequence that confers specific binding to a TIMP3 antigen, in particular a peptide comprising the amino acid sequence of SEQ ID NO:1 (e.g. to SLGDWGAEA (SEQ ID NO: 2)), is shown in SEQ ID NO: 82.

For example, appropriate (functional) Vα domain CDR3 amino acid sequences may have at least 80% sequence identity to SEQ ID NO: 82, i.e. they may have at least 80%, at least 82%,at least 88%, at least 94%, or 100% sequence identity to SEQ ID NO: 82.

In one example, the CDR3 of the Vα domain comprises or consists of the amino acid sequence of SEQ ID NO: 82. In examples where the TCR Vα domain CDR3 has the amino acid sequence of SEQ ID NO: 82, the CDR3 may be encoded by any appropriate nucleic acid sequence.

The encoded TCR Vα domain may comprise, in addition to the specified CDR3, a CDR1 comprising an amino acid sequence of SEQ ID NO: 80, or a functional variant thereof (i.e. wherein the variant retains the ability to specifically bind to a peptide comprising the amino acid sequence of SEQ ID NO:1 (e.g. the peptide shown in SEQ ID NO:2)).

For example, appropriate functional Vα domain CDR1 amino acid sequences may have at least 80% sequence identity to SEQ ID NO: 80, i.e. it may have at least 80%, at least 85%, or 100% sequence identity to SEQ ID NO: 80. In other words, appropriate functional Vα domain CDR1 amino acid sequences may vary from the sequence shown in SEQ ID NO: 80 by one or several amino acids.

In one example, the CDR1 of the Vα domain comprises or consists of the amino acid sequence of SEQ ID NO: 80. In examples where the TCR Vα domain CDR1 has the amino acid sequence of SEQ ID NO: 80, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 81, or a functional variant thereof (i.e. wherein the variant retains the ability to specifically bind to HLA-A*02, most preferably HLA-A*02:01).

For example, appropriate functional Vα domain CDR2 amino acid sequences may have at least 80% sequence identity to SEQ ID NO: 81, i.e. it may have at least 80%, at least 87%, or 100% sequence identity to SEQ ID NO: 81. In other words, appropriate (functional) Vα domain CDR2 amino acid sequences may vary from the sequence shown in SEQ ID NO: 81 by one or several amino acids. In one example, the CDR2 of the Vα domain comprises or consists of the amino acid sequence of SEQ ID NO: 81. In examples where the TCR Vα domain CDR2 has the amino acid sequence of SEQ ID NO: 81, the CDR2 may be encoded by any appropriate nucleic acid sequence.

The encoded TCR Vα domain may therefore comprise the CDRs mentioned in detail above (by SEQ ID specifically i.e. SEQ ID NO:82, SEQ ID NO: 80 and SEQ ID NO: 81, or functional variants thereof), with appropriate intervening sequences between the CDRs.

The encoded TCR Vα domain may comprise an amino acid sequence of SEQ ID NO: 86, or a functional variant thereof (i.e. wherein the variant TCR Vα domain retains the ability to specifically bind to a peptide comprising the amino acid sequence of SEQ ID NO:1 (e.g. the peptide shown in SEQ ID NO:2) when part of a binding protein described herein).

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 86, whilst retaining the ability to specifically bind to a peptide comprising the amino acid sequence of SEQ ID NO:1 (e.g. the peptide shown in SEQ ID NO:2). In other words, a functional TCR Vα domain with one or several amino acid substitutions compared to the sequence of SEQ ID NO: 86 is also encompassed. The variability in sequence compared to SEQ ID NO: 86 may all be in regions of the TCR Vα domain that do not form CDRs (i.e. the variant may have the CDRs of SEQ ID NO: 82, SEQ ID NO: 80 and/or SEQ ID NO: 81, and still have 25% (or less) sequence variability compared to SEQ ID NO:86). In other words, the sequence of the CDRs of SEQ ID NO: 86 may be retained whilst the rest of the sequence is varied, as appropriate within the “at least 75% identity” parameters specified above.

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 86, wherein the TCR Vα domain comprises a CDR3 having an amino acid sequence of SEQ ID NO: 82. In this example, the TCR Vα domain CDR1 may have an amino acid sequence of SEQ ID NO: 80 and the TCR Vα domain CDR2 may have an amino acid sequence of SEQ ID NO: 81.

As another example, the encoded TCR Vα domain may comprise an amino acid sequence having the amino acid sequence of SEQ ID NO: 86, with 0 to 10 (or 0 to 5) amino acid substitutions, insertions or deletions, wherein the TCR Vα domain comprises a CDR3 having an amino acid sequence of SEQ ID NO: 82. In this example, the TCR Vα domain CDR1 may have an amino acid sequence of SEQ ID NO: 80 and the TCR Vα domain CDR2 may have an amino acid sequence of SEQ ID NO: 81.

In examples where the TCR Vα domain has the amino acid sequence of SEQ ID NO: 86, the TCR Vα domain may be encoded by the nucleic acid sequence of SEQ ID NO: 87, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

In one example, the nucleic acid composition provided herein comprises a nucleic acid sequence that encodes a TCR Vα domain comprising a CDR3 amino acid sequence having at least 80% sequence identity to SEQ ID NO:82, or a functional fragment thereof.

In another example, the CDR3 of the Vα domain of a nucleic acid composition provided herein comprises or consists of the amino acid sequence of SEQ ID NO: 82.

In another example, the Vα domain of a nucleic acid composition provided herein comprises an amino acid sequence having at least 80% sequence identity to, comprising, or consisting of, SEQ ID NO: 86.

As provided above, the inventors identified TCR clone TCR-6 which interacts with SLGDWGAEA (SEQ ID NO: 2) in the context of HLA-A*02:01. The sequences provided herein that correspond to TCR clone TCR-6 are SEQ ID NO:s 80 to 89.

An example of an appropriate TCR Vβ domain CDR3 amino acid sequence that confers specific binding to a TIMP3 antigen, in particular a peptide comprising the amino acid sequence of SEQ ID NO: 1 (e.g. to SLGDWGAEA (SEQ ID NO: 2)), is shown in SEQ ID NO:85. As would be clear to a person of skill in the art, variants of the amino acid sequence shown in SEQ ID NO:85 may also be functional (i.e. retain their ability to confer specific binding to a peptide comprising the amino acid sequence of SEQ ID NO:1 (e.g. the peptide shown in SEQ ID NO:2) when the CDR3 is part of TCR Vβ domain).

For example, appropriate (functional) Vβ domain CDR3 amino acid sequences may have at least 80% sequence identity to SEQ ID NO: 85, i.e. they may have at least 80%, at least 83%, at least 91%, or 100% sequence identity to SEQ ID NO: 85. In other words, appropriate (functional) Vβ domain CDR3 amino acid sequences may vary from the sequence shown in SEQ ID NO: 85 by one or several (e.g. two) amino acids.

In one example, the CDR3 of the Vβ domain comprises or consists of the amino acid sequence of SEQ ID NO: 85. In examples where the TCR Vβ domain CDR3 has the amino acid sequence of SEQ ID NO:85, the CDR3 may be encoded by any appropriate nucleic acid sequence.

The encoded TCR Vβ domain may comprise, in addition to the specified CDR3, a CDR1 comprising an amino acid sequence of SEQ ID NO: 83, or a functional variant thereof (i.e. wherein the variant retains the ability to specifically bind to a peptide comprising the amino acid sequence of SEQ ID NO:1 (e.g. the peptide shown in SEQ ID NO:2)).

For example, appropriate functional Vβ domain CDR1 amino acid sequences may have at least 80% sequence identity to SEQ ID NO: 83, i.e. it may have at least 80%, or 100% sequence identity to SEQ ID NO: 83. In other words, appropriate (functional) Vβ domain CDR1 amino acid sequences may vary from the sequence shown in SEQ ID NO:83 by one or several amino acids.

In one example, the CDR1 of the Vβ domain comprises or consists of the amino acid sequence of SEQ ID NO: 83. In examples where the TCR Vα domain CDR1 has the amino acid sequence of SEQ ID NO:83, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 84, or a functional variant thereof (i.e. wherein the variant retains the ability to specifically bind to HLA-A*02, most preferably HLA-A*02:01).

For example, appropriate functional Vβ domain CDR2 amino acid sequences may have at least 80% sequence identity to SEQ ID NO: 84, i.e. it may have at least 80%, at least 83%, or 100% sequence identity to SEQ ID NO: 84. In other words, appropriate (functional) Vβ domain CDR2 amino acid sequences may vary from the sequence shown in SEQ ID NO: 84 by one or several amino acids.

In one example, the CDR2 of the Vβ domain comprises or consists of the amino acid sequence of SEQ ID NO: 84. In examples where the TCR Vβ domain CDR2 has the amino acid sequence of SEQ ID NO:84, the CDR2 may be encoded by any appropriate nucleic acid sequence.

The encoded TCR Vβ domain may therefore comprise the CDRs mentioned in detail above (by SEQ ID specifically i.e. SEQ ID NO:85, SEQ ID NO: 83 and SEQ ID NO: 84, or functional variants thereof), with appropriate intervening sequences between the CDRs.

The encoded TCR Vβ domain may have an amino acid sequence of SEQ ID NO: 88, or a functional variant thereof (i.e. wherein the variant TCR Vβ domain retains the ability to specifically bind to a peptide comprising the amino acid sequence of SEQ ID NO:1 (e.g. the peptide shown in SEQ ID NO:2)) when part of a binding protein described herein).

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 88, whilst retaining the ability to specifically bind to a peptide comprising the amino acid sequence of SEQ ID NO:1 (e.g. the peptide shown in SEQ ID NO:2). In other words, a functional TCR Vβ domain with one or several amino acid substitutions compared to the sequence of SEQ ID NO: 88 is also encompassed. The variability in sequence compared to SEQ ID NO:88 may all be in regions of the TCR Vβ domain that do not form CDRs (i.e. the variant may have the CDRs of SEQ ID NO: 85, SEQ ID NO: 83 and/or SEQ ID NO: 84, and still have 25% (or less) sequence variability compared to SEQ ID NO: 88). In other words, the sequence of the CDRs of SEQ ID NO: 88 may be retained whilst the rest of the sequence is varied, as appropriate within the “at least 75% identity” parameters specified above.

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 88, wherein the TCR Vβ domain comprises a CDR3 having an amino acid sequence of SEQ ID NO: 85. In this example, the TCR Vβ domain CDR1 may have an amino acid sequence of SEQ ID NO:83 and the TCR Vβ domain CDR2 may have an amino acid sequence of SEQ ID NO: 84.

In examples where the TCR Vβ domain has the amino acid sequence of SEQ ID NO:88, the TCR Vβ domain may be encoded by the nucleic acid sequence of SEQ ID NO: 89, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

In an example, the nucleic acid composition provided herein comprises a nucleic acid sequence that encodes a TCR Vβ domain comprising a CDR3 amino acid sequence having at least 80% sequence identity to SEQ ID NO:85, or a functional fragment thereof.

In another example, the CDR3 of the Vβ domain of a nucleic acid composition provided herein comprises or consists of the amino acid sequence of SEQ ID NO:85.

In a further example, the Vβ domain of a nucleic acid composition provided herein comprises an amino acid sequence having at least 80% sequence identity to, comprising, or consisting of, SEQ ID NO: 88.

The TCR Vβ domain sequences derived from TCR clone TCR-6 discussed above are particularly compatible with the TCR Vα domain sequences derived from TCR clone TCR-6 discussed elsewhere herein.

Accordingly, in one example, a nucleic acid composition described herein encodes a binding protein specific for a peptide comprising the amino acid sequence of SEQ ID NO:1 having TCR Vα domain comprising a CDR3 amino acid sequence having at least 80% sequence identity to SEQ ID NO:82, or a functional fragment thereof; and a nucleic acid sequence that encodes a TCR Vβ domain comprising a CDR3 amino acid sequence having at least 80% sequence identity to SEQ ID NO:85, or a functional fragment thereof.

In a particular example, a nucleic acid composition described herein encodes a binding protein specific for a peptide comprising the amino acid sequence of SEQ ID NO:1 having a TCR Vα domain with a CDR3 amino acid sequence comprising or consisting of the amino acid sequence of SEQ ID NO: 82; and a TCR Vβ domain with a CDR3 comprising or consisting of the amino acid sequence of SEQ ID NO:85. In addition, the peptide comprising the amino acid sequence of SEQ ID NO:1 may comprise or consist of the sequence shown in SEQ ID NO: 2. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 86; and the Vβ domain may comprise an amino acid sequence having at least 80% sequence identity to, comprising, or consisting of, SEQ ID NO: 88. In one example, the Vα domain comprises the amino acid sequence of SEQ ID NO: 86 and the Vβ domain comprises the amino acid sequence of SEQ ID NO: 88. In such cases, the Vα domain may be encoded by a nucleic acid sequence comprising the sequence of SEQ ID NO: 87; and the Vβ domain may be encoded by a nucleic acid sequence comprising the sequence of SEQ ID NO: 89.

In this particular example, the TCR Vα domain may include a CDR1 amino acid sequence comprising or consisting of the amino acid sequence of SEQ ID NO:80 and a CDR2 amino acid sequence comprising or consisting of the amino acid sequence of SEQ ID NO:81. Furthermore, the TCR Vβ domain may include a CDR1 amino acid sequence comprising or consisting of the amino acid sequence of SEQ ID NO:83 and a CDR2 amino acid sequence comprising or consisting of the amino acid sequence of SEQ ID NO: 84.

For the avoidance of doubt, this particular example encompasses components of TCR clone TCR-6 exemplified herein. The different components of TCR clone TCR-6 and their respective SEQ ID Nos are summarised in Table 11 below.

As stated in more detail elsewhere herein, the nucleic acid composition described herein encodes both a TCR Vα domain and a TCR Vβ domain, which form the binding protein that is capable of specifically binding to a TIMP3 antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(vi) TCR Clone TCR-7: TCR Components that Interact with SLGDVGAEA (SEQ ID NO: 2)

As provided elsewhere herein, the inventors have also identified TCR clone TCR-7 which interacts with SLGDWGAEA (SEQ ID NO: 2) in the context of HLA-A*02:01. The sequences provided herein that correspond to TCR clone TCR-7 are SEQ ID NOs 90 to 99.

In one embodiment, an isolated nucleic acid composition that encodes a TIMP3 antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:92, or a functional fragment thereof; and a nucleic acid sequence that encodes a TCR Vβ domain comprising a CDR3 amino acid sequence having at least 80% sequence identity to SEQ ID NO: 95, or a functional fragment thereof, wherein the CDR3 sequences together specifically bind to a peptide comprising the amino acid sequence of SEQ ID NO:1 (e.g. when the peptide is complexed with HLA).

An example of an appropriate TCR Vα domain CDR3 amino acid sequence that confers specific binding to a TIMP3 antigen, in particular a peptide comprising the amino acid sequence of SEQ ID NO:1 (e.g. to SLGDWGAEA (SEQ ID NO: 2)), is shown in SEQ ID NO: 92.

For example, appropriate (functional) Vα domain CDR3 amino acid sequences may have at least 80% sequence identity to SEQ ID NO: 92, i.e. they may have at least 80%, at least 84%, at least 92%, or 100% sequence identity to SEQ ID NO: 92. In other words, appropriate (functional) Vα domain CDR3 amino acid sequences may vary from the sequence shown in SEQ ID NO: 92 by one or several (e.g. two etc) amino acids.

In one example, the CDR3 of the Vα domain comprises or consists of the amino acid sequence of SEQ ID NO: 92. In examples where the TCR Vα domain CDR3 has the amino acid sequence of SEQ ID NO: 92, the CDR3 may be encoded by any appropriate nucleic acid sequence.

The encoded TCR Vα domain may comprise, in addition to the specified CDR3, a CDR1 comprising an amino acid sequence of SEQ ID NO: 90, or a functional variant thereof (i.e. wherein the variant retains the ability to specifically bind to a peptide comprising the amino acid sequence of SEQ ID NO:1 (e.g. the peptide shown in SEQ ID NO:2)).

For example, appropriate functional Vα domain CDR1 amino acid sequences may have at least 80% sequence identity to SEQ ID NO: 90, i.e. it may have at least 80%, at least 83%, or 100% sequence identity to SEQ ID NO: 90. Suitably, percent identity is calculated as the percentage of identity to the entire length of the reference sequence (e.g. SEQ ID NO: 90). In other words, appropriate functional Vα domain CDR1 amino acid sequences may vary from the sequence shown in SEQ ID NO: 90 by one or several amino acids.

In one example, the CDR1 of the Vα domain comprises or consists of the amino acid sequence of SEQ ID NO: 90. In examples where the TCR Vα domain CDR1 has the amino acid sequence of SEQ ID NO: 90, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 91, or a functional variant thereof (i.e. wherein the variant retains the ability to specifically bind to HLA-A*02, most preferably HLA-A*02:01).

For example, appropriate functional Vα domain CDR2 amino acid sequences may have at least 80% sequence identity to SEQ ID NO: 91, i.e. it may have at least 80%, at least 83%, or 100% sequence identity to SEQ ID NO: 91. Suitably, percent identity is calculated as the percentage of identity to the entire length of the reference sequence (e.g. SEQ ID NO: 91). In other words, appropriate (functional) Vα domain CDR2 amino acid sequences may vary from the sequence shown in SEQ ID NO: 91 by one or several amino acids.

In one example, the CDR2 of the Vα domain comprises or consists of the amino acid sequence of SEQ ID NO: 91. In examples where the TCR Vα domain CDR2 has the amino acid sequence of SEQ ID NO: 91, the CDR2 may be encoded by any appropriate nucleic acid sequence.

The encoded TCR Vα domain may therefore comprise the CDRs mentioned in detail above (by SEQ ID specifically i.e. SEQ ID NO:92, SEQ ID NO: 90 and SEQ ID NO: 91, or functional variants thereof), with appropriate intervening sequences between the CDRs.

The encoded TCR Vα domain may comprise an amino acid sequence of SEQ ID NO: 96, or a functional variant thereof (i.e. wherein the variant TCR Vα domain retains the ability to specifically bind to a peptide comprising the amino acid sequence of SEQ ID NO:1 (e.g. the peptide shown in SEQ ID NO:2)) when part of a binding protein described herein).

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 96, whilst retaining the ability to specifically bind to a peptide comprising the amino acid sequence of SEQ ID NO:1 (e.g. the peptide shown in SEQ ID NO:2). In other words, a functional TCR Vα domain with one or several amino acid substitutions compared to the sequence of SEQ ID NO: 96 is also encompassed. The variability in sequence compared to SEQ ID NO: 96 may all be in regions of the TCR Vα domain that do not form CDRs (i.e. the variant may have the CDRs of SEQ ID NO: 92, SEQ ID NO: 90 and/or SEQ ID NO: 91, and still have 25% (or less) sequence variability compared to SEQ ID NO:96). In other words, the sequence of the CDRs of SEQ ID NO: 96 may be retained whilst the rest of the sequence is varied, as appropriate within the “at least 75% identity” parameters specified above.

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 96, wherein the TCR Vα domain comprises a CDR3 having an amino acid sequence of SEQ ID NO: 92. In this example, the TCR Vα domain CDR1 may have an amino acid sequence of SEQ ID NO: 90 and the TCR Vα domain CDR2 may have an amino acid sequence of SEQ ID NO: 91.

As another example, the encoded TCR Vα domain may comprise an amino acid sequence having the amino acid sequence of SEQ ID NO: 96, with 0 to 10 (or 0 to 5) amino acid substitutions, insertions or deletions, wherein the TCR Vα domain comprises a CDR3 having an amino acid sequence of SEQ ID NO: 92. In this example, the TCR Vα domain CDR1 may have an amino acid sequence of SEQ ID NO: 90 and the TCR Vα domain CDR2 may have an amino acid sequence of SEQ ID NO: 91.

In examples where the TCR Vα domain has the amino acid sequence of SEQ ID NO: 96, the TCR Vα domain may be encoded by the nucleic acid sequence of SEQ ID NO: 97, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above. In one example, the nucleic acid composition provided herein comprises a nucleic acid sequence that encodes a TCR Vα domain comprising a CDR3 amino acid sequence having at least 80% sequence identity to SEQ ID NO:92, or a functional fragment thereof.

In another example, the CDR3 of the Vα domain of a nucleic acid composition provided herein comprises or consists of the amino acid sequence of SEQ ID NO: 92.

In another example, the Vα domain of a nucleic acid composition provided herein comprises an amino acid sequence having at least 80% sequence identity to, comprising, or consisting of, SEQ ID NO: 96.

As provided above, the inventors identified TCR clone TCR-7 which interacts with SLGDWGAEA (SEQ ID NO: 2) in the context of HLA-A*02:01. The sequences provided herein that correspond to TCR clone TCR-7 are SEQ ID NOs 90 to 99.

An example of an appropriate TCR Vβ domain CDR3 amino acid sequence that confers specific binding to a TIMP3 antigen, in particular a peptide comprising the amino acid sequence of SEQ ID NO:1 (e.g. the peptide shown in SEQ ID NO:2), is shown in SEQ ID NO:95.

For example, appropriate (functional) Vβ domain CDR3 amino acid sequences may have at least 80% sequence identity to SEQ ID NO: 95, i.e. they may have at least 80%, at least 86%, at least 93%, or 100% sequence identity to SEQ ID NO: 95. Suitably, percent identity is calculated as the percentage of identity to the entire length of the reference sequence (e.g. SEQ ID NO: 95).

In other words, appropriate (functional) Vβ domain CDR3 amino acid sequences may vary from the sequence shown in SEQ ID NO: 95 by one or several (e.g. two) amino acids.

In one example, the CDR3 of the Vβ domain comprises or consists of the amino acid sequence of SEQ ID NO: 95. In examples where the TCR Vβ domain CDR3 has the amino acid sequence of SEQ ID NO:95, the CDR3 may be encoded by any appropriate nucleic acid sequence.

The encoded TCR Vβ domain may comprise, in addition to the specified CDR3, a CDR1 comprising an amino acid sequence of SEQ ID NO: 93, or a functional variant thereof (i.e. wherein the variant retains the ability to specifically bind to a peptide comprising the amino acid sequence of SEQ ID NO:1 (e.g. the peptide shown in SEQ ID NO:2).

For example, appropriate functional Vβ domain CDR1 amino acid sequences may have at least 80% sequence identity to SEQ ID NO: 93, i.e. it may have at least 80%, or 100% sequence identity to SEQ ID NO: 93. Suitably, percent identity is calculated as the percentage of identity to the entire length of the reference sequence (e.g. SEQ ID NO: 93). In other words, appropriate (functional) Vβ domain CDR1 amino acid sequences may vary from the sequence shown in SEQ ID NO:93 by one or several amino acids.

In one example, the CDR1 of the Vβ domain comprises or consists of the amino acid sequence of SEQ ID NO: 93. In examples where the TCR Vα domain CDR1 has the amino acid sequence of SEQ ID NO:93, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 94, or a functional variant thereof (i.e. wherein the variant retains the ability to specifically bind to HLA-A*02, most preferably HLA-A*02:01).

For example, appropriate functional Vβ domain CDR2 amino acid sequences may have at least 80% sequence identity to SEQ ID NO: 94, i.e. it may have at least 80%, at least 83%, or 100% sequence identity to SEQ ID NO: 94. In other words, appropriate (functional) Vβ domain CDR2 amino acid sequences may vary from the sequence shown in SEQ ID NO: 94 by one or several amino acids.

In one example, the CDR2 of the Vβ domain comprises or consists of the amino acid sequence of SEQ ID NO: 94. In examples where the TCR Vβ domain CDR2 has the amino acid sequence of SEQ ID NO:94, the CDR2 may be encoded by any appropriate nucleic acid sequence.

The encoded TCR Vβ domain may therefore comprise the CDRs mentioned in detail above (by SEQ ID specifically i.e. SEQ ID NO:95, SEQ ID NO: 93 and SEQ ID NO: 94, or functional variants thereof), with appropriate intervening sequences between the CDRs.

The encoded TCR Vβ domain may have an amino acid sequence of SEQ ID NO: 98, or a functional variant thereof (i.e. wherein the variant TCR Vβ domain retains the ability to specifically bind to a peptide comprising the amino acid sequence of SEQ ID NO:1 (e.g. the peptide shown in SEQ ID NO:2) when part of a binding protein described herein).

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 98, whilst retaining the ability to specifically bind to a peptide comprising the amino acid sequence of SEQ ID NO:1 (e.g. the peptide shown in SEQ ID NO:2). In other words, a functional TCR Vβ domain with one or several amino acid substitutions compared to the sequence of SEQ ID NO: 98 is also encompassed. The variability in sequence compared to SEQ ID NO:98 may all be in regions of the TCR Vβ domain that do not form CDRs (i.e. the variant may have the CDRs of SEQ ID NO: 95, SEQ ID NO: 93 and/or SEQ ID NO: 94, and still have 25% (or less) sequence variability compared to SEQ ID NO: 98). In other words, the sequence of the CDRs of SEQ ID NO: 98 may be retained whilst the rest of the sequence is varied, as appropriate within the “at least 75% identity” parameters specified above.

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 98, wherein the TCR Vβ domain comprises a CDR3 having an amino acid sequence of SEQ ID NO: 95. In this example, the TCR Vβ domain CDR1 may have an amino acid sequence of SEQ ID NO:93 and the TCR Vβ domain CDR2 may have an amino acid sequence of SEQ ID NO: 94.

In examples where the TCR Vβ domain has the amino acid sequence of SEQ ID NO:98, the TCR Vβ domain may be encoded by the nucleic acid sequence of SEQ ID NO: 99, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

In an example, the nucleic acid composition provided herein comprises a nucleic acid sequence that encodes a TCR Vβ domain comprising a CDR3 amino acid sequence having at least 80% sequence identity to SEQ ID NO:95, or a functional fragment thereof.

In another example, the CDR3 of the Vβ domain of a nucleic acid composition provided herein comprises or consists of the amino acid sequence of SEQ ID NO:95.

In a further example, the Vβ domain of a nucleic acid composition provided herein comprises an amino acid sequence having at least 80% sequence identity to, comprising, or consisting of, SEQ ID NO: 98.

The TCR Vβ domain sequences derived from TCR clone TCR-7 discussed above are particularly compatible with the TCR Vα domain sequences derived from TCR clone TCR-7 discussed elsewhere herein.

Accordingly, in one example, a nucleic acid composition described herein encodes a binding protein specific for a peptide comprising the amino acid sequence of SEQ ID NO:1 having TCR Vα domain comprising a CDR3 amino acid sequence having at least 80% sequence identity to SEQ ID NO:92, or a functional fragment thereof; and a nucleic acid sequence that encodes a TCR Vβ domain comprising a CDR3 amino acid sequence having at least 80% sequence identity to SEQ ID NO:95, or a functional fragment thereof.

In a particular example, a nucleic acid composition described herein encodes a binding protein specific for a peptide comprising the amino acid sequence of SEQ ID NO:1 having a TCR Vα domain with a CDR3 amino acid sequence comprising or consisting of the amino acid sequence of SEQ ID NO: 92; and a TCR Vβ domain with a CDR3 comprising or consisting of the amino acid sequence of SEQ ID NO:95. In addition, the peptide comprising the amino acid sequence of SEQ ID NO:1 antigen may comprise or consist of the sequence shown in SEQ ID NO: 2. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 96; and the Vβ domain may comprise an amino acid sequence having at least 80% sequence identity to, comprising, or consisting of, SEQ ID NO: 98. In one example, the Vα domain comprises the amino acid sequence of SEQ ID NO: 96 and the Vβ domain comprises the amino acid sequence of SEQ ID NO: 98. In such cases, the Vα domain may be encoded by a nucleic acid sequence comprising the sequence of SEQ ID NO: 97; and the Vβ domain may be encoded by a nucleic acid sequence comprising the sequence of SEQ ID NO: 99.

In this particular example, the TCR Vα domain may include a CDR1 amino acid sequence comprising or consisting of the amino acid sequence of SEQ ID NO:90 and a CDR2 amino acid sequence comprising or consisting of the amino acid sequence of SEQ ID NO:91. Furthermore, the TCR Vβ domain may include a CDR1 amino acid sequence comprising or consisting of the amino acid sequence of SEQ ID NO:93 and a CDR2 amino acid sequence comprising or consisting of the amino acid sequence of SEQ ID NO: 94.

For the avoidance of doubt, this particular example encompasses components of TCR clone TCR-7 exemplified herein. The different components of TCR clone TCR-7 and their respective SEQ ID Nos are summarised in Table 12 below.

As stated in more detail elsewhere herein, the nucleic acid composition described herein encodes both a TCR Vα domain and a TCR Vβ domain, which form the binding protein that is capable of specifically binding to a TIMP3 antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(vii) TCR Clone TCR-9: TCR Components that Interact with SLGDVGAEA (SEQ ID NO: 2)

As provided elsewhere herein, the inventors have also identified TCR clone TCR-9 which interacts with SLGDWGAEA (SEQ ID NO: 2) in the context of HLA-A*02:01. The sequences provided herein that correspond to TCR clone TCR-9 are SEQ ID NOs 100 to 109.

In one embodiment, an isolated nucleic acid composition that encodes a TIMP3 antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:102, or a functional fragment thereof; and a nucleic acid sequence that encodes a TCR Vβ domain comprising a CDR3 amino acid sequence having at least 80% sequence identity to SEQ ID NO: 105, or a functional fragment thereof, wherein the CDR3 sequences together specifically bind to a peptide comprising the amino acid sequence of SEQ ID NO:1 (e.g. when the peptide is complexed with HLA).

An example of an appropriate TCR Vα domain CDR3 amino acid sequence that confers specific binding to a TIMP3 antigen, in particular a peptide comprising the amino acid sequence of SEQ ID NO:1 (e.g. to SLGDWGAEA (SEQ ID NO: 2)), is shown in SEQ ID NO: 102.

For example, appropriate (functional) Vα domain CDR3 amino acid sequences may have at least 80% sequence identity to SEQ ID NO: 102, i.e. they may have at least 80%, at least 84%, at least 92%, or 100% sequence identity to SEQ ID NO: 102. In other words, appropriate (functional) Vα domain CDR3 amino acid sequences may vary from the sequence shown in SEQ ID NO: 102 by one or several (e.g. two etc) amino acids.

In one example, the CDR3 of the Vα domain comprises or consists of the amino acid sequence of SEQ ID NO: 102. In examples where the TCR Vα domain CDR3 has the amino acid sequence of SEQ ID NO: 102, the CDR3 may be encoded by any appropriate nucleic acid sequence.

The encoded TCR Vα domain may comprise, in addition to the specified CDR3, a CDR1 comprising an amino acid sequence of SEQ ID NO: 100, or a functional variant thereof (i.e. wherein the variant retains the ability to specifically bind to a peptide comprising the amino acid sequence of SEQ ID NO:1 (e.g. the peptide shown in SEQ ID NO:2)).

For example, appropriate functional Vα domain CDR1 amino acid sequences may have at least 80% sequence identity to SEQ ID NO: 100, i.e. it may have at least 80%, at least 83%, or 100% sequence identity to SEQ ID NO: 100. In other words, appropriate functional Vα domain CDR1 amino acid sequences may vary from the sequence shown in SEQ ID NO: 100 by one or several amino acids.

In one example, the CDR1 of the Vα domain comprises or consists of the amino acid sequence of SEQ ID NO: 100. In examples where the TCR Vα domain CDR1 has the amino acid sequence of SEQ ID NO: 100, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 101, or a functional variant thereof (i.e. wherein the variant retains the ability to specifically bind to HLA-A*02, most preferably HLA-A*02:01).

For example, appropriate functional Vα domain CDR2 amino acid sequences may have at least 80% sequence identity to SEQ ID NO: 101, i.e. it may have at least 80%, at least 83%, or 100% sequence identity to SEQ ID NO: 101. In other words, appropriate (functional) Vα domain CDR2 amino acid sequences may vary from the sequence shown in SEQ ID NO: 101 by one or several amino acids.

In one example, the CDR2 of the Vα domain comprises or consists of the amino acid sequence of SEQ ID NO: 101. In examples where the TCR Vα domain CDR2 has the amino acid sequence of SEQ ID NO: 101, the CDR2 may be encoded by any appropriate nucleic acid sequence.

The encoded TCR Vα domain may therefore comprise the CDRs mentioned in detail above (by SEQ ID specifically i.e. SEQ ID NO:102, SEQ ID NO: 100 and SEQ ID NO: 101, or functional variants thereof), with appropriate intervening sequences between the CDRs.

The encoded TCR Vα domain may comprise an amino acid sequence of SEQ ID NO: 106, or a functional variant thereof (i.e. wherein the variant TCR Vα domain retains the ability to specifically bind to a peptide comprising the amino acid sequence of SEQ ID NO:1 (e.g. the peptide shown in SEQ ID NO:2)) when part of a binding protein described herein).

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 106, whilst retaining the ability to specifically bind to a peptide comprising the amino acid sequence of SEQ ID NO:1 (e.g. the peptide shown in SEQ ID NO:2). In other words, a functional TCR Vα domain with one or several amino acid substitutions compared to the sequence of SEQ ID NO: 106 is also encompassed.

The variability in sequence compared to SEQ ID NO: 106 may all be in regions of the TCR Vα domain that do not form CDRs (i.e. the variant may have the CDRs of SEQ ID NO: 102, SEQ ID NO: 100 and/or SEQ ID NO: 101, and still have 25% (or less) sequence variability compared to SEQ ID NO:106). In other words, the sequence of the CDRs of SEQ ID NO: 106 may be retained whilst the rest of the sequence is varied, as appropriate within the “at least 75% identity” parameters specified above.

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 106, wherein the TCR Vα domain comprises a CDR3 having an amino acid sequence of SEQ ID NO: 102. In this example, the TCR Vα domain CDR1 may have an amino acid sequence of SEQ ID NO: 100 and the TCR Vα domain CDR2 may have an amino acid sequence of SEQ ID NO: 101.

As another example, the encoded TCR Vα domain may comprise an amino acid sequence having the amino acid sequence of SEQ ID NO: 106, with 0 to 10 (or 0 to 5) amino acid substitutions, insertions or deletions, wherein the TCR Vα domain comprises a CDR3 having an amino acid sequence of SEQ ID NO: 102. In this example, the TCR Vα domain CDR1 may have an amino acid sequence of SEQ ID NO: 100 and the TCR Vα domain CDR2 may have an amino acid sequence of SEQ ID NO: 101.

In examples where the TCR Vα domain has the amino acid sequence of SEQ ID NO: 106, the TCR Vα domain may be encoded by the nucleic acid sequence of SEQ ID NO: 107, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

In one example, the nucleic acid composition provided herein comprises a nucleic acid sequence that encodes a TCR Vα domain comprising a CDR3 amino acid sequence having at least 80% sequence identity to SEQ ID NO:102, or a functional fragment thereof.

In another example, the CDR3 of the Vα domain of a nucleic acid composition provided herein comprises or consists of the amino acid sequence of SEQ ID NO: 102.

In another example, the Vα domain of a nucleic acid composition provided herein comprises an amino acid sequence having at least 80% sequence identity to, comprising, or consisting of, SEQ ID NO: 106.

As provided above, the inventors identified TCR clone TCR-9 which interacts with SLGDWGAEA (SEQ ID NO: 2) in the context of HLA-A*02:01. The sequences provided herein that correspond to TCR clone TCR-9 are SEQ ID NOs 100 to 109.

An example of an appropriate TCR Vβ domain CDR3 amino acid sequence that confers specific binding to a TIMP3 antigen, in particular a peptide comprising the amino acid sequence of SEQ ID NO: 1 (e.g. to SLGDWGAEA (SEQ ID NO: 2)), is shown in SEQ ID NO:105. As would be clear to a person of skill in the art, variants of the amino acid sequence shown in SEQ ID NO:105 may also be functional (i.e. retain their ability to confer specific binding to a peptide comprising the amino acid sequence of SEQ ID NO:1 (e.g. the peptide shown in SEQ ID NO:2) when the CDR3 is part of TCR Vβ domain).

For example, appropriate (functional) Vβ domain CDR3 amino acid sequences may have at least 80% sequence identity to SEQ ID NO: 105, i.e. they may have at least 80%, at least 86%, at least 93%, or 100% sequence identity to SEQ ID NO: 105. In other words, appropriate (functional) Vβ domain CDR3 amino acid sequences may vary from the sequence shown in SEQ ID NO: 105 by one or several (e.g. two) amino acids.

In one example, the CDR3 of the Vβ domain comprises or consists of the amino acid sequence of SEQ ID NO: 105. In examples where the TCR Vβ domain CDR3 has the amino acid sequence of SEQ ID NO:105, the CDR3 may be encoded by any appropriate nucleic acid sequence.

The encoded TCR Vβ domain may comprise, in addition to the specified CDR3, a CDR1 comprising an amino acid sequence of SEQ ID NO: 103, or a functional variant thereof (i.e. wherein the variant retains the ability to specifically bind to a peptide comprising the amino acid sequence of SEQ ID NO:1 (e.g. the peptide shown in SEQ ID NO:2).

For example, appropriate functional Vβ domain CDR1 amino acid sequences may have at least 80% sequence identity to SEQ ID NO: 103, i.e. it may have at least 80%, or 100% sequence identity to SEQ ID NO: 103. In other words, appropriate (functional) Vβ domain CDR1 amino acid sequences may vary from the sequence shown in SEQ ID NO:103 by one or several amino acids.

In one example, the CDR1 of the Vβ domain comprises or consists of the amino acid sequence of SEQ ID NO: 103. In examples where the TCR Vα domain CDR1 has the amino acid sequence of SEQ ID NO:103, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 104, or a functional variant thereof (i.e. wherein the variant retains the ability to specifically bind to HLA-A*02, most preferably HLA-A*02:01).

For example, appropriate functional Vβ domain CDR2 amino acid sequences may have at least 80% sequence identity to SEQ ID NO: 104, i.e. it may have at least 80%, at least 83%, or 100% sequence identity to SEQ ID NO: 104. In other words, appropriate (functional) Vβ domain CDR2 amino acid sequences may vary from the sequence shown in SEQ ID NO: 104 by one or several amino acids.

In one example, the CDR2 of the Vβ domain comprises or consists of the amino acid sequence of SEQ ID NO: 104. In examples where the TCR Vβ domain CDR2 has the amino acid sequence of SEQ ID NO:104, the CDR2 may be encoded by any appropriate nucleic acid sequence.

The encoded TCR Vβ domain may therefore comprise the CDRs mentioned in detail above (by SEQ ID specifically i.e. SEQ ID NO:105, SEQ ID NO: 103 and SEQ ID NO: 104, or functional variants thereof), with appropriate intervening sequences between the CDRs.

The encoded TCR Vβ domain may have an amino acid sequence of SEQ ID NO: 108, or a functional variant thereof (i.e. wherein the variant TCR Vβ domain retains the ability to specifically bind to a peptide comprising the amino acid sequence of SEQ ID NO:1 (e.g. the peptide shown in SEQ ID NO:2)) when part of a binding protein described herein).

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 108, whilst retaining the ability to specifically bind to a peptide comprising the amino acid sequence of SEQ ID NO:1 (e.g. the peptide shown in SEQ ID NO:2). In other words, a functional TCR Vβ domain with one or several amino acid substitutions compared to the sequence of SEQ ID NO: 108 is also encompassed. The variability in sequence compared to SEQ ID NO:108 may all be in regions of the TCR Vβ domain that do not form CDRs (i.e. the variant may have the CDRs of SEQ ID NO: 105, SEQ ID NO: 103 and/or SEQ ID NO: 104, and still have 25% (or less) sequence variability compared to SEQ ID NO: 108). In other words, the sequence of the CDRs of SEQ ID NO: 108 may be retained whilst the rest of the sequence is varied, as appropriate within the “at least 75% identity” parameters specified above.

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 108, wherein the TCR Vβ domain comprises a CDR3 having an amino acid sequence of SEQ ID NO: 105. In this example, the TCR Vβ domain CDR1 may have an amino acid sequence of SEQ ID NO:103 and the TCR Vβ domain CDR2 may have an amino acid sequence of SEQ ID NO: 104.

In examples where the TCR Vβ domain has the amino acid sequence of SEQ ID NO:108, the TCR Vβ domain may be encoded by the nucleic acid sequence of SEQ ID NO: 109, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

In an example, the nucleic acid composition provided herein comprises a nucleic acid sequence that encodes a TCR Vβ domain comprising a CDR3 amino acid sequence having at least 80% sequence identity to SEQ ID NO:105, or a functional fragment thereof.

In another example, the CDR3 of the Vβ domain of a nucleic acid composition provided herein comprises or consists of the amino acid sequence of SEQ ID NO:105.

In a further example, the Vβ domain of a nucleic acid composition provided herein comprises an amino acid sequence having at least 80% sequence identity to, comprising, or consisting of, SEQ ID NO: 108.

The TCR Vβ domain sequences derived from TCR clone TCR-9 discussed above are particularly compatible with the TCR Vα domain sequences derived from TCR clone TCR-9 discussed elsewhere herein.

Accordingly, in one example, a nucleic acid composition described herein encodes a binding protein specific for a peptide comprising the amino acid sequence of SEQ ID NO:1 having TCR Vα domain comprising a CDR3 amino acid sequence having at least 80% sequence identity to SEQ ID NO:102, or a functional fragment thereof; and a nucleic acid sequence that encodes a TCR Vβ domain comprising a CDR3 amino acid sequence having at least 80% sequence identity to SEQ ID NO:105, or a functional fragment thereof.

In a particular example, a nucleic acid composition described herein encodes a binding protein specific for a peptide comprising the amino acid sequence of SEQ ID NO:1 having a TCR Vα domain with a CDR3 amino acid sequence comprising or consisting of the amino acid sequence of SEQ ID NO: 102; and a TCR Vβ domain with a CDR3 comprising or consisting of the amino acid sequence of SEQ ID NO:105. In addition, the peptide comprising the amino acid sequence of SEQ ID NO:1 may comprise or consist of the sequence shown in SEQ ID NO: 2. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 106; and the Vβ domain may comprise an amino acid sequence having at least 80% sequence identity to, comprising, or consisting of, SEQ ID NO: 108. In one example, the Vα domain comprises the amino acid sequence of SEQ ID NO: 106 and the Vβ domain comprises the amino acid sequence of SEQ ID NO: 108. In such cases, the Vα domain may be encoded by a nucleic acid sequence comprising the sequence of SEQ ID NO: 107; and the Vβ domain may be encoded by a nucleic acid sequence comprising the sequence of SEQ ID NO: 109.

In this particular example, the TCR Vα domain may include a CDR1 amino acid sequence comprising or consisting of the amino acid sequence of SEQ ID NO:100 and a CDR2 amino acid sequence comprising or consisting of the amino acid sequence of SEQ ID NO:101. Furthermore, the TCR Vβ domain may include a CDR1 amino acid sequence comprising or consisting of the amino acid sequence of SEQ ID NO:103 and a CDR2 amino acid sequence comprising or consisting of the amino acid sequence of SEQ ID NO: 104.

For the avoidance of doubt, this particular example encompasses components of TCR clone TCR-9 exemplified herein. The different components of TCR clone TCR-9 and their respective SEQ ID NOs are summarised in Table 13 below.

As stated in more detail elsewhere herein, the nucleic acid composition described herein encodes both a TCR Vα domain and a TCR Vβ domain, which form the binding protein that is capable of specifically binding to a TIMP3 antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

Methods of Generating Binding Proteins (e.g. TCRs)

A method of generating a binding protein that is capable of specifically binding to a peptide containing a TIMP3 antigen comprising the amino acid sequence of SEQ ID NO: 1 and does not bind to a peptide that does not contain the TIMP3 antigen comprising the amino acid sequence of SEQ ID NO: 1 is also provided, comprising contacting a nucleic acid composition (or vector system) described herein with a cell under conditions in which the nucleic acid composition is incorporated and expressed by the cell.

In the context of the binding proteins described herein, the TIMP3 antigen comprises or consists of a sequence comprising an amino acid sequence selected from the group consisting of: SEQ ID NO: 1-27, 110 or 111, or a functional fragment or variant thereof. The method may be carried out on the (host) cell ex vivo or in vitro. Alternatively, the method may be performed in vivo, wherein the nucleic acid composition (or vector system) is administered to the subject and is contacted with the cel in vivo, under conditions in which the nucleic acid sequence is incorporated and expressed by the cell to generate the binding protein. In one example, the method is not a method of treatment of the human or animal body. Appropriate in vivo, in vitro and ex vivo methods for contacting a nucleic acid sequence (or vector systems) with a cell under conditions in which the nucleic acid sequence (or vector) is incorporated and expressed by the cell are well known, as described elsewhere herein.

As stated elsewhere herein, the binding protein comprise a TCR, an antigen binding fragment of a TCR, a ImmTAC or a chimeric antigen receptor (CAR). Further details are provided elsewhere herein.

The binding proteins described herein may be used therapeutically, as described elsewhere herein. Furthermore, the binding proteins may be used in a diagnostic setting, e.g. to detect the presence of TIMP3 presented in the context of an appropriate HLA at the cell surface of diseased/malignant tissues.

### Use of a Peptide According to the Invention for Identifying a Therapeutic Binding Protein

Use of a peptide comprising an amino acid sequence of SEQ ID NO: 1, or a complex according to the invention, for identifying a therapeutic binding protein is also provided herein. Use of a peptide comprising an amino acid sequence of any one of SEQ ID NOs: 2-21, preferably any one of SEQ ID NOs: 2-5, more preferably SEQ ID NO: 2, or a complex according to the invention, for identifying a therapeutic binding protein is also provided herein.

As disclosed herein, a peptide or complex according to the invention can be used to identify therapeutic binding proteins. For example, as disclosed in the Examples, T cell receptors can be identified that may enable therapeutic properties. Other suitable methods for identifying a therapeutic binding protein using a peptide or complex according to the invention are known in the art and can be suitably employed by the skilled person.

Preferably, the complex according to the invention is a complex comprising:
a) a peptide comprising the amino acid sequence of SEQ ID NO: 1, and
b) a binding agent that specifically binds to a peptide comprising the amino acid sequence of SEQ ID NO: 1; optionally wherein the binding agent is an HLA-A*02 molecule, further optionally wherein the binding agent is an HLA-A*02:01 molecule.

Preferably, the complex according to the invention is a complex comprising:
a) a peptide comprising the amino acid sequence of any one of SEQ ID NOs: 2-21, and
b) a binding agent that specifically binds to a peptide comprising the amino acid sequence of any one of the respective SEQ ID NOs: 2-21; optionally wherein the binding agent is an HLA-A*02 molecule, further optionally wherein the binding agent is an HLA-A*02:01 molecule.

Preferably, the complex is:
a. a SLGDVGAEA:HLA-A*02 complex, a SLGDWGAEV:HLA-A*02 complex, a SLGDVGAEI:HLA-A*02 complex, a SLGDVGAEL:HLA-A*02 complex, a SLGDVGAER:HLA-A*02 complex, a SLGDWGAEN:HLA-A*02 complex, a SLGDVGAED:HLA-A*02 complex, a SLGDVGAEC:HLA-A*02 complex, a SLGDVGAEQ:HLA-A*02 complex, a SLGDVGAEE:HLA-A*02 complex, a SLGDWGAEG:HLA-A*02 complex, a SLGDWGAEH:HLA-A*02 complex, a SLGDVGAEK:HLA-A*02 complex, a SLGDVGAEM:HLA-A*02 complex, a SLGDWGAEF:HLA-A*02 complex, a SLGDVGAEP:HLA-A*02 complex, a SLGDWGAES:HLA-A*02 complex, a SLGDVGAET:HLA-A*02 complex, a SLGDVGAEW:HLA-A*02 complex or a SLGDVGAEY:HLA-A*02 complex, optionally wherein the complex is a SLGDVGAEA:HLA-A*02 complex, a SLGDVGAEV:HLA-A*02 complex, a SLGDVGAE:HLA-A*02 complex or a SLGDVGAEL:HLA-A*02 complex, further optionally wherein the complex is a SLGDVGAEA:HLA-A*02 complex, a SLGDVGAEV:HLA-A*02 complex or a SLGDVGAEI:HLA-A*02 complex; or
b. a SLGDWGAEA:HLA-A*02:01 complex, a SLGDWGAEV:HLA-A*02:01 complex, a SLGDWGAEI:HLA-A*02:01 complex, a SLGDWGAEL:HLA-A*02:01 complex, a SLGDVGAER:HLA-A*02:01 complex, a SLGDVGAEN:HLA-A*02:01 complex, a SLGDVGAED:HLA-A*02:01 complex, a SLGDVGAEC:HLA-A*02:01 complex, a SLGDVGAEQ:HLA-A*02:01 complex, a SLGDVGAEE:HLA-A*02:01 complex, a SLGDVGAEG:HLA-A*02:01 complex, a SLGDVGAEH:HLA-A*02:01 complex, a SLGDVGAEK:HLA-A*02:01 complex, a SLGDVGAEM:HLA-A*02:01 complex, a SLGDVGAEF:HLA-A*02:01 complex, a SLGDVGAEP:HLA-A*02:01 complex, a SLGDVGAES:HLA-A*02:01 complex, a SLGDVGAET:HLA-A*02:01 complex, a SLGDVGAEW:HLA-A*02:01 complex or a SLGDVGAEY:HLA-A*02:01 complex, optionally wherein the complex is a SLGDVGAEA:HLA-A*02:01 complex, a SLGDWGAEV:HLA-A*02:01 complex, a SLGDVWGAEI:HLA-A*02:01 complex or a SLGDWGAEL:HLA-A*02:01 complex, further optionally wherein the complex is a SLGDVGAEA:HLA-A*02:01 complex, a SLGDVGAEV:HLA-A*02:01 complex or a SLGDVGAE:HLA-A*02:01 complex.

Preferably, the use of a peptide or a complex according to the invention, wherein the therapeutic binding protein is capable of preventing or treating a pre-cancer, a cancer or a viral infection associated with impaired HLA class I antigen presentation.

Preferably, the use of a peptide or a complex according to the invention, wherein the therapeutic binding protein comprises: a TCR, an antigen binding fragment of a TCR, or a chimeric antigen receptor (CAR), or an ImmTAC; or the therapeutic binding protein is an antibody.

Preferably, the use of a peptide or a complex according to the invention, wherein:
(a) the peptide comprises an amino acid sequence selected from the group consisting of SEQ ID NO: 2-5; or
(b) the complex is selected from the group consisting of: a SLGDWGAEA:HLA-A*02 complex, a SLGDVNGAEV:HLA-A*02 complex, a SLGDVNGAE:HLA-A*02 complex; or a SLGDVNGAEL:HLA-A*02 complex.

### Combinations of Vaccine and Modified Cells

A vaccine comprising the peptide according to the invention; and a modified cell transformed, transfected or transduced with a nucleic acid described herein, a nucleic acid composition described herein, a nucleic acid sequence described herein, or a vector described herein; for use as a medicament is also provided herein.

Administering to a subject in need thereof a vaccine comprising the peptide according to the invention and a modified cell described herein expressing a binding moiety that binds the peptide according to the invention is expected to be more effective for treatment of the subject.

Suitably, the vaccine and/or the modified cell may be in the form of a pharmaceutical composition. Suitably, the vaccine may be administered to a subject in need thereof prior to, during, and/or after administration of the modified cell. Any of the vaccines and modified cells described herein may suitably be used in the combination. The binding moiety may suitably be expressed by the nucleic acid, nucleic acid composition, nucleic acid sequence, or vector described herein.

Suitably, the binding moiety may be a binding protein, comprising for example a TCR described herein, an antigen binding fragment of a TCR described herein, a CAR described herein, or an ImmTAC described herein. Suitably, the nucleic acid, nucleic acid composition, nucleic acid sequence, or vector may encode a TIMP3 antigen-specific binding protein comprising a TCR described herein, an antigen binding fragment of a TCR described herein, a CAR described herein, or an ImmTAC described herein. Suitably, the nucleic acid composition may encode a TIMP3 antigen-specific binding protein having a TCR α chain variable (Vα) domain and a TCR β chain variable (Vβ) domain, the composition comprising: a nucleic acid sequence that encodes a TCR Vα domain comprising a CDR3 amino acid sequence; and a nucleic acid sequence that encodes a TCR Vβ domain comprising a CDR3 amino acid sequence; wherein the CDR3 sequences together specifically bind to a peptide comprising the amino acid sequence of SEQ ID NO: 1.

Suitably, the vaccine and the modified cell may be for use in the prevention or treatment of a pre-cancer, a cancer or a viral infection associated with impaired HLA class I antigen presentation in a human subject. Suitably, the pre-cancer, cancer, or viral infection may be a pre-cancer, cancer, or viral infection with impaired peptide processing machinery. Suitably, the pre-cancer, cancer, or viral infection may be associated with reduced or low TAP1 or TAP2 protein expression, activity, level, or stability.

### General Definitions

As used herein, the term “TIMP3 antigen” or “TIMP3 peptide antigen” or “TIMP3 containing peptide antigen” refers to a naturally or synthetically produced peptide portion of a TIMP3 protein ranging in length from about 8 amino acids, about 9 amino acids, about 10 amino acids, preferably up to about 35 amino acids, which can form a complex with a MHC (e.g., HLA) molecule, and a binding protein of this disclosure specific for a TIMP3 peptide:MHC (e.g., HLA) complex can specifically bind to such as complex. Typically, for the purposes of this disclosure, the TIMP3 peptide antigen comprises or consists of an amino acid sequence selected from the group consisting of: SEQ ID NO: 1-27, 110 or 111. Additionally, for the purposes of this disclosure, the TIMP3 peptide antigen:HLA complex typically comprises a peptide:HLA complex selected from the group consisting of: a SX₁GDVWGAEX₂:HLA-A*02:01 complex (wherein X₁ and X₂ are any amino acid), a SLGDVWGAEA:HLA-A*02:01 complex, a SLGDVWGAEV:HLA-A*02:01 complex, a SLGDVGAEI:HLA-A*02:01 complex, a SLGDVGAEL:HLA-A*02:01 complex, a SLGDVGAER:HLA-A*02:01 complex, a SLGDVGAEN:HLA-A*02:01 complex, a SLGDVGAED:HLA-A*02:01 complex, a SLGDVGAEC:HLA-A*02:01 complex, a SLGDVGAEQ:HLA-A*02:01 complex, a SLGDVGAEE:HLA-A*02:01 complex, a SLGDVGAEG:HLA-A*02:01 complex, a SLGDVGAEH:HLA-A*02:01 complex, a SLGDVGAEK:HLA-A*02:01 complex, a SLGDVGAEM:HLA-A*02:01 complex, a SLGDVGAEF:HLA-A*02:01 complex, a SLGDVGAEP:HLA-A*02:01 complex, a SLGDVGAES:HLA-A*02:01 complex, a SLGDVGAET:HLA-A*02:01 complex, a SLGDWGAEW:HLA-A*02:01 complex, and a SLGDWGAEY:HLA-A*02:01 complex, preferably selected from the group consisting of a SX₁GDVWGAEX₂:HLA-A*02:01 complex (wherein X₁ and X₂ are any amino acid), a SLGDVGAEA:HLA-A*02:01 complex, a SLGDWGAEV:HLA-A*02:01 complex, a SLGDWGAEI:HLA-A*02:01 complex and a SLGDWGAEL:HLA-A*02:01 complex, more preferably selected from the group consisting of a SX₁GDVWGAEX₂:HLA-A*02:01 complex (wherein X₁ and X₂ are any amino acid), a SLGDWGAEA:HLA-A*02:01 complex, a SLGDVGAEV:HLA-A*02:01 complex and a SLGDVGAE:HLA-A*02:01 complex. Foreach of the complexes listed in the previous sentence, the HLA-A*02:01 molecule may instead also be HLA-A*02, HLA-A*02:02, HLA-A*02:03, HLA-A*02:04, or HLA-A*02:09.

The term “binding agent that specifically binds to a peptide comprising the amino acid sequence of SEQ ID NO: 1,” as used herein, refers to a binding agent, that specifically binds to a peptide comprising the amino acid sequence of SEQ ID NO: 1 (or to a peptide antigen:HLA complex where the peptide comprises the amino acide sequence of SEQ ID NO: 1, e.g., on a cell surface), and does not bind a peptide sequence that does not include the peptide comprising the amino acid sequence of SEQ ID NO: 1. Typically, for the purposes of this disclosure, the peptide comprises or consists of an amino acid sequence selected from the group consisting of: SEQ ID NO: 1-27, 110 or 111, and the peptide antigen:HLA complex comprises a peptide:HLA complex selected from the group consisting of a SX₁GDVWGAEX₂:HLA-A*02:01 complex (wherein X₁ and X₂ are any amino acid), a SLGDVGAEA:HLA-A*02:01 complex, a SLGDWGAEV:HLA-A*02:01 complex, a SLGDWGAEI:HLA-A*02:01 complex, a SLGDWGAEL:HLA-A*02:01 complex, a SLGDVGAER:HLA-A*02:01 complex, a SLGDVGAEN:HLA-A*02:01 complex, a SLGDVGAED:HLA-A*02:01 complex, a SLGDVGAEC:HLA-A*02:01 complex, a SLGDVGAEQ:HLA-A*02:01 complex, a SLGDVGAEE:HLA-A*02:01 complex, a SLGDVGAEG:HLA-A*02:01 complex, a SLGDVGAEH:HLA-A*02:01 complex, a SLGDVGAEK:HLA-A*02:01 complex, a SLGDVGAEM:HLA-A*02:01 complex, a SLGDVGAEF:HLA-A*02:01 complex, a SLGDVGAEP:HLA-A*02:01 complex, a SLGDVGAES:HLA-A*02:01 complex, a SLGDVGAET:HLA-A*02:01 complex, a SLGDWGAEW:HLA-A*02:01 complex, and a SLGDWGAEY:HLA-A*02:01 complex, preferably selected from the group consisting of a SX₁GDVWGAEX₂:HLA-A*02:01 complex (wherein X₁ and X₂ are any amino acid), a SLGDVGAEA:HLA-A*02:01 complex, a SLGDWGAEV:HLA-A*02:01 complex, a SLGDWGAEI:HLA-A*02:01 complex and a SLGDWGAEL:HLA-A*02:01 complex, more preferably selected from the group consisting of: a SX₁GDVWGAEX₂:HLA-A*02:01 complex (wherein X₁ and X₂ are any amino acid), a SLGDWGAEA:HLA-A*02:01 complex, a SLGDVWGAEV:HLA-A*02:01 complex and a SLGDVWGAEI:HLA-A*02:01 complex, as appropriate. For each of the complexes listed in the previous sentence, the HLA-A*02:01 molecule may instead also be HLA-A*02, HLA-A*02:02, HLA-A*02:03, HLA-A*02:04, or HLA-A*02:09.

The term “TIMP3 antigen-specific binding protein,” as used herein, refers to a protein or polypeptide, such as a TCR or CAR, that specifically binds to a TIMP3 peptide antigen comprising the amino acid sequence of SEQ ID NO: 1 (or to a peptide antigen:HLA complex, where the peptide comprises the amino acid sequence of SEQ ID NO: 1 e.g., on a cell surface), and does not bind a peptide sequence that does not include the TIMP3 peptide antigen comprising the amino acid sequence of SEQ ID NO: 1.

In certain embodiments, a TIMP3-specific binding protein specifically binds to a TIMP3 peptide antigen (or a TIMP3 peptide antigen:HLA complex) with a Kd of less than about 10⁻⁸ M, less than about 10⁻⁹ M, less than about 10⁻¹⁰ M, less than about 10⁻¹¹ M, less than about 10⁻¹² M, or less than about 10⁻¹³ M, or with an affinity that is about the same as, at least about the same as, or is greater than at or about the affinity exhibited by an exemplary TIMP3-specific binding protein provided herein, such as any of the TIMP3-specific TCRs provided herein, for example, as measured by the same assay. In certain embodiments, a TIMP3-specific binding protein comprises a TIMP3-specific immunoglobulin superfamily binding protein or binding portion thereof.

The selective binding may be in the context of TIMP3 antigen presentation by HLA-A*02:01. In other words, in certain embodiments, a binding protein that “specifically binds to a TIMP3 antigen” may only do so when it is being presented (i.e. it is bound by) by a specific HLA or is in an equivalent structural formation as when it is being presented by the specific HLA.

Accordingly, in certain examples, a binding protein that “specifically binds to a TIMP3 antigen”, may only do so when it is being presented (i.e. it is bound by) by HLA-A*02:01 or is in an equivalent structural formation as when it is being presented by HLA-A*02:01. In other examples, a binding protein that “specifically binds to a TIMP3 antigen”, may only do so when it is being presented (i.e. it is bound by) by HLA-A*02:02 or is in an equivalent structural formation as when it is being presented by HLA-A*02:02. In yet other examples, a binding protein that “specifically binds to a TIMP3 antigen”, may only do so when it is being presented (i.e. it is bound by) by HLA-A*02:03 or is in an equivalent structural formation as when it is being presented by HLA-A*02:03. In further examples, a binding protein that “specifically binds to a TIMP3 antigen”, may only do so when it is being presented (i.e. it is bound by) by HLA-A*02:04 or is in an equivalent structural formation as when it is being presented by HLA-A*02:04. In other examples, a binding protein that “specifically binds to a TIMP3 antigen”, may only do so when it is being presented (i.e. it is bound by) by HLA-A*02:09 or is in an equivalent structural formation as when it is being presented by HLA-A*02:09. In other examples, a binding protein that “specifically binds to a TIMP3 antigen”, may only do so when it is being presented (i.e. it is bound by) by HLA-A*02 or is in an equivalent structural formation as when it is being presented by HLA-A*02.

By “specifically bind(s) to” as it relates to a T cell receptor, or as it refers to a recombinant T cell receptor, nucleic acid fragment, variant, or analog, or a modified cell, such as, for example, the TIMP3 T cell receptors, and TIMP3-expressing modified cells herein, is meant that the T cell receptor, or fragment thereof, recognizes, or binds selectively to a TIMP3 antigen comprising the amino acid sequence of SEQ ID NO: 1 (e.g. wherein the TIMP3 antigen comprises an amino acid sequence selected from the group consisting of SEQ ID NO: 1-27, 110 or 111). Under certain conditions, for example, in an immunoassay, for example an immunoassay discussed herein, the T cell receptor binds to a TIMP3 antigen comprising the amino acid sequence of SEQ ID NO: 1 (e.g. an amino acid sequence selected from the group consisting of SEQ ID NO: 1-27, 110 or 111) and does not bind in a significant amount to other polypeptides not comprising the amino acid sequence of SEQ ID NO: 1. Thus the T cell receptor may bind to a TIMP3 antigen (e.g. an amino acid sequence selected from the group consisting of: SEQ ID NO: 1-27, 110 or 111) with at least 10, 100, or 1000, fold more affinity than to a control antigenic polypeptide. This binding may also be determined indirectly in the context of a modified T cell that expresses a nucleic acid or vector of the invention (i.e. a CAR T cell or T cell expressing a TCR specific for the sequence of SEQ ID NO: 1). In assays such as, for example, an assay discussed herein, the modified cell is specifically reactive against a cell presenting a peptide comprising the amino acid sequence of SEQ ID NO: 1. Thus, the modified TIMP3-TCR expressing T cell may bind to a TIMP3 expressing cell with at least 10, 100, or 1000, fold more reactivity when compared to its reactivity against a control cell line that is not expressing TIMP3.

As used herein “specifically binds” or “specific for” refers to an association or union of a binding agent, for example a binding protein (e.g., TCR receptor) or a binding domain (or fusion protein thereof), to a target molecule with an affinity or Kₐ (i.e., an equilibrium association constant of a particular binding interaction with units of 1/M) equal to or greater than 10⁵M⁻¹ (which equals the ratio of the on-rate [kₒₙ] to the off-rate [k_{off}] for this association reaction), while not significantly associating or uniting with any other molecules or components in a sample. Binding proteins or binding domains (or fusion proteins thereof) may be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³M⁻¹. “Low affinity” binding proteins or binding domains refer to those binding proteins or binding domains having a Kα of up to 10⁷ M⁻¹. up to 10⁶ M⁻¹, up to 10⁵ M⁻¹. Alternatively, affinity can be defined as an equilibrium dissociation constant (K_{d}) of a particular binding interaction with units of M (e.g., 10⁻⁵ M to 10⁻¹³ M).

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof. In certain embodiments, enhanced affinity TCRs can be codon optimized to enhance expression in a particular host cell, such as a cell of the immune system, a inducible pluripotent stem cell (iPSC), a hematopoietic stem cell, a T cell, a primary T cell, a T cell line, a NK cell, or a natural killer T cell (Scholten et al, Clin. Immunol. 119: 135, 2006). The T cell can be a CD4+ or a CD8+ T cell, or gamma-delta T cell.

In some examples, a protein and a gene encoding said protein may be referred to using the same term. In examples where a protein and a gene encoding said protein are referred to using the same term, a person of skill in the art would readily be able to determine whether the protein or the gene was being referred to depending on the context in which the term was mentioned. Typically, gene names are written in italics.

A “non-essential” (or “non-critical”) amino acid residue is a residue that can be altered from the wild-type sequence of (e.g., the sequence identified by SEQ ID NO 1, 2, or 3 herein) without abolishing or, more preferably, without substantially altering a biological activity, whereas an “essential” (or “critical”) amino acid residue results in such a change. For example, amino acid residues that are conserved are predicted to be particularly non-amenable to alteration, except that amino acid residues within the hydrophobic core of domains can generally be replaced by other residues having approximately equivalent hydrophobicity without significantly altering activity.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential (or non-critical) amino acid residue in a protein is preferably replaced with another amino acid residue from the same side chain family. Alternatively, in another embodiment, mutations can be introduced randomly, and the resultant mutants can be screened for activity to identify mutants that retain activity.

Calculations of sequence homology or identity (the terms are used interchangeably herein) between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

Alternatively, the percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-410). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used. See <http://www.ncbi.nlm.nih.gov>.

The polypeptides and nucleic acid molecules described herein can have amino acid sequences or nucleic acid sequences sufficiently or substantially identical to the sequences identified by SEQ ID NOs 1 to 111. The terms “sufficiently identical” or “substantially identical” are used herein to refer to a first amino acid or nucleotide sequence that contains a sufficient or minimum number of identical or equivalent (e.g. with a similar side chain) amino acid residues or nucleotides to a second amino acid or nucleotide sequence such that the first and second amino acid or nucleotide sequences have a common structural domain or common functional activity. In other words, amino acid sequences or nucleic acid sequences having one or several (e.g. two, three, four etc) amino acid or nucleic acid substitutions compared to the corresponding sequences identified by a SEQ ID NO may be sufficiently or substantially identical to the sequences identified by the SEQ ID NO (provided that they retain the requisite functionality). In such examples, the one or several (e.g. two, three, four etc) amino acid or nucleic acid substitutions may be conservative substitutions. For example, amino acid or nucleotide sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical.

TCR sequences are defined according to IMGT. See the LeFranc references herein for further details i.e. [1] Lefranc M.-P. “Unique database numbering system for immunogenetic analysis” Immunology Today, 18: 509 (1997). [2] Lefranc M.-P. “The IMGT unique numbering for immunoglobulins, T cell Receptors and Ig-like domains” The immunologist, 7, 132-136 (1999). [3] Lefranc M.-P. et al. “IMGT unique numbering for immunoglobulin and Tcell receptor variable domains and Ig superfamily V-like domains” Dev. Comp. Immunol., 27, 55-77 (2003). [4] Lefranc M.-P. et al. “IMGT unique numbering for immunoglobulin and T cell receptor constant domains and Ig superfamily C-like domains” Dev. Comp. Immunol., 2005, 29, 185-203 PMID: 15572068.

As used herein, the term “ex vivo” refers to “outside” the body. The term “in vitro” can be used to encompass “ex vivo” components, compositions and methods.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLES

### Example 1

### Material and Methods

### Cell Culture

Tumor cells were cultured in IMDM medium (Gibco) supplemented with 100 ug/mL streptomycin, 100 U/mL penicillin, 2 mM L-glutamine (Invitrogen) and 8% FCS (Gibco). Genetic disruption of the TAP1, or TIMP3 gene in human tumor cell lines was performed with CRISPR/CAS9 and described before (Marijt et al. 2018). In brief, single guide RNAs (sgRNAs) targeting exon 1 of the human TAP1 gene (sgTAP1: 5′-GCT GCT ACT TCT CGC CGA CT-3′ (SEQ ID NO: 29)), or targeting exon 1 of the TIMP3 gene (TIMP3₁₅₋₂₃: 5′-GCACGATGAGCCCGAGCCAA-3′ (SEQ ID NO: 30)) were designed and cloned into the lentiCRI SPR v2 vector. Virus particles were generated by co-transfecting sgRNA/CAS9 containing plasmid together with PAX2/pMD2.G packaging vectors into HEK293T cells using Lipofectamine 2000 (Thermo Fisher). Tumor cells were incubated with medium containing the virus particles for 24 h, and transduced tumor cells were selected with puromycin selection (Gibco). TAP1 KO efficiency was analyzed by measuring surface HLA-ABC (w6/32; BioLegend) expression using flow cytometry. Polyclonal TAP KO cell lines were generated by FACS sorting the HLA-I low cell population. CRISPR/CAS9 TAP-WT control cells were generated by FACS sorting the HLA-I high cell population of the polyclonal bulk. Clones were made of the TAP/TIMP3 KO tumor cells by limited dilution.

T cells were cultured in IMDM medium (Gibco) supplemented with 2 mM L-glutamine, 10% human serum (Sanquin), and 100U/mL IL-2 (proleukine, Novartis). T cells were stimulated every 10-14 days using synthetic short peptide (Genscript) or 800 ng/ml PHA (Phytohaemagglutinin) (Murex Biotech), supplemented with 100 U/ml IL-2 and IL-7 (5 ng/mL), and a feeder mix containing irradiated PBMCs (1×10⁶ cells, 50 Gy), and EBV-JY cells (1×10⁵ cells, 75 Gy). All cell types were maintained in humidified air incubator at 37° C. and 5% CO₂.

### GPCR Analysis

RNA was isolated from cells with the NucleoSpin RNA Plus kit (Bioke). cDNA was made with the high capacity RNA-to-cDNA kit (Applied Biosystems). The efficiency of the gene knock out in the tumor cell bulk or clones was analyzed by qPCR using a CFX384 Real-Time System C1000 Thermal Cycler (BioRad). (primers TIMP3 fw: CAAGATGCCCCATGTGCAGT (SEQ ID NO: 31), rev: CTCTCCACGAAGTTGCACAG (SEQ ID NO: 32); TAP1: fw: CTTGCAGGGAGAGGTGTTTG (SEQ ID NO: 33), rev: GAGCATGATCCCCAAGAGAC (SEQ ID NO: 34)) Measurements were performed in triplicates. Ct-values were normalized to the expression of the housekeeping genes ARF5 (fw: TGCTGATGAACTCCAGAAGATGC (SEQ ID NO: 35), rev: CGGCTGCGTAAGTGCTGTAG (SEQ ID NO: 36)) and CPSF6 (fw: AAGATTGCCTTCATGGAATTGAG (SEQ ID NO: 37), rev: TCGTGATCTACTATGGTCCCTCTCT (SEQ ID NO: 38)).

### Generation of TEIPP-Specific T Cell Bulk Cultures

Buffy coats from healthy donors were obtained from Sanquin (Sanquin blood facility). Informed consent was given in writing by all participants. PBMCs were isolated using a ficoll gradient (density 1.077 g/ml). Approximately 500×10⁶ PBMCs were incubated with PE-labeled pHLA-A*02:01 tetramers (Flex-T™ HLA-A*02:01 Monomer UVX van Biolegend) for 30 min at RT. Anti-PE magnetic beads (MACS) were used to pull out pHLA-A*02:01 tetramer-positive cells over a MACS LS column as instructed by the manufacturer (Miltenyi Biotech). T cells were cultured in complete IMDM containing 10% human serum (Sanquin blood facility) and 100 U/ml IL-2 (Proleukine; Novartis). Bulk T cells were stimulated every 2 weeks with a mixture of T cells (106), 1 μg/ml synthetic peptide (SLGDWGAEA (SEQ ID NO: 2) (TIMP3)), irradiated PBMCs (10 cells, 50 Gray), and EBV-JY (10W cells, 75 Gray) in complete T cell culture medium supplemented with 100 U/ml IL-2 in 24-well plates (Costar). Culture medium was replenished every 2-3 d with fresh complete T cell medium.

### Cross-Presentation of Synthetic Long Peptides by MoDC

HLA-A*02:01 positive PBMCs were isolated from buffy-coats from consented donors (Sanquin bloodbank, Amsterdam), using a gradient ficoll layer. PBMCs were incubated with anti-CD14 magnetic beads for 20 min at 4° C. and the CD14 positive monocytes were isolated using magnetic separation columns (miltenyi). CD14+ monocyte were cultured in RPMI medium supplemented with 10% FCS, GM-CSF (800 units/ml), and IL-4 (500 units/ml) for 6 days to generate immature monocyte-derived dendritic cells. On day 6, the immature moDCs were incubated with synthetic long peptide (10 μM, Genscript) for 2h, followed by overnight incubation with LPS (20 ng/ml). Matured and loaded moDCs (30,000 cells) were washed and co-cultured with previously established tetramer positive T cells (50,000 cells) for 16 h. Direct presentation of synthetic long peptides was tested by continuous incubation with TIMP3-specific CD8 T cell bulk for 16 h in the absence of moDC. Cytokine release by T cells was then measured in supernatants by ELISA.

### T Cell Receptor Sequencing

TIMP3₁₅₋₂₃-specific CD8+ T cells from different sources of PBMC (lung cancer patients and healthy donors) were single cell sorted into iCapture plates (iRepertoire) on the basis of CD8 and TIMP3_{15-23V} tetramer. Plates were stored at −80° C., and send to irepertoire for paired TCR alpha/beta sequencing. Single cell sorting was done using a BD Ara III™ FACS. Single cell sequencing was performed by Amplicon-Rescued Multiplex (ARM)-PCR technology at the irepertoire headquarters according to manufacturer's instructions. In brief, nested inside and outside gene-specific primers are used in combined RT-PCR round 1 followed by target amplicon rescue and PCR round 2 using iDual PCR plates. The libraries are sequenced with the Illumina MiSeq platform using the MiSeq Reagent Nano Kit v2 or MiSeq reagent kit v2 (500 cycles). TCR sequencing results were analyzed using the iPair analyzer platform. Full-length cDNA transcripts for murinized TCRs for both TCR-alpha and TCR-beta chains were cloned into a retroviral pMP71 flex expression vector.

### Retrovirus Production

Platinum-Amphotropic retrovirus production (Plat-A) retroviral packing cells (Cell Biolabs) were used for retrovirus production. Plat-A cells were seeded in 6-well plates and incubated overnight until fully attached. Next, the cells were transfected with 2.5 μg pMP71_TCR vector using 5 μl lipofectamine 3000 reagent (Thermofisher). Next day, medium was refreshed, and retrovirus supernatant was harvested after 24 h. Next, retrovirus supernatant was spun down to remove cells and debris and subsequently incubated overnight at 4° C. with lenti-X concentrator (Clontech; 3 volumes retrovirus with 1 volume Lenti-X). Next day, supernatant was spun down for 45 minutes at 1500×g at 4° C., after which the pellet was gently dissolved in 50 μl PBS (for each 6 well) and concentrated virus was either stored at −80° C. in single-use aliquots or directly used for T cell transduction.

### T Cell Transduction and Culture of TEIPP-Specific T Cell Lines

CD8+ T cells were purified from PBMC using magnetic bead isolation (Miltenyi), and activated by stimulating them with aCD3/aCD28 expander beads (Thermofisher) at a 3:1 T cell:bead ratio in IMDM+ 8% human serum (Sanquin; hereafter referred to as T cell medium) in the presence of 100 U/ml IL-2, 10 ng/ml IL-7 and 5 ng/ml IL-15. After 48 h, 0.5-1×10⁶ activated CD8 T cells were plated in a retronectin (Takara) coated 24-well in 800 μl T cell medium together with 50 μl concentrated retrovirus supernatant and 100 U/ml IL-2. Subsequently, CD8 T cells and retrovirus containing supernatant was spun down for 90 min at 430×g at 24° C. to increase the efficiency of transduction. 24 h after transduction, CD8+ T cells were harvested, spun down for 5 minutes at 1600 rpm to remove the virus, and cells were resuspended in fresh T cell medium containing 100 U/ml IL-2. After 96 hours, transduction efficiency was determined by combined tetramer and murine TCRb antibody staining. Bulk T cells containing TCR transduced T cells were replenished every 2-3 d with fresh complete T cell medium until sufficient number (>5×10e6) of T cells were obtained for further TCR enrichment by TCRb-mediated MACS. To this end, CD8+ T cells were incubated for 30 minutes at RT with 1:200 TCRb-PE antibody, washed, and subsequently incubated with anti-PE microbeads for an additional 20 minutes at 4° C. Next, TCRb+ cells were isolated by separation over two consecutive MS columns, and subsequently expanded using irradiated PBMC (50 Gray) and EBV-JY cells (75 Gray) in complete T cell medium supplemented with 800 ng/ml PHA (Murex Biotech) and 100 U/ml IL-2. Culture medium was replenished every 2-3 d with fresh complete T cell medium.

### T Cell Reactivity Analysis

TCR-transduced or bulk-cultured TEIPP T cells were tested for their ability to selectively recognize the indicated TEIPP epitope presented on TAP impaired tumors by co-culturing them for 18 hours with wild-type (wt), TAP-KO and/or TAP/TIMP3-KO tumor cells at a 5-to-1 target-to-T cell ratio, after which supernatants were harvested and analyzed for cytokine production by GM-CSF, CCL4 and/or IFNg ELISA. Unstimulated and peptide-stimulated T cells were used as negative and positive controls. For some experiments, also endogenous TEIPP antigen-expressing healthy cells such as Epstein-Barr virus (EBV) transformed B-cells, monocytes or monocyte-derived dendritic cells were included as additional controls. Test were performed in triplo in 96 well round bottom plates at 1:1 target to effector ratio.

### Functional T Cell Affinity Analysis

Functional T cell affinity was determined by measuring GM-CSF and CCL4 production of activated T cells recognizing TEIPP-peptide pulsed HLA-A02:01+JY target cells. To this end, JY target cells were pulsed with increasing concentrations of peptide (10 pg/ml-10 ug/ml) in serum-free medium for 2 hours at 37° C., after which T cells were directly added in T cell medium and incubated overnight at 37° C. Next day, supernatant was harvested and GM-CSF and CCL4 production was determined by ELISA. For each TCR, the IC50 value was determined by calculating the peptide concentration corresponding to 50% cytokine production of the maximum response using Graphpad Prism non-linear regression with dose-response-inhibition equations (four-parameter dose-response curve). Each peptide concentration was tested in triplo with 4,000 T cells and 20,000 target cells per well in a 96 well round-bottom plate.

### Flow Cytometry Analysis

Tetramer staining on CD8 T cells was performed as described (Marjt et al. 2018). In brief, CD8+ T cells were first stained with zombie Aqua fluorescent dye (Biolegend) according to manufacturer's instruction to exclude dead cells. Next, cells were stained with PE-labeled TIMP3₁₅₋₂₃ tetramers for 30 minutes at RT in the dark, followed by anti-CD4 (clone SK-3, BD), anti-CD8 (clone SK-1, BD) and murine anti-TCRβ (clone H57-597, biolegend) antibody staining for 30 min at 4° C.

MoDCs were stained with anti-CD1a (clone H1149, BD), anti-CD14 (clone M5E2, BD), anti-CD80 (clone L307.4, BD), anti-CD83 (clone HB15e, BD), anti-CD86 (clone IT2.2, biolegend), and anti HLA-DR (clone G46-6, BD) antibodies for 30 min at 4° C. and washed three times with cold PBS/BSA. Samples were acquired using a BD LSRFortessa™ flow cytometry system and analyzed using FlowJo software (Tree Star).

### Classification of Gene Expression Distribution

| RNA cancer | |
|---|---|
| specificity: | Description |
| Enriched | NX level in a particular tissue/region/cell type at least four times any |
| | other tissue/region/cell type |
| Group enriched | NX levels of a group (of 2-5 tissues or 2-10 cell types or 2-5 brain |
| | regions) at least four times any other tissue/region/cell type |
| Enhanced | NX levels of a group (of 1-5 tissues or 1-10 cell types or 1-5 brain |
| | regions) at least four times the mean of other tissue/region/cell types |
| Low specificity | NX ≥ 1 in at least one tissue/region/cell type but not elevated in any |
| | tissue/region/cell type |
| Not detected | NX < 1 in all tissue/cell/region types |

### RNA Cancer Distribution:

| | |
|---|---|
| Detected in | Detected in a single tissue/region/cell type |
| single | |
| Detected in | Detected in more than one but less than one third of tissue/region/cell |
| some | types |
| Detected in | Detected in at least a third but not all tissue/region/cell types |
| many | |
| Detected in all | Detected in all tissue/region/cell types |
| Not detected | Not detected NX < 1 in all tissue/region/cell type |

### Results

### Screening for HLA-A*02:01-Restricted TEIPPS.

36 peptides (designated 101 to 136) were selected for their capacity to activate CD8+ T cells present in peripheral blood mononuclear cells (PBMC) of maximal 5 healthy donors, following the protocol described earlier (Marnjt et al., 2018). For 21 of the 36 peptides, the PBMC of at least 3 out of 5 donors showed in vitro expansion of HLA-A*02:01-specific peptide multimer-positive CD8+ T cells. After an in vitro expansion and enrichment of the responding T cell cultures, 18 of the 21 cases could be tested for their capacity to preferentially recognize TAP-negative tumor cells over TAP-positive tumor cells. In the end, 3 cultures showed a consistent recognition pattern and could be classified as TEIPP peptides in HLA-A*02:01. These cultures were stimulated with peptide 105, 113, or 124. Identifying data about peptide 124 is provided in Table 2. Notably peptide 105 is the earlier identified LRPAP1-derived TEIPP peptide FLGPWPAAS (SEQ ID NO: 39), reported in Manijt et al. (2018), confirming the inventors' previous study. Finally, the number of healthy donors tested for responsiveness to peptide 124 was increased, leading to the outgrowth of p124-specific Tcells in 10 donors, showing the strong immunogenicity of the peptide and the general availability of T cells in the human T-cell repertoire to this peptide.

TABLE 2
Identifying data of new HLA-A*02:01-restricted TEIPP peptides

| | | | Amino | | | | | | |
|---|---|---|---|---|---|---|---|---|---|
| | Amino | | acids | Predicted | | | | RNA | RNA |
| | acid | | in | Affinity | Eluted | Tumor | Ubiquitous | distribution | specificity |
| Nr | Sequence | Gene¹ | sequence | HLA-A*02:01 | from | IEDB | expression | cancer | cancer |
| 124 | SLGDWGAEA | TIMP3 | 15-23 | 70 nM | ND | 2 | Yes | in many | Low |
| | (SEQ ID NO: 2) | | | | | | (low) | | |

¹TIMP3, TIMP Metallopeptidase Inhibitor 3

### Peptide 124-Specific CDB+ T Cell Cultures Recognize TAP-Impaired Tumor Cells and not Healthy Cells.

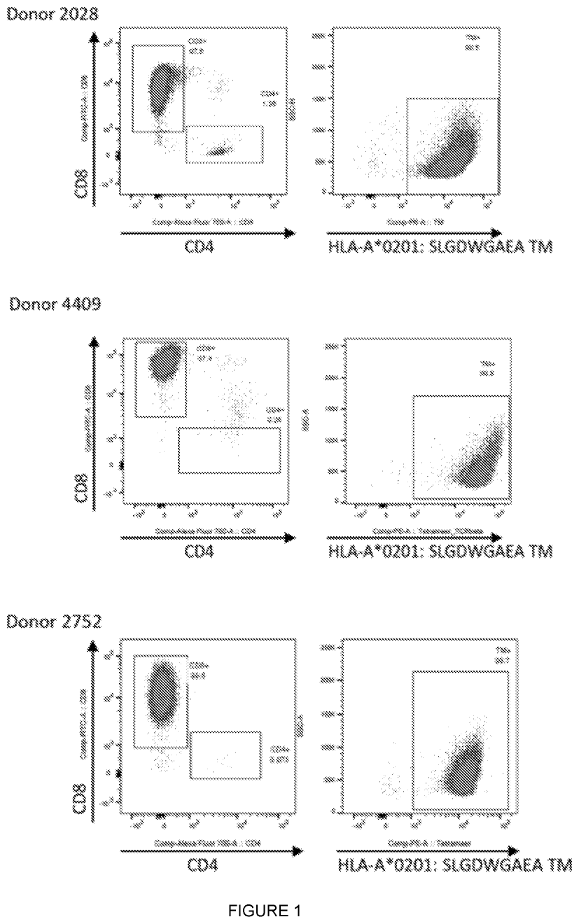

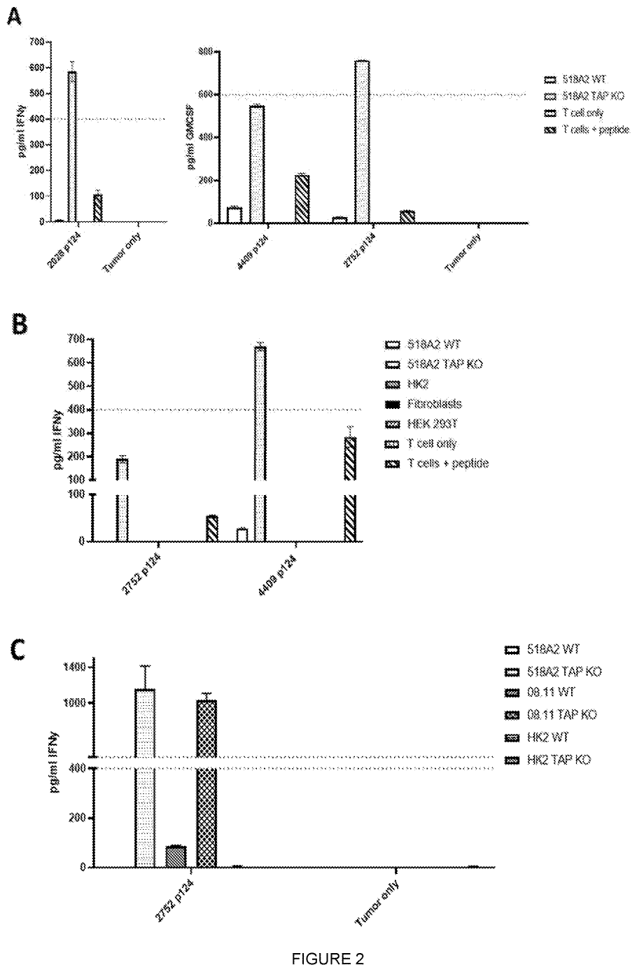

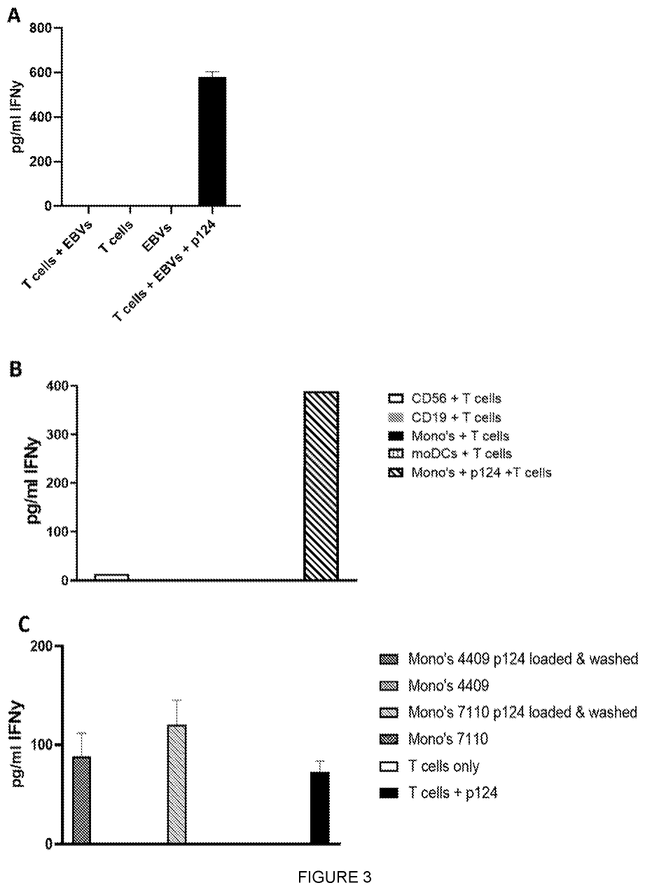

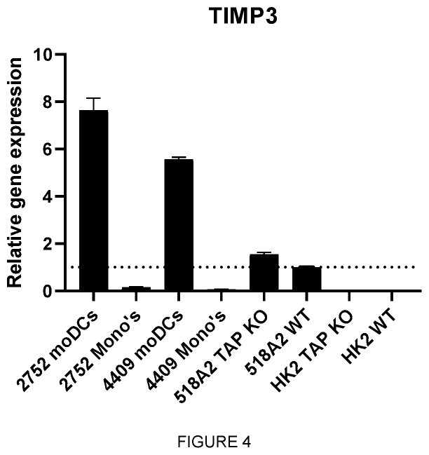

Three different peptide 124-specific Tcell cultures comprising >97% CD8+ T cells that stained for >99% positive with peptide 124-loaded HLA-A*0201 tetramers (FIG. 1) were used. Peptide 124-specific T cells preferentially recognized 518A2 melanoma and 08.11 melanoma cells when TAP is knocked out, but not non-tumor cells such as fibroblasts, HEK-293T cells, or HK2 cells (FIG. 2). In addition, neither recognition of EBV transformed B-cells (FIG. 3A), nor of CD56+cells (NK or T cells), CD19+B-cells, monocytes that were isolated from PBMC or monocyte-derived DCs (FIGS. 3B and 3C) was observed, despite the fact that DCs have a much stronger expression of TIMP3 than 518A2 melanoma cells (FIG. 4). The p124-specific CD8 T cells did not recognize HK2 TAP KO cells, fitting with the undetectable RNA levels of TIMP3 (FIG. 4).

These data clearly show that peptide 124 specific CD8+ T cells preferentially recognize tumor cells displaying lower levels of TAP, and as such peptide 124 can also be classified as a genuine TEIPP.

### Peptide 124-Specific CDB+ T Cell Cultures are TIMP3-Specific.

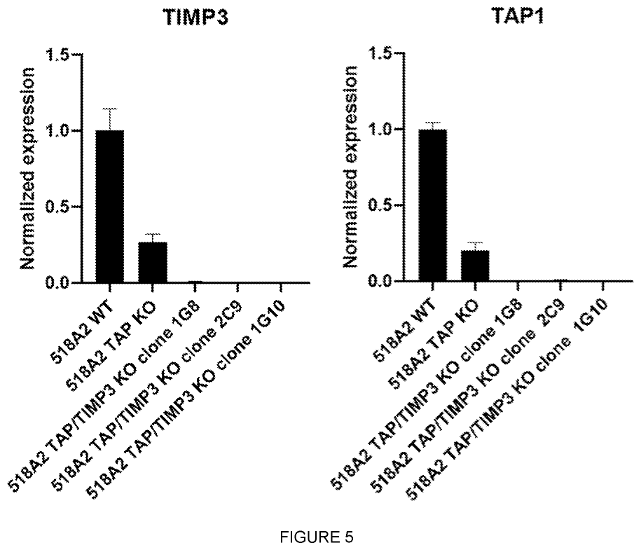

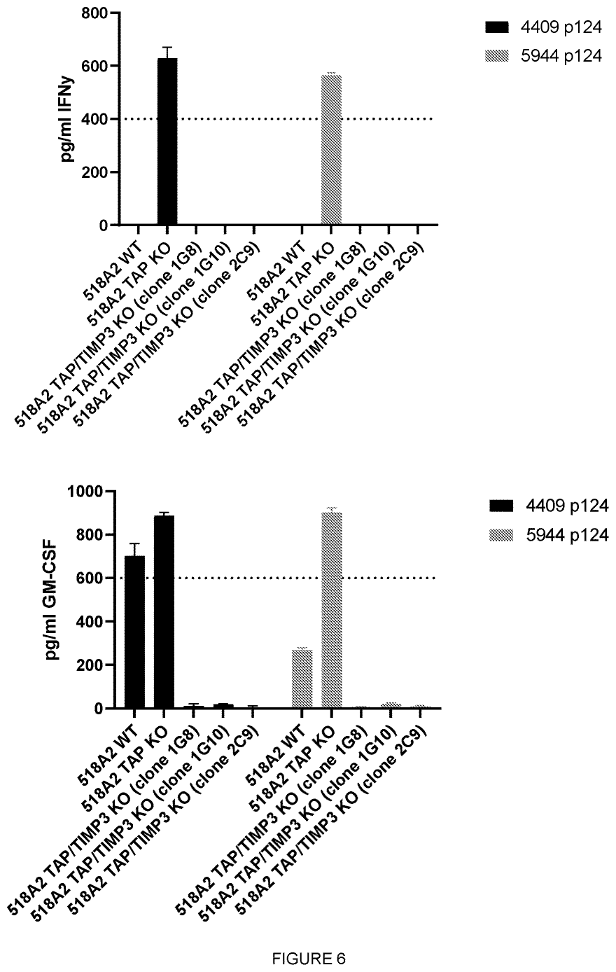

An important feature for the clinical application of TEIPP-specific T cells is the absence of cross-reactivity to peptides-derived from other proteins present in cells. Therefore, to test for the possibility that p124-specific CD8 T cells also recognize peptides not encoded by TIMP3, this gene encoding peptide 124 was knocked out in 518A2 TAPKO melanoma cells, resulting in 3 different clones (FIG. 5). Analysis of the reactivity of p124-specific CD8+ T cells showed a complete absence of recognition when stimulated with 518A2 TAP KO TIMP3 KO cells, while 518A2 were recognized, especially when TAP was knocked out (518A2 TAP KO; FIG. 6). This shows that peptide 124-specific T cells are truly specific for the TIMP3 derived peptide SLGDNGAEA.

The TIMP3-Derived Peptide SLGDWGAEA is Directly Processed from a Longer Peptide Sequence.

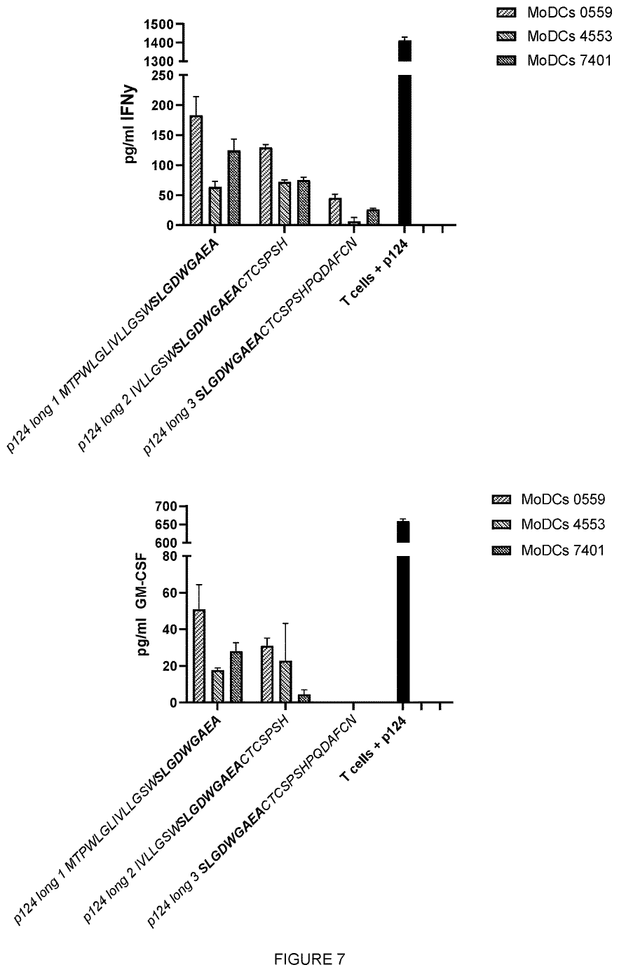

The synthetic long peptide (SLP) platform is highly immunogenic and successfully applied in therapeutic SLP vaccination for HPV-induced cancers (Kenter et al. 2009; van Poelgeest et al. 2016). Therefore, the inventors examined the efficiency of cross-presentation of long versions of the TIMP3-derived signal peptide SLGDWGAEA (SEQ ID NO: 2) in dendritic cells, using natural flanking amino acids extending the amino-terminus, the carboxy-terminus, or both ends (Table 3). Monocyte-derived dendritic cells (moDC) were incubated with these three SLPs (P124 long 1-3; SEQ ID NOs: 22-24), then matured, and used as targets for the p124-specific CD8 T cell culture to assess correct processing and HLA-A2 presentation of the minimal TEIPP epitope. Despite the fact that the c-terminus was an alanine, the TIMP3-derived signal peptide SLGDWGAEA (SEQ ID NO: 2) was cross-presented by DCs from a longer peptide to p124-specific CD8 T cells (FIG. 7). Exogenous pulsing of the short p124 SLGDWGAEA (SEQ ID NO: 2) peptide also stimulated the T cells (FIG. 7). Specifically, when the T-cell epitope was in the middle or at the c-terminus of the synthetic long peptide (P124 long 1 and 2) the T cells were activated. No recognition was observed when the T-cell epitope was placed at the n-terminal part of the long peptide (P124 long 3), suggesting that too many flanking amino acids at the c-terminal part of the epitope blocked its processing by DCs.

TABLE 3
Synthetic long peptides used for assessing
cross-presentation of the wild type and
c-terminal anchor replaced amino acid
variant of peptide 124.

| Name | SEQ ID NO | Amino-acid sequence¹ |
|---|---|---|
| P124 long 1 | 22 | MTPWLGLIVLLGSWSLGDWGAEA |
| P124 long 2 | 23 | IVLLGSWSLGDWGAEACTCSPSH |
| P124 long 3 | 24 | SLGDWGAEACTCSPSHPQDAFCN |
| P124 long V1-3 | 25 | WLGLIVLLGSWSLGDWGAEV |
| P124 long V1-4 | 110 | LGLIVLLGSWSLGDWGAEV |
| P124 long V1-6 | 111 | LIVLLGSWSLGDWGAEV |
| P124 long V2 | 26 | IVLLGSWSLGDWGAEVCTCSPSH |
| P124 long V3 | 27 | SLGDWGAEVCTCSPSHPQDAFCN |

¹The amino acid sequence of the TIMP3-derived peptide is provided, the T cell epitope (peptide 124) is shown in bold. The c-terminal amino-acid substitution is underlined.

C-Terminal Anchor Replacement from Alanine to Valine in the TIMP3-Derived Peptide SLGDWGAEA Impairs Cross-Presentation of the T-Cell Epitope from Long Peptide Sequences.

Cross-presentation of long peptides by dendritic cells involves endocytosis, cytosolic cleavage of the SLP into short peptides by the proteasome, transport over the ER membrane by TAP and loading onto MHC-1 molecules (van Hall & van der Burg, 2012; Rosalia et al. 2013). The inventors analyzed the predicted binding affinity of the wild type peptide and its variants. This revealed that substitution of alanine (A) to a valine (V), but also to a leucine (L) or isoleucine (1), would greatly improve the binding affinity of this peptide to HLA-A*0201. For HLA-A*02:01, the C-terminal alanine is not the most optimal anchor residue as shown for the predicted binding affinity, ranking and binding level of this peptide (Table 4).

TABLE 4
Predicted binding affinity of SLGDWGAEA and
c-terminal anchor variants in HLA-A*02:01.

| | SEQ | Binding | | | |
|---|---|---|---|---|---|
| | ID | affinity | Rank | Binding | Recognition |
| Sequence | NO | (nM)¹ | (%) | level | (IC50)² |
| SLGDWGAEA | 2 | 69.9 | 2 | Weak | 92.525 ng/ml |
| SLGDWGAEV | 3 | 11.4 | 0.5 | Strong | 1.471 ng/ml |
| SLGDWGAEL | 5 | 30.4 | 1.5 | Strong | ND |
| SLGDWGAEI | 4 | 46.3 | 1.5 | Strong | 284.929 ng/ml |
| SLGDWGAEM | 15 | 126 | 3 | Weak | ND |

¹The prediction values are given in nM IC50 values and as % Rank to a set of 200.000 random natural peptides. Strong and weak binding peptides are indicated. Prediction by NetMHCcons1.1 (https://services.healthtech.dtu.dk/service.php?NetMHCcons-1.1);
²The concentration of peptide for half maximal interferon-gamma production of SLGDWGAEA-specific T cells stimulated with the different variant peptides.
ND, not detectable

The C-terminus docks into the F-pocket and is important for binding to MHC-1 molecules, but does not directly interact with T cell receptors (TCR) and might thus be replaced. The inventors investigated if exchanges into another amino acids would result in a more efficiently processed epitope. First, they examined whether these substitutions would interfere with T cell receptor interaction. The inventors exogenously pulsed the short minimal peptides of Table 4 with the C-terminal valine (V), leucine (L), isoleucine (I), or methionine (M) on HLA-A*0201 positive EBV transformed B-cells and measured the activation of the peptide 124-specific CD8+ T cell culture. No response was observed for the two M-variant and L-variant peptides, despite the fact that the latter was predicted to transform the weak-binding wild type peptide to a strong binding peptide. Substituting the c-terminal alanine to valine improved the stimulation of p124-specific T cells by 61-fold, specifically at the lower peptide concentration range, suggesting that this substitution would be beneficial for cross-presentation.

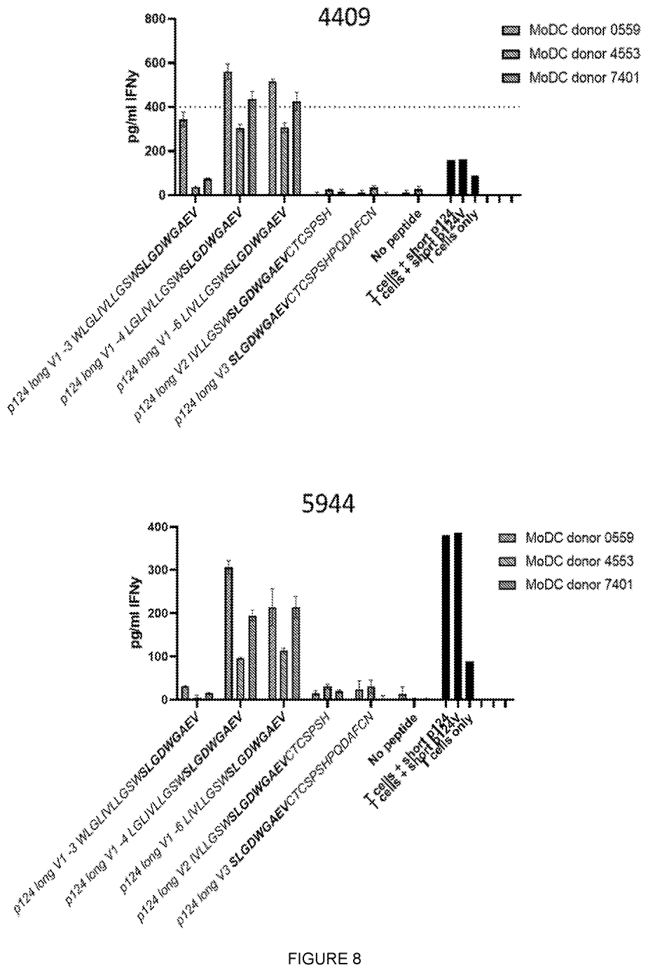

Therefore, the cross-presentation of the V-variant was evaluated as synthetic long peptide extended with natural flanking amino acids (Table 3). After uptake and processing of the five different variants (P124 long V1-3, V1-4, V1-6, V2 and V3; SEQ ID NOs: 25, 110, 111, 26 and 27), moDCs were co-cultured with the p124-specific T cell cultures and cytokine production was measured (FIG. 8). Cross-presentation of the TIMP3-derived V-variant SLGDWGAEV peptide was less optimal when the epitope was placed at the c-terminal part of the longer peptide (i.e. P124 long V1-3; SEQ ID NO: 25), indicated by a less consistent presentation to p124-specific T cells by DC from three different donors, and cross-presentation was lost when the epitope was placed in the middle of the long peptide (FIG. 8). The lack of recognition of DCs not loaded with peptide reiterates that moDCs, despite the high expression of TIMP3 (FIG. 4), are not recognized. The data indicates that c-terminal anchor replacement from an alanine to a valine, not necessarily improves and may even impair cross-presentation of TEIPP epitopes. However, a further shortening of the N-terminal part of the longer peptide P124 long V1-3 (SEQ ID NO: 25) surprisingly resulted in enhanced T cell stimulation and more efficient cross-presentation by monocyte-derived dendritic cells (P124 long V1-4 and V1-6 (SEQ ID NOs: 110 and 111), FIG. 8). Surprisingly, this specific amino acid change combined with alterations in the flanking sequence allows the peptide to be used as a more effective vaccine.

### Identification of Unique HLA-A02:01-Restricted TIMP3₁₅₋₂₃ CD8 T Cell Receptors.

Above the inventors reported the identification of the HLA-A*02:01-restricted TIMP3₁₅₋₂₃ CD8 T cell epitope, present in the signal sequence of the TIMP3 protein. CD8 T cells reactive to this peptide preferentially recognize tumor cells displaying lower levels of TAP. Here, the inventors single cell sorted CD8 T cells specifically binding to MHC multimers of HLA-A*02:01-molecules presenting the peptide TIMP3₁₅₋₂₃ SLGDVWGAEA from 4 different TIMP3₁₅₋₂₃-specific bulk T-cell cultures displaying preferentially recognition of tumor cells with low levels of TAP. Then the TCR-alpha and TCR-beta chains were DNA sequenced in order to identify a series of unique T-cell receptors. This revealed the presence of 9 different T cell clones (Table 5) of which 7 displayed a productive and unique T-cell receptor (Tables 7-13) amongst a total of 80 TCRs. In the culture of donor 4409 only 3 unique T-cell receptors were identified, in which TCR #3 dominated. In the culture of donor 2028, 2 unique T-cell receptors were identified but TCR #4 clearly dominated. In the culture of donor 2752, 3 unique T-cell receptors were identified but only one (TCR #6) dominated. In the culture of donor 5944 only one TCR was found multiple times (TCR #9). The four dominating TCRs (Table 5), most likely represent the T cells responsible for the TIMP3₁₅₋₂₃ reactivity found in the bulk T cell cultures they were sorted from.

TABLE 5
Isolation of unique TCR sequences to the HLA-
A*02:01-restricted TIMP3 peptide SLGDWGAEA

| TCR | | Times |
|---|---|---|
| sequences | Sample Name | identified |
| 1 | Donor_4409_p124 | 1 |
| | Donor_4409_p124 | |
| 2 | Donor_4409_p124 | 2 |
| | Donor_4409_p124 | |
| 3 | Donor_4409_p124 | 8 |
| | Donor_4409_p124 | |
| 4 | Donor_2028_p124 | 38 |
| | Donor_2028_p124 | |
| 5 | Donor_2028_p124 | 1 |
| | Donor_2028_p124 | |
| 6 | Donor_2752_p124 | 22 |
| | Donor_2752_p124 | |
| 7 | Donor_2752_p124 | 3 |
| | Donor_2752_p124 | |
| 8 | Donor_2752_p124 | 1 |
| | Donor_2752_p124 | |
| 9 | Donor_5944_p124 | 4 |
| | Donor_5944_p124 | |

### Additional Screening Indicates the Validity of a Majority of the Identified TCRs

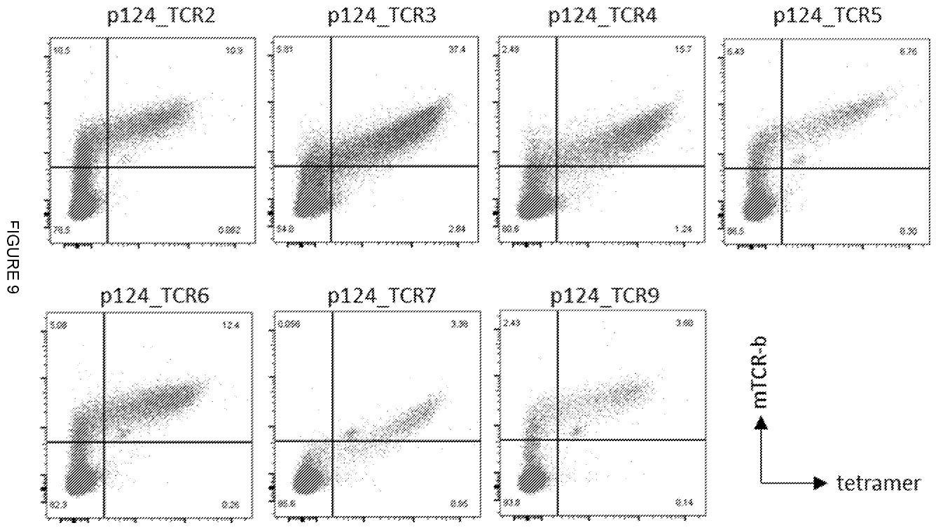

The TCRs as identified by the inventors above were subjected to additional screening experiments. The TCR-alpha and TCR-beta chains of each respective identified TCR were cloned into a retroviral expression vector, which was used to transduce T cells, resulting in the generation of T cells expressing the respective TCRs for peptide TIMP3₁₅₋₂₃ SLGDVNGAEA. The introduced genes contain the murine TCR-Cβ domain which enhances correct pairing of transgenic alpha and beta chains. These T cells were analyzed via flow cytometry. Expression of this domain is confirmed in TCR-transduced T cells after incubation with murine TCRb antibody and the specificity of the T cells after binding MHC multimers of HLA-A*02:01-molecules presenting the peptide TIMP3₁₅₋₂₃ SLGDWGAEA (FIG. 9). All of the transduced T cells produced a sufficient signal and were considered to valid. An interpretation of the results of the experiments of FIG. 9 is presented in Table 6, wherein ‘+’ indicates a positive, sufficient signal and ‘+/−’ indicates a sufficient signal.

TABLE 6
Interpretation of signal strengths assessed in FIG. 9

| TCR | TCRb+ | Tetramer+ | TCR validity |
|---|---|---|---|
| TCR_2 | + | + | + |
| TCR_3 | + | + | + |
| TCR_4 | + | + | + |
| TCR_5 | + | + | + |
| TCR_6 | + | + | + |
| TCR_7 | +/− | + | + |
| TCR_9 | + | + | + |

The readers attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Sequences
SEQ ID NO: 1
SX₁GDWGAEX₂,
wherein X₁ and X₂ are any amino acid
(i.e A or R or N or D or
C or Q or E or G or H or I or L or K or M or
F or P or O or S or U or T or W or Y or V).
SEQ ID NO: 2
SLGDWGAEA
SEQ ID NO: 3
SLGDWGAEV
SEQ ID NO: 4
SLGDWGAEI
SEQ ID NO: 5
SLGDWGAEL
SEQ ID NO: 6
SLGDWGAER
SEQ ID NO: 7
SLGDWGAEN
SEQ ID NO: 8
SLGDWGAED
SEQ ID NO: 9
SLGDWGAEC
SEQ ID NO: 10
SLGDWGAEE
SEQ ID NO: 11
SLGDWGAEQ
SEQ ID NO: 12
SLGDWGAEG
SEQ ID NO: 13
SLGDWGAEH
SEQ ID NO: 14
SLGDWGAEK
SEQ ID NO: 15
SLGDWGAEM
SEQ ID NO: 16
SLGDWGAEF
SEQ ID NO: 17
SLGDWGAEP
SEQ ID NO: 18
SLGDWGAES
SEQ ID NO: 19
SLGDWGAET
SEQ ID NO: 20
SLGDWGAEW
SEQ ID NO: 21
SLGDWGAEY
SEQ ID NO: 22
MTPWLGLIVLLGSWSLGDWGAEA
SEQ ID NO: 23
IVLLGSWSLGDWGAEACTCSPSH
SEQ ID NO: 24
SLGDWGAEACTCSPSHPQDAFCN
SEQ ID NO: 25
WLGLIVLLGSWSLGDWGAEV
(note that:
SEQ ID NO: 110 is LGLIVLLGSWSLGDWGAEV;
and SEQ ID NO: 111 is LIVLLGSWSLGDWGAEV,
therefore SEQ ID NO: 110 and 111 are
present within SEQ ID NO: 25)
SEQ ID NO: 26
IVLLGSWSLGDWGAEVCTCSPSH
SEQ ID NO: 27
SLGDWGAEVCTCSPSHPQDAFCN
SEQ ID NO: 28
MTPWLGLIVLLGSWSLGDWGAEACTCSPSHPQDAFCNSDIVIR
AKVVGKKLVKEGPFGTLVYTIKQMKMYRGFTKMPHVQYIHTEA
SESLCGLKLEVNKYQYLLTGRVYDGKMYTGLCNFVERWDQLTL
SQRKGLNYRYHLGCNCKIKSCYYLPCFVTSKNECLWTDMLSNF
GYPGYQSKHYACIRQKGGYCSWYRGWAPPDKSIINATDP
SEQ ID NO: 29
5′-GCT GCT ACT TCT CGC CGA CT-3′
SEQ ID NO: 30
5′-GCACGATGAGCCCGAGCCAA-3′
SEQ ID NO: 31
CAAGATGCCCCATGTGCAGT
SEQ ID NO: 32
CTCTCCACGAAGTTGCACAG
SEQ ID NO: 33
CTTGCAGGGAGAGGTGTTTG
SEQ ID NO: 34
GAGCATGATCCCCAAGAGAC
SEQ ID NO: 35
TGCTGATGAACTCCAGAAGATGC
SEQ ID NO: 36
CGGCTGCGTAAGTGCTGTAG
SEQ ID NO: 37
AAGATTGCCTTCATGGAATTGAG
SEQ ID NO: 38
TCGTGATCTACTATGGTCCCTCTCT
SEQ ID NO: 39
FLGPWPAAS

TABLE 7
TCR-2; target peptide SLGDWGAEA (SEQ ID NO 2); target gene TIMP3 A*02:01

| SEQ | TCR | AA | |
|---|---|---|---|
| ID | POLY- | or | |
| NO | PEPTIDE | NT | SEQUENCE |
| 40 | α CDR1 | AA | VGISA |
| 41 | α CDR2 | AA | SGHVS |
| 42 | α CDR3 | AA | AVGGGADGLT |
| 43 | β CDR1 | AA | LSSGK |
| 44 | β CDR2 | AA | FQNEAQ |
| 45 | β CDR3 | AA | ASSLVGRGYEQF |
| 46 | α VJ | AA | KNEVEQSPQNLTAQEGEFITINCSYSVGISALHWLQQHPGGGIVSLFML |
| | | | SSGKKKHGRLIATINIQEKHSSLHITASHPRDSAVYICAVGGGADGLTFG |
| | | | KGTHLIIQP |
| 47 | α VJ | NT | atttatcacaatcaactgcagttactcggtaggaataagtgccttacactggctgcaa |
| | | | cagcatccaggaggaggCATTGTTTCCTTGTTTATGCTGAGCTCAGGGAAGAAGAAGC |
| | | | ATGGAAGATTAATTGCCACAATAAACATACAGGAAAAGCACAGCTCC |
| | | | CTGCACATCACAGCCTCCCATCCCAGAGACTCTGCCGTCTACATCTG |
| | | | TGCTGTCGGAGGAGGTGCTGACGGACTCACCTttggcaaagggactcatcta |
| | | | atcatccagccctatatccagaaccctgaccctgccgtgtaccagctgagagact |
| 48 | β VDJ | AA | MGTRLLCWVVLGFLGTDHTGAGVSQSPRYKVAKRGQDVALRCDPISG |
| | | | HVSLFWYQQALGQGPEFLTYFQNEAQLDKSGLPSDRFFAERPEGSVST |
| | | | LKIQRTQQEDSAVYLCASSLVGRGYEQFFGPGTRLTVL |
| 49 | β VDJ | NT | tagctctcaggtgtgatccaatttcgggtcatgtatcccttttttggtaccaacaggc |
| | | | cctggggcaggggccagagtttctgacttatttccagaatGAAGCTCAACTAGACAAA |
| | | | TCGGGGCTGCCCAGTGATCGCTTCTTTGCAGAAAGGCCTGAGGGATCCGTCTCCACTC |
| | | | TGAAGATCCAGCGCACACAGCAGGAGGACTCCGCCGTGTATCTCTGTGCCAGCAGCTT |
| | | | AGTGGGCAGGggatacgagcagttcttcgggccagggacacggctcaccgtgctagag |
| | | | gacctgaaaaacgtgttcccacccgaggtcgctgtgtttgagccatcaga |

Sequences for TCR-2; target peptide SLGDWGAEA (SEQ ID NO: 2); target gene TIMP3 A*02:01.

TABLE 8
TCR-3; target peptide SLGDWGAEA (SEQ ID NO: 2); target gene TIMP3 A*02:01

| SEQ | TCR | AA | |
|---|---|---|---|
| ID | POLYPE | or | |
| NO | PTIDE | NT | SEQUENCE |
| 50 | α CDR1 | AA | YGATPY |
| 51 | α CDR2 | AA | YFSGDTLV |
| 52 | α CDR3 | AA | AVGPNNNDMR |
| 53 | β CDR1 | AA | SEHNR |
| 54 | β CDR2 | AA | FQNEAQ |
| 55 | β CDR3 | AA | ASSRGTDTQY |
| 56 | α VJ | AA | AQSVTQPDIHITVSEGASLELRCNYSYGATPYLFWYVQSPGQGLQLLLKYF |
| | | | SGDTLVQGIKGFEAEFKRSQSSFNLRKPSVHWSDAAEYFCAVGPNNNDM |
| | | | RFGAGTRLTVKP |
| 57 | α VJ | NT | cactggagttgagatgtaactattcctatggggcaacaccttatctcttctggtatgtccag |
| | | | tcccccggccaaggcctccagctGCTCCTGAAGTACTTTTCAGGAGACACTCTGGTTCAAGG |
| | | | CATTAAAGGCTTTGaggctgaatttaagaggagtcaatcttccttcaatctgaggaaaccct |
| | | | ctgtgcattggagtgatgctgctgagtacttctgtgctgtgggtccgaataacaatgacatg |
| | | | cgctttggagcagggaccagactgacagtaaaaccaaatatccagaaccctgaccctgccgt |
| | | | gtaccagctgagagact |
| 58 | β VDJ | AA | MGTSLLCWMALCLLGADHADTGVSQNPRHKITKRGQNVTFRCDPISEHNR |
| | | | LYWYRQTLGQGPEFLTYFQNEAQLEKSRLLSDRFSAERPKGSFSTLEIQR |
| | | | TEQGDSAMYLCASSRGTDTQYFGPGTRLTVL |
| 59 | β VDJ | NT | taactttcaggtgtgatccaatttctgaacacaaccgcctttattggtaccgacagaccctg |
| | | | gggcagggcccagagtttctgacttacttcCAGAATGAAGCTCAACTAGAAAAATCAAGGCT |
| | | | GCTCAGTGATCGGTTCTCTGCAGAGAGGCCTAAGGGATCTTTCTCCACCTTGGAGATCCAGC |
| | | | GCACAGAGCAGGGGGACTCGGCCATGTATCTCTGTGCCAGCAGCCGGGGGACAGAtacgcag |
| | | | tattttggcccaggcacccggctgacagtgctcgaggacctgaaaaacgtgttcccacccga |
| | | | ggtcgctgtgtttgagccatcaga |

Sequences for TCR-3; target peptide SLGDWGAEA (SEQ ID NO: 2); target gene TIMP3 A*02:01.

TABLE 9
TCR-4; target peptide SLGDWGAEA (SEQ ID NO: 2); target gene TIMP3 A*02:01

| SEQ | TCR | AA | |
|---|---|---|---|
| ID | POLY- | or | |
| NO | PEPTIDE | NT | SEQUENCE |
| 60 | α CDR1 | AA | DSVNN |
| 61 | α CDR2 | AA | IPSGT |
| 62 | α CDR3 | AA | AVRDDKII |
| 63 | β CDR1 | AA | SEHNR |
| 64 | β CDR2 | AA | FQNEAQ |
| 65 | β CDR3 | AA | ASSFATEAF |
| 66 | α VJ | AA | GIQVEQSPPDLILQEGANSTLRCNFSDSVNNLQWFHQNPWGQLINLFYIPS |
| | | | GTKQNGRLSATTVATERYSLLYISSSQTTDSGVYFCAVRDDKIIFGKGTRLH |
| | | | ILP |
| 67 | α VJ | NT | tccacgctgcggtgcaatttttctgactctgtgaacaatttgcagtggtttc |
| | | | atcaaaacccttgGGGACAGCTCATCAACCTGTTTTACATTCCCTCAGGGAC |
| | | | AAAACAGAATGGAAGATTAAGCGCCACGACTGTCGCTACGGAACGCTACAGC |
| | | | TTATTGTACATTTCCTCTTCCCAGACCACAGACTCAGGCGTTTATTTCTGTG |
| | | | CTGTGAGAGATGACAAGATCATCTTTGGAAAAGGgacacgacttcatattct |
| | | | ccccaatatccagaaccctgaccctgccgtgtaccagctgagagact |
| 68 | β VDJ | AA | MGTSLLCWMALCLLGADHADTGVSQNPRHKITKRGQNVTFRCDPISEHNR |
| | | | LYWYRQTLGQGPEFLTYFQNEAQLEKSRLLSDRFSAERPKGSFSTLEIQR |
| | | | TEQGDSAMYLCASSFATEAFFGQGTRLTVV |
| 69 | β VDJ | NT | taactttcaggtgtgatccaatttctgaacacaaccgcctttattggtac |
| | | | cgacagaccctggggcagggcccagagtTTCTGACTTACTTCCAGAATGA |
| | | | AGCTCAACTAGAAAAATCAAGGCTGCTCAGTGATCGGTTCTCTGCAGAGA |
| | | | GGCCTAAGGGATCTTTCTCCACCTTGGAGATCCAGCGCACAGAGCAGGGG |
| | | | GACTCGGCCATGTATCTCTGTGCCAGCAGCTTTGCAACTGAAgctttctt |
| | | | tggacaaggcaccagactcacagttgtagaggacctgaacaaggtgttcc |
| | | | cacccgaggtcgctgtgttt |

Sequences for TCR-4; target peptide SLGDWGAEA (SEQ ID NO: 2); target gene TIMP3 A*02:01.

TABLE 10
TCR-5; target peptide SLGDWGAEA (SEQ ID NO: 2); target gene TIMP3 A*02:01

| SEQ | TCR | AA | |
|---|---|---|---|
| ID | POLY- | or | |
| NO | PEPTIDE | NT | SEQUENCE |
| 70 | α CDR1 | AA | TSENNYY |
| 71 | α CDR2 | AA | QEAYKQQN |
| 72 | α CDR3 | AA | AFTINTGTASKLT |
| 73 | β CDR1 | AA | SGHTA |
| 74 | β CDR2 | AA | FQGNSA |
| 75 | β CDR3 | AA | ASRDYSYNEQF |
| 76 | α VJ | AA | AQTVTQSQPEMSVQEAETVTLSCTYDTSENNYYLFWYKQPPSRQMILV |
| | | | IRQEAYKQQNATENRFSVNFQKAAKSFSLKISDSQLGDTAMYFCAFTIN |
| | | | TGTASKLTFGTGTRLQVTL |
| 77 | α VJ | NT | tgtgaccctgagttgcacatatgacaccagtgagaataattattatttgttctggtacaagcagcctccc |
| | | | agcaggcagatgattctcgttattcgCCAAGAAGCTTATAAGCAACAGAATGCAAC |
| | | | GGAGAATCGTTTCTCTGTGAACTTCCAGAAAGCAGCCAAATCCTTCA |
| | | | GTCTCAAGATCTCAGACTCACAGCTGGGGGACACTGCGATGTATTTC |
| | | | TGTGCTTTCACAATAAATACCGgcactgccagtaaactcacctttgggactggaacaa |
| | | | gacttcaggtcacgctcgatatccagaaccctgaccctgccgtgtaccagctgagagact |
| 78 | β VDJ | AA | MGTRLLFWVAFCLLGADHTGAGVSQSPSNKVTEKGKDVELRCDPISGH |
| | | | TALYWYRQSLGQGLEFLIYFQGNSAPDKSGLPSDRFSAERTGGSVSTL |
| | | | TIQRTQQEDSAVYLCASRDYSYNEQFFGPGTRLTVL |
| 79 | β VDJ | NT | agctctcaggtgtgatccaatttcaggtcatactgccctttactggtaccgacagagcctggggcaggg |
| | | | cctggagtttttaaTTTACTTCCAAGGCAACAGTGCACCAGACAAATCAGGG |
| | | | CTGCCCAGTGATCGCTTCTCTGCAGAGAGGACTGGGGGATCCGTCT |
| | | | CCACTCTGACGATCCAGCGCACACAGCAGGAGGACTCGGCCGTGTA |
| | | | TCTCTGTGCCAGCAGAGACTACTCCTACAatgagcagttcttcgggccagggac |
| | | | acggctcaccgtgctagaggacctgaaaaacgtgttcccacccgaggtcgctgtgttt |

Sequences for TCR-5; target peptide SLGDWGAEA (SEQ ID NO: 2); target gene TIMP3 A*02:01.

TABLE 11
TCR-6; target peptide SLGDWGAEA (SEQ ID NO: 2); target gene TIMP3 A*02:01

| SEQ | TCR | AA | |
|---|---|---|---|
| ID | POLY- | or | |
| NO | PEPTIDE | NT | SEQUENCE |
| 80 | α CDR1 | AA | YGATPY |
| 81 | α CDR2 | AA | YFSGDTLV |
| 82 | α CDR3 | AA | AVGPNNNDMR |
| 83 | β CDR1 | AA | SEHNR |
| 84 | β CDR2 | AA | FQNEAQ |
| 85 | β CDR3 | AA | ASSLGGNEQF |
| 86 | α VJ | AA | AQSVTQPDIHITVSEGASLELRCNYSYGATPYLFWYVQSPGQGLQLLLK |
| | | | YFSGDTLVQGIKGFEAEFKRSQSSFNLRKPSVHWSDAAEYFCAVGPNN |
| | | | NDMRFGAGTRLTVKP |
| 87 | α VJ | NT | actggagttgagatgtaactattcctatggggcaacaccttatctcttctggtatgtcc |
| | | | agtcccccggccaaggcctccagctGCTCCTGAAGTACTTTTCAGGAGACACTCTGGTT |
| | | | CAAGGCATTAAAGGCTTTGAGGCTGAATTTAAGAGGAGTCAATCTTCCTTC |
| | | | AATCTGAGGAAACCCTCTGTGCATTGGAGTGATGCTGCTGAGTACTT |
| | | | CTGTGCTGTGGGGCCGAATAACAATGacatgcgctttggagcagggaccagactg |
| | | | acagtaaaaccaaatatccagaaccctgaccctgccgtgtaccagctgagagact |
| 88 | β VDJ | AA | MGTSLLCWMALCLLGADHADTGVSQNPRHKITKRGQNVTFRCDPISEH |
| | | | NRLYWYRQTLGQGPEFLTYFQNEAQLEKSRLLSDRFSAERPKGSFSTL |
| | | | EIQRTEQGDSAMYLCASSLGGNEQFFGPGTRLTVL |
| 89 | β VDJ | NT | taactttcaggtgtgatccaatttctgaacacaaccgcctttattggtaccgacagacc |
| | | | ctggggcagggcccagagtttcTGACTTACTTCCAGAATGAAGCTCAACTAGAAAAATC |
| | | | AAGGCTGCTCAGTGATCGGTTCTCTGCAGAGAGGCCTAAGGGATCTTT |
| | | | CTCCACCTTGGAGATCCAGCGCACAGAGCAGGGGGACTCGGCCAT |
| | | | GTATCTCTGTGCCAGCAGCTTAGGGGGGAATgagcagttcttcgggccaggga |
| | | | cacggctcaccgtgctagaggacctgaaaaacgtgttcccacccgaggtcgctgtgttt |

Sequences for TCR-6; target peptide SLGDWGAEA (SEQ ID NO: 2); target gene TIMP3 A*02:01.

TABLE 12
TCR-7; target peptide SLGDWGAEA (SEQ ID NO:2); target gene TIMP3 A*02:01

| SEQ | TCR | AA | |
|---|---|---|---|
| ID | POLY- | or | |
| NO | PEPTIDE | NT | SEQUENCE |
| 90 | α CDR1 | AA | ATGYPS |
| 91 | α CDR2 | AA | ATKADDK |
| 92 | α CDR3 | AA | ALLPSNTGKLI |
| 93 | β CDR1 | AA | SEHNR |
| 94 | β CDR2 | AA | FQNEAQ |
| 95 | β CDR3 | AA | ASSSPTVYEQY |
| 96 | α VJ | AA | GNSVTQMEGPVTLSEEAFLTINCTYTATGYPSLFWYVQYPGEGLQLLLK |
| | | | ATKADDKGSNKGFEATYRKETTSFHLEKGSVQVSDSAVYFCALLPSNT |
| | | | GKLIFGQGTTLQVKP |
| 97 | α VJ | NT | cctgactataaactgcacgtacacagccacaggatacccttcccttttctggtat |
| | | | gtccaatatcctggagaaggtctacagctCCTCCTGAAAGCCACGAAGGCTGATG |
| | | | ACAAGGGAAGCAACAAAGGTTTTGAAGCCACATACCGTAAAGAAACCACTTCTTT |
| | | | CCACTTGGAGAAAGGCTCAGTTCAAGTGTCAGACTCAGCGGTGTACTTC |
| | | | TGTGCTCTGCTGCCTAGCAACACAGGCAaactaatctttgggcaagggacaacttt |
| | | | acaagtaaaaccagatatccagaaccctgaccctgccgtgtaccagctgagagact |
| 98 | β VDJ | AA | MGTSLLCWMALCLLGADHADTGVSQNPRHKITKRGQNVTFRCDPISEH |
| | | | NRLYWYRQTLGQGPEFLTYFQNEAQLEKSRLLSDRFSAERPKGSFSTL |
| | | | EIQRTEQGDSAMYLCASSSPTVYEQYFGPGTRLTVT |
| 99 | β VDJ | NT | gtaactttcaggtgtgatccaatttctgaacacaaccgcctttattggtaccgaca |
| | | | gaccctggggcagggcccagagtttctgaCTTACTTCCAGAATGAAGCTCAACTAG |
| | | | AAAAATCAAGGCTGCTCAGTGATCGGTTCTCTGCAGAGAGGCCTAAGGGATCTTT |
| | | | CTCCACCTTGGAGATCCAGCGCACAGAGCAGGGGGACTCGGCCATGTATCT |
| | | | CTGTGCCAGCAGCTCACCCACCGTctacgagcagtacttcgggccgggcaccagg |
| | | | ctcacggtcacagaggacctgaaaaacgtgttcccacccgaggtcgctgtgttt |

Sequences for TCR-7; target peptide SLGDWGAEA (SEQ ID NO: 2); target gene TIMP3 A*02:01.

TABLE 13
TCR-9; target peptide SLGDWGAEA (SEQ ID NO:2); target gene TIMP3 A*02:01

| SEQ | TCR | AA | |
|---|---|---|---|
| ID | POLY- | or | |
| NO | PEPTIDE | NT | SEQUENCE |
| 100 | α CDR1 | AA | YGATPY |
| 101 | α CDR2 | AA | YFSGDTLV |
| 102 | α CDR3 | AA | AVGPNNNDMR |
| 103 | β CDR1 | AA | SEHNR |
| 104 | β CDR2 | AA | FQNEAQ |
| 105 | β CDR3 | AA | ASSLATDTQY |
| 106 | α VJ | AA | AQSVTQPDIHITVSEGASLELRCNYSYGATPYLFWYVQSPGQGLQLLLK |
| | | | YFSGDTLVQGIKGFEAEFKRSQSSFNLRKPSVHWSDAAEYFCAVGPNN |
| | | | NDMRFGAGTRLTVKP |
| 107 | α VJ | NT | actggagttgagatgtaactattcctatggggcaacaccttatctcttctggt |
| | | | atgtccagtcccccggccaaggcctccagctGCTCCTGAAGTACTTTTCAGGA |
| | | | GACACTCTGGTTCAAGGCATTAAAGGCTTTGAGGCTGAATTTAAGAGGAGTCA |
| | | | ATCTTCCTTCAATCTGAGGAAACCCTCTGTGCATTGGAGTGATGCTGCTGAGT |
| | | | ACTTCTGTGCTGTGGGCCCCAATAACAATGacatgcgctttggagcagggacc |
| | | | agactgacagtaaaaccaaatatccagaaccctgaccctgccgtgtaccagct |
| | | | gagagact |
| 108 | β VDJ | AA | MGTSLLCWMALCLLGADHADTGVSQNPRHKITKRGQNVTFRCDPISEH |
| | | | NRLYWYRQTLGQGPEFLTYFQNEAQLEKSRLLSDRFSAERPKGSFSTL |
| | | | EIQRTEQGDSAMYLCASSLATDTQYFGPGTRLTVL |
| 109 | β VDJ | NT | taactttcaggtgtgatccaatttctgaacacaaccgcctttattggtaccga |
| | | | cagaccctggggcagggcccagagtttcTGACTTACTTCCAGAATGAAGCTCA |
| | | | ACTAGAAAAATCAAGGCTGCTCAGTGATCGGTTCTCTGCAGAGAGGCCTAAGG |
| | | | GATCTTTCTCCACCTTGGAGATCCAGCGCACAGAGCAGGGGGACTCGGCCAT |
| | | | GTATCTCTGTGCCAGCAGCTTAGCGACAGATacgcagtattttggcccaggc |
| | | | acccggctgacagtgctcgaggacctgaaaaacgtgttcccacccgaggtcg |
| | | | ctgtgttt |

Sequences for TCR-9; target peptide SLGDWGAEA (SEQ ID NO: 2); target gene TIMP3 A*02:01.

### REFERENCES

1. Robbins, P. F., et al. Mining exomic sequencing data to identify mutated antigens recognized by adoptively transferred tumor-reactive T cells. Nat Med 19, 747-752 (2013).
2. Schumacher, T. N. & Schreiber, R. D. Neoantigens in cancer immunotherapy. Science 348, 69-74 (2015).
3. Ritter, C., et. al. Epigenetic priming restores the HLA class-I antigen processing machinery expression in Merkel cell carcinoma. Sci Rep 7, 2290 (2017).
4. Setiadi, A. F., et al. Epigenetic control of the immune escape mechanisms in malignant carcinomas. Mol Cell Biol 27, 7886-7894 (2007).
5. Garrdo, F., Aptsiaurn, N., Doorduijn, E. M., Garcia Lora, A. M. & van Hall, T. The urgent need to recover MHC class I in cancers for effective immunotherapy. Curr Opin Immunol 39, 44-51(2016).
6. Van Hall, T., et al. Selective cytotoxic T-lymphocyte targeting of tumor immune escape variants. Nat Med 12, 417-424 (2006).
7. Seidel, U. J., Oliveira, C. C., Lampen, M. H. & Hall, T. A novel category of antigens enabling CTL immunity to tumor escape variants: Cinderella antigens. Cancer Immunol Immunother 61, 119-125 (2012).
8. Doorduijn, E. M., et al. TAP-independent self-peptides enhance T cell recognition of immune-escaped tumors. J Clin Invest 126, 784-794 (2016).
9. Doorduijn, E. M., etaL. T cells specific for a TAP-independent self-peptide remain naive in tumor-bearing mice and are fully exploitable for therapy. Oncoimmunology 7, el1382793 (2018).
10. Weinzierl, A. O., et al., Features of TAP-independent MHC class I ligands revealed by quantitative mass spectrometry. Eur. J. Immunol. 38, 1503-1510 (2008).
11. Marijt, K. A. et al. Identification of non-mutated neoantigens presented by TAP-deficient tumors. J Exp Med 215, 2325-2337, doi:10.1084/jem.20180577 (2018).
12. Kenter, G. G. et al. Vaccination against HPV-16 oncoproteins for vulvar intraepithelial neoplasia. N Engl J Med 361, 1838-1847, doi:10.1056/NEJMoa0810097 (2009).
13. Toes, R. E., Offringa, R., Blom, R. J., Melief, C. J. & Kast, W. M. Peptide vaccination can lead to enhanced tumor growth through specific T-cell tolerance induction. Proc Natl Acad Sci USA 93, 7855-7860 (1996).
14. Bijker, M. S. et al. CD8+ CTL priming by exact peptide epitopes in incomplete Freund's adjuvant induces a vanishing CTL response, whereas long peptides induce sustained CTL reactivity. J Immunol 179, 5033-5040 (2007).
15. Rosalia, R. A. et al. Dendritic cells process synthetic long peptides better than whole protein, improving antigen presentation and T-cell activation. Eur J Immunol 43, 2554-2565, doi:10.1002/eji.201343324 (2013).
16. van der Burg S H et al. Hum Immunol. 1995 December; 44(4):189-98. An HLA class I peptide-binding assay based on competition for binding to class I molecules on intact human B cells. Identification of conserved HIV-1 polymerase peptides binding to HLA-A*0301.
17. van der Burg S H et al. J Immunol. 1996 May 1; 156(9):3308-14, Immunogenicity of peptides bound to MHC class I molecules depends on the MHC-peptide complex stability.
18. Ossendorp, F., Mengedé, E., Camps, M., Filius, R. and Melief, C. (1998). Specific T Helper Cell Requirement for Optimal Induction of Cytotoxic T Lymphocytes against Major Histocompatibility Complex Class I I Negative Tumors. The Journal of Experimental Medicine, 187(5), pp. 693-702.
19. Beyranvand Nejad, E., van der Sluis, T., van Duikeren, S., Yagita, H., Janssen, G., van Veelen, P., Melief, C., van der Burg, S. and Arens, R. (2016). Tumor Eradication by Cisplatin Is Sustained by CD80/86-Mediated Costimulation of CD8+ T Cells. Cancer Research, 76(20), pp. 6017-6029.
20. Varypataki, E., van der Maaden, K., Bouwstra, J., Ossendorp, F. and Jiskoot, W. (2015). Cationic Liposomes Loaded with a Synthetic Long Peptide and Poly(I:C): a Defined Adjuvanted Vaccine for Induction of Antigen-Specific T Cell Cytotoxicity. The AAPS Journal, 17(1), pp. 216-226.
21. Masuko, K., Wakita, D., Togashi, Y., Kita, T., Kitamura, H. and Nishimura, T. (2015). Artificially synthesized helper/killer-hybrid epitope long peptide (H/K-HELP): Preparation and immunological analysis of vaccine efficacy. Immunology Letters, 163(1), pp. 102-112.
22. Chen, X., Li, D., Zhong, X., Chen, B., Duan, Z. and Wen, J. (2016). Induction of multiple cytotoxic T lymphocyte responses in mice by a multiepitope DNA vaccine against dengue virus serotype 1. Microbiology and Immunology, 60(12), pp. 835-845.
23. Zom G G, Willems M M J H P, Khan S, van der Sluis T C, Kleinovink J W, Camps MGM, van der Marel G A, Filippov D V, Melief C J M, Ossendorp F. Novel TLR2-binding adjuvant induces enhanced T cell responses and tumor eradication. J Immunother Cancer. 2018 Dec. 12; 6(1):146.
24. Zom G G, Welters M J, Loof N M, Goedemans R, Lougheed S, Valentijn R R, Zandvliet M L, Meeuwenoord N J, Melief C J, de Gruijl T D, Van der Marel G A, Filippov D V, Ossendorp F, Van der Burg S H. TLR2 ligand-synthetic long peptide conjugates effectively stimulate tumor-draining lymph node T cells of cervical cancer patients. Oncotarget. 2016 Oct. 11; 7(41):67087-67100.
25. Monie, T. P., Bryant, C. E., et al. 2009: Activating immunity: Lessons from the TLRs and NLRs. Trends Biochem. Sci. 34(11), 553-561.
26. Gay, N., and Gangloff, M. (2007): Structure and function of Toll receptors and their ligands. Annu. Rev. Biochem. 76, 141-165.
27. Spohn, R., Buwitt-Beckmann, U., et al. (2004): Synthetic lipopeptide adjuvants and Toll-like receptor 2-Structure-activity relationships. Vaccine 22(19), 2494-2499).
28. Bryant, C. E., Spring, D. R., et al. (2010). The molecular basis of the host response to lipopolysaccharide. Nat Rev. Microbiol. 8(1), 8-14).
29. Barbalat R, Lau L, Locksley R M, Barton G M. Toll-like receptor 2 on inflammatory monocytes induces type I interferon in response to viral but not bacterial ligands. Nat Immunol. 2009: 10(11):1200-7).
30. Akira S, Uematsu S, Takeuchi O. Pathogen recognition and innate immunity. Cell. Feb. 24, 2006: 124(4):783-801.
31. Kumar H, Kawai T, Akira S. Toll-like receptors and innate immunity. Biochem Biophys Res Commun. Oct. 30, 2009 388(4):621-5.
32. Ressing M E et al.; Differential binding of viral peptides to HLA-A2 alleles. Implications for human papillomavirus type 16 E7 peptide-based vaccination against cervical carcinoma Eur J Immunol. 1999 April; 29(4):1292-303.
33. Monjezi R et al.; Enhanced CAR T-cell engineering using non-viral Sleeping Beauty transposition from minicircle vectors. Leukemia 31(1), 186-194, 2016.
34. Jones B S et al.; Improving the safety of cell therapy products by suicide gene transfer. Front Pharmacol. (2014) 5:254.
35. Sambrook et al (1989) Molecular Cloning, A Laboratory Manual, Cold Spring Harbor Laboratory, Cold Spring Harbor, N. Y.
36. Ausubel et al (1987) Current Protocols in Molecular Biology, John Wiley and Sons, Inc., NY.
37. Cohen et al (1972) Proc. Natl. Acad. Sci. USA 69, 2110.
38. Luchansky et al (1988) Mol. Microbiol. 2, 637-646.
39. Van Hall T, van der Burg S H. Mechanisms of peptide vaccination in mouse models: tolerance, immunity, and hyperreactivity. Adv Immunol. 2012; 114:51-76.
40. Van Poelgeest M I et al. Vaccination against Oncoproteins of HPV16 for Noninvasive Vulvar/Vaginal Lesions: Lesion Clearance Is Related to the Strength of the T-Cell Response. Clin Cancer Res. 2016 May 15; 22(10):2342-50.
41. Rahimian S, Fransen M F, Kleinovink J W, Christensen J R, Amidi M, Hennink W E, Ossendorp F; Polymeric nanoparticles for co-delivery of synthetic long peptide antigen and poly IC as therapeutic cancer vaccine formulation. J Control Release 2015 Apr. 10; 203:16-22.

1. A vaccine comprising:
(a) a peptide comprising the amino acid sequence of SEQ ID NO: 1, or a nucleic acid encoding the peptide; and
(b) a pharmaceutically acceptable excipient, adjuvant, diluent and/or carrier.

2. The vaccine of claim 1, wherein the peptide comprises the amino acid sequence of any one of SEQ ID NOs: 2-27, 110 or 111;
optionally wherein the peptide comprises an amino acid sequence selected from the group consisting of: SLGDWGAEA (SEQ ID NO: 2), SLGDWGAEV (SEQ ID NO: 3), SLGDWGAEI (SEQ ID NO: 4) and SLGDVGAEL (SEQ ID NO: 5);
further optionally wherein the peptide comprises an amino acid sequence selected from the group consisting of:
SLGDWGAEA (SEQ ID NO: 2), SLGDWGAEV (SEQ ID NO: 3) and SLGDWGAEI (SEQ ID NO: 4), or
SLGDWGAEV (SEQ ID NO: 3), SLGDWGAEI (SEQ ID NO: 4), and SLGDWGAEL (SEQ ID NO: 5).

3. The vaccine of claim 1 or claim 2, wherein the peptide comprises the amino acid sequence of SLGDVGAEV (SEQ ID NO: 3).

4. The vaccine of any one of claims 1 to 3, wherein:
(a) the peptide has no more than 35 amino acids;
(b) the peptide consists of the amino acid sequence of any one of SEQ ID NOs: 2-27, 110 or 111;
optionally wherein the peptide consists of the amino acid sequence of any one of SEQ ID NOs: 2-5 or SEQ ID NOs: 22 to 27, 110 or 111;
further optionally wherein the peptide consists of the amino acid sequence of any one of SEQ ID NOs: 2-4, or any one of SEQ ID NOs: 3-5; and/or
c) the peptide consists of from 10 to 35 amino acids and comprises the amino acid sequence of any one of SEQ ID NOs: 2-27, 110 or 111;
optionally wherein the peptide comprises the amino acid sequence of any one of SEQ ID NOs: 2-5;
further optionally wherein the peptide comprises the amino acid sequence of any one of SEQ ID NOs: 2-4, or any one of SEQ ID NOs: 3-5;
further optionally wherein the peptide comprises the amino acid sequence of any one of SEQ ID Nos: 22 to 27, 110 or 111.

5. The vaccine of any preceding claim, wherein the peptide is conjugated to an immune stimulatory compound, optionally wherein the immune stimulatory compound comprises a TLR ligand.

6. A complex comprising:
a) a peptide comprising the amino acid sequence of SEQ ID NO: 1, and
b) a binding agent that specifically binds to a peptide comprising the amino acid sequence of SEQ ID NO: 1;
optionally wherein the binding agent is an HLA-A*02 molecule, further optionally wherein the binding agent is an HLA-A*02:01 molecule.

7. The complex of claim 6, wherein the complex is:
a. a SLGDVGAEA:HLA-A*02 complex, a SLGDWGAEV:HLA-A*02 complex, a SLGDVGAE:HLA-A*02 complex, a SLGDVGAEL:HLA-A*02 complex, a SLGDVGAER:HLA-A*02 complex, a SLGDWGAEN:HLA-A*02 complex, a SLGDVGAED:HLA-A*02 complex, a SLGDVGAEC:HLA-A*02 complex, a SLGDVGAEQ:HLA-A*02 complex, a SLGDVGAEE:HLA-A*02 complex, a SLGDWGAEG:HLA-A*02 complex, a SLGDWGAEH:HLA-A*02 complex, a SLGDVGAEK:HLA-A*02 complex, a SLGDVGAEM:HLA-A*02 complex, a SLGDWGAEF:HLA-A*02 complex, a SLGDVGAEP:HLA-A*02 complex, a SLGDWGAES:HLA-A*02 complex, a SLGDVGAET:HLA-A*02 complex, a SLGDVGAEW:HLA-A*02 complex or a SLGDVGAEY:HLA-A*02 complex, optionally wherein the complex is a SLGDVGAEA:HLA-A*02 complex, a SLGDVGAEV:HLA-A*02 complex, a SLGDVGAE:HLA-A*02 complex or a SLGDVGAEL:HLA-A*02 complex, further optionally wherein the complex is a SLGDVGAEA:HLA-A*02 complex, a SLGDVGAEV:HLA-A*02 complex or a SLGDVGAEI:HLA-A*02 complex; or
b. a SLGDWGAEA:HLA-A*02:01 complex, a SLGDWGAEV:HLA-A*02:01 complex, a SLGDVGAE:HLA-A*02:01 complex, a SLGDVGAEL:HLA-A*02:01 complex, a SLGDVGAER:HLA-A*02:01 complex, a SLGDVGAEN:HLA-A*02:01 complex, a SLGDWGAED:HLA-A*02:01 complex, a SLGDWGAEC:HLA-A*02:01 complex, a SLGDVGAEQ:HLA-A*02:01 complex, a SLGDVGAEE:HLA-A*02:01 complex, a SLGDVGAEG:HLA-A*02:01 complex, a SLGDVGAEH:HLA-A*02:01 complex, a SLGDVGAEK:HLA-A*02:01 complex, a SLGDVGAEM:HLA-A*02:01 complex, a SLGDVGAEF:HLA-A*02:01 complex, a SLGDVGAEP:HLA-A*02:01 complex, a SLGDVGAES:HLA-A*02:01 complex, a SLGDVGAET:HLA-A*02:01 complex, a SLGDVGAEW:HLA-A*02:01 complex or a SLGDVGAEY:HLA-A*02:01 complex, optionally wherein the complex is a SLGDVGAEA:HLA-A*02:01 complex, a SLGDWGAEV:HLA-A*02:01 complex, a SLGDWNGAEI:HLA-A*02:01 complex or a SLGDWGAEL:HLA-A*02:01 complex, further optionally wherein the complex is a SLGDWGAEA:HLA-A*02:01 complex, a SLGDVGAEV:HLA-A*02:01 complex or a SLGDWGAEI:HLA-A*02:01 complex.

8. A cell loaded with or expressing:
(i) a peptide comprising the amino acid sequence of SEQ ID NO: 1; or
(ii) the complex according to claim 6 or 7,
optionally wherein the cell is an antigen presenting cell, further optionally wherein the antigen presenting cell is selected from a macrophage, dendritic cell, a monocyte, a B-cell or a synthetic form of antigen presenting cell.

9. The cell according to claim 8, wherein the peptide comprises the amino acid sequence of any one of SEQ ID NOs: 2-21;
optionally wherein the peptide comprises an amino acid sequence selected from the group consisting of SLGDWGAEA (SEQ ID NO: 2), SLGDWGAEV (SEQ ID NO: 3), SLGDWGAEI (SEQ ID NO: 4) and SLGDVWGAEL (SEQ ID NO: 5);
further optionally wherein the peptide comprises an amino acid sequence selected from the group consisting of: SLGDWGAEA (SEQ ID NO: 2), SLGDWGAEV (SEQ ID NO: 3) and SLGDWGAEI (SEQ ID NO: 4); or the group consisting of SLGDWGAEV (SEQ ID NO: 3), SLGDWGAEI (SEQ ID NO: 4), and SLGDWGAEL (SEQ ID NO: 5).

10. The cell according to claim 8 or 9, wherein:
a) the peptide has no more than 35 amino acids;
b) the peptide consists of the amino acid sequence of any one of SEQ ID NOs: 2-21;
optionally wherein the peptide consists of the amino acid sequence of any one of SEQ ID NOs: 2-5;
further optionally wherein the peptide consists of the amino acid sequence of any one of SEQ ID NOs: 2-4 or any one of SEQ ID NOs: 3-5; and/or
c) the peptide consists of from 10 to 35 amino acids and comprises the amino acid sequence of any one of SEQ ID NOs: 2-21;
optionally wherein the peptide comprises of the amino acid sequence of any one of SEQ ID NOs: 2-5;
further optionally wherein the peptide comprises of the amino acid sequence of any one of SEQ ID NOs: 2-4 or any one of SEQ ID NOs: 3-5.

11. An isolated nucleic acid composition that encodes a TIMP3 antigen-specific binding protein having a TCR α chain variable (Na) domain and a TCR β chain variable (Vβ) domain, the composition comprising: a nucleic acid sequence that encodes a TCR Vα domain comprising a CDR3 amino acid sequence; and a nucleic acid sequence that encodes a TCR Vβ domain comprising a CDR3 amino acid sequence; wherein the CDR3 sequences together specifically bind to a peptide comprising the amino acid sequence of SEQ ID NO: 1.

12. The isolated nucleic acid composition of claim 11, wherein the composition comprises:
(i) a nucleic acid sequence that encodes a TCR Vα domain comprising a CDR3 amino acid sequence having at least 80% sequence identity to SEQ ID NO:42, or a functional fragment thereof; and a nucleic acid sequence that encodes a TCR Vβ domain comprising a CDR3 amino acid sequence having at least 80% sequence identity to SEQ ID NO:45, or a functional fragment thereof, wherein the CDR3 sequences together specifically bind to a peptide comprising the amino acid sequence of SEQ ID NO:1; or
(ii) a nucleic acid sequence that encodes a TCR Vα domain comprising a CDR3 amino acid sequence having at least 80% sequence identity to SEQ ID NO:52, or a functional fragment thereof; and a nucleic acid sequence that encodes a TCR Vβ domain comprising a CDR3 amino acid sequence having at least 80% sequence identity to SEQ ID NO: 55, or a functional fragment thereof, wherein the CDR3 sequences together specifically bind to a peptide comprising the amino acid sequence of SEQ ID NO:1; or
(iii) a nucleic acid sequence that encodes a TCR Vα domain comprising a CDR3 amino acid sequence having at least 80% sequence identity to SEQ ID NO:62, or a functional fragment thereof; and a nucleic acid sequence that encodes a TCR Vβ domain comprising a CDR3 amino acid sequence having at least 80% sequence identity to SEQ ID NO:65, or a functional fragment thereof, wherein the CDR3 sequences together specifically bind to a peptide comprising the amino acid sequence of SEQ ID NO:1; or
(iv) a nucleic acid sequence that encodes a TCR Vα domain comprising a CDR3 amino acid sequence having at least 80% sequence identity to SEQ ID NO:72, or a functional fragment thereof; and a nucleic acid sequence that encodes a TCR Vβ domain comprising a CDR3 amino acid sequence having at least 80% sequence identity to SEQ ID NO: 75, or a functional fragment thereof, wherein the CDR3 sequences together specifically bind to a peptide comprising the amino acid sequence of SEQ ID NO:1; or
(v) a nucleic acid sequence that encodes a TCR Vα domain comprising a CDR3 amino acid sequence having at least 80% sequence identity to SEQ ID NO:82, or a functional fragment thereof; and a nucleic acid sequence that encodes a TCR Vβ domain comprising a CDR3 amino acid sequence having at least 80% sequence identity to SEQ ID NO: 85, or a functional fragment thereof, wherein the CDR3 sequences together specifically bind to a peptide comprising the amino acid sequence of SEQ ID NO:1; or
(vi) a nucleic acid sequence that encodes a TCR Vα domain comprising a CDR3 amino acid sequence having at least 80% sequence identity to SEQ ID NO:92, or a functional fragment thereof; and a nucleic acid sequence that encodes a TCR Vβ domain comprising a CDR3 amino acid sequence having at least 80% sequence identity to SEQ ID NO: 95, or a functional fragment thereof, wherein the CDR3 sequences together specifically bind to a peptide comprising the amino acid sequence of SEQ ID NO:1; or
(vii) a nucleic acid sequence that encodes a TCR Vα domain comprising a CDR3 amino acid sequence having at least 80% sequence identity to SEQ ID NO:102, or a functional fragment thereof; and a nucleic acid sequence that encodes a TCR Vβ domain comprising a CDR3 amino acid sequence having at least 80% sequence identity to SEQ ID NO: 105, or a functional fragment thereof, wherein the CDR3 sequences together specifically bind to a peptide comprising the amino acid sequence of SEQ ID NO:1.

13. The isolated nucleic acid composition of claim 12, wherein:
(i) the CDR3 of the Vα domain comprises or consists of the amino acid sequence of SEQ ID NO: 42, and the CDR3 of the Vβ domain comprises or consists of the amino acid sequence of SEQ ID NO:45; or
(ii) the CDR3 of the Vα domain comprises or consists of the amino acid sequence of SEQ ID NO: 52, and the CDR3 of the Vβ domain comprises or consists of the amino acid sequence of SEQ ID NO:55; or
(iii) the CDR3 of the Vα domain comprises or consists of the amino acid sequence of SEQ ID NO: 62, and the CDR3 of the Vβ domain comprises or consists of the amino acid sequence of SEQ ID NO:65; or
(iv) the CDR3 of the Vα domain comprises or consists of the amino acid sequence of SEQ ID NO: 72, and the CDR3 of the Vβ domain comprises or consists of the amino acid sequence of SEQ ID NO:75; or
(v) the CDR3 of the Vα domain comprises or consists of the amino acid sequence of SEQ ID NO: 82, and the CDR3 of the Vβ domain comprises or consists of the amino acid sequence of SEQ ID NO:85; or
(vi) the CDR3 of the Vα domain comprises or consists of the amino acid sequence of SEQ ID NO: 92, and the CDR3 of the Vβ domain comprises or consists of the amino acid sequence of SEQ ID NO:95; or
(vii) the CDR3 of the Vα domain comprises or consists of the amino acid sequence of SEQ ID NO: 102, and the CDR3 of the Vβ domain comprises or consists of the amino acid sequence of SEQ ID NO:105.

14. The isolated nucleic acid composition of any one of claims 11 to 13, wherein the peptide comprises an amino acid sequence selected from the group consisting of: SEQ ID NOs: 2-21, optionally wherein the peptide comprises an amino acid sequence selected from the group consisting of SEQ ID NOs: 2-5, further optionally selected from the group consisting of: SEQ ID NOs: 2-4 or from the group consisting of: SEQ ID NO: 3-5.

15. The isolated nucleic acid composition of any one of claims 11 to 14, wherein the encoded binding protein is capable of specifically binding to a peptide:HLA complex selected from the group consisting of
a. a SX₁GDVWGAEX₂:HLA-A*02 complex (wherein X₁ and X₂ are any amino acid), a SLGDVGAEA:HLA-A*02 complex, a SLGDWGAEV:HLA-A*02 complex, a SLGDVGAEI:HLA-A*02 complex, a SLGDVGAEL:HLA-A*02 complex, a SLGDWGAER:HLA-A*02 complex, a SLGDVGAEN:HLA-A*02 complex, a SLGDVGAED:HLA-A*02 complex, a SLGDVGAEC:HLA-A*02 complex, a SLGDVGAEQ:HLA-A*02 complex, a SLGDVGAEE:HLA-A*02 complex, a SLGDVGAEG:HLA-A*02 complex, a SLGDVGAEH:HLA-A*02 complex, a SLGDVGAEK:HLA-A*02 complex, a SLGDWGAEM:HLA-A*02 complex, a SLGDVGAEF:HLA-A*02 complex, a SLGDVGAEP:HLA-A*02 complex, a SLGDVGAES:HLA-A*02 complex, a SLGDVGAET:HLA-A*02 complex, a SLGDWGAEW-HLA-A*02 complex, and a SLGDVGAEY:HLA-A*02 complex, optionally wherein the peptide:HLA complex is selected from the group consisting of: a SX₁GDVWGAEX₂:HLA-A*02 complex (wherein X₁ and X₂ are any amino acid), a SLGDWGAEA:HLA-A*02 complex, a SLGDWGAEV:HLA-A*02 complex, a SLGDVWGAEI:HLA-A*02 complex and a SLGDWGAEL:HLA-A*02 complex, further optionally wherein the peptide:HLA complex is selected from the group consisting of a SX₁GDWGAEX₂:HLA-A*02 complex (wherein X₁ and X₂ are any amino acid), a SLGDWNGAEA:HLA-A*02 complex, a SLGDWGAEV:HLA-A*02 complex and a SLGDWGAEI:HLA-A*02 complex; or
b. a SX₁GDWNGAEX₂:HLA-A*02:01 complex (wherein X₁ and X₂ are any amino acid), a SLGDWGAEA:HLA-A*02:01 complex, a SLGDWGAEV:HLA-A*02:01 complex, a SLGDWGAEI:HLA-A*02:01 complex, a SLGDWGAEL:HLA-A*02:01 complex, a SLGDWGAER:HLA-A*02:01 complex, a SLGDWGAEN:HLA-A*02:01 complex, a SLGDWNGAED:HLA-A*02:01 complex, a SLGDWNGAEC:HLA-A*02:01 complex, a SLGDWGAEQ:HLA-A*02:01 complex, a SLGDWGAEE:HLA-A*02:01 complex, a SLGDWGAEG:HLA-A*02:01 complex, a SLGDWGAEH:HLA-A*02:01 complex, a SLGDWGAEK:HLA-A*02:01 complex, a SLGDWGAEM:HLA-A*02:01 complex, a SLGDWGAEF:HLA-A*02:01 complex, a SLGDWGAEP:HLA-A*02:01 complex, a SLGDWGAES:HLA-A*02:01 complex, a SLGDWGAET:HLA-A*02:01 complex, a SLGDWNGAEW:HLA-A*02:01 complex, and a SLGDVGAEY:HLA-A*02:01 complex, optionally wherein the peptide:HLA complex is selected from the group consisting of: a SX₁GDVWGAEX₂:HLA-A*02:01 complex (wherein X₁ and X₂ are any amino acid), a SLGDVGAEA:HLA-A*02:01 complex, a SLGDWGAEV:HLA-A*02:01 complex, a SLGDVGAEI:HLA-A*02:01 complex and a SLGDWGAEL:HLA-A*02:01 complex, further optionally wherein the peptide:HLA complex is selected from the group consisting of: a SX₁GDVWGAEX₂:HLA-A*02:01 complex (wherein X₁ and X₂ are any amino acid), a SLGDVGAEA:HLA-A*02:01 complex, a SLGDVGAEV:HLA-A*02:01 complex and a SLGDVGAEI:HLA-A*02:01 complex.

16. The isolated nucleic acid composition of any one of claims 11 to 15, wherein the CDR3 sequences together specifically bind to the peptide when it is complexed with HLA-A*02:01.

17. The isolated nucleic acid composition of any one of claims 11 to 16, wherein the encoded binding protein comprises a TCR, an antigen binding fragment of a TCR, a chimeric antigen receptor (CAR), or an ImmTAC.

18. The isolated nucleic acid composition of claim 17, wherein the antigen binding fragment of a TCR is a single chain TCR (scTCR) or a chimeric TCR dimer in which the antigen binding fragment of the TCR is linked to an alternative transmembrane and intracellular signalling domain.

19. An isolated nucleic acid encoding a chimeric antigen receptor protein comprising a target binding moiety that specifically binds to a peptide comprising the amino acid sequence of SEQ ID NO: 1.

20. The isolated nucleic acid of claim 19, wherein the peptide comprises the amino acid sequence of any one of SEQ ID NOs: 2-21, optionally wherein the peptide comprises the amino acid sequence of any one of SEQ ID NOs: 2-5, further optionally wherein the peptide comprises the amino acid sequence of any one of SEQ ID NOs: 2-4 or any one of SEQ ID NOs: 3-5.

21. A modified cell transformed, transfected or transduced with a nucleic acid comprising a nucleic acid sequence encoding the peptide of any one of claims 8 to 10, a nucleic acid composition of any one of claims 11 to 18, or a nucleic acid of claim 19 or 20.

22. The modified cell of claim 21, wherein the modified cell is selected from the group consisting of a CD8 T cell, a CD4 T cell, an NK cell, an NKT cell, a gamma-delta T cell, an innate lymphoid cell (ILC), a hematopoietic stem cell, a progenitor cell, a T cell line or a NK-92 cell line, optionally wherein the modified cell is a human cell.

23. A pharmaceutical composition comprising a nucleic acid, a nucleic acid composition, a complex, or a cell according to any preceding claim, and a pharmaceutically acceptable excipient, adjuvant, diluent and/or carrier.

24. The pharmaceutical composition of claim 23, wherein the composition is formulated as a vaccine.

25. The vaccine according to any one of claims 1 to 5, and/or the pharmaceutical composition according to claim 23 or 24, for use as a medicament.

26. The vaccine and/or pharmaceutical composition for use according to claim 25, in the prevention or treatment of a pre-cancer, a cancer or a viral infection associated with impaired HLA class I antigen presentation in a human subject.

27. The vaccine and/or pharmaceutical composition for use according to claim 26, wherein the pre-cancer or cancer is a pre-cancer or cancer with impaired peptide processing machinery.

28. The vaccine and/or pharmaceutical composition for use according to claim 26 or 27, wherein the pre-cancer, cancer, or viral infection is associated with reduced or low TAP1 or TAP2 protein expression, activity, level, or stability.

29. The vaccine according to any one of claims 1 to 5, and/or the pharmaceutical composition according to claim 23 or 24, for use in treating or preventing a pre-cancer, a cancer or viral infection associated with impaired HLA class I antigen presentation in a human subject, wherein the subject has been identified as having a pre-cancer, a cancer or viral infection associated with impaired HLA class I antigen presentation by the presence of:
a. a SX₁GDVWGAEX₂:HLA-A*02 complex (wherein X₁ and X₂ are any amino acid), a SLGDVGAEA:HLA-A*02 complex, a SLGDWGAEV:HLA-A*02 complex, a SLGDVGAEI:HLA-A*02 complex, a SLGDVGAEL:HLA-A*02 complex, a SLGDWGAER:HLA-A*02 complex, a SLGDVGAEN:HLA-A*02 complex, a SLGDVGAED:HLA-A*02 complex, a SLGDVGAEC:HLA-A*02 complex, a SLGDVGAEQ:HLA-A*02 complex, a SLGDVGAEE:HLA-A*02 complex, a SLGDVGAEG:HLA-A*02 complex, a SLGDVGAEH:HLA-A*02 complex, a SLGDVGAEK:HLA-A*02 complex, a SLGDWGAEM:HLA-A*02 complex, a SLGDVGAEF:HLA-A*02 complex, a SLGDVGAEP:HLA-A*02 complex, a SLGDVGAES:HLA-A*02 complex, a SLGDVGAET:HLA-A*02 complex, a SLGDWGAEW-HLA-A*02 complex or a SLGDVGAEY:HLA-A*02 complex in a sample isolated from the subject, optionally a SLGDWGAEA:HLA-A*02 complex, a SLGDVGAEV:HLA-A*02 complex, a SLGDVGAEI:HLA-A*02 complex or a SLGDVGAEL:HLA-A*02 complex in a sample isolated from the subject, further optionally a SLGDWNGAEA:HLA-A*02 complex, a SLGDWGAEV:HLA-A*02 complex or a SLGDWGAEI:HLA-A*02 complex in a sample isolated from the subject; or
b. a SX₁GDWNGAEX₂:HLA-A*02:01 complex (wherein X₁ and X₂ are any amino acid), a SLGDVGAEA:HLA-A*02:01 complex, a SLGDWGAEV:HLA-A*02:01 complex, a SLGDVGAE:HLA-A*02:01 complex, a SLGDWGAEL:HLA-A*02:01 complex, a SLGDVGAER:HLA-A*02:01 complex, a SLGDWGAEN:HLA-A*02:01 complex, a SLGDVGAED:HLA-A*02:01 complex, a SLGDWGAEC:HLA-A*02:01 complex, a SLGDVGAEQ:HLA-A*02:01 complex, a SLGDWGAEE:HLA-A*02:01 complex, a SLGDVGAEG:HLA-A*02:01 complex, a SLGDWGAEH:HLA-A*02:01 complex, a SLGDVGAEK:HLA-A*02:01 complex, a SLGDWGAEM:HLA-A*02:01 complex, a SLGDVGAEF:HLA-A*02:01 complex, a SLGDWGAEP:HLA-A*02:01 complex, a SLGDVGAES:HLA-A*02:01 complex, a SLGDWGAET:HLA-A*02:01 complex, a SLGDVGAEW:HLA-A*02:01 complex or a SLGDWGAEY:HLA-A*02:01 complex in a sample isolated from the subject, optionally a SLGDWNGAEA:HLA-A*02:01 complex, a SLGDVGAEV:HLA-A*02:01 complex, a SLGDWGAEI:HLA-A*02:01 complex or a SLGDWGAEL:HLA-A*02:01 complex in a sample isolated from the subject, further optionally a SLGDVGAEA:HLA-A*02:01 complex, a SLGDWGAEV:HLA-A*02:01 complex or a SLGDVGAE:HLA-A*02:01 complex in a sample isolated from the subject.

30. A method of treating a condition in a human subject in need thereof, the method comprising administering to the subject a therapeutically effective amount of the vaccine according to any one of claims 1 to 5, and/or the pharmaceutical composition according to claim 23 or 24.

31. The method of claim 30, for the prevention or treatment of a pre-cancer, a cancer or a viral infection associated with impaired HLA class I antigen presentation.

32. A method of generating a T cell receptor, comprising contacting the nucleic acid composition of any one of claims 11 to 18 with a cell under conditions in which the nucleic acid composition is incorporated and expressed by the cell to generate the T cell receptor that specifically binds to a peptide comprising the amino acid sequence of SEQ ID NO: 1.

33. The method of claim 32, wherein the peptide comprises the amino acid sequence of any one of SEQ ID NOs: 2-21, optionally wherein the peptide comprises the amino acid sequence of any one of SEQ ID NOs: 2-5, further optionally wherein the peptide comprises the amino acid sequence of any one of SEQ ID NOs: 2-4 or any one of SEQ ID NOs: 3-5.

34. The method of claim 32 or 33, wherein the method is ex vivo.

35. Use of a peptide comprising an amino acid sequence of SEQ ID NO: 1, or a complex according to claim 6 or 7, for identifying a therapeutic binding protein.

36. The use according to claim 35, wherein the therapeutic binding protein is capable of preventing or treating a pre-cancer, a cancer or a viral infection associated with impaired HLA class I antigen presentation.

37. The use according to claim 35 or 36, wherein the therapeutic binding protein comprises:
a TCR, an antigen binding fragment of a TCR, or a chimeric antigen receptor (CAR), or an ImmTAC; or the therapeutic binding protein is an antibody.

38. The use according to any one of claims 35 to 37, wherein:
(a) the peptide comprises an amino acid sequence selected from the group consisting of SEQ ID NO: 2-5; or
(b) the complex is selected from the group consisting of: a SLGDWGAEA:HLA-A*02 complex, a SLGDVNGAEV:HLA-A*02 complex, a SLGDVNGAEI:HLA-A*02 complex; or a SLGDVNGAEL:HLA-A*02 complex.
